(19) United States
(12) Patent Application Publication
(10) Pub. No.: US 2026/0317309 A1
(43) Pub. Date: Oct. 15, 2026

(54) N-HETEROCYLIC GPCR RECEPTOR AGONISTS, PHARMACEUTICAL COMPOSITIONS COMPRISING THE SAME, AND METHODS FOR THEIR USE

(71) Applicant: Carmot Therapeutics, Inc., Berkeley, CA (US)

(72) Inventors: Xiaohui Du, Berkeley, CA (US); Ray Fucini, Berkeley, CA (US); Sumanta Garai, Berkeley, CA (US); Xiang Zhou, Berkeley, CA (US); Chien-Hung Yeh, Berkeley, CA (US); Joseph Vacca, Berkeley, CA (US); David Lloyd, Berkeley, CA (US); Stig Hansen, Berkeley, CA (US)

(21) Appl. No.: 18/998,544

(22) PCT Filed: Jul. 26, 2023
(86) PCT No.: PCT/US2023/071000

§ 371 Date: Jan. 27, 2025

Related U.S. Application Data
(60) Provisional application No. 63/392,857, filed on Jul. 27, 2022.

(51) Int. Cl. C07D 405/14; A61K 31/4545; A61K 31/506; C07D 401/14; C07D 409/14; C07D 471/04

(52) U.S. Cl. CPC C07D 405/14; A61K 31/4545; A61K 31/506; C07D 401/14; C07D 409/14; C07D 471/04

(57) ABSTRACT

Provided herein are GLP-1 receptor modulator compounds, pharmaceutical compositions, methods of their preparation, and methods of their use in treatment, and/or diagnosis.

### FIELD

Provided herein are GLP-1 receptor modulator compounds; pharmaceutical compositions comprising the compounds; methods of producing the compounds; and methods of using the compounds, and compositions for therapy. The compounds, and compositions are useful, for instance, in methods of treatment, and prevention of metabolic diseases or conditions, methods of detection of metabolic diseases or conditions, and methods of diagnosis of metabolic diseases or conditions.

### BACKGROUND

Diabetes is a serious chronic disease that occurs when the pancreas does not produce enough insulin, or when the body cannot effectively use the insulin it produces. Complications of diabetes include, damage to the heart, blood vessels, eyes, kidneys, and nerves. Diabetes can increase risk of heart disease, and stroke. The results include serious effects on quality of life, health, and mortality. WHO Global Report on Diabetes, 2016, World Health Organization. As of 2017, approximately 462 million individuals worldwide, about 6.28% of the population was affected by type 2 diabetes, and this prevalence was increasing measurably. Khan et al., 2020, J. Epidemiol. Glob. Health 10 (1): 107-111. The global economic burden of diabetes in 2015 was estimated to be $1.3T, and estimated to increase to $2.1T by 2030. Bommer et al., 2018, Diabetes Care 41 (5): 963-970. Approximately 90-95% of all diabetes cases are type 2 diabetes. Tripathi & Srivastava, 2016, Med. Sci. Monit. 12 (7): RA130-147.

The glucagon-like peptide-1 receptor (GLP-1 receptor, or GLP1R) has emerged as a potential target for treating type 2 diabetes. Its ligand, glucagon-like peptide-1 (GLP-1) enhances glucose-induced insulin secretion, and increases insulin synthesis among many other effects. Doyle and Egan, 2007, Pharmacol. Ther. 113 (3): 546-593. GLP-1 is known to delay gastric emptying, suppress food intake, increase satiety, and reduce weight in humans. Shah and Vella, 2014 Rev Endocr Metab Disord. 15 (3): 181-187. Activating the GLP-1 receptor has been shown to have beneficial effects on insulin secretion and the maintenance of beta cell glucose sensing, transcription, synthesis, proliferation, and survival. Doyle and Egan, 2007, supra. While the GLP-1 receptor is a promising therapeutic target, only a handful of GLP-1 receptor drugs have been approved to date, and most, or all of these are peptide, or polypeptide drugs.

There is a need for additional therapies for treating metabolic diseases, and conditions, like type 2 diabetes. Small molecules targeting GLP-1 receptor should provide safe, stable, and easy to administer therapeutics for metabolic diseases, and conditions such as type 2 diabetes.

### SUMMARY

Provided herein are GLP-1 receptor modulator compounds of Formulas (LVII)-(LXIV), and sub-formulas thereof, compositions comprising the compounds, methods of producing the compounds, and methods of using the compounds, and compositions in treatment, and diagnosis. The compounds of Formula (LVII)-(LXIV), and embodiments thereof, are useful for modulating the activity of GLP-1 receptor. In certain embodiments, the compounds can be used to agonize the activity of GLP-1 receptor. In certain embodiments, the compounds can be used for treating diseases, or conditions modulated by GLP-1 receptor.

In one aspect, provided are compounds of Formula (LVII), (LVIII), (LIX), (LX), (LXI), or (LXII), and compounds 552, 553, 561, 562, 572, 573, 576, 578, 581, 640, and 641; or a pharmaceutically acceptable salt, or stereoisomer thereof:

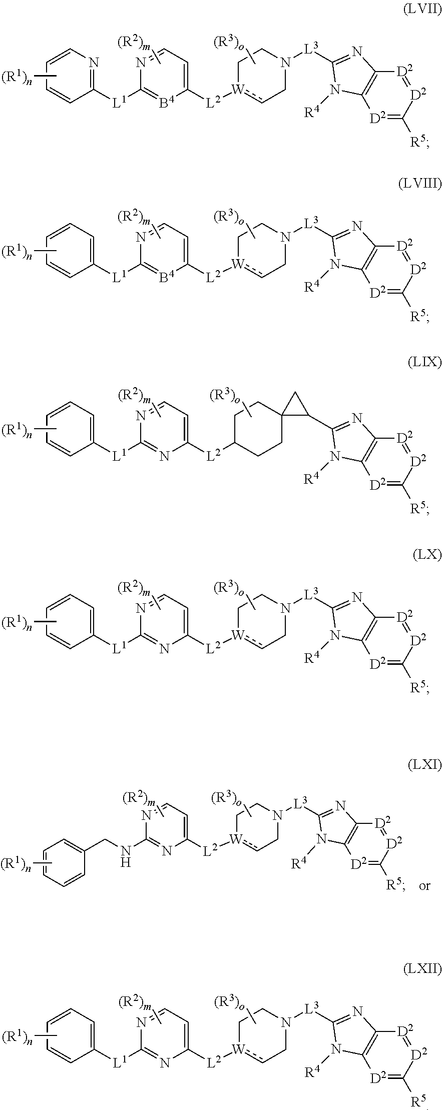

In certain embodiments, when the compound is according to Formula (LVII):
B⁴ is —CH—, —C(R²)—, or —N—;
D¹ is —CH— or —N—;
D² is —CH—, —N—, —C(CH₃)—;
D⁴ is —CH— or —N—;
W is N, CR¹⁴, or C; when W is CR¹⁴, the adjacent dashed line indicates a single bond; when W is C, the adjacent dashed line indicates a double bond or L² is —C(H)═;
L¹ is selected from the group consisting of —O—, —CH₂—, —NH—, —N(Me)-, —N(Et)-, —OCH₂—, —OC(H,Me)-, —CH₂O—, —NHCH₂—, —CH₂NH—, —N(Me)CH₂—, and —SO₂NH—;
L² is selected from the group consisting of a bond, —C(O)—, —CH₂—, —C(H)═, SO₂—, —O—, —NH—, —N(Me)-, and

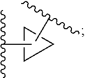

L³ is —CH₂—, —C(O)—, or —CH(CH₃)—;
each R¹ is independently selected from the group consisting of alkyl, substituted alkyl, halo, cyano, azido, alkoxy, haloalkoxy, and R¹¹R¹²NCO—;
each R² is independently selected from the group consisting of alkyl, substituted alkyl, halo, cyano, azido, alkoxy, haloalkoxy, and R¹¹R¹²NCO—;
each R³ is independently selected from the group consisting of alkyl, substituted alkyl, halo, cyano, azido, alkoxy, haloalkoxy, and R¹¹R¹²NCO—;
R⁴ is selected from the group consisting of alkyl, substituted alkyl, unsubstituted arylalkyl, substituted arylalkyl, heteroalkyl, substituted heteroalkyl, unsubstituted heteroarylalkyl, substituted heteroarylalkyl, unsubstituted heterocycloalkyl, and substituted heterocycloalkyl;
R⁵ is selected from the group consisting of H, —COOH, —CH₂COOH, —(CH₂)₂COOH, —COCF₃, —C(OH)CF₃, —CONHCN, —CONHOH, CONHOMe, —CONHSO₂N(Me)₂, —PO₃H₂, —PO(Me)(OH), —SO₃H, —SO₂NH₂, —SO₂NHMe, —B(OH)₂,

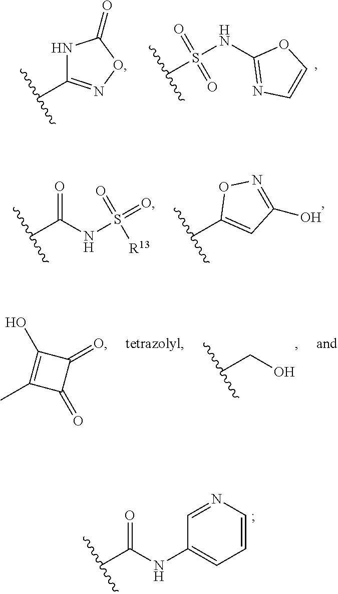

each R¹¹ is hydrogen, or alkyl;
each R¹² is hydrogen, or alkyl;
R¹³ is hydrogen, alkyl, or -cyclopropyl;
R¹⁴ is hydrogen or alkyl;
n is an integer from 0 to 4;
m is an integer from 0 to 3; and
o is an integer from 0 to 4.

In certain embodiments, when the compound is according to Formula (LVIII), R³ is independently selected from the group consisting of —CH₂CH₃, —CH₂F, —CH₂CN, and —CF₃; and all the other variables are as in Formula (LVII); wherein when B⁴ is —C— and R³ is —CH₂F, then the compound is not

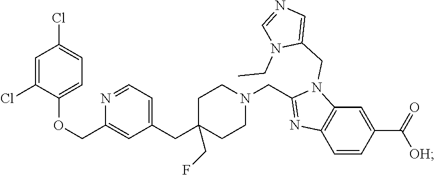 [SMILES: CCn1cncc1Cn1c(CN2CCC(CF)(Cc3ccnc(COc4ccc(Cl)cc4Cl)c3)CC2)nc2ccc(C(=O)O)cc21]

wherein when B⁴ is —N— and R³ is —CH₂F, then the compound is not

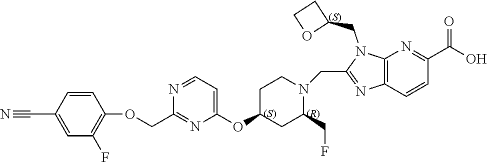

In certain embodiments, when the compound is according to Formula (LIX), the variables are as in Formula (LVII).

In certain embodiments, when the compound is according to Formula (LX), R⁴ is selected from the group consisting of: (1,1-dioxidothietan-2-yl)methyl, ((R)-1,1-dioxidothietan-2-yl)methyl, ((S)-1,1-dioxidothietan-2-yl)methyl,

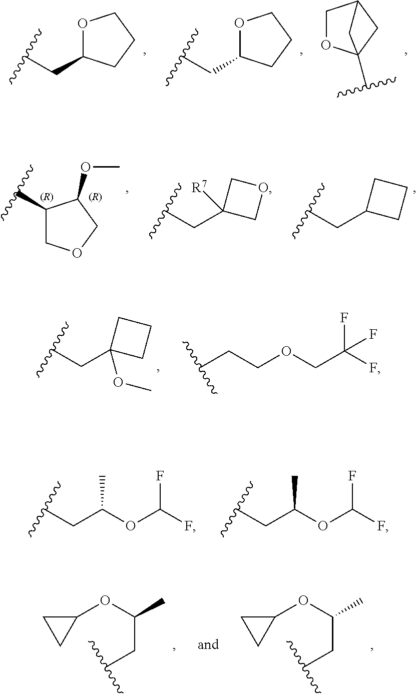

wherein R⁷ is —CH₂F or —CHF₂; and all the other variables are as in Formula (LVII).

In certain embodiments, the compound is according to Formula (LXI), and all the other variables are as in Formula (LVII).

In certain embodiments, when the compound is according to Formula (LXII), either (a), (b), (c), (d), or (e):
(a) wherein when D¹ is —N— and D² and D⁴ are —CH—, then the compound is not one of the following compounds.

| Com- | |
|---|---|
| pound | |
| No. | Structure |
| 486 | 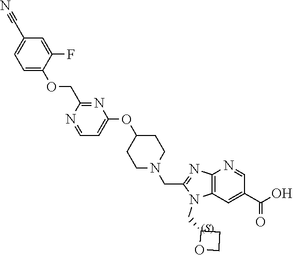 |
| 512 | 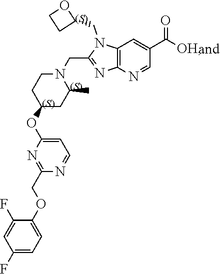 |
| 513 | 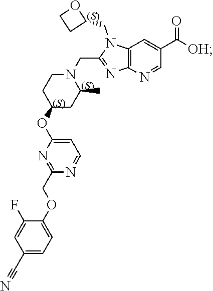 |

and all the other variables are as in Formula (LVII);
or
   (b) wherein D¹ and D⁴ are —CH— and D² is —N— and all the other variables are as in Formula (I);
or
   (c) wherein D¹ is —CH—, D⁴ is —CH— or —N—, and D² is —C(CH₃); and all the other variables are as in Formula (LVII);
or
   (d) wherein D¹, D², and D⁴ are —CH— or —N—, wherein zero or one of D¹, D², and D⁴ is —N— and R⁵ is selected from group consisting of: —H, —CH₂OH, —CH₂COOH, —(CH₂)₂COOH,

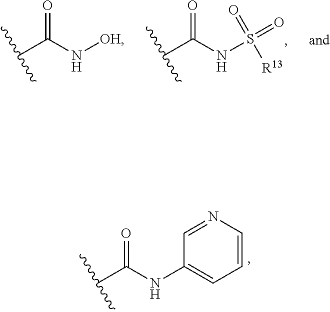

and R¹³ is —CH₃ or -cyclopropyl; and all the other variables are as in Formula (LVII);
or
   (e) D¹, D², and D⁴ are —CH— or —N—, wherein zero or one of D¹, D², and D⁴ is —N and L³ is —CH(CH₃)—; and all the other variables are as in Formula (LVII).

In certain embodiments, provided are compounds 552, 553, 558, 561, 562, 572, 573, 576, 578, 581, 590, and 591:

| Com- | |
|---|---|
| pound | |
| No. | Structure |
| 552 | 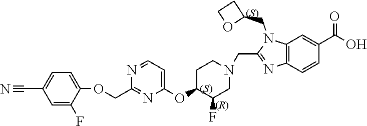 |
| 553 | 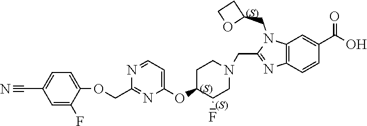 |
| 558 | 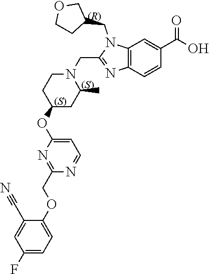 |
| 561 | 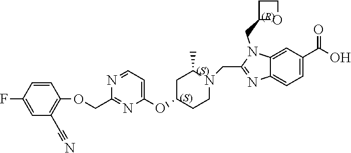 |
| 562 | 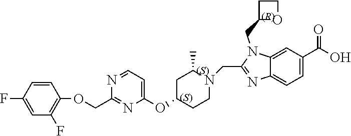 |
| 572 | 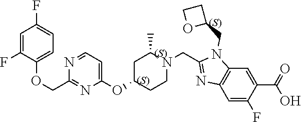 |
| 573 | 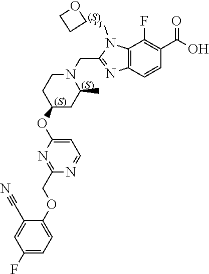 |
| 576 | 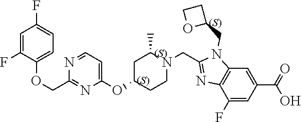 |
| 578 | 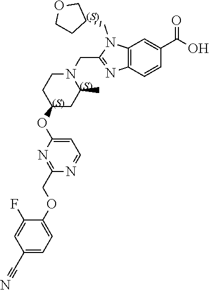 |
| 581 | 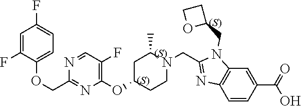 |
| 640 | 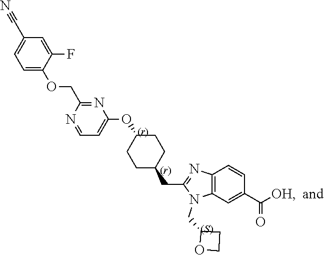 |
| 641 | 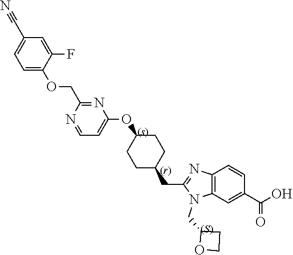 |

In certain embodiments, when the compound is according to Formula (LVII), (LVIII), (LIX), (LX), or (LXII), L¹ is selected from the group consisting of —NH—, —N(Me)-, —N(Et)-, —OCH₂—, —OC(H,Me)-, —CH₂O—, —OCH₂(cyclopropyl)-, —O—CH₂—CH₂—, —CH₂—O—CH₂—, —CH₂—O—C((CH₃)₂), —NHCH₂—, —N(Me)CH₂—, and —SO₂NH—.

In one aspect, provided is a compound according to Formula LXIII or a pharmaceutically acceptable salt, or stereoisomer thereof:

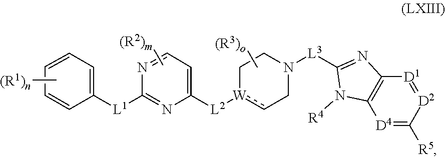

wherein:
D¹ is —CH— or —N—;
D² is —CH—, —N—, —C(CH₃)—;
D⁴ is —CH— or —N—;
W is N, CR¹⁴, or C; when W is CR¹⁴, the adjacent dashed line indicates a single bond; when W is C, the adjacent dashed line indicates a double bond or L² is —C(H)═;
L¹ is selected from the group consisting of —O—, —CH₂—, —NH—, —N(Me)-, —N(Et)-, —OCH₂—, —OC(H,Me)-, —CH₂O—, —NHCH₂—, —CH₂NH—, —N(Me)CH₂—, and —SO₂NH—;
L² is selected from the group consisting of a bond, —C(O)—, —CH₂—, —C(H)═, SO₂—, —O—, —NH—, —N(Me)-, and

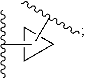

L³ is —CH₂—, —C(O)—, or —CH(CH₃)—;
each R¹ is independently selected from the group consisting of alkyl, substituted alkyl, halo, cyano, azido, alkoxy, haloalkoxy, and R¹¹R¹²NCO—;
each R² is independently selected from the group consisting of alkyl, substituted alkyl, halo, cyano, azido, alkoxy, haloalkoxy, and R¹¹R¹²NCO—;
each R³ is independently selected from the group consisting of alkyl, substituted alkyl, halo, cyano, azido, alkoxy, haloalkoxy, and R¹¹R¹²NCO—;
R⁴ is selected from the group consisting of: (1,1-dioxidothietan-2-yl)methyl, ((R)-1,1-dioxidothietan-2-yl)methyl, ((S)-1,1-dioxidothietan-2-yl)methyl,

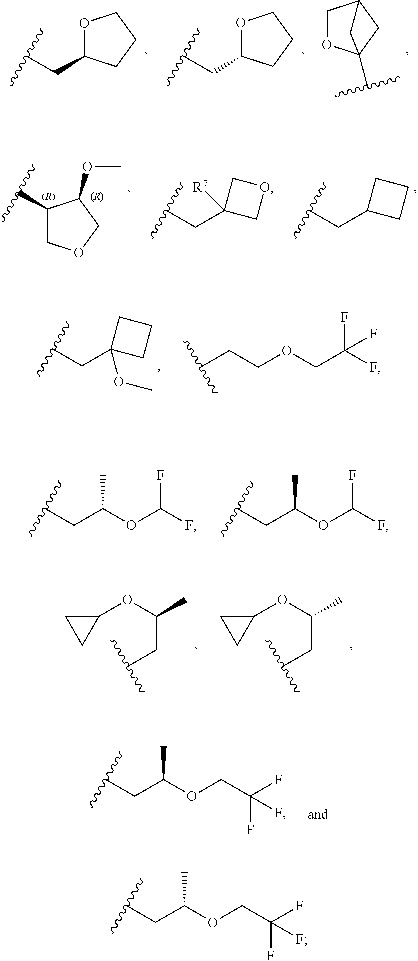

R⁵ is selected from the group consisting of —H, —CH₂OH, —COOH, —CH₂COOH, —(CH₂)₂COOH, —COCF₃, —C(OH)CF₃, —CONHCN, —CONHOH, CONHOMe, —CONHSO₂N(Me)₂, —PO₃H₂, —PO(Me)(OH), —SO₃H, —SO₂NH₂, —SO₂NHMe, —B(OH)₂,

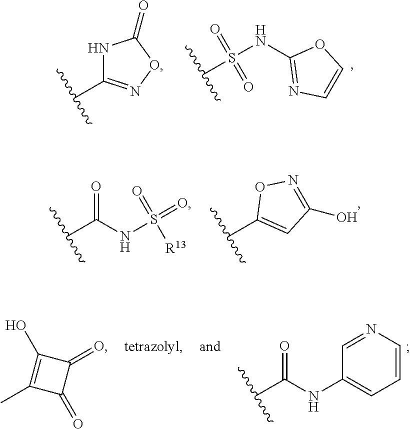

R⁷ is —CH₂F or —CHF₂;
each R¹¹ is hydrogen, or alkyl;
each R¹² is hydrogen, or alkyl;
R¹³ is hydrogen, alkyl, or -cyclopropyl;
R¹⁴ is hydrogen or alkyl;
n is an integer from 0 to 4;
m is an integer from 0 to 3; and
o is an integer from 0 to 4.

In one aspect, provided is a compound according to Formula LXIV or a pharmaceutically acceptable salt, or stereoisomer thereof:

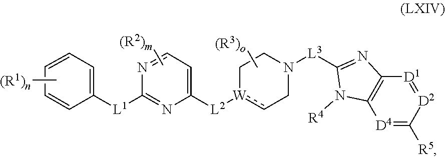

wherein:
D¹ is —CH— or —N—;
D² is —CH—, —N—, —C(CH₃)—;
D⁴ is —CH— or —N—;
W is N, CR¹⁴, or C; when W is CR¹⁴, the adjacent dashed line indicates a single bond; when W is C, the adjacent dashed line indicates a double bond or L² is —C(H)═;
L¹ is selected from the group consisting of —O—, —CH₂—, —NH—, —N(Me)-, —N(Et)-, —OCH₂—, —OC(H,Me)-, —CH₂O—, —NHCH₂—, —CH₂NH—, —N(Me)CH₂—, and —SO₂NH—;
L² is selected from the group consisting of a bond, —C(O)—, —CH₂—, —C(H)═, SO₂—, —O—, —NH—, —N(Me)-, and

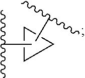

L³ is —CH₂—, —C(O)—, or —CH(CH₃)—;
each R¹ is independently selected from the group consisting of alkyl, substituted alkyl, halo, cyano, azido, alkoxy, haloalkoxy, and R¹¹R¹²NCO—;
each R² is independently selected from the group consisting of alkyl, substituted alkyl, halo, cyano, azido, alkoxy, haloalkoxy, and R¹¹R¹²NCO—;
each R³ is independently selected from the group consisting of alkyl, substituted alkyl, halo, cyano, azido, alkoxy, haloalkoxy, and R¹¹R¹²NCO—;
R⁴ is alkyl, substituted alkyl, unsubstituted arylalkylene, substituted arylalkylene, heteroalkyl, substituted heteroalkyl, unsubstituted heteroarylalkylene, substituted heteroarylalkylene, unsubstituted heterocycloalkyl, or substituted heterocycloalkyl;
R⁵ is selected from the group consisting of H, CH₂OH, —CH₂COOH, —(CH₂)₂COOH,

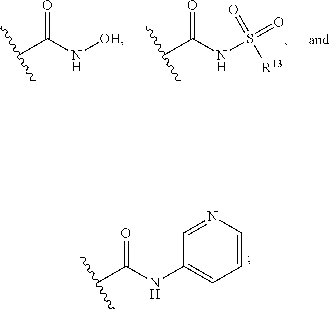

R⁷ is —CH₂F or —CHF₂;
each R¹¹ is hydrogen, or alkyl;
each R¹² is hydrogen, or alkyl;
R¹³ is hydrogen, alkyl, or -cyclopropyl;
R¹⁴ is hydrogen or alkyl;
n is an integer from 0 to 4;
m is an integer from 0 to 3; and
o is an integer from 0 to 4.

In certain aspects, the compounds are useful in methods of treatment and prevention of metabolic diseases, and conditions, methods of detection of metabolic diseases, and conditions, and methods of diagnosis of metabolic diseases, and conditions.

In another aspect, provided are compositions comprising a compound of Formula (LVII)-(LXIV). In some embodiments, the compositions are pharmaceutical compositions. Any suitable pharmaceutical composition may be used. In a further aspect, provided herein is a kit comprising the compound of Formula (LVII)-(LXIV), or embodiments thereof, or a pharmaceutical composition thereof.

In another aspect, provided herein are methods of using the compounds or the compositions described herein. In some embodiments, the methods are for treatment. In some embodiments, the methods are diagnostic methods. In some embodiments, the methods are analytical methods. In some embodiments, the compounds, or compositions described herein are used to treat a disease, or condition. In some aspects, the disease, or condition is selected from metabolic diseases, or conditions. In certain embodiments, the disease is type 2 diabetes.

Also provided herein is the use of compounds described herein, and compositions thereof, for the treatment of a metabolic disease, or condition. Also provided herein is the use of compounds described herein, and compositions thereof, for the treatment of type 2 diabetes.

### DETAILED DESCRIPTION

Described herein are GLP-1 receptor compounds useful for treating metabolic diseases or conditions, such as type 2 diabetes.

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity, and/or ready reference. The techniques and procedures described or referenced herein are generally well understood, and are commonly employed using conventional methodologies by those skilled in the art. As appropriate, procedures involving the use of commercially available kits, and reagents are generally carried out in accordance with manufacturer-defined protocols, and conditions unless otherwise noted.

As used herein, the singular forms “a,” “an,” and “the” include the plural referents unless the context clearly indicates otherwise.

The term “about” indicates and encompasses an indicated value, and a range above and below that value. In certain embodiments, the term “about” indicates the designated value ±10%, ±5%, or ±1%. In certain embodiments, the term “about” indicates the designated value ±one standard deviation of that value. In certain embodiments, for example, logarithmic scales (e.g., pH), the term “about” indicates the designated value ±0.3, ±0.2, or ±0.1.

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

“Alkoxy” and “alkoxyl,” refer to the group —OR″ where R″ is alkyl or cycloalkyl. Alkoxy groups include, in certain embodiments, methoxy, ethoxy, n-propoxy, isopropoxy, n-butoxy, tert-butoxy, sec-butoxy, n-pentoxy, n-hexoxy, 1,2-dimethylbutoxy, and the like.

The term “alkoxyamine,” as used herein, refers to the group -alkylene-O—NH₂, wherein alkylene is as defined herein. In some embodiments, alkoxyamine groups can react with aldehydes to form oxime residues. Examples of alkoxyamine groups include —CH₂CH₂—O—NH₂ and —CH₂—O—NH₂.

The term “alkyl,” as used herein, unless otherwise specified, refers to a saturated straight, or branched hydrocarbon. In certain embodiments, the alkyl group is a primary, secondary, or tertiary hydrocarbon. In certain embodiments, the alkyl group includes one to ten carbon atoms (i.e., C₁ to C₁₀ alkyl). In certain embodiments, the alkyl is a lower alkyl, for example, C₁₋₆ alkyl, and the like. In certain embodiments, the alkyl group is selected from the group consisting of methyl, ethyl, propyl, isopropyl, butyl, isobutyl, secbutyl, t-butyl, pentyl, isopentyl, neopentyl, hexyl, isohexyl, 3-methylpentyl, 2,2-dimethylbutyl, and 2,3-dimethylbutyl. In certain embodiments, “substituted alkyl” refers to an alkyl substituted with one, two, or three groups independently selected from a halogen (e.g., fluoro (F), chloro (Cl), bromo (Br), or iodo (I)), alkyl, haloalkyl, hydroxyl, amino, alkylamino, alkoxy, aryl, heteroaryl, cycloalkyl, cyano, oxo, alkyne, and heterocycloalkylalkylene. In some embodiments, alkyl is unsubstituted.

The term “alkylene,” as used herein, unless otherwise specified, refers to a divalent alkyl group, as defined herein. “Substituted alkylene” refers to an alkylene group substituted as described herein for alkyl. In some embodiments, alkylene is unsubstituted.

“Alkenyl” refers to an olefinically unsaturated hydrocarbon group, in certain embodiments, having up to about eleven carbon atoms, or from two to six carbon atoms (e.g., “lower alkenyl”), which can be straight-chained or branched, and having at least one, or from one to two sites of olefinic unsaturation. “Substituted alkenyl” refers to an alkenyl group substituted as described herein for alkyl.

“Alkenylene” refers to a divalent alkenyl as defined herein. Lower alkenylene is, for example, C₂-C₆-alkenylene.

“Alkynyl” refers to acetylenically unsaturated hydrocarbon groups, in certain embodiments, having up to about eleven carbon atoms, or from two to six carbon atoms (e.g., “lower alkynyl”), which can be straight-chained or branched, and having at least one, or from one to two sites of acetylenic unsaturation. Non-limiting examples of alkynyl groups include acetylene (—C≡CH), propargyl (—CH₂C≡CH), and the like. “Substituted alkynyl” refers to an alkynyl group substituted as described herein for alkyl.

“Alkynylene” refers to a divalent alkynyl as defined herein. Lower alkynylene is, for example, C₂-C₆-alkynylene.

“Amino” refers to —NH₂.

The term “alkylamino,” as used herein, and unless otherwise specified, refers to the group —NHR″ where R″ is, for example, C₁₋₁₀alkyl, as defined herein. In certain embodiments, alkylamino is C₁₋₆alkylamino.

The term “dialkylamino,” as used herein, and unless otherwise specified, refers to the group —NR″R″ where, each R″ is independently C₁₋₁₀alkyl, as defined herein. In certain embodiments, dialkylamino is di-C₁₋₆alkylamino.

The term “aryl,” as used herein, and unless otherwise specified, refers to phenyl, biphenyl, or naphthyl. The term includes both substituted and unsubstituted moieties. An aryl group can be substituted with any described moiety including, but not limited to, one or more moieties (e.g., in some embodiments one, two, or three moieties) selected from the group consisting of halogen (e.g., fluoro (F), chloro (Cl), bromo (Br), or iodo (I)), alkyl, haloalkyl, hydroxyl, amino, alkylamino, arylamino, alkoxy, aryloxy, nitro, cyano, sulfonic acid, sulfate, phosphonic acid, phosphate, and phosphonate, wherein each moiety is independently either unprotected, or protected as necessary, as would be appreciated by those skilled in the art (e.g., Greene, et al., Protective Groups in Organic Synthesis, John Wiley and Sons, Second Edition, 1991); and wherein the aryl in the arylamino and aryloxy substituents are not further substituted.

The term “arylamino,” as used herein, and unless otherwise specified, refers to an —NR′R″ group where R′ is hydrogen, or C₁-C₆-alkyl; and R″ is aryl, as defined herein.

The term “arylene,” as used herein, and unless otherwise specified, refers to a divalent aryl group, as defined herein.

The term “aryloxy,” as used herein, and unless otherwise specified, refers to an —OR group where R is aryl, as defined herein.

“Alkarylene” refers to an arylene group, as defined herein, wherein the aryl ring is substituted with one or two alkyl groups. “Substituted alkarylene” refers to an alkarylene, as defined herein, where the arylene group is further substituted, as defined herein for aryl.

“Aralkylene” refers to analkyl-arylene- or arylene-alkyl, for instance, a —C₁-C₂ alkyl-arylene-, -arylene-C₁-C₂ alkyl-, or —C₁-C₂ alkyl-arylene-C₁-C₂ alkyl-group, where arylene is as defined herein. “Substituted aralkylene” refers to an aralkylene, as defined herein, where the aralkylene group is substituted, as defined herein for aryl. “Substituted C₁-C₂ alkyl” refers to a C₁-C₂ alkyl group that is substituted as defined herein for an alkyl.

“Arylalkylene” refers to -alkyl-arylene- or arylene-alkyl, for instance, a —C₁-C₂ alkyl-arylene-, -arylene-C₁-C₂ alkyl-, or —C₁-C₂ alkyl-arylene-C₁-C₂ alkyl-group, where arylene is as defined herein. “Substituted arylalkylene” refers to an arylalkylene, as defined herein, where the arylalkylene group is substituted, as defined herein for aryl. “Substituted C₁-C₂ alkyl” refers to a C₁-C₂ alkyl group that is substituted as defined herein for an alkyl.

“Carboxyl” or “carboxy” refers to —C(O)OH or —COOH.

The term “cycloalkyl,” as used herein, unless otherwise specified, refers to a saturated cyclic hydrocarbon. In certain embodiments, the cycloalkyl group may be a saturated, and/or bridged, and/or non-bridged, and/or a fused bicyclic group. In certain embodiments, the cycloalkyl group includes three to ten carbon atoms (i.e., C₃ to C₁₀ cycloalkyl). In some embodiments, the cycloalkyl has from three to fifteen carbons (C₃₋₁₅), from three to ten carbons (C₃₋₁₀), from three to seven carbons (C₃₋₇), or from three to six carbons (C₃-C₆) (i.e., “lower cycloalkyl”). In certain embodiments, the cycloalkyl group is cyclopropyl, cyclobutyl, cyclopentyl, cyclohexyl, cyclohexylmethyl, cycloheptyl, bicyclo[2.1.1]hexyl, bicyclo[2.2.1]heptyl, decalinyl, or adamantyl.

The term “cycloalkylene,” as used herein refers to a divalent cycloalkyl group, as defined herein. In certain embodiments, the cycloalkylene group is cyclopropylene

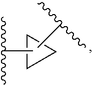

cyclobutylene

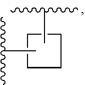

cyclopentylene

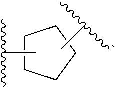

cyclohexylene

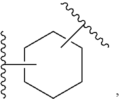

cycloheptylene

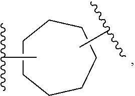

and the like. Lower cycloalkylene refers to a C₃-C₆-cycloalkylene.

The term “cycloalkylalkyl,” as used herein, unless otherwise specified, refers to an alkyl group, as defined herein, substituted with one or two cycloalkyl, as defined herein.

The term “ester,” as used herein, refers to —C(O)OR, or —COOR, where R is alkyl, as defined herein.

The term “haloalkyl” refers to an alkyl group, as defined herein, substituted with one, or more halogen atoms (e.g., in some embodiments one, two, three, four, or five) which are independently selected.

The term “heteroalkyl” refers to an alkyl, as defined herein, in which one or more carbon atoms are replaced by heteroatoms. As used herein, “heteroalkenyl” refers to an alkenyl, as defined herein, in which one, or more carbon atoms are replaced by heteroatoms. As used herein, “heteroalkynyl” refers to an alkynyl, as defined herein, in which one, or more carbon atoms are replaced by heteroatoms. Suitable heteroatoms include, but are not limited to, nitrogen (N), oxygen (O), and sulfur(S) atoms. Heteroalkyl, heteroalkenyl, and heteroalkynyl are optionally substituted. Examples of heteroalkyl moieties include, but are not limited to, aminoalkyl, sulfonylalkyl, and sulfinylalkyl. Examples of heteroalkyl moieties also include, but are not limited to, methylamino, methylsulfonyl, and methylsulfinyl. “Substituted heteroalkyl” refers to heteroalkyl substituted with one, two, or three groups independently selected from halogen (e.g., fluoro (F), chloro (Cl), bromo (Br), or iodo (I)), alkyl, haloalkyl, hydroxyl, amino, alkylamino, and alkoxy. In some embodiments, a heteroalkyl group may comprise one, two, three, or four heteroatoms. Those of skill in the art will recognize that a 4-membered heteroalkyl may generally comprise one, or two heteroatoms, a 5- or 6-membered heteroalkyl may generally comprise one, two, or three heteroatoms, and a 7- to 10-membered heteroalkyl may generally comprise one, two, three, or four heteroatoms.

The term “heteroalkylene,” as used herein, refers to a divalent heteroalkyl, as defined herein. “Substituted heteroalkylene” refers to a divalent heteroalkyl, as defined herein, substituted as described for heteroalkyl.

The term “heterocycloalkyl” refers to a monovalent, monocyclic, or multicyclic non-aromatic ring system, wherein one, or more of the ring atoms are heteroatoms independently selected from oxygen (O), sulfur(S), and nitrogen (N) (e.g., where the nitrogen, or sulfur atoms may be optionally oxidized, and the nitrogen atoms may be optionally quaternized) and the remaining ring atoms of the non-aromatic ring are carbon atoms. In certain embodiments, heterocycloalkyl is a monovalent, monocyclic, or multicyclic fully-saturated ring system. In certain embodiments, the heterocycloalkyl group has from three to twenty, from three to fifteen, from three to ten, from three to eight, from four to seven, from four to eleven, or from five to six ring atoms. The heterocycloalkyl may be attached to a core structure at any heteroatom or carbon atom which results in the creation of a stable compound. In certain embodiments, the heterocycloalkyl is a monocyclic, bicyclic, tricyclic, or tetracyclic ring system, which may include a fused or bridged ring system and in which the nitrogen or sulfur atoms may be optionally oxidized, and/or the nitrogen atoms may be optionally quaternized. In some embodiments, heterocycloalkyl radicals include, but are not limited to, 2,5-diazabicyclo[2.2.2]octanyl, decahydroisoquinolinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, heterocycloalkyl may also be optionally substituted as described herein. In certain embodiments, heterocycloalkyl is substituted with one, two, or three groups independently selected from halogen (e.g., fluoro (F), chloro (Cl), bromo (Br), or iodo (I)), alkyl, haloalkyl, hydroxyl, amino, alkylamino, oxo, cyano, and alkoxy. In some embodiments, a heterocycloalkyl group may comprise one, two, three, or four heteroatoms. Those of skill in the art will recognize that a 4-membered heterocycloalkyl may generally comprise one or two heteroatoms, a 5 or 6-membered heterocycloalkyl may generally comprise one, two, or three heteroatoms, and a 7- to 10-membered heterocycloalkyl may generally comprise one, two, three, or four heteroatoms.

“Heterocycloalkylene” refers to a divalent heterocycloalkyl as defined herein.

The term “heteroaryl” refers to a monovalent monocyclic aromatic group, and/or multicyclic aromatic group, wherein at least one aromatic ring contains one, or more heteroatoms independently selected from oxygen, sulfur, and nitrogen in the ring. Each ring of a heteroaryl group can contain one, or two oxygen atoms, one, or two sulfur atoms, and/or one to four nitrogen atoms, provided that the total number of heteroatoms in each ring is four, or less and each ring contains at least one carbon atom. In certain embodiments, the heteroaryl has from five to twenty, from five to fifteen, or from five to ten ring atoms. A heteroaryl may be attached to the rest of the molecule via a nitrogen or a carbon atom. In some embodiments, monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, triazolyl, thiadiazolyl, thiazolyl, thienyl, tetrazolyl, and triazinyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may also be optionally substituted as described herein. “Substituted heteroaryl” is a heteroaryl substituted as defined for aryl.

The term “heteroarylene” refers to a divalent heteroaryl group, as defined herein. “Substituted heteroarylene” is a heteroarylene substituted as defined for aryl.

The term “heteroarylalkylene” refers to -alkyl-heteroarylene- or -heteroarylene-alkyl, for instance, a —C₁-C₂ alkyl-heteroarylene-, -heteroarylene-C₁-C₂ alkyl-, or —C₁-C₂ alkyl-heteroarylene-C₁-C₂ alkyl-group, where heteroarylene is as defined herein. “Substituted heteroarylalkylene” refers to a heteroarylalkylene, as defined herein, where the heteroarylalkylene group is substituted, as defined herein for aryl. “Substituted C₁-C₂ alkyl” refers to a C₁-C₂ alkyl group that is substituted as defined herein for an alkyl.

As used herein, an “oxo” refers to ═O.

The term “protecting group,” as used herein, and unless otherwise specified, refers to a group that is added to an oxygen, nitrogen, or phosphorus atom to prevent its further reaction, or for other purposes. A wide variety of oxygen, and nitrogen protecting groups are known to those skilled in the art of organic synthesis. (See, e.g., Greene, et al., Protective Groups in Organic Synthesis, John Wiley and Sons, Fourth Edition, 2006, which is incorporated herein by reference).

“Pharmaceutically acceptable salt” refers to any salt of a compound provided herein which retains its biological properties, and which is not toxic or otherwise undesirable for pharmaceutical use. Such salts may be derived from a variety of organic, and inorganic counter-ions well known in the art. Such salts include, but are not limited to (1) acid addition salts formed with organic, or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, glutamic, hydroxynaphthoic, salicylic, stearic, cyclohexylsulfamic, quinic, muconic acid, and the like; or (2) salts formed when an acidic proton present in the parent compound either (a) is replaced by a metal ion, for example, an alkali metal ion, an alkaline earth ion, or an aluminum ion, or alkali metal, or alkaline earth metal hydroxides, such as sodium, potassium, calcium, magnesium, aluminum, lithium, zinc, and barium hydroxide, or ammonia; or (b) coordinates with an organic base, such as aliphatic, alicyclic, or aromatic organic amines, including, without limitation, ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, procaine, N-benzylphenethylamine, N-methylglucamine piperazine, tris(hydroxymethyl)-aminomethane, tetramethylammonium hydroxide, and the like.

Pharmaceutically acceptable salts further include, by way of example and without limitation, sodium, potassium, calcium, magnesium, ammonium, and tetraalkylammonium salts, and the like, and when the compound contains a basic functionality, salts of non-toxic organic, or inorganic acids, such as hydrohalides, for example, hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, glutamate, hydroxynaphthoate, salicylate, stearate, cyclohexylsulfamate, quinate, muconate, and the like.

The term “substantially free of” or “substantially in the absence of” with respect to a composition refers to a composition that includes at least 85%, or 90% by weight, in certain embodiments 95%, 98%, 99%, or 100% by weight; or in certain embodiments, 95%, 98%, 99%, or 100% of the designated enantiomer or diastereomer of a compound. In certain embodiments, in the methods and compounds provided herein, the compounds are substantially free of one of two enantiomers. In certain embodiments, in the methods and compounds provided herein, the compounds are substantially free of one of two diastereomers. In certain embodiments, in the methods, and compounds provided herein, the compounds are substantially free of enantiomers (i.e., a racemic, or 50:50 mixture of compounds).

Similarly, the term “isolated” with respect to a composition refers to a composition that includes at least 85%, 90%, 95%, 98%, or 99% to 100% by weight, of the compound, the remainder comprising other chemical species, enantiomers or diastereomers.

“Solvate” refers to a compound provided herein, or a salt thereof, that further includes a stoichiometric, or non-stoichiometric amount of solvent bound by non-covalent intermolecular forces. Where the solvent is water, the solvate is a hydrate.

“Isotopic composition” refers to the amount of each isotope present for a given atom, and “natural isotopic composition” refers to the naturally occurring isotopic composition, or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as hydrogen (H), the position is understood to have hydrogen at its natural isotopic composition.

“Isotopic enrichment” refers to the percentage of incorporation of an amount of a specific isotope at a given atom in a molecule in the place of that atom's natural isotopic abundance. For example, deuterium (D) enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Isotopically enriched” refers to an atom having an isotopic composition other than the natural isotopic composition of that atom. “Isotopically enriched” may also refer to a compound containing at least one atom having an isotopic composition other than the natural isotopic composition of that atom.

As used herein, “alkyl,” “alkylene,” “alkylamino,” “dialkylamino,” “cycloalkyl,” “aryl,” “arylene,” “alkoxy,” “amino,” “carboxyl,” “heterocycloalkyl,” “heteroaryl,” “heteroarylene,” “carboxyl,” and “amino acid” groups optionally comprise deuterium (D) at one, or more positions where hydrogen (H) atoms are present, and wherein the deuterium composition of the atom or atoms is other than the natural isotopic composition.

Also as used herein, “alkyl,” “alkylene,” “alkylamino,” “dialkylamino,” “cycloalkyl,” “aryl,” “arylene,” “alkoxy,” “amino,” “carboxyl,” “heterocycloalkyl,” “heteroaryl,” “heteroarylene,” “carboxyl,” and “amino acid” groups optionally comprise carbon-13 (¹³C) at an amount other than the natural isotopic composition.

As used herein, term “EC₅₀” refers to a dosage, concentration, or amount of a particular test compound that elicits a dose-dependent response at 50% of maximal expression of a particular response that is induced, provoked, or potentiated by the particular test compound.

As used herein, and unless otherwise specified, the term “IC₅₀” refers to an amount, concentration, or dosage of a particular test compound that achieves a 50% inhibition of a maximal response in an assay that measures such response.

As used herein, the terms “subject”, and “patient” are used interchangeably. The terms “subject”, and “subjects” refer to an animal, such as a mammal including a non-primate (e.g., a cow, pig, horse, cat, dog, rat, and mouse), and a primate (e.g., a monkey, such as a cynomolgous monkey, a chimpanzee, and a human), and in certain embodiments, a human. In certain embodiments, the subject is a farm animal (e.g., a horse, a cow, a pig, etc.), or a pet (e.g., a dog or a cat). In certain embodiments, the subject is a human.

As used herein, the terms “therapeutic agent”, and “therapeutic agents” refer to any agent(s) which can be used in the treatment, or prevention of a disorder, or one or more symptoms thereof. In certain embodiments, the term “therapeutic agent” includes a compound provided herein. In certain embodiments, a therapeutic agent is an agent which is known to be useful for, or has been, or is currently being used for the treatment, or prevention of a disorder, or one, or more symptoms thereof.

“Therapeutically effective amount” refers to an amount of a compound, or composition that, when administered to a subject for treating a condition, is sufficient to effect such treatment for the condition. A “therapeutically effective amount” can vary depending on, inter alia, the compound, the disease or disorder, and its severity, and the age, weight, etc., of the subject to be treated.

“Treating” or “treatment” of any disease, or disorder refers, in certain embodiments, to ameliorating a disease, or disorder that exists in a subject. In another embodiment, “treating”, or “treatment” includes ameliorating at least one physical parameter, which may be indiscernible by the subject. In yet another embodiment, “treating”, or “treatment” includes modulating the disease, or disorder, either physically (e.g., stabilization of a discernible symptom), or physiologically (e.g., stabilization of a physical parameter), or both. In yet another embodiment, “treating”, or “treatment” includes delaying, or preventing the onset of the disease, or disorder, or delaying, or preventing recurrence of the disease, or disorder. In yet another embodiment, “treating”, or “treatment” includes the reduction or elimination of either the disease, or disorder, or retarding the progression of the disease, or disorder, or of one, or more symptoms of the disease, or disorder, or reducing the severity of the disease, or disorder, or of one, or more symptoms of the disease, or disorder.

As used herein, the terms “prophylactic agent”, and “prophylactic agents” as used refer to any agent(s) which can be used in the prevention of a disorder, or one or more symptoms thereof. In certain embodiments, the term “prophylactic agent” includes a compound provided herein. In certain other embodiments, the term “prophylactic agent” does not refer a compound provided herein. For example, a prophylactic agent is an agent which is known to be useful for, or has been or is currently being used to prevent, or impede the onset, development, progression, and/or severity of a disorder.

As used herein, the phrase “prophylactically effective amount” refers to the amount of a therapy (e.g., prophylactic agent) which is sufficient to result in the prevention, or reduction of the development, recurrence, or onset of one or more symptoms associated with a disorder, or to enhance or improve the prophylactic effect(s) of another therapy (e.g., another prophylactic agent).

### Compounds of Formulae (LVII)-(LXIV)

Herein are GLP-1 receptor compounds useful for modulating one, or more properties of GLP-1 receptor. The compounds can be prepared as described herein and used for therapy, or diagnosis. In certain embodiments, the therapy is the treatment of a metabolic disease or condition. In certain embodiments, the therapy is the treatment of type 2 diabetes.

The embodiments described herein include the recited compounds as well as pharmaceutically acceptable salts, hydrates, solvates, stereoisomers, tautomers, and/or mixtures thereof.

In certain embodiments provided herein is a compound of Formula (LVII), or a pharmaceutically acceptable salt, or stereoisomer thereof:

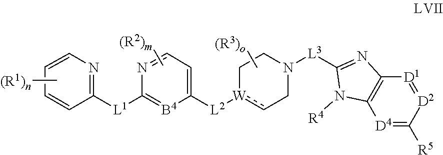

wherein:
B⁴ is —CH—, —C(R²)—, or —N—;
D¹ is —CH— or —N—;
D² is —CH—, —N—, or —C(CH₃)—;
D⁴ is —CH— or —N;
W is N, CR¹⁴, or C;
when W is CR¹⁴, the adjacent dashed line indicates a single bond;
when W is C, the adjacent dashed line indicates a double bond or L² is —C(H)═; L¹ is selected from the group consisting of —O—, —CH₂—, —NH—, —N(Me)-, —N(Et)-, —OCH₂—, —OC(H,Me)-, —CH₂O—, —CH₂NH—, —NHCH₂—, —N(Me)CH₂—, and —SO₂NH—;
L² is selected from the group consisting of a bond, —C(O)—, —CH₂—, —C(H)═, SO₂—, —O—, —NH—, —N(Me)-, and

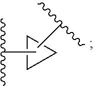

L³ is —CH₂—, —C(O)—, or —CH(CH₃)—;
each R¹ is independently selected from the group consisting of alkyl, substituted alkyl, halo, cyano, azido, alkoxy, haloalkoxy, and R¹¹R¹²NCO—;
each R² is independently selected from the group consisting of alkyl, substituted alkyl, halo, cyano, azido, alkoxy, haloalkoxy, and R¹¹R¹²NCO—;
each R³ is independently selected from the group consisting of alkyl, substituted alkyl, halo, cyano, azido, alkoxy, haloalkoxy, and R¹¹R¹²NCO—;
R⁴ is selected from the group consisting of alkyl, substituted alkyl, unsubstituted arylalkyl, substituted arylalkyl, heteroalkyl, substituted heteroalkyl, unsubstituted heteroarylalkyl, substituted heteroarylalkyl, unsubstituted heterocycloalkyl, and substituted heterocycloalkyl;
R⁵ is selected from the group consisting of —H, —CH₂OH, —COOH, —CH₂COOH, —(CH₂)₂COOH, —COCF₃, —C(OH)CF₃, —CONHCN, —CONHOH, CONHOMe, —CONHSO₂N(Me)₂, —PO₃H₂, —PO(Me)(OH), —SO₃H, —SO₂NH₂, —SO₂NHMe, —B(OH)₂,

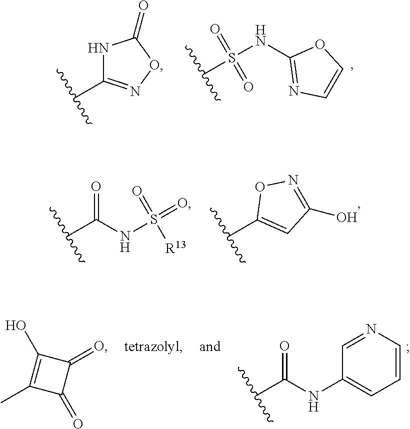

each R¹¹ is hydrogen, or alkyl;
each R¹² is hydrogen, or alkyl;
R¹³ is hydrogen, alkyl, or -cyclopropyl;
R¹⁴ is hydrogen or alkyl;
n is an integer from 0 to 4;
m is an integer from 0 to 3; and
o is an integer from 0 to 4.

In certain embodiments, the compound of Formula (LVII) is selected from compounds 554, 566, 567, 595, 597, 602-611, 613, 615-617, 619, 620, 622, 624-632, and 634-636 in Table 2.

In certain embodiments provided herein is a compound of Formula (LVIII), or a pharmaceutically acceptable salt, or stereoisomer thereof:

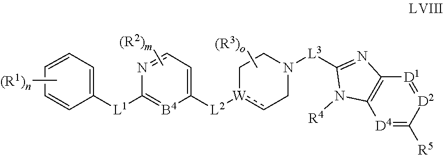

wherein:
B⁴ is —CH—, —C(R²)—, or —N—;
D¹ is —CH— or —N—;
D² is —CH—, —N—, or —C(CH₃)—;
D⁴ is —CH— or —N;
W is N, CR¹⁴, or C;
when W is CR¹⁴, the adjacent dashed line indicates a single bond;
when W is C, the adjacent dashed line indicates a double bond or L² is —C(H)═; L¹ is selected from the group consisting of —O—, —CH₂—, —NH—, —N(Me)-, —N(Et)-, —OCH₂—, —OC(H,Me)-, —CH₂O—, —CH₂NH—, —NHCH₂—, —N(Me)CH₂—, and —SO₂NH—;
L² is selected from the group consisting of a bond, —C(O)—, —CH₂—, —C(H)═, SO₂—, —O—, —NH—, —N(Me)-, and

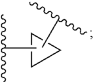

L³ is —CH₂—, —C(O)—, or —CH(CH₃)—;
each R¹ is independently selected from the group consisting of alkyl, substituted alkyl, halo, cyano, azido, alkoxy, haloalkoxy, and R¹¹R¹²NCO—; each R² is independently selected from the group consisting of alkyl, substituted alkyl, halo, cyano, azido, alkoxy, haloalkoxy, and R¹¹R¹²NCO—;
each R³ is independently selected from the group consisting of —CH₂CH₃, —CH₂F, —CH₂CN, and —CF₃;
R⁴ is selected from the group consisting of alkyl, substituted alkyl, unsubstituted arylalkylene, substituted arylalkyl, heteroalkyl, substituted heteroalkyl, unsubstituted heteroarylalkyl, substituted heteroarylalkyl, unsubstituted heterocycloalkyl, and substituted heterocycloalkyl;
R⁵ is selected from the group consisting of —H, —CH₂OH, —COOH, —CH₂COOH, —(CH₂)₂COOH, —COCF₃, —C(OH)CF₃, —CONHCN, —CONHOH, CONHOMe, —CONHSO₂N(Me)₂, —PO₃H₂, —PO(Me)(OH), —SO₃H, —SO₂NH₂, —SO₂NHMe, —B(OH)₂,

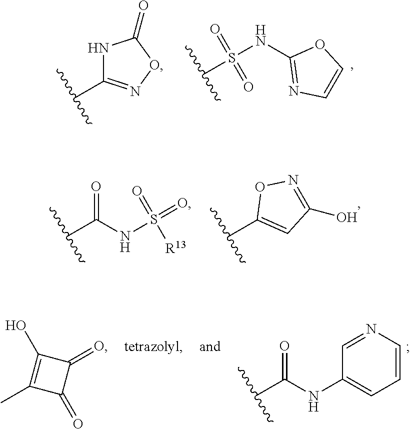

each R¹¹ is hydrogen, or alkyl;
each R¹² is hydrogen, or alkyl;
R¹³ is hydrogen, alkyl, or -cyclopropyl;
R¹⁴ is hydrogen or alkyl;
n is an integer from 0 to 4;
m is an integer from 0 to 3; and
o is an integer from 0 to 4.

In certain embodiments, when B⁴ is —C— and R³ is —CH₂F, then the compound accordingly to Formula (LVIII) is not

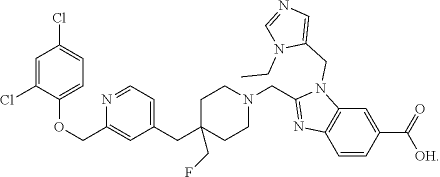 [SMILES: CCn1cncc1Cn1c(CN2CCC(CF)(Cc3ccnc(COc4ccc(Cl)cc4Cl)c3)CC2)nc2ccc(C(=O)O)cc21]

In certain embodiments, when B⁴ is —N— and R³ is —CH₂F, then the compound accordingly to Formula (LVIII) is not

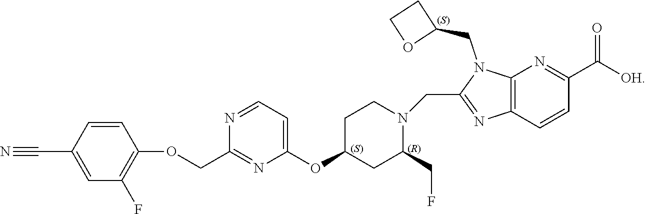

In certain embodiments, the compound of Formula (LVIII) is selected from compounds 555, 557, 586, 587, and 599 in Table 2.

In certain embodiments provided herein is a compound of Formula (LIX), or a pharmaceutically acceptable salt, or stereoisomer thereof:

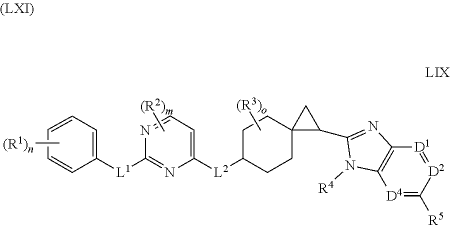

wherein:
D¹ is —CH— or —N—;
D² is —CH—, —N—, or —C(CH₃)—;
D⁴ is —CH— or —N—;
L¹ is selected from the group consisting of —O—, —CH₂—, —NH—, —N(Me)-, —N(Et)-, —OCH₂—, —OC(H,Me)-, —CH₂O—, —CH₂NH—, —NHCH₂—, —N(Me)CH₂—, and —SO₂NH—;
L² is selected from the group consisting of a bond, —C(O)—, —CH₂—, —C(H)═, SO₂—, —O—, —NH—, —N(Me)-, and

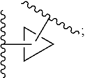

each R¹ is independently selected from the group consisting of alkyl, substituted alkyl, halo, cyano, azido, alkoxy, haloalkoxy, and R¹¹R¹²NCO—;
each R² is independently selected from the group consisting of alkyl, substituted alkyl, halo, cyano, azido, alkoxy, haloalkoxy, and R¹¹R¹²NCO—;
each R³ is independently selected from the group consisting of alkyl, substituted alkyl, halo, cyano, azido, alkoxy, haloalkoxy, and R¹¹R¹²NCO—;
R⁴ is selected from the group consisting of alkyl, substituted alkyl, unsubstituted arylalkyl, substituted arylalkyl, heteroalkyl, substituted heteroalkyl, unsubstituted heteroarylalkyl, substituted heteroarylalkyl, unsubstituted heterocycloalkyl, and substituted heterocycloalkyl;
R⁵ is selected from the group consisting of —H, —CH₂OH, —COOH, —CH₂COOH, —(CH₂)₂COOH, —COCF₃, —C(OH)CF₃, —CONHCN, —CONHOH, CONHOMe, —CONHSO₂N(Me)₂, —PO₃H₂, —PO(Me)(OH), —SO₃H, —SO₂NH₂, —SO₂NHMe, —B(OH)₂,

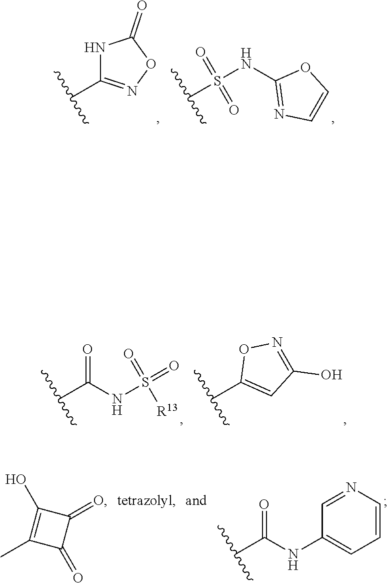

each R¹¹ is hydrogen, or alkyl;
each R¹² is hydrogen, or alkyl; R¹³ is hydrogen, alkyl, or -cyclopropyl;
R¹⁴ is hydrogen or alkyl;
n is an integer from 0 to 4;
m is an integer from 0 to 3; and
o is an integer from 0 to 4.

In certain embodiments, the compound of Formula (LIX) is compound 571 in Table 2.

In certain embodiments provided herein is a compound of Formula (LX), or a pharmaceutically acceptable salt, or stereoisomer thereof:

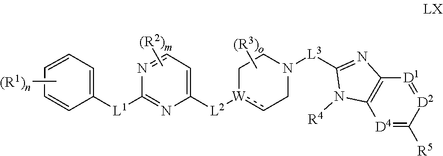

wherein:
D¹ is —CH— or —N—;
D² is —CH—, —N—, or —C(CH₃)—;
D⁴ is —CH— or —N—;
W is N,
CR¹⁴, or C;
when W is CR¹⁴, the adjacent dashed line indicates a single bond;
when W is C, the adjacent dashed line indicates a double bond or L² is —C(H)═;
L¹ is selected from the group consisting of —O—, —CH₂—, —NH—, —N(Me)-, —N(Et)-, —OCH₂—, —OC(H,Me)-, —CH₂O—, —NHCH₂—, —CH₂NH—, —N(Me)CH₂—, and —SO₂NH—;
L² is selected from the group consisting of a bond, —C(O)—, —CH₂—, —C(H)═, SO₂—, —O—, —NH—, —N(Me)-, and

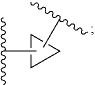

L³ is —CH₂—, —C(O)—, or —CH(CH₃)—;
each R¹ is independently selected from the group consisting of alkyl, substituted alkyl, halo, cyano, azido, alkoxy, haloalkoxy, and R¹¹R¹²NCO—;
each R² is independently selected from the group consisting of alkyl, substituted alkyl, halo, cyano, azido, alkoxy, haloalkoxy, and R¹¹R¹²NCO—;
R³ is independently selected from the group consisting of —CH₂CH₃, —CH₂F, and —CH₂CN;
R⁴ is selected from the group consisting of (1,1-dioxidothietan-2-yl)methyl, ((R)-1,1-dioxidothietan-2-yl)methyl, ((S)-1,1-dioxidothietan-2-yl)methyl,

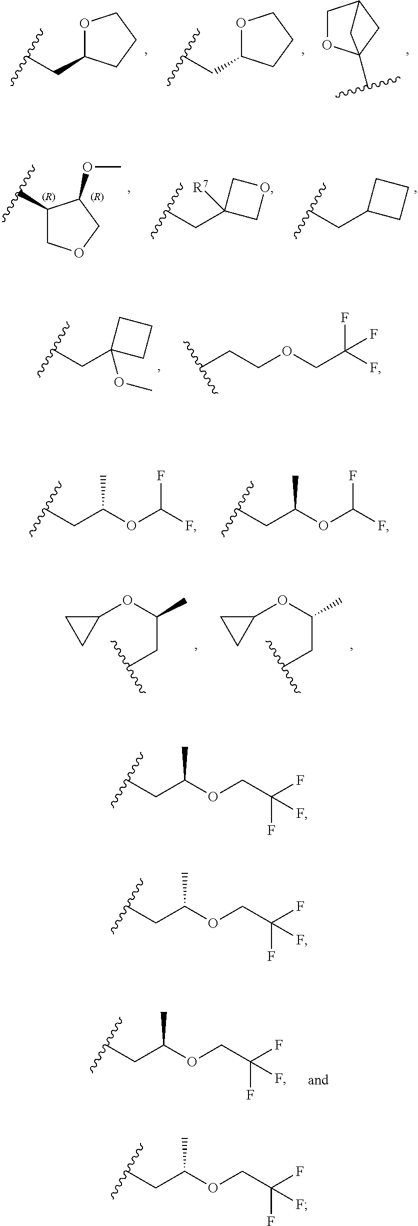

R⁵ is selected from the group consisting of —H, —CH₂OH, —COOH, —CH₂COOH, —(CH₂)₂COOH, —COCF₃, —C(OH)CF₃, —CONHCN, —CONHOH, CONHOMe, —CONHSO₂N(Me)₂, —PO₃H₂, —PO(Me)(OH), —SO₃H, —SO₂NH₂, —SO₂NHMe, —B(OH)₂,

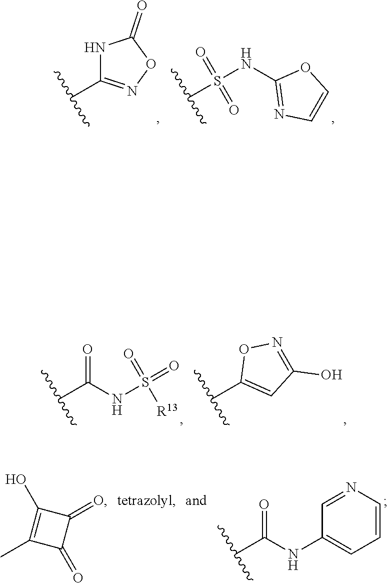

R⁷ is —CH₂F or —CHF₂; each
R¹¹ is hydrogen, or alkyl; each
R¹² is hydrogen, or alkyl;
R¹³ is hydrogen, alkyl, or -cyclopropyl;
R¹⁴ is hydrogen or alkyl;
n is an integer from 0 to 4;
m is an integer from 0 to 3; and
o is an integer from 0 to 4.

In certain embodiments, the compound of Formula (LX) is selected from compounds 570, 575, 579, 582, 583, 584, 588, 589-592, 600, 601, 612, 618, and 621 in Table 2.

In certain embodiments provided herein is a compound of Formula (LXIII), or a pharmaceutically acceptable salt, or stereoisomer thereof:

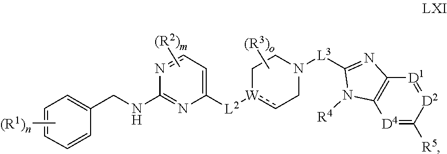

wherein
D¹ is —CH— or —N—;
D² is —CH—, —N—, or —C(CH₃)—;
D⁴ is —CH— or —N—;
L² is selected from the group consisting of a bond, —C(O)—, —CH₂—, —C(H)═, SO₂—, —O—, —NH—, —N(Me)-, and

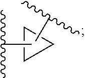

L³ is —CH₂—, —C(O)—, or —CH(CH₃)—;
each R¹ is independently selected from the group consisting of alkyl, substituted alkyl, halo, cyano, azido, alkoxy, haloalkoxy, and R¹¹R¹²NCO—;
each R² is independently selected from the group consisting of alkyl, substituted alkyl, halo, cyano, azido, alkoxy, haloalkoxy, and R¹¹R¹²NCO—;
each R³ is independently selected from the group consisting of alkyl, substituted alkyl, halo, cyano, azido, alkoxy, haloalkoxy, and R¹¹R¹²NCO—;
R⁴ is selected from the group consisting of alkyl, substituted alkyl, unsubstituted arylalkyl, substituted arylalkyl, heteroalkyl, substituted heteroalkyl, unsubstituted heteroarylalkyl, substituted heteroarylalkyl, unsubstituted heterocycloalkyl, and substituted heterocycloalkyl;
R⁵ is selected from the group consisting of —H, —CH₂OH, —COOH, —CH₂COOH, —(CH₂)₂COOH, —COCF₃, —C(OH)CF₃, —CONHCN, —CONHOH, CONHOMe, —CONHSO₂N(Me)₂, —PO₃H₂, —PO(Me)(OH), —SO₃H, —SO₂NH₂, —SO₂NHMe, —B(OH)₂,

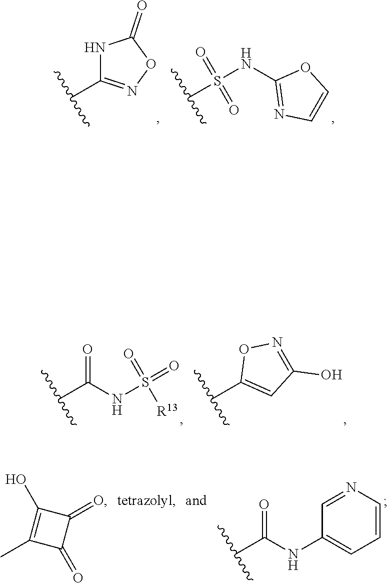

each R¹¹ is hydrogen, or alkyl;
each R¹² is hydrogen, or alkyl;
R¹³ is hydrogen, alkyl, or -cyclopropyl;
R¹⁴ is hydrogen or alkyl;
n is an integer from 0 to 4;
m is an integer from 0 to 3; and
o is an integer from 0 to 4.

In certain embodiments, the compound of Formula (LXI) is compound 614 in Table 2.

In certain embodiments provided herein is a compound of Formula (LXII), or a pharmaceutically acceptable salt, or stereoisomer thereof:

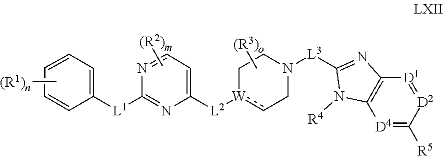

wherein:
D¹ is —CH— or —N—;
D² is —CH—, —N—, or —C(CH₃)—;
D⁴ is —CH— or —N—;
W is N, CR¹⁴, or C;
when W is CR¹⁴, the adjacent dashed line indicates a single bond;
when W is C, the adjacent dashed line indicates a double bond or L² is —C(H)═;
L¹ is selected from the group consisting of —O—, —CH₂—, —NH—, —N(Me)-, —N(Et)-, —OCH₂—, —OC(H,Me)-, —CH₂O—, —NHCH₂—, —CH₂NH—, —N(Me)CH₂—, and —SO₂NH—; L² is selected from the group consisting of a bond, —C(O)—, —CH₂—, —C(H)═, SO₂—, —O—, —NH—, —N(Me)-, and

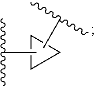

L³ is —CH₂—, —C(O)—, or —CH(CH₃)—;
each R¹ is independently selected from the group consisting of alkyl, substituted alkyl, halo, cyano, azido, alkoxy, haloalkoxy, and R¹¹R¹²NCO—;
each R² is independently selected from the group consisting of alkyl, substituted alkyl, halo, cyano, azido, alkoxy, haloalkoxy, and R¹¹R¹²NCO—;
each R³ is independently selected from the group consisting of alkyl, substituted alkyl, halo, cyano, azido, alkoxy, haloalkoxy, and R¹¹R¹²NCO—;
R⁴ is selected from the group consisting of alkyl, substituted alkyl, unsubstituted arylalkyl, substituted arylalkyl, heteroalkyl, substituted heteroalkyl, unsubstituted heteroarylalkyl, substituted heteroarylalkyl, unsubstituted heterocycloalkyl, and substituted heterocycloalkyl;
R⁵ is selected from the group consisting of —H, —CH₂OH—COOH, —CH₂COOH, —(CH₂)₂COOH, —COCF₃, —C(OH)CF₃, —CONHCN, —CONHOH, CONHOMe, —CONHSO₂N(Me)₂, —PO₃H₂, —PO(Me)(OH), —SO₃H, —SO₂NH₂, —SO₂NHMe, —B(OH)₂,

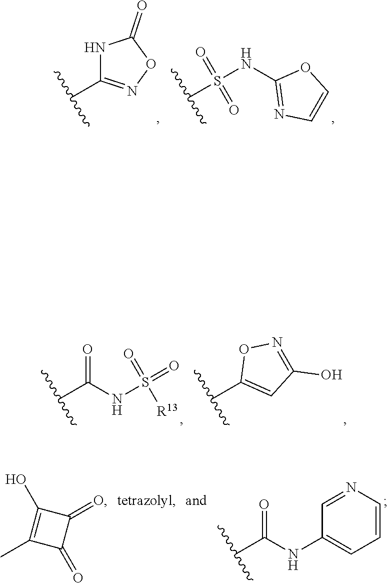

each R¹¹ is hydrogen, or alkyl;
each R¹² is hydrogen, or alkyl;
R¹³ is hydrogen, alkyl, or -cyclopropyl;
R¹⁴ is hydrogen or alkyl;
n is an integer from 0 to 4;
m is an integer from 0 to 3; and
o is an integer from 0 to 4.

In certain embodiments, a compound of Formula (LXII) is option (a), (b), (c), (d), or (e). In certain embodiments, option (a), when D¹ is —N— and D² and D⁴ are —CH—, then the compound is not one of the following compounds.

| Com- | |
|---|---|
| pound No. | Structure |
| 486 | 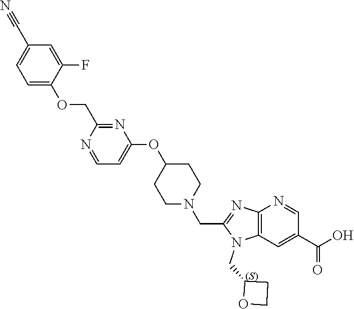 |
| 512 | 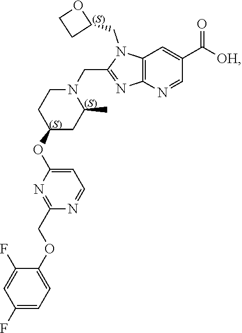 |
| | and |
| 513 | 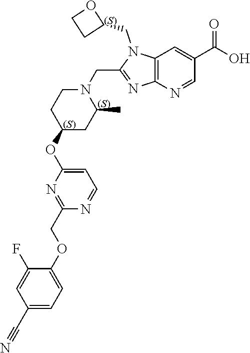 |

In certain embodiments, option (b), D¹ and D⁴ are —CH— and D² is —N—. In certain embodiments, option (c), D¹ and D⁴ are —CH— and D² is —C(CH₃). In certain embodiments, option (d), D¹, D², and D⁴ are —CH— and R⁵ is selected from group consisting of: —H, —CH₂OH—CH₂COOH, —(CH₂)₂COOH,

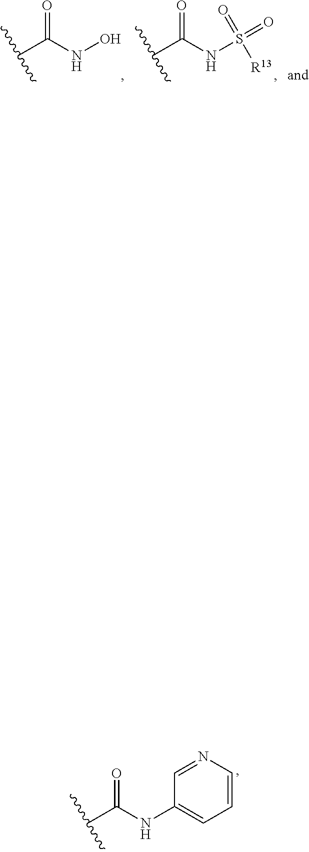

wherein R¹³ is —CH₃ or -cyclopropyl. In certain embodiments, option (e), D¹, D², and D⁴ are —CH— and L³ is —C(H)CH₃—. In certain embodiments, option (e), zero or one of D¹, D², and D⁴ is —N— and L³ is —C(H)CH₃—. In certain embodiments, the compound of Formula (LXII) is selected from compounds 556, 559, 560, 563-565, 568, 569, 574, 580, 585, 623, 633, and 637-639 in Table 2.

In certain embodiments, provided is a compound of any of Formulas (LVII-LXIV), or a pharmaceutically salt, tautomer, stereoisomer, and/or mixture of stereoisomers thereof, wherein each R² and R³ is H, or F.

In certain embodiments, provided is a compound of any of Formulas (LVII-LXIV), or a pharmaceutically salt, tautomer, stereoisomer, and/or mixture of stereoisomers thereof, wherein m is 0; and o is 0.

In certain embodiments, provided is a compound of any of Formulas (LVII-LXIV), or a pharmaceutically salt, tautomer, stereoisomer, and/or mixture of stereoisomers thereof, wherein W is N, or CH.

In certain embodiments, provided is a compound of any of Formulas (LVII, LVIII, LX, and LXII), or a pharmaceutically salt, tautomer, stereoisomer, and/or mixture of stereoisomers thereof, wherein W is C.

In certain embodiments, provided is a compound of any of Formulas (LVII, LVIII, LX, and LXII), or a pharmaceutically salt, tautomer, stereoisomer, and/or mixture of stereoisomers thereof, wherein W is CH.

In certain embodiments, provided is a compound of any of Formulas (LVII, LVIII, LX, and LXII), or a pharmaceutically salt, tautomer, stereoisomer, and/or mixture of stereoisomers thereof, wherein W is N.

In certain embodiments, provided is a compound of any of Formulas (LVII, LVIII, LX, and LXII), or a pharmaceutically salt, tautomer, stereoisomer, and/or mixture of stereoisomers thereof, wherein W is CR¹⁴.

In certain embodiments, provided is a compound of any of Formulas (LVII-LXIV), or a pharmaceutically salt, tautomer, stereoisomer, and/or mixture of stereoisomers thereof, wherein R⁴ is selected from the group consisting of azetidine-3-yl-methyl, (3R)-azetidine-3-yl-methyl, (3S)-azetidine-3-yl-methyl, (S)-buta-4-yl-1,3-diol, butan-1-yl-one, 1-(cyanomethyl)-cycloprop-1-yl-methyl, 3-(difluoromethyl)oxetan-3-yl-methyl, (1,1-dioxidothietan-2-yl)methyl, ((R)-1,1-dioxidothietan-2-yl)methyl, ((S)-1,1-dioxidothietan-2-yl)methyl, (1-ethyl-1H-imidazol-5-yl)-methyl, 1-(Fluoromethyl)-cycloprop-1-yl-methyl, 3-fluorooxetan-3-yl-methyl, 1-hydroxyl-cycloprop-1-yl-methyl, isoxazol-5-yl-methyl, (3R,4R)-4-methoxytetrahydrofuran-3-yl, (1-methyl-1H-imidazol-5-yl)-methyl, 1-methoxy-cycloprop-1yl-methyl, methoxyethan-2-yl, 1-(2-methoxyethyl)-cycloprop-1-yl-methyl, {[1-(1,2-oxazole-3-carbonyl)azetidin-2-yl]methyl}, {[(2R)-1-(1,2-oxazole-3-carbonyl)azetidin-2-yl]methyl}, {[(2S)-1-(1,2-oxazole-3-carbonyl)azetidin-2-yl]methyl}, oxetan-2-yl-methyl, (2S)-oxetan-2-yl-methyl, oxelan-3-yl-methyl, (3R)-oxelan-3-yl-methyl, 1,3-oxazol-2-yl-methyl, (S)-1,3-oxazol-2-yl-methyl, (R)-1,3-oxazol-2-yl-methyl, 2-oxabicyclo[2.1.1]hexa-1-yl-methyl, 1-cyclobutylmethyl, (R)-2-cyclopropoxypropyl, (S)-2-cyclopropoxypropyl, (2R)-2-(difluoromethoxy) propyl, (2S)-2-(difluoromethoxy) propyl, (2S)-oxolan-2-yl-methyl, (1-methoxycyclobutyl)methyl, (2R)-2-(2,2,2-trifluoroethoxy) propyl, (2S)-2-(2,2,2-trifluoroethoxy) propyl, and 2-(2,2,2-trifluoroethoxy)ethyl.

In certain embodiments, provided is a compound of any of Formulas (LVII-LXIV), or a pharmaceutically salt, tautomer, stereoisomer, and/or mixture of stereoisomers thereof, wherein R⁴ is selected from the group consisting of oxetan-2-yl-methyl, (1-ethyl-1H-imidazol-5-yl)-methyl, oxelan-3-yl-methyl and 1,3-oxazol-2-yl-methyl.

In certain embodiments, provided is a compound of any of Formulas (LVII-LXIV), or a pharmaceutically salt, tautomer, stereoisomer, and/or mixture of stereoisomers thereof, wherein R⁴ is selected from the group consisting of oxetan-2-yl-methyl, and (1-ethyl-1H-imidazol-5-yl)-methyl.

In certain embodiments, provided is a compound of any of Formulas (LVII-LXIV), or a pharmaceutically salt, tautomer, stereoisomer, and/or mixture of stereoisomers thereof, wherein R⁴ is oxetan-2-yl-methyl.

In certain embodiments, provided is a compound of any of Formulas (LVII-LXIV), or a pharmaceutically salt, tautomer, stereoisomer, and/or mixture of stereoisomers thereof, wherein R⁴ is (2S)-oxetan-2-yl-methyl.

In certain embodiments, provided is a compound of any of Formulas (LVII-LXIV), or a pharmaceutically salt, tautomer, stereoisomer, and/or mixture of stereoisomers thereof, wherein R⁴ is (1-ethyl-1H-imidazol-5-yl)-methyl.

In certain embodiments, provided is a compound of any of Formulas (LVII-LXIV), or a pharmaceutically salt, tautomer, stereoisomer, and/or mixture of stereoisomers thereof, wherein R⁴ is selected from the group consisting of(S)-buta-4-yl-1,3-diol, butan-1-yl-one, 1-(cyanomethyl)-cycloprop-1-yl-methyl, (1,1-dioxidothietan-2-yl)methyl, ((R)-1,1-dioxidothietan-2-yl)methyl, ((S)-1,1-dioxidothietan-2-yl)methyl, 1-(Fluoromethyl)-cycloprop-1-yl-methyl, 1-hydroxyl-cycloprop-1-yl-methyl, isoxazol-5-yl-methyl, (1-methyl-1H-imidazol-5-yl)-methyl, 1-methoxy-cycloprop-1yl-methyl, methoxyethan-2-yl, 1-(2-methoxyethyl)-cycloprop-1-yl-methyl, 2-oxabicyclo[2.1.1]hexa-1-yl-methyl, 1-cyclobutylmethyl, (R)-2-cyclopropoxypropyl, (S)-2-cyclopropoxypropyl, (2R)-2-(difluoromethoxy) propyl, (2S)-2-(difluoromethoxy) propyl, (2S)-oxolan-2-yl-methyl, (1-methoxycyclobutyl)methyl, (2R)-2-(2,2,2-trifluoroethoxy) propyl, (2S)-2-(2,2,2-trifluoroethoxy) propyl, and 2-(2,2,2-trifluoroethoxy)ethyl.

In certain embodiments, provided is a compound of any of Formulas (LVII-LXIV), or a pharmaceutically salt, tautomer, stereoisomer, and/or mixture of stereoisomers thereof, wherein R⁴ is (S)-buta-4-yl-1,3-diol.

In certain embodiments, provided is a compound of any of Formulas (LVII-LXIV), or a pharmaceutically salt, tautomer, stereoisomer, and/or mixture of stereoisomers thereof, wherein R⁴ is 3-(difluoromethyl)oxetan-3-yl-methyl.

In certain embodiments, provided is a compound of any of Formulas (LVII-LXIV), or a pharmaceutically salt, tautomer, stereoisomer, and/or mixture of stereoisomers thereof, wherein R⁴ is ((R)-1,1-dioxidothietan-2-yl)methyl.

In certain embodiments, provided is a compound of any of Formulas (LVII-LXIV), or a pharmaceutically salt, tautomer, stereoisomer, and/or mixture of stereoisomers thereof, wherein R⁴ is ((S)-1,1-dioxidothietan-2-yl)methyl.

In certain embodiments, provided is a compound of any of Formulas (LVII-LXIV), or a pharmaceutically salt, tautomer, stereoisomer, and/or mixture of stereoisomers thereof, wherein R⁴ is 3-fluorooxetan-3-yl-methyl.

In certain embodiments, provided is a compound of any of Formulas (LVII-LXIV), or a pharmaceutically salt, tautomer, stereoisomer, and/or mixture of stereoisomers thereof, wherein R⁴ is (3R,4R)-4-methoxytetrahydrofuran-3-yl.

In certain embodiments, provided is a compound of any of Formulas (LVII-LXIV), or a pharmaceutically salt, tautomer, stereoisomer, and/or mixture of stereoisomers thereof, wherein R⁴ is 1,3-oxazol-2-yl-methyl.

In certain embodiments, provided is a compound of any of Formulas (LVII-LXIV), or a pharmaceutically salt, tautomer, stereoisomer, and/or mixture of stereoisomers thereof, wherein R⁴ is (S)-1,3-oxazol-2-yl-methyl.

In certain embodiments, provided is a compound of any of Formulas (LVII-LXIV), or a pharmaceutically salt, tautomer, stereoisomer, and/or mixture of stereoisomers thereof, wherein R⁴ is (R)-1,3-oxazol-2-yl-methyl.

In certain embodiments, provided is a compound of any of Formulas (LVII-LXIV), or a pharmaceutically salt, tautomer, stereoisomer, and/or mixture of stereoisomers thereof, wherein R⁴ is 2-oxabicyclo[2.1.1]hexa-1-yl-methyl.

In certain embodiments, provided is a compound of any of Formulas (LVII-LXIV), or a pharmaceutically salt, tautomer, stereoisomer, and/or mixture of stereoisomers thereof, wherein R⁵ is —COOH, or —COOMe.

In certain embodiments, provided is a compound of any of Formulas (LVII-LXIV), or a pharmaceutically salt, tautomer, stereoisomer, and/or mixture of stereoisomers thereof, wherein R⁵ is —COOH.

In certain embodiments, provided is a compound of any of Formulas (LVII-LXIV), or a pharmaceutically salt, tautomer, stereoisomer, and/or mixture of stereoisomers thereof, wherein R⁵ is —CH₂COOH.

In certain embodiments, provided is a compound of any of Formulas (LVII-LXIV), or a pharmaceutically salt, tautomer, stereoisomer, and/or mixture of stereoisomers thereof, wherein R⁵ is —(CH₂)₂COOH.

In certain embodiments, provided is a compound of any of Formulas (LVII-LXIV), or a pharmaceutically salt, tautomer, stereoisomer, and/or mixture of stereoisomers thereof, wherein R⁵ is tetrazolyl.

In certain embodiments, provided is a compound of any of Formulas (LVII-LXIV), or a pharmaceutically salt, tautomer, stereoisomer, and/or mixture of stereoisomers thereof, wherein R⁵ is 1H-1,2,3,4-tetrazol-5-yl.

In certain embodiments, provided is a compound of any of Formulas (LVII-LXIV), or a pharmaceutically salt, tautomer, stereoisomer, and/or mixture of stereoisomers thereof, wherein R⁵ is

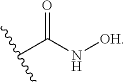 [SMILES: *C(=O)NO]

In certain embodiments, provided is a compound of any of Formulas (LVII-LXIV), or a pharmaceutically salt, tautomer, stereoisomer, and/or mixture of stereoisomers thereof, wherein R⁵ is

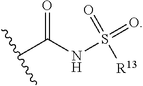

wherein R¹³ is —CH₃ or -cyclopropyl. In certain embodiments, R¹³ is —CH₃. In certain embodiments, R¹³ is -cyclopropyl.

In certain embodiments, provided is a compound of any of Formulas (LVII-LXIV), or a pharmaceutically salt, tautomer, stereoisomer, and/or mixture of stereoisomers thereof, wherein R⁵ is —H.

In certain embodiments, provided is a compound of any of Formulas (LVII-LXIV), or a pharmaceutically salt, tautomer, stereoisomer, and/or mixture of stereoisomers thereof, wherein R⁵ is —CH₂OH.

In certain embodiments, provided is a compound of any of Formulas (LVII-LXIV), or a pharmaceutically salt, tautomer, stereoisomer, and/or mixture of stereoisomers thereof, wherein R⁵ is

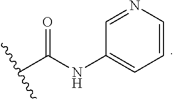 [SMILES: *C(=O)Nc1cccnc1]

In certain embodiments, when the compound is according to Formula (LVII), (LVIII), (LIX), (LX), or (LXII), L¹ is selected from the group consisting of —NH—, —N(Me)-, —N(Et)-, —OCH₂—, —OC(H,Me)-, —CH₂O—, —OCH₂(cyclopropyl)-, —O—CH₂—CH₂—, —CH₂—O—CH₂—, —CH₂—O—C((CH₃)₂), —NHCH₂—, —CH₂NH—, —N(Me)CH₂—, and —SO₂NH—.

In certain embodiments, provided is a compound of any of Formulas (LVII)-(LXIV), or a pharmaceutically salt, tautomer, stereoisomer, and/or mixture of stereoisomers thereof, wherein L² is —CH₂—, —C(O)—, or —SO₂—.

In certain embodiments, provided is a compound of any of Formulas (LVII)-(LXIV), or a pharmaceutically salt, tautomer, stereoisomer, and/or mixture of stereoisomers thereof, wherein L² is a bond, —C(O)—, —CH₂—, —C(H)═, —SO₂—, —O—, —CF₂—, —CHF—, or —CH(OH)—.

In certain embodiments, provided is a compound of any of Formulas (LVII)-(LXIV), or a pharmaceutically salt, tautomer, stereoisomer, and/or mixture of stereoisomers thereof, wherein L² is —O—.

In certain embodiments, provided is a compound of any of Formulas (LVII)-(LXIV), or a pharmaceutically salt, tautomer, stereoisomer, and/or mixture of stereoisomers thereof, wherein L² is —CH₂—.

In certain embodiments, provided is a compound of Formula (LXII)-(LXIV), or a pharmaceutically salt, tautomer, stereoisomer, and/or mixture of stereoisomers thereof, wherein L² is —O—.

In certain embodiments, provided is a compound of any of Formulas (LVII)-(LXIV), or a pharmaceutically salt, tautomer, stereoisomer, and/or mixture of stereoisomers thereof, wherein L³ is —C(O)—.

In certain embodiments, provided is a compound of any of Formulas (LVII)-(LXIV), or a pharmaceutically salt, tautomer, stereoisomer, and/or mixture of stereoisomers thereof, wherein L³ is —CH₂—.

In certain embodiments, provided is a compound of any of Formulas (LVII)-(LXIV), or a pharmaceutically salt, tautomer, stereoisomer, and/or mixture of stereoisomers thereof, wherein L³ is —CH(CH₃)—.

In certain embodiments, provided is a compound of any of Formulas (LVII)-(LXIV), or a pharmaceutically salt, tautomer, stereoisomer, and/or mixture of stereoisomers thereof, wherein L¹ is —CH₂O—; and L² is —CH₂— or —CO—.

In certain embodiments, provided is a compound of any of Formulas (LVII)-(LXIV), or a pharmaceutically salt, tautomer, stereoisomer, and/or mixture of stereoisomers thereof, wherein L¹ is —CH₂O—; and L³ is —C(O)—.

In certain embodiments, provided is a compound of any of Formulas (LVII)-(LXIV), or a pharmaceutically salt, tautomer, stereoisomer, and/or mixture of stereoisomers thereof, wherein L¹ is —OCH₂— and L² is —O—.

In certain embodiments, provided is a compound of any of Formulas (LVII)-(LXIV), or a pharmaceutically salt, tautomer, stereoisomer, and/or mixture of stereoisomers thereof, wherein L¹ is —CH₂O— and L² is —O—.

In certain embodiments, provided is a compound of any of Formulas (LVII)-(LXIV), or a pharmaceutically salt, tautomer, stereoisomer, and/or mixture of stereoisomers thereof, wherein L¹ is —CH₂O—; and L³ is —CH₂—.

In certain embodiments, provided is a compound of any of Formulas (LVII)-(LXIV), or a pharmaceutically salt, tautomer, stereoisomer, and/or mixture of stereoisomers thereof, wherein L¹ is —CH₂O—; and L³ is —CH(CH₃)—.

In certain embodiments, provided is a compound of any of Formulas (LVII)-(LXIV), or a pharmaceutically salt, tautomer, stereoisomer, and/or mixture of stereoisomers thereof, wherein L¹ is —O—CH₂—; L² is —O—; and L³ is —CH₂—.

In certain embodiments, provided is a compound of any of Formulas (LVII)-(LXIV), or a pharmaceutically salt, tautomer, stereoisomer, and/or mixture of stereoisomers thereof, wherein L¹ is —O—CH₂—; L² is —O—; and L³ is —CH(CH₃)—.

In certain embodiments, provided is a compound of any of Formula (LXIII), or a pharmaceutically salt, tautomer, stereoisomer, and/or mixture of stereoisomers thereof, wherein L¹ is —O—CH₂—; L² is —O—; and L³ is —CH₂—.

In certain embodiments, provided is a compound of Formula (LVII)-(LXIV), or a pharmaceutically salt, tautomer, stereoisomer, and/or mixture of stereoisomers thereof, wherein L¹ is —O—CH₂—.

In certain embodiments, provided is a compound of Formula (LVII)-(LXIV), or a pharmaceutically salt, tautomer, stereoisomer, and/or mixture of stereoisomers thereof, wherein L¹ is —O—.

In certain embodiments, provided is a compound of any of Formulas (LVII)-(LXIV), or a pharmaceutically salt, tautomer, stereoisomer, and/or mixture of stereoisomers thereof, wherein L¹ is —CH₂—.

In certain embodiments, provided is a compound of any of Formulas (LVII)-(LXIV), or a pharmaceutically salt, tautomer, stereoisomer, and/or mixture of stereoisomers thereof, wherein L¹ is —OCH₂—.

In certain embodiments, provided is a compound of any of Formulas (LVII)-(LXIV), or a pharmaceutically salt, tautomer, stereoisomer, and/or mixture of stereoisomers thereof, wherein L¹ is —CH₂O—.

In certain embodiments, provided is a compound of any of Formulas (LVII)-(LXIV), or a pharmaceutically salt, tautomer, stereoisomer, and/or mixture of stereoisomers thereof, wherein L¹ is —OCH₂(cyclopropyl).

In certain embodiments, provided is a compound of any of Formulas (LVII)-(LXIV), or a pharmaceutically salt, tautomer, stereoisomer, and/or mixture of stereoisomers thereof, wherein L¹ is —CH₂—O—CH₂—.

In certain embodiments, provided is a compound of any of Formulas (LVII)-(LXIV), or a pharmaceutically salt, tautomer, stereoisomer, and/or mixture of stereoisomers thereof, wherein L¹ is —NH—.

In certain embodiments, provided is a compound of any of Formulas (LVII)-(LXIV), or a pharmaceutically salt, tautomer, stereoisomer, and/or mixture of stereoisomers thereof, wherein L¹ is —N(Me)-.

In certain embodiments, provided is a compound of any of Formulas (LVII)-(LXIV), or a pharmaceutically salt, tautomer, stereoisomer, and/or mixture of stereoisomers thereof, wherein L¹ is —N(Et)-.

In certain embodiments, provided is a compound of any of Formulas (LVII)-(LXIV), a pharmaceutically salt, tautomer, stereoisomer, and/or mixture of stereoisomers thereof, wherein L¹ is —OC(H,Me)-.

In certain embodiments, provided is a compound of any of Formulas (LVII)-(LXIV), or a pharmaceutically salt, tautomer, stereoisomer, and/or mixture of stereoisomers thereof, wherein L¹ is —NHCH₂—.

In certain embodiments, provided is a compound of any of Formulas (LVII)-(LXIV), or a pharmaceutically salt, tautomer, stereoisomer, and/or mixture of stereoisomers thereof, wherein L¹ is —N(Me)CH₂—.

In certain embodiments, provided is a compound of any of Formulas (LVII)-(LXIV), or a pharmaceutically salt, tautomer, stereoisomer, and/or mixture of stereoisomers thereof, wherein L¹ is —SO₂NH—.

In certain embodiments, provided is a compound of any of Formulas (LVII)-(LXIV), or a pharmaceutically salt, tautomer, stereoisomer, and/or mixture of stereoisomers thereof, wherein L¹ is —CH₂NH—.

In certain embodiments of any of Formulas (LVII)-(LXIV), L¹ is —O—; and L² is —C(O)—. In certain embodiments of any of Formulas (LVII)-(LXIV), L¹ is —O—; and L² is —CH₂—. In certain embodiments of any of Formulas (LVII)-(LXIV), L¹ is —O—; and L² is —C(H)═. In certain embodiments of any of Formulas (LVII)-(LXIV), L¹ is —O—; and L² is —SO₂—. In certain embodiments of any of Formulas (LVII)-(LXIV), L¹ is —O—; and L² is —NH—. In certain embodiments of any of Formulas (LVII)-(LXIV), L¹ is —O—; and L² is —N(Me)-. In certain embodiments of any of Formulas (LVII)-(LXIV), L¹ is —O—; L² is —C(O)—; and L³ is —CH₂—. In certain embodiments of any of Formulas (LVII)-(LXIV), L¹ is —O—; L² is —CH₂—; and L³ is —CH₂—. In certain embodiments of any of Formulas (LVII)-(LXIV), L¹ is —O—; L² is —C(H)═; and L³ is —CH₂—. In certain embodiments of any of Formulas (LVII)-(LXIV), L¹ is —O—; L² is —SO₂—; and L³ is —CH₂—. In certain embodiments of any of Formulas (LVII)-(LXIV), L¹ is —O—; L² is —NH—; and L³ is —CH₂—. In certain embodiments of any of Formulas (LVII)-(LXIV), L¹ is —O—; L² is —N(Me)-; and L³ is —CH₂—. In certain embodiments of any of Formulas (LVII)-(LXIV), L¹ is —O—; L² is —C(O)—; L³ is —CH₂—; and R² and R³ are each H. In certain embodiments of any of Formulas ((LVII)-(LXIV), L¹ is —O—; L² is —CH₂—; L³ is —CH₂—; and R² and R³ are each H. In certain embodiments of any of Formulas (LVII)-(LXIV), L¹ is —O—; L² is —C(H)—; L³ is —CH₂—; and R² and R³ are each H. In certain embodiments of any of Formulas (LVII)-(LXIV), L¹ is —O—; L² is —SO₂—; L³ is —CH₂—; and R² and R³ are each H. In certain embodiments of any of Formulas (LVII)-(LXIV), L¹ is —O—; L² is —NH—; L³ is —CH₂—; and R² and R³ are each H. In certain embodiments of any of Formulas (LVII)-(LXIV), L¹ is —O—; L² is —N(Me)-; L³ is —CH₂—; and R² and R³ are each H. In certain embodiments of any of Formulas (LVII)-(LXIV), L¹ is —O—; L² is —C(O)—; L³ is —CH₂—; R² and R³ are each H; and R⁴ is (2S)-oxetan-2-yl-methyl. In certain embodiments of any of Formulas (LVII)-(LXIV), L¹ is —O—; L² is —CH₂—; L³ is —CH₂—; R² and R³ are each H; and R⁴ is (2S)-oxetan-2-yl-methyl. In certain embodiments of any of Formulas (LVII)-(LXIV), L¹ is —O—; L² is —C(H)═; L³ is —CH₂—; R² and R³ are each H; and R⁴ is (2S)-oxetan-2-yl-methyl. In certain embodiments of any of Formulas (LVII)-(LXIV), L¹ is —O—; L² is —SO₂—; L³ is —CH₂—; R² and R³ are each H; and R⁴ is (2S)-oxetan-2-yl-methyl. In certain embodiments of any of Formulas (LVII)-(LXIV), L¹ is —O—; L² is —NH—; L³ is —CH₂—; R² and R³ are each H; and R⁴ is (2S)-oxetan-2-yl-methyl. In certain embodiments of any of Formulas (LVII)-(LXIV), L¹ is —O—; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —O—; L² is —C(O)—; L³ is —CH₂—; R² and R³ are each H; and R⁴ is (1-ethyl-1H-imidazol-5-yl)-methyl. In certain embodiments of any of Formulas (LVII)-(LXIV), L¹ is —O—; L² is —CH₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —O—; L² is —C(H)═; L³ is —CH₂—; R² and R³ are each H; and R⁴ is (1-ethyl-1H-imidazol-5-yl)-methyl. In certain embodiments of any of Formulas (LVII)-(LXIV), L¹ is —O—; L² is —SO₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —O—; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —O—; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —O—; L² is —C(O)—; L³ is —CH₂—; R² and R³ are each H; R⁴ is (2S)-oxetan-2-yl-methyl; and R⁵ is —COOH. In certain embodiments of any of Formulas (LVII)-(LXIV), is —O—; L² is —CH₂—; L³ is —CH₂—; R² and R³ are each H; R⁴ is (2S)-oxetan-2-yl-methyl; and R⁵ is —COOH. In certain embodiments of any of Formulas (LVIII)-(LXII), L¹ is —O—; L² is —C(H)═; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —O—; L² is —SO₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —O—; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —O—; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —O—; L² is —C(O)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —O—; L² is —CH₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —O—; L² is —C(H)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —O—; L² is —SO₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —O—; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —O—; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —O—; L² is —C(O)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —CH₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —C(H)═; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —SO₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —C(O)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —O—; L² is —CH₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —O—; L² is —C(H)═; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —O—; L² is —SO₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —O—; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), is L¹-O—; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), is (1-methyl-1H-imidazol-5-yl)-methyl. In certain embodiments of any of Formulas ((LVII)-(LXII), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is in certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —O—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —CH₂O—; and L² is —C(O)—. In certain embodiments of any of Formulas (LVII)-(LXIV), L¹ is —CH₂O—; and L² is —CH₂—. In certain embodiments of any of Formulas (LVII)-(LXIV), L¹ is —CH₂O—; and L² is —C(H)═. In certain embodiments of any of Formulas (LVII)-(LXIV), L¹ is —CH₂O—; and L² is —SO₂—. In certain embodiments of any of Formulas (LVII)-(LXIV), L¹ is —CH₂O—; and L² is —NH—. In certain embodiments of any of Formulas (LVII)-(LXIV), L¹ is —CH₂O—; and L² is —N(Me)-. In certain embodiments of any of Formulas (LVII)-(LXIV), L¹ is —CH₂O—; L² is —C(O)—; and L³ is —CH₂—. In certain embodiments of any of Formulas (LVII)-(LXIV), L¹ is —CH₂O—; L² is —CH₂—; and L³ is —CH₂—. In certain embodiments of any of Formulas (LVII)-(LXIV), L¹ is —CH₂O—; L² is —C(H)═; and L³ is —CH₂—. In certain embodiments of any of Formulas (LVII)-(LXIV), L¹ is —CH₂O—; L² is —SO₂—; and L³ is —CH₂—. In certain embodiments of any of Formulas (LVII)-(LXIV), L¹ is —CH₂O—; L² is —NH—; and L³ is —CH₂—. In certain embodiments of any of Formulas (LVII)-(LXIV), L¹ is —CH₂O—; L² is —N(Me)-; and L³ is —CH₂—. In certain embodiments of any of Formulas (LVII)-(LXIV), L¹ is —CH₂O—; L² is —C(O)—; L³ is —CH₂—; and R² and R³ are each H. In certain embodiments of any of Formulas (LVII)-(LXIV), L¹ is —CH₂O—; L² is —CH₂—; L³ is —CH₂—; and R² and R³ are each H. In certain embodiments of any of Formulas (LVII)-(LXIV), L¹ is —CH₂O—; L² is —C(H)═; L³ is —CH₂—; and R² and R³ are each H. In certain embodiments of any of Formulas (LVII)-(LXIV), L¹ is —CH₂O—; L² is —SO₂—; L³ is —CH₂—; and R² and R³ are each H. In certain embodiments of any of Formulas (LVII)-(LXIV), L¹ is —CH₂O—; L² is —NH—; L³ is —CH₂—; and R² and R³ are each H. In certain embodiments of any of Formulas (LVII)-(LXIV), L¹ is —CH₂O—; L² is —N(Me)-; L³ is —CH₂—; and R² and R³ are each H. In certain embodiments of any of Formulas (LVII)-(LXIV), L¹ is —CH₂O—; L² is —C(O)—; L³ is —CH₂—; R² and R³ are each H; and R⁴ is (2S)-oxetan-2-yl-methyl. In certain embodiments of any of Formulas (LVII)-(LXIV), L¹ is —CH₂O—; L² is —CH₂—; L³ is —CH₂—; R² and R³ are each H; and R⁴ is (2S)-oxetan-2-yl-methyl. In certain embodiments of any of Formulas (LVII)-(LXIV), L¹ is —CH₂O—; L² is —C(H)═; L³ is —CH₂—; R² and R³ are each H; and R⁴ is (2S)-oxetan-2-yl-methyl. In certain embodiments of any of Formulas (LVII)-(LXIV), L¹ is —CH₂O—; L² is —SO₂—; L³ is —CH₂—; R² and R³ are each H; and R⁴ is (2S)-oxetan-2-yl-methyl. In certain embodiments of any of Formulas (LVII)-(LXIV), L¹ is —CH₂O—; L² is —NH—; L³ is —CH₂—; R² and R³ are each H; and R⁴ is (2S)-oxetan-2-yl-methyl. In certain embodiments of any of Formulas (LVII)-(LXIV), L¹ is —CH₂O—; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —CH₂O—; L² is —C(O)—; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂O—; L² is —CH₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂O—; L² is —C(H)═; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂O—; L² is —SO₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂O—; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂O—; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂O—; L² is —C(O)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂O—; L² is —CH₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂O—; L² is —C(H)═; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂O—; L² is —SO₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂O—; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂O—; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂O—; L² is —C(O)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂O—; L² is —CH₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂O—; L² is —C(H)═; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂O—; L² is —SO₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂O—; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂O—; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂O—; L² is —C(O)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CH₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —C(H)═; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —SO₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —NH—; L³ is —CH₂—; and R² and R³ are each H. In certain embodiments of any of Formulas (LVII)-(LXIV), L¹ is —CH₂O—; L² is —N(Me)-; L³ is —CH₂—; and R² and R³ are each H. In certain embodiments of any of Formulas (LVII)-(LXIV), L¹ is —CH₂O—; L² is —C(O)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CH₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —CH₂O—; L² is —NH—; L³ is —CH₂—; and R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —N(Me)-; L³ is —CH₂—; and R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —C(H)═; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —CH₂O—; L² is —SO₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is in certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —OCH₂—; and L² is —C(O)—. In certain embodiments of any of Formulas (LVII)-(LXIV), L¹ is —OCH₂—; and L² is —CH₂—. In certain embodiments of any of Formulas (LVII)-(LXIV), L¹ is —OCH₂—; and L² is —C(H)═. In certain embodiments of any of Formulas (LVII)-(LXIV), L¹ is —OCH₂—; and L² is —SO₂—. In certain embodiments of any of Formulas (LVII)-(LXIV), L¹ is —OCH₂—; and L² is —NH—. In certain embodiments of any of Formulas (LVII)-(LXIV), L¹ is —OCH₂—; and L² is —N(Me)-. In certain embodiments of any of Formulas (LVII)-(LXIV), L¹ is —OCH₂—; L² is —C(O)—; and L³ is —CH₂—. In certain embodiments of any of Formulas (LVII)-(LXIV), L¹ is —OCH₂—; L² is —CH₂—; and L³ is —CH₂—. In certain embodiments of any of Formulas (LVII)-(LXIV), L¹ is —OCH₂—; L² is —C(H)═; and L³ is —CH₂—. In certain embodiments of any of Formulas (LVII)-(LXIV), L¹ is —OCH₂—; L² is —SO₂—; and L³ is —CH₂—. In certain embodiments of any of Formulas (LVII)-(LXIV), L¹ is —OCH₂—; L² is —NH—; and L³ is —CH₂—. In certain embodiments of any of Formulas (LVII)-(LXIV), L¹ is —OCH₂—; L² is —N(Me)-; and L³ is —CH₂—. In certain embodiments of any of Formulas (LVII)-(LXIV), L¹ is —OCH₂—; L² is —C(O)—; L³ is —CH₂—; and R² and R³ are each H. In certain embodiments of any of Formulas (LVII)-(LXIV), L¹ is —OCH₂—; L² is —CH₂—; L³ is —CH₂—; and R² and R³ are each H. In certain embodiments of any of Formulas (LVII)-(LXIV), L¹ is —OCH₂—; L² is —C(H)═; L³ is —CH₂—; and R² and R³ are each H. In certain embodiments of any of Formulas (LVII)-(LXIV), L¹ is —OCH₂—; L² is —SO₂—; L³ is —CH₂—; and R² and R³ are each H. In certain embodiments of any of Formulas (LVII)-(LXIV), L¹ is —OCH₂—; L² is —NH—; L³ is —CH₂—; and R² and R³ are each H. In certain embodiments of any of Formulas (LVII)-(LXIV), L¹ is —OCH₂—; L² is —N(Me)-; L³ is —CH₂—; and R² and R³ are each H. In certain embodiments of any of Formulas (LVII)-(LXIV), L¹ is —OCH₂—; L² is —C(O)—; L³ is —CH₂—; R² and R³ are each H; and R⁴ is (2S)-oxetan-2-yl-methyl. In certain embodiments of any of Formulas (LVII)-(LXIV), L¹ is —OCH₂—; L² is —CH₂—; L³ is —CH₂—; R² and R³ are each H; and R⁴ is (2S)-oxetan-2-yl-methyl. In certain embodiments of any of Formulas (LVII)-(LXIV), L¹ is —OCH₂—; L² is —C(H)═; L³ is —CH₂—; R² and R³ are each H; and R⁴ is (2S)-oxetan-2-yl-methyl. In certain embodiments of any of Formulas (LVII)-(LXIV), L¹ is —OCH₂—; L² is —SO₂—; L³ is —CH₂—; R² and R³ are each H; and R⁴ is (2S)-oxetan-2-yl-methyl. In certain embodiments of any of Formulas (LVII)-(LXIV), L¹ is —OCH₂—; L² is —NH—; L³ is —CH₂—; R² and R³ are each H; and R⁴ is (2S)-oxetan-2-yl-methyl. In certain embodiments of any of Formulas (LVII)-(LXIV), L¹ is —OCH₂—; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —OCH₂—; L² is —C(O)—; L³ is —CH₂—; R² and R³ are each H; and R⁴ is (1-ethyl-1H-imidazol-5-yl)-methyl. In certain embodiments of any of Formulas (LVII)-(LXIV), L¹ is —OCH₂—; L² is —CH₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —OCH₂—; L² is —C(H)═; L³ is —CH₂—; R² and R³ are each H; and R⁴ is (1-ethyl-1H-imidazol-5-yl)-methyl. In certain embodiments of any of Formulas (LVII)-(LXIV), L¹ is —OCH₂—; L² is —SO₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —OCH₂—; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —OCH₂—; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —OCH₂—; L² is —C(O)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OCH₂—; L² is —CH₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OCH₂—; L² is —C(H)═; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OCH₂—; L² is —SO₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OCH₂—; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OCH₂—; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OCH₂—; L² is —C(O)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OCH₂—; L² is —CH₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OCH₂—; L² is —C(H)═; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OCH₂—; L² is —SO₂—; L³ is —CH₂—; R² and R³ are each H; R⁴ is (1-ethyl-1H-imidazol-5-yl)-methyl; and R⁵ is —COOH. In certain embodiments of any of (LVII)-(LXIV), L¹ is —OCH₂—; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OCH₂—; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OCH₂—; L² is —C(O)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CH₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —C(H)═; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —SO₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —C(O)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CH₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OCH₂—; L² is —C(H)═; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OCH₂—; L² is —SO₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OCH₂—; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OCH₂—; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is in certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is(S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —NH— and L² is —C(O)—. In certain embodiments of any of Formulas (LVII)-(LXIV), L¹ is —NH— and L² is —CH₂—. In certain embodiments of any of Formulas (LVII)-(LXIV), L¹ is —NH—; and L² is —C(H)—. In certain embodiments of any of Formulas (LVII)-(LXIV), L¹ is —NH—; and L² is —SO₂—. In certain embodiments of any of Formulas (LVII)-(LXIV), L¹ is —NH—; and L² is —NH—. In certain embodiments of any of Formulas (LVII)-(LXIV), L¹ is —NH—; and L² is —N(Me)-. In certain embodiments of any of Formulas (LVII)-(LXIV), L¹ is —NH—; L² is —C(O)—; and L³ is —CH₂—. In certain embodiments of any of Formulas (LVII)-(LXIV), L¹ is —NH—; L² is —CH₂—; and L³ is —CH₂—. In certain embodiments of any of Formulas (LVII)-(LXIV), L¹ is —NH—; L² is —C(H)═; and L³ is —CH₂—. In certain embodiments of any of Formulas (LVII)-(LXIV), L¹ is —NH—; L² is —SO₂—; and L³ is —CH₂—. In certain embodiments of any of Formulas (LVII)-(LXIV), L¹ is —NH—; L² is —NH—; and L³ is —CH₂—. In certain embodiments of any of Formulas (LVII)-(LXIV), L¹ is —NH—; L² is —N(Me)-; and L³ is —CH₂—. In certain embodiments of any of Formulas (LVII)-(LXIV), L¹ is —NH—; L² is —C(O)—; L³ is —CH₂—; and R² and R³ are each H. In certain embodiments of any of Formulas (LVII)-(LXIV), L¹ is —NH—; L² is —CH₂—; L³ is —CH₂—; and R² and R³ are each H. In certain embodiments of any of Formulas (LVII)-(LXIV), L¹ is —NH—; L² is —C(H)═; L³ is —CH₂—; and R² and R³ are each H. In certain embodiments of any of Formulas (LVII)-(LXIV), L¹ is —NH—; L² is —SO₂—; L³ is —CH₂—; and R² and R³ are each H. In certain embodiments of any of Formulas (LVII)-(LXIV), L¹ is —NH—; L² is —NH—; L³ is —CH₂—; and R² and R³ are each H. In certain embodiments of any of Formulas (LVII)-(LXIV), L¹ is —NH—; L² is —N(Me)-; L³ is —CH₂—; and R² and R³ are each H. In certain embodiments of any of Formulas (LVII)-(LXIV), L¹ is —NH—; L² is —C(O)—; L³ is —CH₂—; R² and R³ are each H; and R⁴ is (2S)-oxetan-2-yl-methyl. In certain embodiments of any of Formulas (LVII)-(LXIV), L¹ is —NH—; L² is —CH₂—; L³ is —CH₂—; R² and R³ are each H; and R⁴ is (2S)-oxetan-2-yl-methyl. In certain embodiments of any of Formulas (LVII)-(LXIV), L¹ is —NH—; L² is —C(H)—; L³ is —CH₂—; R² and R³ are each H; and R⁴ is (2S)-oxetan-2-yl-methyl. In certain embodiments of any of Formulas (LVII)-(LXIV), L¹ is —NH—; L² is —SO₂—; L³ is —CH₂—; R² and R³ are each H; and R⁴ is (2S)-oxetan-2-yl-methyl. In certain embodiments of any of Formulas (LVII)-(LXIV), L¹ is —NH—; L² is —NH—; L³ is —CH₂—; R² and R³ are each H; and R⁴ is (2S)-oxetan-2-yl-methyl. In certain embodiments of any of Formulas (LVII)-(LXIV), L¹ is —NH—; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —NH—; L² is —C(O)—; L³ is —CH₂—; R² and R³ are each H; and R⁴ is (1-ethyl-1H-imidazol-5-yl)-methyl. In certain embodiments of any of Formulas (LVII)-(LXIV), L¹ is —NH—; L² is —CH₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —NH—; L² is —C(H)═; L³ is —CH₂—; R² and R³ are each H; and R⁴ is (1-ethyl-1H-imidazol-5-yl)-methyl. In certain embodiments of any of Formulas (LVII)-(LXIV), L¹ is —NH—; L² is —SO₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —NH—; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —NH—; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —NH—; L² is —C(O)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NH—; L² is —CH₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NH—; L² is —C(H)═; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NH—; L² is —SO₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NH—; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NH—; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NH—; L² is —C(O)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NH—; L² is —CH₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NH—; L² is —C(H)═; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NH—; L² is —SO₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NH—; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NH—; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NH—; L² is —C(O)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —CH₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —C(H)═; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —SO₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —C(O)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NH—; L² is —CH₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NH—; L² is —C(H)═; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NH—; L² is —SO₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NH—; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NH—; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is in certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —N(Me)- and L² is —C(O)—. In certain embodiments of any of Formulas (LVII)-(LXIV), L¹ is —N(Me)- and L² is —CH₂—. In certain embodiments of any of Formulas (LVII)-(LXIV), L¹ is —N(Me)- and L² is —SO₂—. In certain embodiments of any of Formulas (LVII)-(LXIV), L¹ is —N(Me)-; and L² is —NH—. In certain embodiments of any of Formulas (LVII)-(LXIV), L¹ is —N(Me)-; and L² is —N(Me)-. In certain embodiments of any of Formulas (LVII)-(LXIV), L¹ is —N(Me)-; L² is —C(O)—; and L³ is —CH₂—. In certain embodiments of any of Formulas (LVII)-(LXIV), L¹ is —N(Me)-; L² is —CH₂—; and L³ is —CH₂—. In certain embodiments of any of Formulas (LVII)-(LXIV), L¹ is —N(Me)-; L² is —C(H)═; and L³ is —CH₂—. In certain embodiments of any of Formulas (LVII)-(LXIV), L¹ is —N(Me)-; L² is —SO₂—; and L³ is —CH₂—. In certain embodiments of any of Formulas (LVII)-(LXIV), L¹ is —N(Me)-; L² is —NH—; and L³ is —CH₂—. In certain embodiments of any of Formulas (LVII)-(LXIV), L¹ is —N(Me)-; L² is —N(Me)-; and L³ is —CH₂—. In certain embodiments of any of Formulas (LVII)-(LXIV), L¹ is —N(Me)-; L² is —C(O)—; L³ is —CH₂—; and R² and R³ are each H. In certain embodiments of any of Formulas (LVII)-(LXIV), L¹ is —N(Me)-; L² is —CH₂—; L³ is —CH₂—; and R² and R³ are each H. In certain embodiments of any of Formulas (LVII)-(LXIV), L¹ is —N(Me)-; L² is —C(H)═; L³ is —CH₂—; and R² and R³ are each H. In certain embodiments of any of Formulas (LVII)-(LXIV), L¹ is —N(Me)-; L² is —SO₂—; L³ is —CH₂—; and R² and R³ are each H. In certain embodiments of any of (LVII)-(LXIV), L¹ is —N(Me)-; L² is —NH—; L³ is —CH₂—; and R² and R³ are each H. In certain embodiments of any of Formulas (LVII)-(LXIV), L¹ is —N(Me)-; L² is —N(Me)-; L³ is —CH₂—; and R² and R³ are each H. In certain embodiments of any of Formulas (LVII)-(LXIV), L¹ is —N(Me)-; L² is —C(O)—; L³ is —CH₂—; R² and R³ are each H; and R⁴ is (2S)-oxetan-2-yl-methyl. In certain embodiments of any of Formulas (LVII)-(LXIV), L¹ is —N(Me)-; L² is —CH₂—; L³ is —CH₂—; R² and R³ are each H; and R⁴ is (2S)-oxetan-2-yl-methyl. In certain embodiments of any of Formulas (LVII)-(LXIV), L¹ is —N(Me)-; L² is —C(H)═; L³ is —CH₂—; R² and R³ are each H; and R⁴ is (2S)-oxetan-2-yl-methyl. In certain embodiments of any of Formulas (LVII)-(LXIV), L¹ is —N(Me)-; L² is —SO₂—; L³ is —CH₂—; R² and R³ are each H; and R⁴ is (2S)-oxetan-2-yl-methyl. In certain embodiments of any of Formulas (LVII)-(LXIV), L¹ is —N(Me)-; L² is —NH—; L³ is —CH₂—; R² and R³ are each H; and R⁴ is (2S)-oxetan-2-yl-methyl. In certain embodiments of any of Formulas (LVII)-(LXIV), L¹ is —N(Me)-; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —N(Me)-; L² is —C(O)—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)-; L² is —CH₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)-; L² is —C(H)═; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)-; L² is —SO₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)-; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)-; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)-; L² is —C(O)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)-; L² is —CH₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)-; L² is —C(H)═; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)-; L² is —SO₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)-; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)-; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)-; L² is —C(O)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)-; L² is —CH₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)-; L² is —C(H)═; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)-; L² is —SO₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)-; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)-; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)-; L² is —C(O)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CH₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —C(H)═; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —SO₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), is —N(Me)-; L² is —C(O)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CH₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)-; L² is —C(H)═; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)-; L² is —SO₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)-; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)-; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is in certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —N(Et)-; and L² is —C(O)—. In certain embodiments of any of Formulas (LVII)-(LXIII)), L¹ is —N(Et)-; and L² is —CH₂—. In certain embodiments of any of Formulas (LVII)-(LXIV), L¹ is —N(Et)-; and L² is —C(H)═. In certain embodiments of any of Formulas (LVII)-(LXIV), L¹ is —N(Et)-; and L² is —SO₂—. In certain embodiments of any of Formulas (LVII)-(LXIV), is —N(Et)-; and L² is —NH—. In certain embodiments of any of Formulas (LVII)-(LXIV), L¹ is —N(Et)-; and L² is —N(Me)-. In certain embodiments of any of Formulas (LVII)-(LXIV), L¹ is —N(Et)-; L² is —C(O)—; and L³ is —CH₂—. In certain embodiments of any of Formulas (LVII)-(LXIV), L¹ is —N(Et)-; L² is —CH₂—; and L³ is —CH₂—. In certain embodiments of any of Formulas (LVII)-(LXIV), is —N(Et)-; L² is —C(H)═; and L³ is —CH₂—. In certain embodiments of any of Formulas (LVII)-(LXIV), L¹ is —N(Et)-; L² is —SO₂—; and L³ is —CH₂—. In certain embodiments of any of Formulas (LVII)-(LXIV), is —N(Et)-; L² is —NH—; and L³ is —CH₂—. In certain embodiments of any of Formulas (LVII)-(LXIV), L¹ is —N(Et)-; L² is —N(Me)-; and L³ is —CH₂—. In certain embodiments of any of Formulas (LVII)-(LXIV), L¹ is —N(Et)-; L² is —C(O)—; L³ is —CH₂—; and R² and R³ are each H. In certain embodiments of any of Formulas (LVII)-(LXIV), L¹ is —N(Et)-; L² is —CH₂—; L³ is —CH₂—; and R² and R³ are each H. In certain embodiments of any of Formulas (LVII)-(LXIII)), L¹ is —N(Et)-; L² is —C(H)═; L³ is —CH₂—; and R² and R³ are each H. In certain embodiments of any of Formulas (LVII)-(LXIV), L¹ is —N(Et)-; L² is —SO₂—; L³ is —CH₂—; and R² and R³ are each H. In certain embodiments of any of Formulas (LVII)-(LXIV), L¹ is —N(Et)-; L² is —NH—; L³ is —CH₂—; and R² and R³ are each H. In certain embodiments of any of Formulas (LVII)-(LXIV), L¹ is —N(Et)-; L² is —N(Me)-; L³ is —CH₂—; and R² and R³ are each H. In certain embodiments of any of Formulas (LVII)-(LXIV), L¹ is —N(Et)-; L² is —C(O)—; L³ is —CH₂—; R² and R³ are each H; and R⁴ is (2S)-oxetan-2-yl-methyl. In certain embodiments of any of Formulas (LVII)-(LXIV), L¹ is —N(Et)-; L² is —CH₂—; L³ is —CH₂—; R² and R³ are each H; and R⁴ is (2S)-oxetan-2-yl-methyl. In certain embodiments of any of Formulas (LVII)-(LXIV), L¹ is —N(Et)-; L² is —C(H)═; L³ is —CH₂—; R² and R³ are each H; and R⁴ is (2S)-oxetan-2-yl-methyl. In certain embodiments of any of Formulas (LVII)-(LXIV), L¹ is —N(Et)-; L² is —SO₂—; L³ is —CH₂—; R² and R³ are each H; and R⁴ is (2S)-oxetan-2-yl-methyl. In certain embodiments of any of Formulas (LVII)-(LXIV), L¹ is —N(Et)-; L² is —NH—; L³ is —CH₂—; R² and R³ are each H; and R⁴ is (2S)-oxetan-2-yl-methyl. In certain embodiments of any of Formulas (LVII)-(LXIV), L¹ is —N(Et)-; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —N(Et)-; L² is —C(O)—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Et)-; L² is —CH₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Et)-; L² is —C(H)═; L³ is —CH₂—; R² and R³ are each H; and R⁴ is (1-ethyl-1H-imidazol-5-yl)-methyl. In certain embodiments of any of Formulas (LVII)-(LXIV), is —N(Et)-; L² is —SO₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Et)-; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Et)-; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Et)-; L² is —C(O)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Et)-; L² is —CH₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Et)-; L² is —C(H)═; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Et)-; L² is —SO₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Et)-; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Et)-; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Et)-; L² is —C(O)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Et)-; L² is —CH₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Et)-; L² is —C(H)═; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Et)-; L² is —SO₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Et)-; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Et)-; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Et)-; L² is —C(O)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —CH₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —C(H)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —SO₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —C(O)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), is —N(Et)-; L² is —CH₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Et)-; L² is —C(H)═; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Et)-; L² is —SO₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Et)-; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Et)-; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is in certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), a pharmaceutically salt, tautomer, stereoisomer, and/or mixture of stereoisomers thereof, wherein L² is —N(Me)-.

In certain embodiments of any of Formulas (LVII)-(LXIV), L¹ is —NHCH₂—; and L² is —C(O)—. In certain embodiments of any of Formulas (LVII)-(LXIV), L¹ is —NHCH₂—; and L² is —CH₂—. In certain embodiments of any of Formulas (LVII)-(LXIV), L¹ is —NHCH₂—; and L² is —C(H)═. In certain embodiments of any of Formulas (LVII)-(LXIV), L¹ is —NHCH₂—; and L² is —SO₂—. In certain embodiments of any of Formulas (LVII)-(LXIV), L¹ is —NHCH₂—; and L² is —NH—. In certain embodiments of any of Formulas (LVII)-(LXIV), is —NHCH₂—; and L² is —N(Me)-. In certain embodiments of any of Formulas (LVII)-(LXIV), L¹ is —NHCH₂—; L² is —C(O)—; and L³ is —CH₂—. In certain embodiments of any of Formulas (LVII)-(LXIV), L¹ is —NHCH₂—; L² is —CH₂—; and L³ is —CH₂—. In certain embodiments of any of Formulas (LVII)-(LXIV), is —NHCH₂—; L² is —C(H)═; and L³ is —CH₂—. In certain embodiments of any of Formulas (LVII)-(LXIV), L¹ is —NHCH₂—; L² is —SO₂—; and L³ is —CH₂—. In certain embodiments of any of Formulas (LVII)-(LXIV), L¹ is —NHCH₂—; L² is —NH—; and L³ is —CH₂—. In certain embodiments of any of Formulas (LVII)-(LXIV), L¹ is —NHCH₂—; L² is —N(Me)-; and L³ is —CH₂—. In certain embodiments of any of Formulas (LVII)-(LXIV), L¹ is —NHCH₂—; L² is —C(O)—; L³ is —CH₂—; and R² and R³ are each H. In certain embodiments of any of Formulas (LVII)-(LXIV), L¹ is —NHCH₂—; L² is —CH₂—; L³ is —CH₂—; and R² and R³ are each H. In certain embodiments of any of Formulas (LVII)-(LXIV), L¹ is —NHCH₂—; L² is —C(H)═; L³ is —CH₂—; and R² and R³ are each H. In certain embodiments of any of Formulas (LVII)-(LXIV), L¹ is —NHCH₂—; L² is —SO₂—; L³ is —CH₂—; and R² and R³ are each H. In certain embodiments of any of Formulas (LVII)-(LXIV), L¹ is —NHCH₂—; L² is —NH—; L³ is —CH₂—; and R² and R³ are each H. In certain embodiments of any of Formulas (LVII)-(LXIV), L¹ is —NHCH₂—; L² is —N(Me)-; L³ is —CH₂—; and R² and R³ are each H. In certain embodiments of any of Formulas (LVII)-(LXIV), L¹ is —NHCH₂—; L² is —C(O)—; L³ is —CH₂—; R² and R³ are each H; and R⁴ is (2S)-oxetan-2-yl-methyl. In certain embodiments of any of Formulas (LVII)-(LXIV), L¹ is —NHCH₂—; L² is —CH₂—; L³ is —CH₂—; R² and R³ are each H; and R⁴ is (2S)-oxetan-2-yl-methyl. In certain embodiments of any of Formulas (LVII)-(LXIV), L¹ is —NHCH₂—; L² is —C(H)═; L³ is —CH₂—; R² and R³ are each H; and R⁴ is (2S)-oxetan-2-yl-methyl. In certain embodiments of any of Formulas (LVII)-(LXIV), L¹ is —NHCH₂—; L² is —SO₂—; L³ is —CH₂—; R² and R³ are each H; and R⁴ is (2S)-oxetan-2-yl-methyl. In certain embodiments of any of Formulas (LVII)-(LXIV), L¹ is —NHCH₂—; L² is —NH—; L³ is —CH₂—; R² and R³ are each H; and R⁴ is (2S)-oxetan-2-yl-methyl. In certain embodiments of any of Formulas (LVII)-(LXIV), L¹ is —NHCH₂—; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —NHCH₂—; L² is —C(O)—; L³ is —CH₂—; R² and R³ are each H; and R⁴ is (1-ethyl-1H-imidazol-5-yl)-methyl. In certain embodiments of any of Formulas (LVII)-(LXIV), L¹ is —NHCH₂—; L² is —CH₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —NHCH₂—; L² is —C(H)═; L³ is —CH₂—; R² and R³ are each H; and R⁴ is (1-ethyl-1H-imidazol-5-yl)-methyl. In certain embodiments of any of Formulas (LVII)-(LXIV), L¹ is —NHCH₂—; L² is —SO₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —NHCH₂—; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —NHCH₂—; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —NHCH₂—; L² is —C(O)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NHCH₂—; L² is —CH₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NHCH₂—; L² is —C(H)═; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NHCH₂—; L² is —SO₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NHCH₂—; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NHCH₂—; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NHCH₂—; L² is —C(O)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NHCH₂—; L² is —CH₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NHCH₂—; L² is —C(H)═; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NHCH₂—; L² is —SO₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NHCH₂—; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NHCH₂—; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NHCH₂—; L² is —C(O)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CH₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —C(H)═; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —SO₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —C(O)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CH₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —C(H)═; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —SO₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas ((LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas ((LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas ((LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is in certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —CH₂NH—; and L² is —C(O)—. In certain embodiments of any of Formulas (LVII)-(LXIV), L¹ is —CH₂NH—; and L² is —CH₂—. In certain embodiments of any of Formulas (LVII)-(LXIV), L¹ is —CH₂NH—; and L² is —C(H)═. In certain embodiments of any of Formulas (LVII)-(LXIV), L¹ is —CH₂NH—; and L² is —SO₂—. In certain embodiments of any of Formulas (LVII)-(LXIV), L¹ is —CH₂NH—; and L² is —NH—. In certain embodiments of any of Formulas (LVII)-(LXIV), is —CH₂NH—; and L² is —N(Me)-. In certain embodiments of any of Formulas (LVII)-(LXIV), L¹ is —CH₂NH—; L² is —C(O)—; and L³ is —CH₂—. In certain embodiments of any of Formulas (LVII)-(LXIV), L¹ is —CH₂NH—; L² is —CH₂—; and L³ is —CH₂—. In certain embodiments of any of Formulas (LVII)-(LXIV), is —CH₂NH—; L² is —C(H)═; and L³ is —CH₂—. In certain embodiments of any of Formulas (LVII)-(LXIV), L¹ is —CH₂NH—; L² is —SO₂—; and L³ is —CH₂—. In certain embodiments of any of Formulas (LVII)-(LXIV), L¹ is —CH₂NH—; L² is —NH—; and L³ is —CH₂—. In certain embodiments of any of Formulas (LVII)-(LXIV), L¹ is —CH₂NH—; L² is —N(Me)-; and L³ is —CH₂—. In certain embodiments of any of Formulas (LVII)-(LXIV), L¹ is —CH₂NH—; L² is —C(O)—; L³ is —CH₂—; and R² and R³ are each H. In certain embodiments of any of Formulas (LVII)-(LXIV), L¹ is —CH₂NH—; L² is —CH₂—; L³ is —CH₂—; and R² and R³ are each H. In certain embodiments of any of Formulas (LVII)-(LXIV), L¹ is —CH₂NH—; L² is —C(H)═; L³ is —CH₂—; and R² and R³ are each H. In certain embodiments of any of Formulas (LVII)-(LXIV), L¹ is —CH₂NH—; L² is —SO₂—; L³ is —CH₂—; and R² and R³ are each H. In certain embodiments of any of Formulas (LVII)-(LXIV), L¹ is —CH₂NH—; L² is —NH—; L³ is —CH₂—; and R² and R³ are each H. In certain embodiments of any of Formulas (LVII)-(LXIV), L¹ is —CH₂NH—; L² is —N(Me)-; L³ is —CH₂—; and R² and R³ are each H. In certain embodiments of any of Formulas (LVII)-(LXIV), L¹ is —CH₂NH—; L² is —C(O)—; L³ is —CH₂—; R² and R³ are each H; and R⁴ is (2S)-oxetan-2-yl-methyl. In certain embodiments of any of Formulas (LVII)-(LXIV), L¹ is —CH₂NH—; L² is —CH₂—; L³ is —CH₂—; R² and R³ are each H; and R⁴ is (2S)-oxetan-2-yl-methyl. In certain embodiments of any of Formulas (LVII)-(LXIV), L¹ is —CH₂NH—; L² is —C(H)═; L³ is —CH₂—; R² and R³ are each H; and R⁴ is (2S)-oxetan-2-yl-methyl. In certain embodiments of any of Formulas (LVII)-(LXIV), L¹ is —CH₂NH—; L² is —SO₂—; L³ is —CH₂—; R² and R³ are each H; and R⁴ is (2S)-oxetan-2-yl-methyl. In certain embodiments of any of Formulas (LVII)-(LXIV), L¹ is —CH₂NH—; L² is —NH—; L³ is —CH₂—; R² and R³ are each H; and R⁴ is (2S)-oxetan-2-yl-methyl. In certain embodiments of any of Formulas (LVII)-(LXIV), L¹ is —CH₂NH—; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —CH₂NH—; L² is —C(O)—; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂NH—; L² is —CH₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂NH—; L² is —C(H)═; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂NH—; L² is —SO₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂NH—; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂NH—; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂NH—; L² is —C(O)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂NH—; L² is —CH₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂NH—; L² is —C(H)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂NH—; L² is —SO₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂NH—; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂NH—; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂NH—; L² is —C(O)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂NH—; L² is —CH₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂NH—; L² is —C(H)═; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂NH—; L² is —SO₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂NH—; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂NH—; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂NH—; L² is —C(O)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —CH₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —C(H)═; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —SO₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —C(O)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹-CH₂NH—; L² is —CH₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —C(H)═; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —SO₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas ((LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas ((LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas ((LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is in certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —N(Me)CH₂—; and L² is —C(O)—. In certain embodiments of any of Formulas (LVII)-(LXIV), L¹ is —N(Me)CH₂—; and L² is —CH₂—. In certain embodiments of any of Formulas (LVII)-(LXIV), L¹ is —N(Me)CH₂—; and L² is —C(H)═. In certain embodiments of any of Formulas (LVII)-(LXIV), L¹ is —N(Me)CH₂—; and L² is —SO₂—. In certain embodiments of any of Formulas (LVII)-(LXIV), L¹ is —N(Me)CH₂—; and L² is —NH—. In certain embodiments of any of Formulas (LVII)-(LXIV), L¹ is —N(Me)CH₂—; and L² is —N(Me)-. In certain embodiments of any of Formulas (LVII)-(LXIV), L¹ is —N(Me)CH₂—; L² is —C(O)—; and L³ is —CH₂—. In certain embodiments of any of Formulas (LVII)-(LXIV), L¹ is —N(Me)CH₂—; L² is —CH₂—; and L³ is —CH₂—. In certain embodiments of any of Formulas (LVII)-(LXIV), L¹ is —N(Me)CH₂—; L² is —C(H)═; and L³ is —CH₂—. In certain embodiments of any of Formulas (LVII)-(LXIV), L¹ is —N(Me)CH₂—; L² is —SO₂—; and L³ is —CH₂—. In certain embodiments of any of Formulas (LVII)-(LXIV), L¹ is —N(Me)CH₂—; L² is —NH—; and L³ is —CH₂—. In certain embodiments of any of Formulas (LVII)-(LXIV), L¹ is —N(Me)CH₂—; L² is —N(Me)-; and L³ is —CH₂—. In certain embodiments of any of Formulas (LVII)-(LXIV), L¹ is —N(Me)CH₂—; L² is —C(O)—; L³ is —CH₂—; and R² and R³ are each H. In certain embodiments of any of Formulas (LVII)-(LXIV), L¹ is —N(Me)CH₂—; L² is —CH₂—; L³ is —CH₂—; and R² and R³ are each H. In certain embodiments of any of Formulas (LVII)-(LXIV), L¹ is —N(Me)CH₂—; L² is —C(H)═; L³ is —CH₂—; and R² and R³ are each H. In certain embodiments of any of Formulas (LVII)-(LXIV), L¹ is —N(Me)CH₂—; L² is —SO₂—; L³ is —CH₂—; and R² and R³ are each H. In certain embodiments of any of Formulas (LVII)-(LXIV), L¹ is —N(Me)CH₂—; L² is —NH—; L³ is —CH₂—; and R² and R³ are each H. In certain embodiments of any of Formulas (LVII)-(LXIV), L¹ is —N(Me)CH₂—; L² is —N(Me)-; L³ is —CH₂—; and R² and R³ are each H. In certain embodiments of any of Formulas (LVII)-(LXIV), L¹ is —N(Me)CH₂—; L² is —C(O)—; L³ is —CH₂—; R² and R³ are each H; and R⁴ is (2S)-oxetan-2-yl-methyl. In certain embodiments of any of Formulas (LVII)-(LXIV), L¹ is —N(Me)CH₂—; L² is —CH₂—; L³ is —CH₂—; R² and R³ are each H; and R⁴ is (2S)-oxetan-2-yl-methyl. In certain embodiments of any of Formulas (LVII)-(LXIV), L¹ is —N(Me)CH₂—; L² is —C(H)═; L³ is —CH₂—; R² and R³ are each H; and R⁴ is (2S)-oxetan-2-yl-methyl. In certain embodiments of any of Formulas (LVII)-(LXIV), L¹ is —N(Me)CH₂—; L² is —SO₂—; L³ is —CH₂—; R² and R³ are each H; and R⁴ is (2S)-oxetan-2-yl-methyl. In certain embodiments of any of Formulas (LVII)-(LXIV), L¹ is —N(Me)CH₂—; L² is —NH—; L³ is —CH₂—; R² and R³ are each H; and R⁴ is (2S)-oxetan-2-yl-methyl. In certain embodiments of any of Formulas (LVII)-(LXIV), L¹ is —N(Me)CH₂—; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —N(Me)CH₂—; L² is —C(O)—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)CH₂—; L² is —CH₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)CH₂—; L² is —C(H)═; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)CH₂—; L² is —SO₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)CH₂—; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)CH₂—; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)CH₂—; L² is —C(O)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)CH₂—; L² is —CH₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)CH₂—; L² is —C(H)═; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)CH₂—; L² is —SO₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)CH₂—; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)CH₂—; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)CH₂—; L² is —C(O)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)CH₂—; L² is —CH₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)CH₂—; L² is —C(H)═; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)CH₂—; L² is —SO₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)CH₂—; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)CH₂—; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)CH₂—; L² is —C(O)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CH₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —C(H)═; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —SO₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —C(O)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CH₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —C(H)═; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —SO₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —OC(H,Me)-; and L² is —C(O)—. In certain embodiments of any of Formulas (LVII)-(LXIV), L¹ is —OC(H,Me)-; and L² is —CH₂—. In certain embodiments of any of Formulas (LVII)-(LXIV), L¹ is —OC(H,Me)-; and L² is —C(H)—. In certain embodiments of any of Formulas (LVII)-(LXIV), L¹ is —OC(H,Me)-; and L² is —SO₂—. In certain embodiments of any of Formulas (LVII)-(LXIV), L¹ is —OC(H,Me)-; and L² is —NH—. In certain embodiments of any of Formulas (LVII)-(LXIV), L¹ is —OC(H,Me)-; and L² is —N(Me)-. In certain embodiments of any of Formulas (LVII)-(LXIV), L¹ is —OC(H,Me)-; L² is —C(O)—; and L³ is —CH₂—. In certain embodiments of any of Formulas (LVII)-(LXIV), L¹ is —OC(H,Me)-; L² is —CH₂—; and L³ is —CH₂—. In certain embodiments of any of Formulas (LVII)-(LXIV), L¹ is —OC(H,Me)-; L² is —C(H)═; and L³ is —CH₂—. In certain embodiments of any of Formulas (LVII)-(LXIV), L¹ is —OC(H,Me)-; L² is —SO₂—; and L³ is —CH₂—. In certain embodiments of any of Formulas (LVII)-(LXIV), L¹ is —OC(H,Me)-; L² is —NH—; and L³ is —CH₂—. In certain embodiments of any of Formulas (LVII)-(LXIV), L¹ is —OC(H,Me)-; L² is —N(Me)-; and L³ is —CH₂—. In certain embodiments of any of Formulas (LVII)-(LXIV), L¹ is —OC(H,Me)-; L² is —C(O)—; L³ is —CH₂—; and R² and R³ are each H. In certain embodiments of any of Formulas (LVII)-(LXIV), L¹ is —OC(H,Me)-; L² is —CH₂—; L³ is —CH₂—; and R² and R³ are each H. In certain embodiments of any of Formulas (LVII)-(LXIV), L¹ is —OC(H,Me)-; L² is —C(H)═; L³ is —CH₂—; and R² and R³ are each H. In certain embodiments of any of Formulas (LVII)-(LXIV), L¹ is —OC(H,Me)-; L² is —SO₂—; L³ is —CH₂—; and R² and R³ are each H. In certain embodiments of any of Formulas (LVII)-(LXIV), L¹ is —OC(H,Me)-; L² is —NH—; L³ is —CH₂—; and R² and R³ are each H. In certain embodiments of any of Formulas (LVII)-(LXIV), L¹ is —OC(H,Me)-; L² is —N(Me)-; L³ is —CH₂—; and R² and R³ are each H. In certain embodiments of any of Formulas (LVII)-(LXIV), L¹ is —OC(H,Me)-; L² is —C(O)—; L³ is —CH₂—; R² and R³ are each H; and R⁴ is (2S)-oxetan-2-yl-methyl. In certain embodiments of any of Formulas (LVII)-(LXIV), L¹ is —OC(H,Me)-; L² is —CH₂—; L³ is —CH₂—; R² and R³ are each H; and R⁴ is (2S)-oxetan-2-yl-methyl. In certain embodiments of any of Formulas (LVII)-(LXIV), L¹ is —OC(H,Me)-; L² is —C(H)═; L³ is —CH₂—; R² and R³ are each H; and R⁴ is (2S)-oxetan-2-yl-methyl. In certain embodiments of any of Formulas (LVII)-(LXIV), L¹ is —OC(H,Me)-; L² is —SO₂—; L³ is —CH₂—; R² and R³ are each H; and R⁴ is (2S)-oxetan-2-yl-methyl. In certain embodiments of any of Formulas (LVII)-(LXIV), L¹ is —OC(H,Me)-; L² is —NH—; L³ is —CH₂—; R² and R³ are each H; and R⁴ is (2S)-oxetan-2-yl-methyl. In certain embodiments of any of Formulas (LVII)-(LXIV), L¹ is —OC(H,Me)-; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —OC(H,Me)-; L² is —C(O)—; L³ is —CH₂—; R² and R³ are each H; and R⁴ is (1-ethyl-1H-imidazol-5-yl)-methyl. In certain embodiments of any of Formulas (LVII)-(LXIV), L¹ is —OC(H,Me)-; L² is —CH₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —OC(H,Me)-; L² is —C(H)═; L³ is —CH₂—; R² and R³ are each H; and R⁴ is (1-ethyl-1H-imidazol-5-yl)-methyl. In certain embodiments of any of Formulas (LVII)-(LXIV), L¹ is —OC(H,Me)-; L² is —SO₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —OC(H,Me)-; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —OC(H,Me)-; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —OC(H,Me)-; L² is —C(O)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OC(H,Me)-; L² is —CH₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OC(H,Me)-; L² is —C(H)═; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OC(H,Me)-; L² is —SO₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OC(H,Me)-; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OC(H,Me)-; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OC(H,Me)-; L² is —C(O)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OC(H,Me)-; L² is —CH₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OC(H,Me)-; L² is —C(H)═; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OC(H,Me)-; L² is —SO₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OC(H,Me)-; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OC(H,Me)-; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OC(H,Me)-; L² is —C(O)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CH₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —C(H)═; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —SO₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —C(O)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CH₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —C(H)═; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —SO₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments of any of Formulas (LVII)-(LXIV), R⁴ is (R)-1,3-oxazol-2-yl-methyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —SO₂NH—; and L² is —C(O)—. In certain embodiments of any of Formulas (LVII)-(LXIV), L¹ is —SO₂NH—; and L² is —CH₂—. In certain embodiments of any of Formulas (LVII)-(LXIV), L¹ is —SO₂NH—; and L² is —C(H)═. In certain embodiments of any of Formulas (LVII)-(LXIV), L¹ is —SO₂NH—; and L² is —SO₂—. In certain embodiments of any of Formulas (LVII)-(LXIV), L¹ is —SO₂NH—; and L² is —NH—. In certain embodiments of any of Formulas (LVII)-(LXIV), L¹ is —SO₂NH—; and L² is —N(Me)-. In certain embodiments of any of Formulas (LVII)-(LXIV), L¹ is —SO₂NH—; L² is —C(O)—; and L³ is —CH₂—. In certain embodiments of any of Formulas (LVII)-(LXIV), L¹ is —SO₂NH—; L² is —CH₂—; and L³ is —CH₂—. In certain embodiments of any of Formulas (LVII)-(LXIV), L¹ is —SO₂NH—; L² is —C(H)═; and L³ is —CH₂—. In certain embodiments of any of Formulas (LVII)-(LXIV), L¹ is —SO₂NH—; L² is —SO₂—; and L³ is —CH₂—. In certain embodiments of any of Formulas (LVII)-(LXIV), L¹ is —SO₂NH—; L² is —NH—; and L³ is —CH₂—. In certain embodiments of any of Formulas (LVII)-(LXIV), L¹ is —SO₂NH—; L² is —N(Me)-; and L³ is —CH₂—. In certain embodiments of any of Formulas (LVII)-(LXIV), L¹ is —SO₂NH—; L² is —C(O)—; L³ is —CH₂—; and R² and R³ are each H. In certain embodiments of any of Formulas (LVII)-(LXIV), L¹ is —SO₂NH—; L² is —CH₂—; L³ is —CH₂—; and R² and R³ are each H. In certain embodiments of any of Formulas (LVII)-(LXIV), L¹ is —SO₂NH—; L² is —C(H)═; L³ is —CH₂—; and R² and R³ are each H. In certain embodiments of any of Formulas (LVII)-(LXIV), L¹ is —SO₂NH—; L² is —SO₂—; L³ is —CH₂—; and R² and R³ are each H. In certain embodiments of any of Formulas (LVII)-(LXIV), L¹ is —SO₂NH—; L² is —NH—; L³ is —CH₂—; and R² and R³ are each H. In certain embodiments of any of Formulas (LVII)-(LXIV), L¹ is —SO₂NH—; L² is —N(Me)-; L³ is —CH₂—; and R² and R³ are each H. In certain embodiments of any of Formulas (LVII)-(LXIV), L¹ is —SO₂NH—; L² is —C(O)—; L³ is —CH₂—; R² and R³ are each H; and R⁴ is (2S)-oxetan-2-yl-methyl. In certain embodiments of any of Formulas (LVII)-(LXIV), L¹ is —SO₂NH—; L² is —CH₂—; L³ is —CH₂—; R² and R³ are each H; and R⁴ is (2S)-oxetan-2-yl-methyl. In certain embodiments of any of Formulas (LVII)-(LXIV), L¹ is —SO₂NH—; L² is —C(H)═; L³ is —CH₂—; R² and R³ are each H; and R⁴ is (2S)-oxetan-2-yl-methyl. In certain embodiments of any of Formulas (LVII)-(LXIV), L¹ is —SO₂NH—; L² is —SO₂—; L³ is —CH₂—; R² and R³ are each H; and R⁴ is (2S)-oxetan-2-yl-methyl. In certain embodiments of any of Formulas (LVII)-(LXIV), L¹ is —SO₂NH—; L² is —NH—; L³ is —CH₂—; R² and R³ are each H; and R⁴ is (2S)-oxetan-2-yl-methyl. In certain embodiments of any of Formulas (LVII)-(LXIV), L¹ is —SO₂NH—; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —SO₂NH—; L² is —C(O)—; L³ is —CH₂—; R² and R³ are each H; and R⁴ is (1-ethyl-1H-imidazol-5-yl)-methyl. In certain embodiments of any of Formulas (LVII)-(LXIV), L¹ is —SO₂NH—; L² is —CH₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —SO₂NH—; L² is —C(H)═; L³ is —CH₂—; R² and R³ are each H; and R⁴ is (1-ethyl-1H-imidazol-5-yl)-methyl. In certain embodiments of any of Formulas (LVII)-(LXIV), L¹ is —SO₂NH—; L² is —SO₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —SO₂NH—; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —SO₂NH—; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —SO₂NH—; L² is —C(O)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —SO₂NH—; L² is —CH₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —SO₂NH—; L² is —C(H)═; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —SO₂NH—; L² is —SO₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —SO₂NH—; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —SO₂NH—; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —SO₂NH—; L² is —C(O)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —SO₂NH—; L² is —CH₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —SO₂NH—; L² is —C(H)═; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —SO₂NH—; L² is —SO₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —SO₂NH—; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —SO₂NH—; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —SO₂NH—; L² is —C(O)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CH₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —C(H)═; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —SO₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —C(O)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CH₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —C(H)═; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —SO₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —O—; L² is —C(O)—; L³ is —CH₂—; R² and R³ are each H; and R⁴ is oxelan-3-yl-methyl. In certain embodiments of any of Formulas (LVII)-(LXIV), L¹ is —O—; L² is —CH₂—; L³ is —CH₂—; R² and R³ are each H; and R⁴ is oxelan-3-yl-methyl. In certain embodiments of any of Formulas (LVII)-(LXIV), L¹ is —O—; L² is —C(H)═; L³ is —CH₂—; R² and R³ are each H; and R⁴ is oxelan-3-yl-methyl. In certain embodiments of any of Formulas (LVII)-(LXIV), L¹ is —O—; L² is —SO₂—; L³ is —CH₂—; R² and R³ are each H; and R⁴ is oxelan-3-yl-methyl. In certain embodiments of any of Formulas (LVII)-(LXIV), L¹ is —O—; L² is —NH—; L³ is —CH₂—; R² and R³ are each H; and R⁴ is oxelan-3-yl-methyl. In certain embodiments of any of Formulas (LVII)-(LXIV), L¹ is —O—; L² is —N(Me)-; L³ is —CH₂—; R² and R³ are each H; and R⁴ is oxelan-3-yl-methyl. In certain embodiments of any of Formulas (LVII)-(LXIV), L¹ is —O—; L² is —C(O)—; L³ is —CH₂—; R² and R³ are each H; and R⁴ is 1,3-oxazol-2-yl-methyl. In certain embodiments of any of Formulas (LVII)-(LXIV), L¹ is —O—; L² is —CH₂—; L³ is —CH₂—; R² and R³ are each H; and R⁴ is 1,3-oxazol-2-yl-methyl. In certain embodiments of any of Formulas (LVII)-(LXIV), L¹ is —O—; L² is —C(H)═; L³ is —CH₂—; R² and R³ are each H; and R⁴ is 1,3-oxazol-2-yl-methyl. In certain embodiments of any of Formulas (LVII)-(LXIV), L¹ is —O—; L² is —SO₂—; L³ is —CH₂—; R² and R³ are each H; and R⁴ is 1,3-oxazol-2-yl-methyl. In certain embodiments of any of Formulas (LVII)-(LXIV), L¹ is —O—; L² is —NH—; L³ is —CH₂—; R² and R³ are each H; and R⁴ is 1,3-oxazol-2-yl-methyl. In certain embodiments of any of Formulas (LVII)-(LXIV), L¹ is —O—; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —O—; L² is —C(O)—; L³ is —CH₂—; R² and R³ are each H; R⁴ is oxelan-3-yl-methyl; and R⁵ is —COOH. In certain embodiments of any of Formulas (LVII)-(LXIV), L¹ is —O—; L² is —CH₂—; L³ is —CH₂—; R² and R³ are each H; R⁴ is oxelan-3-yl-methyl; and R⁵ is —COOH. In certain embodiments of any of Formulas (LVII)-(LXIV), L¹ is —O—; L² is —C(H)—; L³ is —CH₂—; R² and R³ are each H; R⁴ is oxelan-3-yl-methyl; and R⁵ is —COOH. In certain embodiments of any of Formulas (LVII)-(LXIV), L¹ is —O—; L² is —SO₂—; L³ is —CH₂—; R² and R³ are each H; R⁴ is oxelan-3-yl-methyl; and R⁵ is —COOH. In certain embodiments of any of Formulas (LVII)-(LXIV), L¹ is —O—; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —O—; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —O—; L² is —C(O)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —O—; L² is —CH₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —O—; L² is —C(H)═; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —O—; L² is —SO₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —O—; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —O—; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —O—; L² is —C(O)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —O—; L² is —CH₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —O—; L² is —C(H)═; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —O—; L² is —SO₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —O—; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —O—; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —O—; L² is —C(O)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —CH₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —C(H)═; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —SO₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —CH₂O—; L² is —C(O)—; L³ is —CH₂—; R² and R³ are each H; and R⁴ is oxelan-3-yl-methyl. In certain embodiments of any of Formulas (LVII)-(LXIV), L¹ is —CH₂O—; L² is —CH₂—; L³ is —CH₂—; R² and R³ are each H; and R⁴ is oxelan-3-yl-methyl. In certain embodiments of any of Formulas (LVII)-(LXIV), L¹ is —CH₂O—; L² is —C(H)═; L³ is —CH₂—; R² and R³ are each H; and R⁴ is oxelan-3-yl-methyl. In certain embodiments of any of Formulas (LVII)-(LXIV), L¹ is —CH₂O—; L² is —SO₂—; L³ is —CH₂—; R² and R³ are each H; and R⁴ is oxelan-3-yl-methyl. In certain embodiments of any of Formulas (LVII)-(LXIV), L¹ is —CH₂O—; L² is —NH—; L³ is —CH₂—; R² and R³ are each H; and R⁴ is oxelan-3-yl-methyl. In certain embodiments of any of Formulas (LVII)-(LXIV), L¹ is —CH₂O—; L² is —N(Me)-; L³ is —CH₂—; R² and R³ are each H; and R⁴ is oxelan-3-yl-methyl. In certain embodiments of any of Formulas (LVII)-(LXIV), L¹ is —CH₂O—; L² is —C(O)—; L³ is —CH₂—; R² and R³ are each H; and R⁴ is 1,3-oxazol-2-yl-methyl. In certain embodiments of any of Formulas (LVII)-(LXIV), L¹ is —CH₂O—; L² is —CH₂—; L³ is —CH₂—; R² and R³ are each H; and R⁴ is 1,3-oxazol-2-yl-methyl. In certain embodiments of any of Formulas (LVII)-(LXIV), L¹ is —CH₂O—; L² is —C(H)═; L³ is —CH₂—; R² and R³ are each H; and R⁴ is 1,3-oxazol-2-yl-methyl. In certain embodiments of any of Formulas (LVII)-(LXIV), L¹ is —CH₂O—; L² is —SO₂—; L³ is —CH₂—; R² and R³ are each H; and R⁴ is 1,3-oxazol-2-yl-methyl. In certain embodiments of any of Formulas (LVII)-(LXIV), L¹ is —CH₂O—; L² is —NH—; L³ is —CH₂—; R² and R³ are each H; and R⁴ is 1,3-oxazol-2-yl-methyl. In certain embodiments of any of Formulas (LVII)-(LXIV), L¹ is —CH₂O—; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —CH₂O—; L² is —C(O)—; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂O—; L² is —CH₂—; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂O—; L² is —C(H)═; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂O—; L² is —SO₂—; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂O—; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂O—; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂O—; L² is —C(O)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂O—; L² is —CH₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂O—; L² is —C(H)═; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂O—; L² is —SO₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂O—; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂O—; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂O—; L² is —C(O)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CH₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —C(H)═; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —SO₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —C(O)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CH₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —C(H)═; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —SO₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl.

In certain embodiments of any of Formulas (LVII)-(LXIV), L¹ is —OCH₂—; L² is —C(O)—; L³ is —CH₂—; R² and R³ are each H; and R⁴ is oxelan-3-yl-methyl. In certain embodiments of any of Formulas (LVII)-(LXIV), L¹ is —OCH₂—; L² is —CH₂—; L³ is —CH₂—; R² and R³ are each H; and R⁴ is oxelan-3-yl-methyl. In certain embodiments of any of Formulas (LVII)-(LXIV), L¹ is —OCH₂—; L² is —C(H)═; L³ is —CH₂—; R² and R³ are each H; and R⁴ is oxelan-3-yl-methyl. In certain embodiments of any of Formulas (LVII)-(LXIV), L¹ is —OCH₂—; L² is —SO₂—; L³ is —CH₂—; R² and R³ are each H; and R⁴ is oxelan-3-yl-methyl. In certain embodiments of any of Formulas (LVII)-(LXIV), L¹ is —OCH₂—; L² is —NH—; L³ is —CH₂—; R² and R³ are each H; and R⁴ is oxelan-3-yl-methyl. In certain embodiments of any of Formulas (LVII)-(LXIV), L¹ is —OCH₂—; L² is —N(Me)-; L³ is —CH₂—; R² and R³ are each H; and R⁴ is oxelan-3-yl-methyl. In certain embodiments of any of Formulas (LVII)-(LXIV), L¹ is —OCH₂—; L² is —C(O)—; L³ is —CH₂—; R² and R³ are each H; and R⁴ is 1,3-oxazol-2-yl-methyl. In certain embodiments of any of Formulas (LVII)-(LXIV), L¹ is —OCH₂—; L² is —CH₂—; L³ is —CH₂—; R² and R³ are each H; and R⁴ is 1,3-oxazol-2-yl-methyl. In certain embodiments of any of Formulas (LVII)-(LXIV), L¹ is —OCH₂—; L² is —C(H)═; L³ is —CH₂—; R² and R³ are each H; and R⁴ is 1,3-oxazol-2-yl-methyl. In certain embodiments of any of Formulas (LVII)-(LXIV), L¹ is —OCH₂—; L² is —SO₂—; L³ is —CH₂—; R² and R³ are each H; and R⁴ is 1,3-oxazol-2-yl-methyl. In certain embodiments of any of Formulas (LVII)-(LXIV), L¹ is —OCH₂—; L² is —NH—; L³ is —CH₂—; R² and R³ are each H; and R⁴ is 1,3-oxazol-2-yl-methyl. In certain embodiments of any of Formulas (LVII)-(LXIV), L¹ is —OCH₂—; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —OCH₂—; L² is —C(O)—; L³ is —CH₂—; R² and R³ are each H; R⁴ is oxelan-3-yl-methyl; and R⁵ is —COOH. In certain embodiments of any of Formulas (LVII)-(LXIV), L¹ is —OCH₂—; L² is —CH₂—; L³ is —CH₂—; R² and R³ are each H; R⁴ is oxelan-3-yl-methyl; and R⁵ is —COOH. In certain embodiments of any of Formulas (LVII)-(LXIV), L¹ is —OCH₂—; L² is —C(H)═; L³ is —CH₂—; R² and R³ are each H; R⁴ is oxelan-3-yl-methyl; and R⁵ is —COOH. In certain embodiments of any of Formulas (LVII)-(LXIV), L¹ is —OCH₂—; L² is —SO₂—; L³ is —CH₂—; R² and R³ are each H; R⁴ is oxelan-3-yl-methyl; and R⁵ is —COOH. In certain embodiments of any of Formulas (LVII)-(LXIV), L¹ is —OCH₂—; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —OCH₂—; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —OCH₂—; L² is —C(O)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OCH₂—; L² is —CH₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OCH₂—; L² is —C(H)═; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OCH₂—; L² is —SO₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OCH₂—; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OCH₂—; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OCH₂—; L² is —C(O)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CH₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —C(H)═; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —SO₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —C(O)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CH₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —C(H)═; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —SO₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —NH—; L² is —C(O)—; L³ is —CH₂—; R² and R³ are each H; and R⁴ is oxelan-3-yl-methyl. In certain embodiments of any of Formulas (LVII)-(LXIV), L¹ is —NH—; L² is —CH₂—; L³ is —CH₂—; R² and R³ are each H; and R⁴ is oxelan-3-yl-methyl. In certain embodiments of any of Formulas (LVII)-(LXIV), L¹ is —NH—; L² is —C(H)═; L³ is —CH₂—; R² and R³ are each H; and R⁴ is oxelan-3-yl-methyl. In certain embodiments of any of Formulas (LVII)-(LXIV), L¹ is —NH—; L² is —SO₂—; L³ is —CH₂—; R² and R³ are each H; and R⁴ is oxelan-3-yl-methyl. In certain embodiments of any of Formulas (LVII)-(LXIV), L¹ is —NH—; L² is —NH—; L³ is —CH₂—; R² and R³ are each H; and R⁴ is oxelan-3-yl-methyl. In certain embodiments of any of Formulas (LVII)-(LXIV), L¹ is —NH—; L² is —N(Me)-; L³ is —CH₂—; R² and R³ are each H; and R⁴ is oxelan-3-yl-methyl. In certain embodiments of any of Formulas (LVII)-(LXIV), L¹ is —NH—; L² is —C(O)—; L³ is —CH₂—; R² and R³ are each H; and R⁴ is 1,3-oxazol-2-yl-methyl. In certain embodiments of any of Formulas (LVII)-(LXIV), L¹ is —NH—; L² is —CH₂—; L³ is —CH₂—; R² and R³ are each H; and R⁴ is 1,3-oxazol-2-yl-methyl. In certain embodiments of any of Formulas (LVII)-(LXIV), L¹ is —NH—; L² is —C(H)═; L³ is —CH₂—; R² and R³ are each H; and R⁴ is 1,3-oxazol-2-yl-methyl. In certain embodiments of any of Formulas (LVII)-(LXIV), L¹ is —NH—; L² is —SO₂—; L³ is —CH₂—; R² and R³ are each H; and R⁴ is 1,3-oxazol-2-yl-methyl. In certain embodiments of any of Formulas (LVII)-(LXIV), L¹ is —NH—; L² is —NH—; L³ is —CH₂—; R² and R³ are each H; and R⁴ is 1,3-oxazol-2-yl-methyl. In certain embodiments of any of Formulas (LVII)-(LXIV), L¹ is —NH—; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —NH—; L² is —C(O)—; L³ is —CH₂—; R² and R³ are each H; R⁴ is oxelan-3-yl-methyl; and R⁵ is —COOH. In certain embodiments of any of Formulas (LVII)-(LXIV), L¹ is —NH—; L² is —CH₂—; L³ is —CH₂—; R² and R³ are each H; R⁴ is oxelan-3-yl-methyl; and R⁵ is —COOH. In certain embodiments of any of Formulas (LVII)-(LXIV), L¹ is —NH—; L² is —C(H)═; L³ is —CH₂—; R² and R³ are each H; R⁴ is oxelan-3-yl-methyl; and R⁵ is —COOH. In certain embodiments of any of Formulas (LVII)-(LXIV), L¹ is —NH—; L² is —SO₂—; L³ is —CH₂—; R² and R³ are each H; R⁴ is oxelan-3-yl-methyl; and R⁵ is —COOH. In certain embodiments of any of Formulas (LVII)-(LXIV), L¹ is —NH—; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —NH—; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —NH—; L² is —C(O)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NH—; L² is —CH₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NH—; L² is —C(H)═; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NH—; L² is —SO₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NH—; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NH—; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NH—; L² is —C(O)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NH—; L² is —CH₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NH—; L² is —C(H)═; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NH—; L² is —SO₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NH—; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NH—; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NH—; L² is —C(O)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —CH₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —C(H)═; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —SO₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —N(Me)-; L² is —C(O)—; L³ is —CH₂—; R² and R³ are each H; and R⁴ is oxelan-3-yl-methyl. In certain embodiments of any of Formulas (LVII)-(LXIV), L¹ is —N(Me)-; L² is —CH₂—; L³ is —CH₂—; R² and R³ are each H; and R⁴ is oxelan-3-yl-methyl. In certain embodiments of any of Formulas (LVII)-(LXIV), L¹ is —N(Me)-; L² is —C(H)═; L³ is —CH₂—; R² and R³ are each H; and R⁴ is oxelan-3-yl-methyl. In certain embodiments of any of Formulas (LVII)-(LXIV), L¹ is —N(Me)-; L² is —SO₂—; L³ is —CH₂—; R² and R³ are each H; and R⁴ is oxelan-3-yl-methyl. In certain embodiments of any of Formulas (LVII)-(LXIV), L¹ is —N(Me)-; L² is —NH—; L³ is —CH₂—; R² and R³ are each H; and R⁴ is oxelan-3-yl-methyl. In certain embodiments of any of Formulas (LVII)-(LXIV), L¹ is —N(Me)-; L² is —N(Me)-; L³ is —CH₂—; R² and R³ are each H; and R⁴ is oxelan-3-yl-methyl. In certain embodiments of any of Formulas (LVII)-(LXIV), L¹ is —N(Me)-; L² is —C(O)—; L³ is —CH₂—; R² and R³ are each H; and R⁴ is 1,3-oxazol-2-yl-methyl. In certain embodiments of any of Formulas (LVII)-(LXIV), L¹ is —N(Me)-; L² is —CH₂—; L³ is —CH₂—; R² and R³ are each H; and R⁴ is 1,3-oxazol-2-yl-methyl. In certain embodiments of any of Formulas (LVII)-(LXIV), L¹ is —N(Me)-; L² is —C(H)═; L³ is —CH₂—; R² and R³ are each H; and R⁴ is 1,3-oxazol-2-yl-methyl. In certain embodiments of any of Formulas (LVII)-(LXIV), L¹ is —N(Me)-; L² is —SO₂—; L³ is —CH₂—; R² and R³ are each H; and R⁴ is 1,3-oxazol-2-yl-methyl. In certain embodiments of any of Formulas (LVII)-(LXIV), L¹ is —N(Me)-; L² is —NH—; L³ is —CH₂—; R² and R³ are each H; and R⁴ is 1,3-oxazol-2-yl-methyl. In certain embodiments of any of Formulas (LVII)-(LXIV), L¹ is —N(Me)-; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —N(Me)-; L² is —C(O)—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)-; L² is —CH₂—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)-; L² is —C(H)═; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)-; L² is —SO₂—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)-; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)-; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)-; L² is —C(O)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)-; L² is —CH₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)-; L² is —C(H)═; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)-; L² is —SO₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)-; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)-; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)-; L² is —C(O)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CH₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —C(H)═; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —SO₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —C(O)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CH₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —C(H)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —SO₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), is —N(Me)-; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —N(Et)-; L² is —C(O)—; L³ is —CH₂—; R² and R³ are each H; and R⁴ is oxelan-3-yl-methyl. In certain embodiments of any of Formulas (LVII)-(LXIV), L¹ is —N(Et)-; L² is —CH₂—; L³ is —CH₂—; R² and R³ are each H; and R⁴ is oxelan-3-yl-methyl. In certain embodiments of any of Formulas (LVII)-(LXIV), L¹ is —N(Et)-; L² is —C(H)═; L³ is —CH₂—; R² and R³ are each H; and R⁴ is oxelan-3-yl-methyl. In certain embodiments of any of Formulas (LVII)-(LXIV), L¹ is —N(Et)-; L² is —SO₂—; L³ is —CH₂—; R² and R³ are each H; and R⁴ is oxelan-3-yl-methyl. In certain embodiments of any of Formulas (LVII)-(LXIV), L¹ is —N(Me)-; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —C(O)—; L³ is —CH₂—; R² and R³ are each H; and R⁴ is 1,3-oxazol-2-yl-methyl. In certain embodiments of any of Formulas (LVII)-(LXIV), L¹ is —N(Et)-; L² is —CH₂—; L³ is —CH₂—; R² and R³ are each H; and R⁴ is 1,3-oxazol-2-yl-methyl. In certain embodiments of any of Formulas (LVII)-(LXIV), L¹ is —N(Et)-; L² is —C(H)═; L³ is —CH₂—; R² and R³ are each H; and R⁴ is 1,3-oxazol-2-yl-methyl. In certain embodiments of any of Formulas (LVII)-(LXIV), L¹ is —N(Et)-; L² is —SO₂—; L³ is —CH₂—; R² and R³ are each H; and R⁴ is 1,3-oxazol-2-yl-methyl. In certain embodiments of any of Formulas (LVII)-(LXIV), L¹ is —N(Et)-; L² is —NH—; L³ is —CH₂—; R² and R³ are each H; and R⁴ is 1,3-oxazol-2-yl-methyl. In certain embodiments of any of Formulas (LVII)-(LXIV), L¹ is —N(Et)-; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —N(Et)-; L² is —C(O)—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Et)-; L² is —CH₂—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Et)-; L² is —C(H)═; L³ is —CH₂—; R² and R³ are each H; R⁴ is oxelan-3-yl-methyl; and R⁵ is —COOH. In certain embodiments of any of Formulas (LVII)-(LXIV), L¹ is —N(Et)-; L² is —SO₂—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Et)-; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Et)-; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —N(Et)-; L² is —C(O)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Et)-; L² is —CH₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Et)-; L² is —C(H)═; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Et)-; L² is —SO₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Et)-; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Et)-; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Et)-; L² is —C(O)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —CH₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —C(H)═; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —SO₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —C(O)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —CH₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —C(H)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —SO₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —NHCH₂—; L² is —C(O)—; L³ is —CH₂—; R² and R³ are each H; and R⁴ is oxelan-3-yl-methyl. In certain embodiments of any of Formulas (LVII)-(LXIV), L¹ is —NHCH₂—; L² is —CH₂—; L³ is —CH₂—; R² and R³ are each H; and R⁴ is oxelan-3-yl-methyl. In certain embodiments of any of Formulas (LVII)-(LXIV), L¹ is —NHCH₂—; L² is —C(H)═; L³ is —CH₂—; R² and R³ are each H; and R⁴ is oxelan-3-yl-methyl. In certain embodiments of any of Formulas (LVII)-(LXIV), L¹ is —NHCH₂—; L² is —SO₂—; L³ is —CH₂—; R² and R³ are each H; and R⁴ is oxelan-3-yl-methyl. In certain embodiments of any of Formulas (LVII)-(LXIV), L¹ is —NHCH₂—; L² is —NH—; L³ is —CH₂—; R² and R³ are each H; and R⁴ is oxelan-3-yl-methyl. In certain embodiments of any of Formulas (LVII)-(LXIV), L¹ is —NHCH₂—; L² is —N(Me)-; L³ is —CH₂—; R² and R³ are each H; and R⁴ is oxelan-3-yl-methyl. In certain embodiments of any of Formulas (LVII)-(LXIV), L¹ is —NHCH₂—; L² is —C(O)—; L³ is —CH₂—; R² and R³ are each H; and R⁴ is 1,3-oxazol-2-yl-methyl. In certain embodiments of any of Formulas (LVII)-(LXIV), L¹ is —NHCH₂—; L² is —CH₂—; L³ is —CH₂—; R² and R³ are each H; and R⁴ is 1,3-oxazol-2-yl-methyl. In certain embodiments of any of Formulas (LVII)-(LXIV), L¹ is —NHCH₂—; L² is —C(H)═; L³ is —CH₂—; R² and R³ are each H; and R⁴ is 1,3-oxazol-2-yl-methyl. In certain embodiments of any of Formulas (LVII)-(LXIV), L¹ is —NHCH₂—; L² is —SO₂—; L³ is —CH₂—; R² and R³ are each H; and R⁴ is 1,3-oxazol-2-yl-methyl. In certain embodiments of any of Formulas (LVII)-(LXIV), L¹ is —NHCH₂—; L² is —NH—; L³ is —CH₂—; R² and R³ are each H; and R⁴ is 1,3-oxazol-2-yl-methyl. In certain embodiments of any of Formulas (LVII)-(LXIV), L¹ is —NHCH₂—; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —NHCH₂—; L² is —C(O)—; L³ is —CH₂—; R² and R³ are each H; R⁴ is oxelan-3-yl-methyl; and R⁵ is —COOH. In certain embodiments of any of Formulas (LVII)-(LXIV), L¹ is —NHCH₂—; L² is —CH₂—; L³ is —CH₂—; R² and R³ are each H; R⁴ is oxelan-3-yl-methyl; and R⁵ is —COOH. In certain embodiments of any of Formulas (LVII)-(LXIV), L¹ is —NHCH₂—; L² is —C(H)═; L³ is —CH₂—; R² and R³ are each H; R⁴ is oxelan-3-yl-methyl; and R⁵ is —COOH. In certain embodiments of any of Formulas (LVII)-(LXIV), L¹ is —NHCH₂—; L² is —SO₂—; L³ is —CH₂—; R² and R³ are each H; R⁴ is oxelan-3-yl-methyl; and R⁵ is —COOH. In certain embodiments of any of Formulas (LVII)-(LXIV), L¹ is —NHCH₂—; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —NHCH₂—; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —NHCH₂—; L² is —C(O)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NHCH₂—; L² is —CH₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NHCH₂—; L² is —C(H)═; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NHCH₂—; L² is —SO₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NHCH₂—; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NHCH₂—; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NHCH₂—; L² is —C(O)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CH₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —C(H)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —SO₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —C(O)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CH₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —C(H)═; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —SO₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —CH₂NH—; L² is —C(O)—; L³ is —CH₂—; R² and R³ are each H; and R⁴ is oxelan-3-yl-methyl. In certain embodiments of any of Formulas (LVII)-(LXIV), L¹ is —CH₂NH—; L² is —CH₂—; L³ is —CH₂—; R² and R³ are each H; and R⁴ is oxelan-3-yl-methyl. In certain embodiments of any of Formulas (LVII)-(LXIV), L¹ is —CH₂NH—; L² is —C(H)═; L³ is —CH₂—; R² and R³ are each H; and R⁴ is oxelan-3-yl-methyl. In certain embodiments of any of Formulas (LVII)-(LXIV), L¹ is —CH₂NH—; L² is —SO₂—; L³ is —CH₂—; R² and R³ are each H; and R⁴ is oxelan-3-yl-methyl. In certain embodiments of any of Formulas (LVII)-(LXIV), L¹ is —CH₂NH—; L² is —NH—; L³ is —CH₂—; R² and R³ are each H; and R⁴ is oxelan-3-yl-methyl. In certain embodiments of any of Formulas (LVII)-(LXIV), L¹ is —CH₂NH—; L² is —N(Me)-; L³ is —CH₂—; R² and R³ are each H; and R⁴ is oxelan-3-yl-methyl. In certain embodiments of any of Formulas (LVII)-(LXIV), L¹ is —CH₂NH—; L² is —C(O)—; L³ is —CH₂—; R² and R³ are each H; and R⁴ is 1,3-oxazol-2-yl-methyl. In certain embodiments of any of Formulas (LVII)-(LXIV), L¹ is —CH₂NH—; L² is —CH₂—; L³ is —CH₂—; R² and R³ are each H; and R⁴ is 1,3-oxazol-2-yl-methyl. In certain embodiments of any of Formulas (LVII)-(LXIV), L¹ is —CH₂NH—; L² is —C(H)═; L³ is —CH₂—; R² and R³ are each H; and R⁴ is 1,3-oxazol-2-yl-methyl. In certain embodiments of any of Formulas (LVII)-(LXIV), L¹ is —CH₂NH—; L² is —SO₂—; L³ is —CH₂—; R² and R³ are each H; and R⁴ is 1,3-oxazol-2-yl-methyl. In certain embodiments of any of Formulas (LVII)-(LXIV), L¹ is —CH₂NH—; L² is —NH—; L³ is —CH₂—; R² and R³ are each H; and R⁴ is 1,3-oxazol-2-yl-methyl. In certain embodiments of any of Formulas (LVII)-(LXIV), L¹ is —CH₂NH—; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —CH₂NH—; L² is —C(O)—; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂NH—; L² is —CH₂—; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂NH—; L² is —C(H)═; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂NH—; L² is —SO₂—; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂NH—; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂NH—; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂NH—; L² is —C(O)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂NH—; L² is —CH₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂NH—; L² is —C(H)═; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂NH—; L² is —SO₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂NH—; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂NH—; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂NH—; L² is —C(O)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —CH₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —C(H)═; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —SO₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —C(O)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —CH₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —C(H)═; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —SO₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —CH₂NH—; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —N(Me)CH₂—; L² is —C(O)—; L³ is —CH₂—; R² and R³ are each H; and R⁴ is oxelan-3-yl-methyl. In certain embodiments of any of Formulas (LVII)-(LXIV), L¹ is —N(Me)CH₂—; L² is —CH₂—; L³ is —CH₂—; R² and R³ are each H; and R⁴ is oxelan-3-yl-methyl. In certain embodiments of any of Formulas (LVII)-(LXIV), L¹ is —N(Me)CH₂—; L² is —C(H)═; L³ is —CH₂—; R² and R³ are each H; and R⁴ is oxelan-3-yl-methyl. In certain embodiments of any of Formulas (LVII)-(LXIV), L¹ is —N(Me)CH₂—; L² is —SO₂—; L³ is —CH₂—; R² and R³ are each H; and R⁴ is oxelan-3-yl-methyl. In certain embodiments of any of Formulas (LVII)-(LXIV), L¹ is —N(Me)CH₂—; L² is —NH—; L³ is —CH₂—; R² and R³ are each H; and R⁴ is oxelan-3-yl-methyl. In certain embodiments of any of Formulas (LVII)-(LXIV), L¹ is —N(Me)CH₂—; L² is —N(Me)-; L³ is —CH₂—; R² and R³ are each H; and R⁴ is oxelan-3-yl-methyl. In certain embodiments of any of Formulas (LVII)-(LXIV), L¹ is —N(Me)CH₂—; L² is —C(O)—; L³ is —CH₂—; R² and R³ are each H; and R⁴ is 1,3-oxazol-2-yl-methyl. In certain embodiments of any of Formulas (LVII)-(LXIV), L¹ is —N(Me)CH₂—; L² is —CH₂—; L³ is —CH₂—; R² and R³ are each H; and R⁴ is 1,3-oxazol-2-yl-methyl. In certain embodiments of any of Formulas (LVII)-(LXIV), L¹ is —N(Me)CH₂—; L² is —C(H)═; L³ is —CH₂—; R² and R³ are each H; and R⁴ is 1,3-oxazol-2-yl-methyl. In certain embodiments of any of Formulas (LVII)-(LXIV), L¹ is —N(Me)CH₂—; L² is —SO₂—; L³ is —CH₂—; R² and R³ are each H; and R⁴ is 1,3-oxazol-2-yl-methyl. In certain embodiments of any of Formulas (LVII)-(LXIV), L¹ is —N(Me)CH₂—; L² is —NH—; L³ is —CH₂—; R² and R³ are each H; and R⁴ is 1,3-oxazol-2-yl-methyl. In certain embodiments of any of Formulas (LVII)-(LXIV), L¹ is —N(Me)CH₂—; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —N(Me)CH₂—; L² is —C(O)—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)CH₂—; L² is —CH₂—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)CH₂—; L² is —C(H)═; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)CH₂—; L² is —SO₂—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)CH₂—; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)CH₂—; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)CH₂—; L² is —C(O)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)CH₂—; L² is —CH₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)CH₂—; L² is —C(H)═; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)CH₂—; L² is —SO₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)CH₂—; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)CH₂—; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)CH₂—; L² is —C(O)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CH₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —C(H)═; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —SO₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —C(O)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CH₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —C(H)═; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —SO₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —OC(H,Me)-; L² is —C(O)—; L³ is —CH₂—; R² and R³ are each H; and R⁴ is oxelan-3-yl-methyl. In certain embodiments of any of Formulas (LVII)-(LXIV), L¹ is —OC(H,Me)-; L² is —CH₂—; L³ is —CH₂—; R² and R³ are each H; and R⁴ is oxelan-3-yl-methyl. In certain embodiments of any of Formulas (LVII)-(LXIV), L¹ is —OC(H,Me)-; L² is —C(H)═; L³ is —CH₂—; R² and R³ are each H; and R⁴ is oxelan-3-yl-methyl. In certain embodiments of any of Formulas (LVII)-(LXIV), L¹ is —OC(H,Me)-; L² is —SO₂—; L³ is —CH₂—; R² and R³ are each H; and R⁴ is oxelan-3-yl-methyl. In certain embodiments of any of Formulas (LVII)-(LXIV), L¹ is —OC(H,Me)-; L² is —NH—; L³ is —CH₂—; R² and R³ are each H; and R⁴ is oxelan-3-yl-methyl. In certain embodiments of any of Formulas (LVII)-(LXIV), L¹ is —OC(H,Me)-; L² is —N(Me)-; L³ is —CH₂—; R² and R³ are each H; and R⁴ is oxelan-3-yl-methyl. In certain embodiments of any of Formulas (LVII)-(LXIV), L¹ is —OC(H,Me)-; L² is —C(O)—; L³ is —CH₂—; R² and R³ are each H; and R⁴ is 1,3-oxazol-2-yl-methyl. In certain embodiments of any of Formulas (LVII)-(LXIV), L¹ is —OC(H,Me)-; L² is —CH₂—; L³ is —CH₂—; R² and R³ are each H; and R⁴ is 1,3-oxazol-2-yl-methyl. In certain embodiments of any of Formulas (LVII)-(LXIV), L¹ is —OC(H,Me)-; L² is —C(H)═; L³ is —CH₂—; R² and R³ are each H; and R⁴ is 1,3-oxazol-2-yl-methyl. In certain embodiments of any of Formulas (LVII)-(LXIV), L¹ is —OC(H,Me)-; L² is —SO₂—; L³ is —CH₂—; R² and R³ are each H; and R⁴ is 1,3-oxazol-2-yl-methyl. In certain embodiments of any of Formulas (LVII)-(LXIV), L¹ is —OC(H,Me)-; L² is —NH—; L³ is —CH₂—; R² and R³ are each H; and R⁴ is 1,3-oxazol-2-yl-methyl. In certain embodiments of any of Formulas (LVII)-(LXIV), L¹ is —OC(H,Me)-; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —OC(H,Me)-; L² is —C(O)—; L³ is —CH₂—; R² and R³ are each H; R⁴ is oxelan-3-yl-methyl; and R⁵ is —COOH. In certain embodiments of any of Formulas (LVII)-(LXIV), L¹ is —OC(H,Me)-; L² is —CH₂—; L³ is —CH₂—; R² and R³ are each H; R⁴ is oxelan-3-yl-methyl; and R⁵ is —COOH. In certain embodiments of any of Formulas (LVII)-(LXIV), L¹ is —OC(H,Me)-; L² is —C(H)═; L³ is —CH₂—; R² and R³ are each H; R⁴ is oxelan-3-yl-methyl; and R⁵ is —COOH. In certain embodiments of any of Formulas (LVII)-(LXIV), L¹ is —OC(H,Me)-; L² is —SO₂—; L³ is —CH₂—; R² and R³ are each H; R⁴ is oxelan-3-yl-methyl; and R⁵ is —COOH. In certain embodiments of any of Formulas (LVII)-(LXIV), L¹ is —OC(H,Me)-; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —OC(H,Me)-; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —OC(H,Me)-; L² is —C(O)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OC(H,Me)-; L² is —CH₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OC(H,Me)-; L² is —C(H)═; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OC(H,Me)-; L² is —SO₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OC(H,Me)-; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OC(H,Me)-; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OC(H,Me)-; L² is —C(O)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CH₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —C(H)═; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —SO₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —C(O)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CH₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —C(H)═; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —SO₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —SO₂NH—; L² is —C(O)—; L³ is —CH₂—; R² and R³ are each H; and R⁴ is oxelan-3-yl-methyl. In certain embodiments of any of Formulas (LVII)-(LXIV), L¹ is —SO₂NH—; L² is —CH₂—; L³ is —CH₂—; R² and R³ are each H; and R⁴ is oxelan-3-yl-methyl. In certain embodiments of any of Formulas (LVII)-(LXIV), L¹ is —SO₂NH—; L² is —C(H)—; L³ is —CH₂—; R² and R³ are each H; and R⁴ is oxelan-3-yl-methyl. In certain embodiments of any of Formulas (LVII)-(LXIV), L¹ is —SO₂NH—; L² is —SO₂—; L³ is —CH₂—; R² and R³ are each H; and R⁴ is oxelan-3-yl-methyl. In certain embodiments of any of Formulas (LVII)-(LXIV), L¹ is —SO₂NH—; L² is —NH—; L³ is —CH₂—; R² and R³ are each H; and R⁴ is oxelan-3-yl-methyl. In certain embodiments of any of Formulas (LVII)-(LXIV), L¹ is —SO₂NH—; L² is —N(Me)-; L³ is —CH₂—; R² and R³ are each H; and R⁴ is oxelan-3-yl-methyl. In certain embodiments of any of Formulas (LVII)-(LXIV), L¹ is —SO₂NH—; L² is —C(O)—; L³ is —CH₂—; R² and R³ are each H; and R⁴ is 1,3-oxazol-2-yl-methyl. In certain embodiments of any of Formulas (LVII)-(LXIV), L¹ is —SO₂NH—; L² is —CH₂—; L³ is —CH₂—; R² and R³ are each H; and R⁴ is 1,3-oxazol-2-yl-methyl. In certain embodiments of any of Formulas (LVII)-(LXIV), L¹ is —SO₂NH—; L² is —C(H)═; L³ is —CH₂—; R² and R³ are each H; and R⁴ is 1,3-oxazol-2-yl-methyl. In certain embodiments of any of Formulas (LVII)-(LXIV), L¹ is —SO₂NH—; L² is —SO₂—; L³ is —CH₂—; R² and R³ are each H; and R⁴ is 1,3-oxazol-2-yl-methyl. In certain embodiments of any of Formulas (LVII)-(LXIV), L¹ is —SO₂NH—; L² is —NH—; L³ is —CH₂—; R² and R³ are each H; and R⁴ is 1,3-oxazol-2-yl-methyl. In certain embodiments of any of Formulas (LVII)-(LXIV), L¹ is —SO₂NH—; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —SO₂NH—; L² is —C(O)—; L³ is —CH₂—; R² and R³ are each H; R⁴ is oxelan-3-yl-methyl; and R⁵ is —COOH. In certain embodiments of any of Formulas (LVII)-(LXIV), L¹ is —SO₂NH—; L² is —CH₂—; L³ is —CH₂—; R² and R³ are each H; R⁴ is oxelan-3-yl-methyl; and R⁵ is —COOH. In certain embodiments of any of Formulas (LVII)-(LXIV), L¹ is —SO₂NH—; L² is —C(H)═; L³ is —CH₂—; R² and R³ are each H; R⁴ is oxelan-3-yl-methyl; and R⁵ is —COOH. In certain embodiments of any of Formulas (LVII)-(LXIV), L¹ is —SO₂NH—; L² is —SO₂—; L³ is —CH₂—; R² and R³ are each H; R⁴ is oxelan-3-yl-methyl; and R⁵ is —COOH. In certain embodiments of any of Formulas (LVII)-(LXIV), L¹ is —SO₂NH—; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —SO₂NH—; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —SO₂NH—; L² is —C(O)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —SO₂NH—; L² is —CH₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —SO₂NH—; L² is —C(H)═; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —SO₂NH—; L² is —SO₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —SO₂NH—; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —SO₂NH—; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —SO₂NH—; L² is —C(O)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CH₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —C(H)═; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —SO₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —C(O)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CH₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —C(H)═; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —SO₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —O—; and L² is —O—. In certain embodiments of any of Formulas (LVII)-(LXIV), L¹ is —O—; and L² is —CF₂—. In certain embodiments of any of Formulas (LVII)-(LXIV), L¹ is —O—; and L² is —CHF—. In certain embodiments of any of Formulas (LVII)-(LXIV), L¹ is —O—; and L² is —CH(OH)—. In certain embodiments of any of Formulas (LVII)-(LXIV), L¹ is —O—; and L² is —NH—. In certain embodiments of any of Formulas (LVII)-(LXIV), L¹ is —O—; and L² is —N(Me)-. In certain embodiments of any of Formulas (LVII)-(LXIV), L¹ is —O—; L² is —O—; and L³ is —CH₂—. In certain embodiments of any of Formulas (LVII)-(LXIV), L¹ is —O—; L² is —CF₂—; and L³ is —CH₂—. In certain embodiments of any of Formulas (LVII)-(LXIV), L¹ is —O—; L² is —CHF—; and L³ is —CH₂—. In certain embodiments of any of Formulas (LVII)-(LXIV), L¹ is —O—; L² is —CH(OH)—; and L³ is —CH₂—. In certain embodiments of any of Formulas (LVII)-(LXIV), L¹ is —O—; L² is —NH—; and L³ is —CH₂—. In certain embodiments of any of Formulas (LVII)-(LXIV), L¹ is —O—; L² is —N(Me)-; and L³ is —CH₂—. In certain embodiments of any of Formulas (LVII)-(LXIV), L¹ is —O—; L² is —O—; L³ is —CH₂—; and R² and R³ are each H. In certain embodiments of any of Formulas (LVII)-(LXIV), L¹ is —O—; L² is —CF₂—; L³ is —CH₂—; and R² and R³ are each H. In certain embodiments of any of Formulas (LVII)-(LXIV), L¹ is —O—; L² is —CHF—; L³ is —CH₂—; and R² and R³ are each H. In certain embodiments of any of Formulas (LVII)-(LXIV), L¹ is —O—; L² is —CH(OH)—; L³ is —CH₂—; and R² and R³ are each H. In certain embodiments of any of Formulas (LVII)-(LXIV), L¹ is —O—; L² is —NH—; L³ is —CH₂—; and R² and R³ are each H. In certain embodiments of any of Formulas (LVII)-(LXIV), L¹ is —O—; L² is —N(Me)-; L³ is —CH₂—; and R² and R³ are each H. In certain embodiments of any of Formulas (LVII)-(LXIV), L¹ is —O—; L² is —O—; L³ is —CH₂—; R² and R³ are each H; and R⁴ is (2S)-oxetan-2-yl-methyl. In certain embodiments of any of Formulas (LVII)-(LXIV), L¹ is —O—; L² is —CF₂—; L³ is —CH₂—; R² and R³ are each H; and R⁴ is (2S)-oxetan-2-yl-methyl. In certain embodiments of any of Formulas (LVII)-(LXIV), L¹ is —O—; L² is —CHF—; L³ is —CH₂—; R² and R³ are each H; and R⁴ is (2S)-oxetan-2-yl-methyl. In certain embodiments of any of Formulas (LVII)-(LXIV), L¹ is —O—; L² is —CH(OH)—; L³ is —CH₂—; R² and R³ are each H; and R⁴ is (2S)-oxetan-2-yl-methyl. In certain embodiments of any of Formulas (LVII)-(LXIV), L¹ is —O—; L² is —NH—; L³ is —CH₂—; R² and R³ are each H; and R⁴ is (2S)-oxetan-2-yl-methyl. In certain embodiments of any of Formulas (LVII)-(LXIV), L¹ is —O—; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —O—; L² is —O—; L³ is —CH₂—; R² and R³ are each H; and R⁴ is (1-ethyl-1H-imidazol-5-yl)-methyl. In certain embodiments of any of Formulas (LVII)-(LXIV), L¹ is —O—; L² is —CF₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —O—; L² is —CHF—; L³ is —CH₂—; R² and R³ are each H; and R⁴ is (1-ethyl-1H-imidazol-5-yl)-methyl. In certain embodiments of any of Formulas (LVII)-(LXIV), L¹ is —O—; L² is —CH(OH)—; L³ is —CH₂—; R² and R³ are each H; and R⁴ is (1-ethyl-1H-imidazol-5-yl)-methyl. In certain embodiments of any of Formulas (LVII)-(LXIV), L¹ is —O—; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —O—; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —O—; L² is —O—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —O—; L² is —CF₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —O—; L² is —CHF—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —O—; L² is —CH(OH)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —O—; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —O—; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —O—; L² is —O—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —O—; L² is —CF₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —O—; L² is —CHF—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —O—; L² is —CH(OH)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —O—; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —O—; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —O—; L² is —O—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —CF₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —CHF—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —CH(OH)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —O—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —O—; L² is —CF₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —O—; L² is —CHF—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —O—; L² is —CH(OH)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —O—; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —O—; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —CH₂O—; and L² is —O—. In certain embodiments of any of Formulas (LVII)-(LXIV), L¹ is —CH₂O—; and L² is —CF₂—. In certain embodiments of any of Formulas (LVII)-(LXIV), L¹ is —CH₂O—; and L² is —CHF—. In certain embodiments of any of Formulas (LVII)-(LXIV), L¹ is —CH₂O—; and L² is —CH(OH)—. In certain embodiments of any of Formulas (LVII)-(LXIV), L¹ is —CH₂O—; and L² is —NH—. In certain embodiments of any of Formulas (LVII)-(LXIV), L¹ is —CH₂O—; and L² is —N(Me)-. In certain embodiments of any of Formulas (LVII)-(LXIV), L¹ is —CH₂O—; L² is —O—; and L³ is —CH₂—. In certain embodiments of any of Formulas (LVII)-(LXIV), L¹ is —CH₂O—; L² is —CF₂—; and L³ is —CH₂—. In certain embodiments of any of Formulas (LVII)-(LXIV), L¹ is —CH₂O—; L² is —CHF—; and L³ is —CH₂—. In certain embodiments of any of Formulas (LVII)-(LXIV), L¹ is —CH₂O—; L² is —CH(OH)—; and L³ is —CH₂—. In certain embodiments of any of Formulas (LVII)-(LXIV), L¹ is —CH₂O—; L² is —O—; L³ is —CH₂—; and R² and R³ are each H. In certain embodiments of any of Formulas (LVII)-(LXIV), L¹ is —CH₂O—; L² is —CF₂—; L³ is —CH₂—; and R² and R³ are each H. In certain embodiments of any of Formulas (LVII)-(LXIV), L¹ is —CH₂O—; L² is —CHF—; L³ is —CH₂—; and R² and R³ are each H. In certain embodiments of any of Formulas (LVII)-(LXIV), L¹ is —CH₂O—; L² is —CH(OH)—; L³ is —CH₂—; and R² and R³ are each H. In certain embodiments of any of Formulas (LVII)-(LXIV), L¹ is —CH₂O—; L² is —NH—; L³ is —CH₂—; and R² and R³ are each H. In certain embodiments of any of Formulas (LVII)-(LXIV), L¹ is —CH₂O—; L² is —N(Me)-; L³ is —CH₂—; and R² and R³ are each H. In certain embodiments of any of Formulas (LVII)-(LXIV), L¹ is —CH₂O—; L² is —O—; L³ is —CH₂—; R² and R³ are each H; and R⁴ is (2S)-oxetan-2-yl-methyl. In certain embodiments of any of Formulas (LVII)-(LXIV), L¹ is —CH₂O—; L² is —CF₂—; L³ is —CH₂—; R² and R³ are each H; and R⁴ is (2S)-oxetan-2-yl-methyl. In certain embodiments of any of Formulas (LVII)-(LXIV), L¹ is —CH₂O—; L² is —CHF—; L³ is —CH₂—; R² and R³ are each H; and R⁴ is (2S)-oxetan-2-yl-methyl. In certain embodiments of any of Formulas (LVII)-(LXIV), L¹ is —CH₂O—; L² is —CH(OH)—; L³ is —CH₂—; R² and R³ are each H; and R⁴ is (2S)-oxetan-2-yl-methyl. In certain embodiments of any of Formulas (LVII)-(LXIV), L¹ is —CH₂O—; L² is —NH—; L³ is —CH₂—; R² and R³ are each H; and R⁴ is (2S)-oxetan-2-yl-methyl. In certain embodiments of any of Formulas (LVII)-(LXIV), L¹ is —CH₂O—; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —CH₂O—; L² is —O—; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂O—; L² is —CF₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂O—; L² is —CHF—; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂O—; L² is —CH(OH)—; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂O—; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂O—; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —CH₂O—; L² is —O—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂O—; L² is —CF₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂O—; L² is —CHF—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂O—; L² is —CH(OH)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂O—; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂O—; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —CH₂O—; L² is —O—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂O—; L² is —CF₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂O—; L² is —CHF—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂O—; L² is —CH(OH)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂O—; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂O—; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —CH₂O—; L² is —O—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CF₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CHF—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CH(OH)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —O—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CF₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CHF—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CH(OH)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —CH₂O—; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —CH₂O—; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —OCH₂—; and L² is —O—. In certain embodiments of any of Formulas (LVII)-(LXIV), L¹ is —OCH₂—; and L² is —CF₂—. In certain embodiments of any of Formulas (LVII)-(LXIV), L¹ is —OCH₂—; and L² is —CHF—. In certain embodiments of any of Formulas (LVII)-(LXIV), L¹ is —OCH₂—; and L² is —CH(OH)—. In certain embodiments of any of Formulas (LVII)-(LXIV), L¹ is —OCH₂—; and L² is —NH—. In certain embodiments of any of Formulas (LVII)-(LXIV), L¹ is —OCH₂—; and L² is —N(Me)-. In certain embodiments of any of Formulas (LVII)-(LXIV), L¹ is —OCH₂—; L² is —O—; and L³ is —CH₂—. In certain embodiments of any of Formulas (LVII)-(LXIV), L¹ is —OCH₂—; L² is —CF₂—; and L³ is —CH₂—. In certain embodiments of any of Formulas (LVII)-(LXIV), L¹ is —OCH₂—; L² is —CHF—; and L³ is —CH₂—. In certain embodiments of any of Formulas (LVII)-(LXIV), L¹ is —OCH₂—; L² is —CH(OH)—; and L³ is —CH₂—. In certain embodiments of any of Formulas (LVII)-(LXIV), L¹ is —OCH₂—; L² is —NH—; and L³ is —CH₂—. In certain embodiments of any of Formulas (LVII)-(LXIV), L¹ is —OCH₂—; L² is —N(Me)-; and L³ is —CH₂—. In certain embodiments of any of Formulas (LVII)-(LXIV), L¹ is —OCH₂—; L² is —O—; L³ is —CH₂—; and R² and R³ are each H. In certain embodiments of any of Formulas (LVII)-(LXIV), L¹ is —OCH₂—; L² is —CF₂—; L³ is —CH₂—; and R² and R³ are each H. In certain embodiments of any of Formulas (LVII)-(LXIV), L¹ is —OCH₂—; L² is —CHF—; L³ is —CH₂—; and R² and R³ are each H. In certain embodiments of any of Formulas (LVII)-(LXIV), L¹ is —OCH₂—; L² is —CH(OH)—; L³ is —CH₂—; and R² and R³ are each H. In certain embodiments of any of Formulas (LVII)-(LXIV), L¹ is —OCH₂—; L² is —NH—; L³ is —CH₂—; and R² and R³ are each H. In certain embodiments of any of Formulas (LVII)-(LXIV), L¹ is —OCH₂—; L² is —N(Me)-; L³ is —CH₂—; and R² and R³ are each H. In certain embodiments of any of Formulas (LVII)-(LXIV), L¹ is —OCH₂—; L² is —O—; L³ is —CH₂—; R² and R³ are each H; and R⁴ is (2S)-oxetan-2-yl-methyl. In certain embodiments of any of Formulas (LVII)-(LXIV), L¹ is —OCH₂—; L² is —CF₂—; L³ is —CH₂—; R² and R³ are each H; and R⁴ is (2S)-oxetan-2-yl-methyl. In certain embodiments of any of Formulas (LVII)-(LXIV), L¹ is —OCH₂—; L² is —CHF—; L³ is —CH₂—; R² and R³ are each H; and R⁴ is (2S)-oxetan-2-yl-methyl. In certain embodiments of any of Formulas (LVII)-(LXIV), L¹ is —OCH₂—; L² is —CH(OH)—; L³ is —CH₂—; R² and R³ are each H; and R⁴ is (2S)-oxetan-2-yl-methyl. In certain embodiments of any of Formulas (LVII)-(LXIV), L¹ is —OCH₂—; L² is —NH—; L³ is —CH₂—; R² and R³ are each H; and R⁴ is (2S)-oxetan-2-yl-methyl. In certain embodiments of any of Formulas (LVII)-(LXIV), L¹ is —OCH₂—; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —OCH₂—; L² is —O—; L³ is —CH₂—; R² and R³ are each H; and R⁴ is (1-ethyl-1H-imidazol-5-yl)-methyl. In certain embodiments of any of Formulas (LVII)-(LXIV), L¹ is —OCH₂—; L² is —CF₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —OCH₂—; L² is —CHF—; L³ is —CH₂—; R² and R³ are each H; and R⁴ is (1-ethyl-1H-imidazol-5-yl)-methyl. In certain embodiments of any of Formulas (LVII)-(LXIV), L¹ is —OCH₂—; L² is —CH(OH)—; L³ is —CH₂—; R² and R³ are each H; and R⁴ is (1-ethyl-1H-imidazol-5-yl)-methyl. In certain embodiments of any of Formulas (LVII)-(LXIV), L¹ is —OCH₂—; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —OCH₂—; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —OCH₂—; L² is —O—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OCH₂—; L² is —CF₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OCH₂—; L² is —CHF—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OCH₂—; L² is —CH(OH)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OCH₂—; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OCH₂—; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OCH₂—; L² is —O—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OCH₂—; L² is —CF₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OCH₂—; L² is —CHF—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OCH₂—; L² is —CH(OH)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OCH₂—; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OCH₂—; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OCH₂—; L² is —O—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CF₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CHF—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CH(OH)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —O—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CF₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CHF—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CH(OH)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OCH₂—; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OCH₂—; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —NH— and L² is —O—. In certain embodiments of any of Formulas (LVII)-(LXIV), L¹ is —NH— and L² is —CF₂—. In certain embodiments of any of Formulas (LVII)-(LXIV), L¹ is —NH—; and L² is —CHF—. In certain embodiments of any of Formulas (LVII)-(LXIV), L¹ is —NH—; and L² is —CH(OH)—. In certain embodiments of any of Formulas (LVII)-(LXIV), L¹ is —NH—; and L² is —NH—. In certain embodiments of any of Formulas (LVII)-(LXIV), L¹ is —NH—; and L² is —N(Me)-. In certain embodiments of any of Formulas (LVII)-(LXIV), L¹ is —NH—; L² is —O—; and L³ is —CH₂—. In certain embodiments of any of Formulas (LVII)-(LXIV), L¹ is —NH—; L² is —CF₂—; and L³ is —CH₂—. In certain embodiments of any of Formulas (LVII)-(LXIV), L¹ is —NH—; L² is —CHF—; and L³ is —CH₂—. In certain embodiments of any of Formulas (LVII)-(LXIV), L¹ is —NH—; L² is —CH(OH)—; and L³ is —CH₂—. In certain embodiments of any of Formulas (LVII)-(LXIV), L¹ is —NH—; L² is —NH—; and L³ is —CH₂—. In certain embodiments of any of Formulas (LVII)-(LXIV), L¹ is —NH—; L² is —N(Me)-; and L³ is —CH₂—. In certain embodiments of any of Formulas (LVII)-(LXIV), L¹ is —NH—; L² is —O—; L³ is —CH₂—; and R² and R³ are each H. In certain embodiments of any of Formulas (LVII)-(LXIV), L¹ is —NH—; L² is —CF₂—; L³ is —CH₂—; and R² and R³ are each H. In certain embodiments of any of Formulas (LVII)-(LXIV), L¹ is —NH—; L² is —CHF—; L³ is —CH₂—; and R² and R³ are each H. In certain embodiments of any of Formulas (LVII)-(LXIV), L¹ is —NH—; L² is —CH(OH)—; L³ is —CH₂—; and R² and R³ are each H. In certain embodiments of any of Formulas (LVII)-(LXIV), L¹ is —NH—; L² is —NH—; L³ is —CH₂—; and R² and R³ are each H. In certain embodiments of any of Formulas (LVII)-(LXIV), L¹ is —NH—; L² is —N(Me)-; L³ is —CH₂—; and R² and R³ are each H. In certain embodiments of any of Formulas (LVII)-(LXIV), L¹ is —NH—; L² is —O—; L³ is —CH₂—; R² and R³ are each H; and R⁴ is (2S)-oxetan-2-yl-methyl. In certain embodiments of any of Formulas (LVII)-(LXIV), L¹ is —NH—; L² is —CF₂—; L³ is —CH₂—; R² and R³ are each H; and R⁴ is (2S)-oxetan-2-yl-methyl. In certain embodiments of any of Formulas (LVII)-(LXIV), L¹ is —NH—; L² is —CHF—; L³ is —CH₂—; R² and R³ are each H; and R⁴ is (2S)-oxetan-2-yl-methyl. In certain embodiments of any of Formulas (LVII)-(LXIV), L¹ is —NH—; L² is —CH(OH)—; L³ is —CH₂—; R² and R³ are each H; and R⁴ is (2S)-oxetan-2-yl-methyl. In certain embodiments of any of Formulas (LVII)-(LXIV), L¹ is —NH—; L² is —NH—; L³ is —CH₂—; R² and R³ are each H; and R⁴ is (2S)-oxetan-2-yl-methyl. In certain embodiments of any of Formulas (LVII)-(LXIV), L¹ is —NH—; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —NH—; L² is —O—; L³ is —CH₂—; R² and R³ are each H; and R⁴ is (1-ethyl-1H-imidazol-5-yl)-methyl. In certain embodiments of any of Formulas (LVII)-(LXIV), L¹ is —NH—; L² is —CF₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —NH—; L² is —CHF—; L³ is —CH₂—; R² and R³ are each H; and R⁴ is (1-ethyl-1H-imidazol-5-yl)-methyl. In certain embodiments of any of Formulas (LVII)-(LXIV), L¹ is —NH—; L² is —CH(OH)—; L³ is —CH₂—; R² and R³ are each H; and R⁴ is (1-ethyl-1H-imidazol-5-yl)-methyl. In certain embodiments of any of Formulas (LVII)-(LXIV), L¹ is —NH—; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —NH—; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —NH—; L² is —O—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NH—; L² is —CF₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NH—; L² is —CHF—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NH—; L² is —CH(OH)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NH—; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NH—; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NH—; L² is —O—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NH—; L² is —CF₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NH—; L² is —CHF—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NH—; L² is —CH(OH)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NH—; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NH—; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NH—; L² is —O—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —CF₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —CHF—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —CH(OH)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —O—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NH—; L² is —CF₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NH—; L² is —CHF—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NH—; L² is —CH(OH)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NH—; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NH—; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —N(Me)- and L² is —O—. In certain embodiments of any of Formulas (LVII)-(LXIV), L¹ is —N(Me)- and L² is —CF₂—. In certain embodiments of any of Formulas (LVII)-(LXIV), L¹ is —N(Me)- and L² is —CHF—. In certain embodiments of any of Formulas (LVII)-(LXIV), L¹ is —N(Me)- and L² is —CH(OH)—. In certain embodiments of any of Formulas (LVII)-(LXIV), L¹ is —N(Me)- and L² is —NH—. In certain embodiments of any of Formulas (LVII)-(LXIV), L¹ is —N(Me)- and L² is —N(Me)-. In certain embodiments of any of Formulas (LVII)-(LXIV), L¹ is —N(Me)-; L² is —O—; and L³ is —CH₂—. In certain embodiments of any of Formulas (LVII)-(LXIV), L¹ is —N(Me)-; L² is —CF₂—; and L³ is —CH₂—. In certain embodiments of any of Formulas (LVII)-(LXIV), L¹ is —N(Me)-; L² is —CHF—; and L³ is —CH₂—. In certain embodiments of any of Formulas (LVII)-(LXIV), L¹ is —N(Me)-; L² is —CH(OH)—; and L³ is —CH₂—. In certain embodiments of any of Formulas (LVII)-(LXIV), L¹ is —N(Me)-; L² is —NH—; and L³ is —CH₂—. In certain embodiments of any of Formulas (LVII)-(LXIV), L¹ is —N(Me)-; L² is —N(Me)-; and L³ is —CH₂—. In certain embodiments of any of Formulas (LVII)-(LXIV), L¹ is —N(Me)-; L² is —O—; L³ is —CH₂—; and R² and R³ are each H. In certain embodiments of any of Formulas (LVII)-(LXIV), L¹ is —N(Me)-; L² is —CF₂—; L³ is —CH₂—; and R² and R³ are each H. In certain embodiments of any of Formulas (LVII)-(LXIV), L¹ is —N(Me)-; L² is —CHF—; L³ is —CH₂—; and R² and R³ are each H. In certain embodiments of any of Formulas (LVII)-(LXIV), L¹ is —N(Me)-; L² is —CH(OH)—; L³ is —CH₂—; and R² and R³ are each H. In certain embodiments of any of Formulas (LVII)-(LXIV), L¹ is —N(Me)-; L² is —NH—; L³ is —CH₂—; and R² and R³ are each H. In certain embodiments of any of Formulas (LVII)-(LXIV), L¹ is —N(Me)-; L² is —N(Me)-; L³ is —CH₂—; and R² and R³ are each H. In certain embodiments of any of Formulas (LVII)-(LXIV), L¹ is —N(Me)-; L² is —O—; L³ is —CH₂—; R² and R³ are each H; and R⁴ is (2S)-oxetan-2-yl-methyl. In certain embodiments of any of Formulas (LVII)-(LXIV), L¹ is —N(Me)-; L² is —CF₂—; L³ is —CH₂—; R² and R³ are each H; and R⁴ is (2S)-oxetan-2-yl-methyl. In certain embodiments of any of Formulas (LVII)-(LXIV), L¹ is —N(Me)-; L² is —CHF—; L³ is —CH₂—; R² and R³ are each H; and R⁴ is (2S)-oxetan-2-yl-methyl. In certain embodiments of any of Formulas (LVII)-(LXIV), L¹ is —N(Me)-; L² is —CH(OH)—; L³ is —CH₂—; R² and R³ are each H; and R⁴ is (2S)-oxetan-2-yl-methyl. In certain embodiments of any of Formulas (LVII)-(LXIV), L¹ is —N(Me)-; L² is —NH—; L³ is —CH₂—; R² and R³ are each H; and R⁴ is (2S)-oxetan-2-yl-methyl. In certain embodiments of any of Formulas (LVII)-(LXIV), L¹ is —N(Me)-; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —N(Me)-; L² is —O—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)-; L² is —CF₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)-; L² is —CHF—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)-; L² is —CH(OH)—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)-; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)-; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)-; L² is —O—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)-; L² is —CF₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)-; L² is —CHF—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)-; L² is —CH(OH)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)-; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)-; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)-; L² is —O—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)-; L² is —CF₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)-; L² is —CHF—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)-; L² is —CH(OH)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)-; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)-; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)-; L² is —O—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CF₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CHF—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CH(OH)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —O—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CF₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CHF—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CH(OH)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)-; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)-; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —N(Et)-; and L² is —O—. In certain embodiments of any of Formulas (LVII)-(LXIV), L¹ is —N(Et)-; and L² is —CF₂—. In certain embodiments of any of Formulas (LVII)-(LXIV), L¹ is —N(Et)-; and L² is —CHF—. In certain embodiments of any of Formulas (LVII)-(LXIV), L¹ is —N(Et)-; and L² is —CH(OH)—. In certain embodiments of any of Formulas (LVII)-(LXIV), L¹ is —N(Et)-; and L² is —NH—. In certain embodiments of any of Formulas (LVII)-(LXIV), L¹ is —N(Et)-; and L² is —N(Me)-. In certain embodiments of any of Formulas (LVII)-(LXIV), L¹ is —N(Et)-; L² is —O—; and L³ is —CH₂—. In certain embodiments of any of Formulas (LVII)-(LXIV), L¹ is —N(Et)-; L² is —CF₂—; and L³ is —CH₂—. In certain embodiments of any of Formulas (LVII)-(LXIV), L¹ is —N(Et)-; L² is —CHF—; and L³ is —CH₂—. In certain embodiments of any of Formulas (LVII)-(LXIV), L¹ is —N(Et)-; L² is —CH(OH)—; and L³ is —CH₂—. In certain embodiments of any of Formulas (LVII)-(LXIV), L¹ is —N(Et)-; L² is —NH—; and L³ is —CH₂—. In certain embodiments of any of Formulas (LVII)-(LXIV), L¹ is —N(Et)-; L² is —N(ME)-; and L³ is —CH₂—. In certain embodiments of any of Formulas (LVII)-(LXIV), L¹ is —N(Et)-; L² is —O—; L³ is —CH₂—; and R² and R³ are each H. In certain embodiments of any of Formulas (LVII)-(LXIV), L¹ is —N(Et)-; L² is —CF₂—; L³ is —CH₂—; and R² and R³ are each H. In certain embodiments of any of Formulas (LVII)-(LXIV), L¹ is —N(Et)-; L² is —CHF—; L³ is —CH₂—; and R² and R³ are each H. In certain embodiments of any of Formulas (LVII)-(LXIV), L¹ is —N(Et)-; L² is —CH(OH)—; L³ is —CH₂—; and R² and R³ are each H. In certain embodiments of any of Formulas (LVII)-(LXIV), L¹ is —N(Et)-; L² is —NH—; L³ is —CH₂—; and R² and R³ are each H. In certain embodiments of any of Formulas (LVII)-(LXIV), L¹ is —N(Et)-; L² is —N(Me)-; L³ is —CH₂—; and R² and R³ are each H. In certain embodiments of any of Formulas (LVII)-(LXIV), L¹ is —N(Et)-; L² is —O—; L³ is —CH₂—; R² and R³ are each H; and R⁴ is (2S)-oxetan-2-yl-methyl. In certain embodiments of any of Formulas (LVII)-(LXIV), L¹ is —N(Et)-; L² is —CF₂—; L³ is —CH₂—; R² and R³ are each H; and R⁴ is (2S)-oxetan-2-yl-methyl. In certain embodiments of any of Formulas (LVII)-(LXIV), L¹ is —N(Et)-; L² is —CHF—; L³ is —CH₂—; R² and R³ are each H; and R⁴ is (2S)-oxetan-2-yl-methyl. In certain embodiments of any of Formulas (LVII)-(LXIV), L¹ is —N(Et)-; L² is —CH(OH)—; L³ is —CH₂—; R² and R³ are each H; and R⁴ is (2S)-oxetan-2-yl-methyl. In certain embodiments of any of Formulas (LVII)-(LXIV), L¹ is —N(Et)-; L² is —NH—; L³ is —CH₂—; R² and R³ are each H; and R⁴ is (2S)-oxetan-2-yl-methyl. In certain embodiments of any of Formulas (LVII)-(LXIV), L¹ is —N(Et)-; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —N(Et)-; L² is —O—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Et)-; L² is —CF₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Et)-; L² is —CHF—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Et)-; L² is —CH(OH)—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Et)-; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Et)-; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Et)-; L² is —O—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Et)-; L² is —CF₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Et)-; L² is —CHF—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Et)-; L² is —CH(OH)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Et)-; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Et)-; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Et)-; L² is —O—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Et)-; L² is —CF₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Et)-; L² is —CHF—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Et)-; L² is —CH(OH)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Et)-; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Et)-; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Et)-; L² is —O—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —CF₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —CHF—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —CH(OH)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —O—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Et)-; L² is —CF₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Et)-; L² is —CHF—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Et)-; L² is —CH(OH)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Et)-; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Et)-; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —NHCH₂—; and L² is —O—. In certain embodiments of any of Formulas (LVII)-(LXIV), L¹ is —NHCH₂—; and L² is —CF₂—. In certain embodiments of any of Formulas (LVII)-(LXIV), L¹ is —NHCH₂—; and L² is —CHF—. In certain embodiments of any of Formulas (LVII)-(LXIV), L¹ is —NHCH₂—; and L² is —CH(OH)—. In certain embodiments of any of Formulas (LVII)-(LXIV), L¹ is —NHCH₂—; and L² is —NH—. In certain embodiments of any of Formulas (LVII)-(LXIV), L¹ is —NHCH₂—; and L² is —N(Me)-. In certain embodiments of any of Formulas (LVII)-(LXIV), L¹ is —NHCH₂—; L² is —O—; and L³ is —CH₂—. In certain embodiments of any of Formulas (LVII)-(LXIV), L¹ is —NHCH₂—; L² is —CF₂—; and L³ is —CH₂—. In certain embodiments of any of Formulas (LVII)-(LXIV), L¹ is —NHCH₂—; L² is —CHF—; and L³ is —CH₂—. In certain embodiments of any of Formulas (LVII)-(LXIV), L¹ is —NHCH₂—; L² is —CH(OH)—; and L³ is —CH₂—. In certain embodiments of any of Formulas (LVII)-(LXIV), L¹ is —NHCH₂—; L² is —NH—; and L³ is —CH₂—. In certain embodiments of any of Formulas (LVII)-(LXIV), L¹ is —NHCH₂—; L² is —N(Me)-; and L³ is —CH₂—. In certain embodiments of any of Formulas (LVII)-(LXIV), L¹ is —NHCH₂—; L² is —O—; L³ is —CH₂—; and R² and R³ are each H. In certain embodiments of any of Formulas (LVII)-(LXIV), L¹ is —NHCH₂—; L² is —CF₂—; L³ is —CH₂—; and R² and R³ are each H. In certain embodiments of any of Formulas (LVII)-(LXIV), L¹ is —NHCH₂—; L² is —CHF—; L³ is —CH₂—; and R² and R³ are each H. In certain embodiments of any of Formulas (LVII)-(LXIV), L¹ is —NHCH₂—; L² is —CH(OH)—; L³ is —CH₂—; and R² and R³ are each H. In certain embodiments of any of Formulas (LVII)-(LXIV), L¹ is —NHCH₂—; L² is —NH—; L³ is —CH₂—; and R² and R³ are each H. In certain embodiments of any of Formulas (LVII)-(LXIV), L¹ is —NHCH₂—; L² is —N(Me)-; L³ is —CH₂—; and R² and R³ are each H. In certain embodiments of any of Formulas (LVII)-(LXIV), L¹ is —NHCH₂—; L² is —O—; L³ is —CH₂—; R² and R³ are each H; and R⁴ is (2S)-oxetan-2-yl-methyl. In certain embodiments of any of Formulas (LVII)-(LXIV), L¹ is —NHCH₂—; L² is —CF₂—; L³ is —CH₂—; R² and R³ are each H; and R⁴ is (2S)-oxetan-2-yl-methyl. In certain embodiments of any of Formulas (LVII)-(LXIV), L¹ is —NHCH₂—; L² is —CHF—; L³ is —CH₂—; R² and R³ are each H; and R⁴ is (2S)-oxetan-2-yl-methyl. In certain embodiments of any of Formulas (LVII)-(LXIV), L¹ is —NHCH₂—; L² is —CH(OH)—; L³ is —CH₂—; R² and R³ are each H; and R⁴ is (2S)-oxetan-2-yl-methyl. In certain embodiments of any of Formulas (LVII)-(LXIV), L¹ is —NHCH₂—; L² is —NH—; L³ is —CH₂—; R² and R³ are each H; and R⁴ is (2S)-oxetan-2-yl-methyl. In certain embodiments of any of Formulas (LVII)-(LXIV), L¹ is —NHCH₂—; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —NHCH₂—; L² is —O—; L³ is —CH₂—; R² and R³ are each H; and R⁴ is (1-ethyl-1H-imidazol-5-yl)-methyl. In certain embodiments of any of Formulas (LVII)-(LXIV), L¹ is —NHCH₂—; L² is —CF₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —NHCH₂—; L² is —CHF—; L³ is —CH₂—; R² and R³ are each H; and R⁴ is (1-ethyl-1H-imidazol-5-yl)-methyl. In certain embodiments of any of Formulas (LVII)-(LXIV), L¹ is —NHCH₂—; L² is —CH(OH)—; L³ is —CH₂—; R² and R³ are each H; and R⁴ is (1-ethyl-1H-imidazol-5-yl)-methyl. In certain embodiments of any of Formulas (LVII)-(LXIV), L¹ is —NHCH₂—; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —NHCH₂—; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —NHCH₂—; L² is —O—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NHCH₂—; L² is —CF₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NHCH₂—; L² is —CHF—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NHCH₂—; L² is —CH(OH)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NHCH₂—; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NHCH₂—; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —NHCH₂—; L² is —O—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NHCH₂—; L² is —CF₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NHCH₂—; L² is —CHF—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NHCH₂—; L² is —CH(OH)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NHCH₂—; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NHCH₂—; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —NHCH₂—; L² is —O—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CF₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CHF—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CH(OH)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —O—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CF₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CHF—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CH(OH)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —N(Me)CH₂—; and L² is —O—. In certain embodiments of any of Formulas (LVII)-(LXIV), L¹ is —N(Me)CH₂—; and L² is —CF₂—. In certain embodiments of any of Formulas (LVII)-(LXIV), L¹ is —N(Me)CH₂—; and L² is —CHF—. In certain embodiments of any of Formulas (LVII)-(LXIV), L¹ is —N(Me)CH₂—; and L² is —CH(OH)—. In certain embodiments of any of Formulas (LVII)-(LXIV), L¹ is —N(Me)CH₂—; and L² is —NH—. In certain embodiments of any of Formulas (LVII)-(LXIV), L¹ is —N(Me)CH₂—; and L² is —N(Me)-. In certain embodiments of any of Formulas (LVII)-(LXIV), L¹ is —N(Me)CH₂—; L² is —O—; and L³ is —CH₂—. In certain embodiments of any of Formulas (LVII)-(LXIV), L¹ is —N(Me)CH₂—; L² is —CF₂—; and L³ is —CH₂—. In certain embodiments of any of Formulas (LVII)-(LXIV), L¹ is —N(Me)CH₂—; L² is —CHF—; and L³ is —CH₂—. In certain embodiments of any of Formulas (LVII)-(LXIV), L¹ is —N(Me)CH₂—; L² is —CH(OH)—; and L³ is —CH₂—. In certain embodiments of any of Formulas (LVII)-(LXIV), L¹ is —N(Me)CH₂—; L² is —NH—; and L³ is —CH₂—. In certain embodiments of any of Formulas (LVII)-(LXIV), L¹ is —N(Me)CH₂—; L² is —N(Me)-; and L³ is —CH₂—. In certain embodiments of any of Formulas (LVII)-(LXIV), L¹ is —N(Me)CH₂—; L² is —O—; L³ is —CH₂—; and R² and R³ are each H. In certain embodiments of any of Formulas (LVII)-(LXIV), L¹ is —N(Me)CH₂—; L² is —CF₂—; L³ is —CH₂—; and R² and R³ are each H. In certain embodiments of any of Formulas (LVII)-(LXIV), L¹ is —N(Me)CH₂—; L² is —CHF—; L³ is —CH₂—; and R² and R³ are each H. In certain embodiments of any of Formulas (LVII)-(LXIV), L¹ is —N(Me)CH₂—; L² is —CH(OH)—; L³ is —CH₂—; and R² and R³ are each H. In certain embodiments of any of Formulas (LVII)-(LXIV), L¹ is —N(Me)CH₂—; L² is —NH—; L³ is —CH₂—; and R² and R³ are each H. In certain embodiments of any of Formulas (LVII)-(LXIV), L¹ is —N(Me)CH₂—; L² is —N(Me)-; L³ is —CH₂—; and R² and R³ are each H. In certain embodiments of any of Formulas (LVII)-(LXIV), L¹ is —N(Me)CH₂—; L² is —O—; L³ is —CH₂—; R² and R³ are each H; and R⁴ is (2S)-oxetan-2-yl-methyl. In certain embodiments of any of Formulas (LVII)-(LXIV), L¹ is —N(Me)CH₂—; L² is —CF₂—; L³ is —CH₂—; R² and R³ are each H; and R⁴ is (2S)-oxetan-2-yl-methyl. In certain embodiments of any of Formulas (LVII)-(LXIV), L¹ is —N(Me)CH₂—; L² is —CHF—; L³ is —CH₂—; R² and R³ are each H; and R⁴ is (2S)-oxetan-2-yl-methyl. In certain embodiments of any of Formulas (LVII)-(LXIV), L¹ is —N(Me)CH₂—; L² is —CH(OH)—; L³ is —CH₂—; R² and R³ are each H; and R⁴ is (2S)-oxetan-2-yl-methyl. In certain embodiments of any of Formulas (LVII)-(LXIV), L¹ is —N(Me)CH₂—; L² is —NH—; L³ is —CH₂—; R² and R³ are each H; and R⁴ is (2S)-oxetan-2-yl-methyl. In certain embodiments of any of Formulas (LVII)-(LXIV), L¹ is —N(Me)CH₂—; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —N(Me)CH₂—; L² is —O—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)CH₂—; L² is —CF₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)CH₂—; L² is —CHF—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)CH₂—; L² is —CH(OH)—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)CH₂—; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)CH₂—; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —N(Me)CH₂—; L² is —O—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)CH₂—; L² is —CF₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)CH₂—; L² is —CHF—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)CH₂—; L² is —CH(OH)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)CH₂—; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)CH₂—; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —N(Me)CH₂—; L² is —O—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)CH₂—; L² is —CF₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)CH₂—; L² is —CHF—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)CH₂—; L² is —CH(OH)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)CH₂—; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)CH₂—; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —N(Me)CH₂—; L² is —O—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CF₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CHF—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CH(OH)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —O—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CF₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CHF—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CH(OH)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —OC(H,Me)-; and L² is —O—. In certain embodiments of any of Formulas (LVII)-(LXIV), L¹ is —OC(H,Me)-; and L² is —CF₂—. In certain embodiments of any of Formulas (LVII)-(LXIV), L¹ is —OC(H,Me)-; and L² is —CHF—. In certain embodiments of any of Formulas (LVII)-(LXIV), L¹ is —OC(H,Me)- and L² is —CH(OH)—. In certain embodiments of any of Formulas (LVII)-(LXIV), L¹ is —OC(H,Me)- and L² is —NH—. In certain embodiments of any of Formulas (LVII)-(LXIV), L¹ is —OC(H,Me)- and L² is —N(Me)-. In certain embodiments of any of Formulas (LVII)-(LXIV), L¹ is —OC(H,Me)-; L² is —O—; and L³ is —CH₂—. In certain embodiments of any of Formulas (LVII)-(LXIV), L¹ is —OC(H,Me)-; L² is —CF₂—; and L³ is —CH₂—. In certain embodiments of any of Formulas (LVII)-(LXIV), L¹ is —OC(H,Me)-; L² is —CHF—; and L³ is —CH₂—. In certain embodiments of any of Formulas (LVII)-(LXIV), L¹ is —OC(H,Me)-; L² is —CH(OH)—; and L³ is —CH₂—. In certain embodiments of any of Formulas (LVII)-(LXIV), L¹ is —OC(H,Me)-; L² is —NH—; and L³ is —CH₂—. In certain embodiments of any of Formulas (LVII)-(LXIV), L¹ is —OC(H,Me)-; L² is —NH (Me)-; and L³ is —CH₂—. In certain embodiments of any of Formulas (LVII)-(LXIV), L¹ is —OC(H,Me)-; L² is —O—; L³ is —CH₂—; and R² and R³ are each H. In certain embodiments of any of Formulas (LVII)-(LXIV), L¹ is —OC(H,Me)-; L² is —CF₂—; L³ is —CH₂—; and R² and R³ are each H. In certain embodiments of any of Formulas (LVII)-(LXIV), L¹ is —OC(H,Me)-; L² is —CHF—; L³ is —CH₂—; and R² and R³ are each H. In certain embodiments of any of Formulas (LVII)-(LXIV), L¹ is —OC(H,Me)-; L² is —CH(OH)—; L³ is —CH₂—; and R² and R³ are each H. In certain embodiments of any of Formulas (LVII)-(LXIV), L¹ is —OC(H,Me)-; L² is —NH—; L³ is —CH₂—; and R² and R³ are each H. In certain embodiments of any of Formulas (LVII)-(LXIV), L¹ is —OC(H,Me)-; L² is —N(Me)-; L³ is —CH₂—; and R² and R³ are each H. In certain embodiments of any of Formulas (LVII)-(LXIV), L¹ is —OC(H,Me)-; L² is —O—; L³ is —CH₂—; R² and R³ are each H; and R⁴ is (2S)-oxetan-2-yl-methyl. In certain embodiments of any of Formulas (LVII)-(LXIV), L¹ is —OC(H,Me)-; L² is —CF₂—; L³ is —CH₂—; R² and R³ are each H; and R⁴ is (2S)-oxetan-2-yl-methyl. In certain embodiments of any of Formulas (LVII)-(LXIV), L¹ is —OC(H,Me)-; L² is —CHF—; L³ is —CH₂—; R² and R³ are each H; and R⁴ is (2S)-oxetan-2-yl-methyl. In certain embodiments of any of Formulas (LVII)-(LXIV), L¹ is —OC(H,Me)-; L² is —CH(OH)—; L³ is —CH₂—; R² and R³ are each H; and R⁴ is (2S)-oxetan-2-yl-methyl. In certain embodiments of any of Formulas (LVII)-(LXIV), L¹ is —OC(H,Me)-; L² is —NH—; L³ is —CH₂—; R² and R³ are each H; and R⁴ is (2S)-oxetan-2-yl-methyl. In certain embodiments of any of Formulas (LVII)-(LXIV), L¹ is —OC(H,Me)-; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —OC(H,Me)-; L² is —O—; L³ is —CH₂—; R² and R³ are each H; and R⁴ is (1-ethyl-1H-imidazol-5-yl)-methyl. In certain embodiments of any of Formulas (LVII)-(LXIV), L¹ is —OC(H,Me)-; L² is —CF₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —OC(H,Me)-; L² is —CHF—; L³ is —CH₂—; R² and R³ are each H; and R⁴ is (1-ethyl-1H-imidazol-5-yl)-methyl. In certain embodiments of any of Formulas (LVII)-(LXIV), L¹ is —OC(H,Me)-; L² is —CH(OH)—; L³ is —CH₂—; R² and R³ are each H; and R⁴ is (1-ethyl-1H-imidazol-5-yl)-methyl. In certain embodiments of any of Formulas (LVII)-(LXIV), L¹ is —OC(H,Me)-; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —OC(H,Me)-; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —OC(H,Me)-; L² is —O—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OC(H,Me)-; L² is —CF₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OC(H,Me)-; L² is —CHF—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OC(H,Me)-; L² is —CH(OH)—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OC(H,Me)-; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L′ is —OC(H,Me)-; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —OC(H,Me)-; L² is —O—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OC(H,Me)-; L² is —CF₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OC(H,Me)-; L² is —CHF—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OC(H,Me)-; L² is —CH(OH)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OC(H,Me)-; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OC(H,Me)-; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —OC(H,Me)-; L² is —O—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CF₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CHF—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CH(OH)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —O—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CF₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CHF—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CH(OH)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —SO₂NH—; and L² is —O—. In certain embodiments of any of Formulas (LVII)-(LXIV), L¹ is —SO₂NH—; and L² is —CF₂—. In certain embodiments of any of Formulas (LVII)-(LXIV), L¹ is —SO₂NH—; and L² is —CHF—. In certain embodiments of any of Formulas (LVII)-(LXIV), L¹ is —SO₂NH—; and L² is —CH(OH)—. In certain embodiments of any of Formulas (LVII)-(LXIV), L¹ is —SO₂NH—; and L² is —NH—. In certain embodiments of any of Formulas (LVII)-(LXIV), L¹ is —SO₂NH—; and L² is —N(Me)-. In certain embodiments of any of Formulas (LVII)-(LXIV), L¹ is —SO₂NH—; L² is —O—; and L³ is —CH₂—. In certain embodiments of any of Formulas (LVII)-(LXIV), L¹ is —SO₂NH—; L² is —CF₂—; and L³ is —CH₂—. In certain embodiments of any of Formulas (LVII)-(LXIV), L¹ is —SO₂NH—; L² is —CHF—; and L³ is —CH₂—. In certain embodiments of any of Formulas (LVII)-(LXIV), L¹ is —SO₂NH—; L² is —CH(OH)—; and L³ is —CH₂—. In certain embodiments of any of Formulas (LVII)-(LXIV), L¹ is —SO₂NH—; L² is —NH—; and L³ is —CH₂—. In certain embodiments of any of Formulas (LVII)-(LXIV), L¹ is —SO₂NH—; L² is —N(Me)-; and L³ is —CH₂—. In certain embodiments of any of Formulas (LVII)-(LXIV), L¹ is —SO₂NH—; L² is —O—; L³ is —CH₂—; and R² and R³ are each H. In certain embodiments of any of Formulas (LVII)-(LXIV), L¹ is —SO₂NH—; L² is —CF₂—; L³ is —CH₂—; and R² and R³ are each H. In certain embodiments of any of Formulas (LVII)-(LXIV), L¹ is —SO₂NH—; L² is —CHF—; L³ is —CH₂—; and R² and R³ are each H. In certain embodiments of any of Formulas (LVII)-(LXIV), L¹ is —SO₂NH—; L² is —CH(OH)—; L³ is —CH₂—; and R² and R³ are each H. In certain embodiments of any of Formulas (LVII)-(LXIV), L¹ is —SO₂NH—; L² is —NH—; L³ is —CH₂—; and R² and R³ are each H. In certain embodiments of any of Formulas (LVII)-(LXIV), L¹ is —SO₂NH—; L² is —N(Me)-; L³ is —CH₂—; and R² and R³ are each H. In certain embodiments of any of Formulas (LVII)-(LXIV), L¹ is —SO₂NH—; L² is —O—; L³ is —CH₂—; R² and R³ are each H; and R⁴ is (2S)-oxetan-2-yl-methyl. In certain embodiments of any of Formulas (LVII)-(LXIV), L¹ is —SO₂NH—; L² is —CF₂—; L³ is —CH₂—; R² and R³ are each H; and R⁴ is (2S)-oxetan-2-yl-methyl. In certain embodiments of any of Formulas (LVII)-(LXIV), L¹ is —SO₂NH—; L² is —CHF—; L³ is —CH₂—; R² and R³ are each H; and R⁴ is (2S)-oxetan-2-yl-methyl. In certain embodiments of any of Formulas (LVII)-(LXIV), L¹ is —SO₂NH—; L² is —CH(OH)—; L³ is —CH₂—; R² and R³ are each H; and R⁴ is (2S)-oxetan-2-yl-methyl. In certain embodiments of any of Formulas (LVII)-(LXIV), L¹ is —SO₂NH—; L² is —NH—; L³ is —CH₂—; R² and R³ are each H; and R⁴ is (2S)-oxetan-2-yl-methyl. In certain embodiments of any of Formulas (LVII)-(LXIV), L¹ is —SO₂NH—; L² is —N(Me)-; L³ is —CH₂—; R² and R³ are each H; and R⁴ is (2S)-oxetan-2-yl-methyl. In certain embodiments of any of Formulas (LVII)-(LXIV), L¹ is —SO₂NH—; L² is —O—; L³ is —CH₂—; R² and R³ are each H; and R⁴ is (1-ethyl-1H-imidazol-5-yl)-methyl. In certain embodiments of any of Formulas (LVII)-(LXIV), L¹ is —SO₂NH—; L² is —CF₂—; L³ is —CH₂—; R² and R³ are each H; and R⁴ is (1-ethyl-1H-imidazol-5-yl)-methyl. In certain embodiments of any of Formulas (LVII)-(LXIV), L¹ is —SO₂NH—; L² is —CHF—; L³ is —CH₂—; R² and R³ are each H; and R⁴ is (1-ethyl-1H-imidazol-5-yl)-methyl. In certain embodiments of any of Formulas (LVII)-(LXIV), L¹ is —SO₂NH—; L² is —CH(OH)—; L³ is —CH₂—; R² and R³ are each H; and R⁴ is (1-ethyl-1H-imidazol-5-yl)-methyl. In certain embodiments of any of Formulas (LVII)-(LXIV), L¹ is —SO₂NH—; L² is —NH—; L³ is —CH₂—; R² and R³ are each H; and R⁴ is (1-ethyl-1H-imidazol-5-yl)-methyl. In certain embodiments of any of Formulas (LVII)-(LXIV), L¹ is —SO₂NH—; L² is —N(Me)-; L³ is —CH₂—; R² and R³ are each H; and R⁴ is (1-ethyl-1H-imidazol-5-yl)-methyl. In certain embodiments of any of Formulas (LVII)-(LXIV), L¹ is —SO₂NH—; L² is —O—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —SO₂NH—; L² is —CF₂—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —SO₂NH—; L² is —CHF—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —SO₂NH—; L² is —CH(OH)—; L³ is —CH₂—; R² and R³ are each H; R⁴ is (2S)-oxetan-2-yl-methyl; and R$ is —COOH. In certain embodiments of any of Formulas (LVII)-(LXIV), L¹ is —SO₂NH—; L² is —NH—; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —SO₂NH—; L² is —N(Me)-; L³ is —CH₂—; R² and R³ are each H; R⁴ is (2S)-oxetan-2-yl-methyl; and R⁵ is —COOH. In certain embodiments of any of Formulas (LVII)-(LXIV), L¹ is —SO₂NH—; L² is —O—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —SO₂NH—; L² is —CF₂—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —SO₂NH—; L² is —CHF—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —SO₂NH—; L² is —CH(OH)—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —SO₂NH—; L² is —NH—; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —SO₂NH—; L² is —N(Me)-; L³ is —CH₂—; R² and R³ are each H; R⁴ is (1-ethyl-1H-imidazol-5-yl)-methyl; and R⁵ is —COOH. In certain embodiments of any of Formulas (LVII)-(LXIV), L¹ is —SO₂NH—; L² is —O—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CF₂—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CHF—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CH(OH)—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —NH—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —N(Me)-; L³ is —CH₂—; R² and R³ are each H; R⁴ is (2S)-oxetan-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —O—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CF₂—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CHF—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CH(OH)—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —NH—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —N(Me)-; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —O—; L² is —O—; L³ is —CH₂—; R² and R³ are each H; and R⁴ is oxelan-3-yl-methyl. In certain embodiments of any of Formulas (LVII)-(LXIV), L¹ is —O—; L² is —CF₂—; L³ is —CH₂—; R² and R³ are each H; and R⁴ is oxelan-3-yl-methyl. In certain embodiments of any of Formulas (LVII)-(LXIV), L¹ is —O—; L² is —CHF—; L³ is —CH₂—; R² and R³ are each H; and R⁴ is oxelan-3-yl-methyl. In certain embodiments of any of Formulas (LVII)-(LXIV), L¹ is —O—; L² is —CH(OH)—; L³ is —CH₂—; R² and R³ are each H; and R⁴ is oxelan-3-yl-methyl. In certain embodiments of any of Formulas (LVII)-(LXIV), L¹ is —O—; L² is —NH—; L³ is —CH₂—; R² and R³ are each H; and R⁴ is oxelan-3-yl-methyl. In certain embodiments of any of Formulas (LVII)-(LXIV), L¹ is —O—; L² is —N(Me)-; L³ is —CH₂—; R² and R³ are each H; and R⁴ is oxelan-3-yl-methyl. In certain embodiments of any of Formulas (LVII)-(LXIV), L¹ is —O—; L² is —O—; L³ is —CH₂—; R² and R³ are each H; and R⁴ is 1,3-oxazol-2-yl-methyl. In certain embodiments of any of Formulas (LVII)-(LXIV), L¹ is —O—; L² is —CF₂—; L³ is —CH₂—; R² and R³ are each H; and R⁴ is 1,3-oxazol-2-yl-methyl. In certain embodiments of any of Formulas (LVII)-(LXIV), L¹ is —O—; L² is —CHF—; L³ is —CH₂—; R² and R³ are each H; and R⁴ is 1,3-oxazol-2-yl-methyl. In certain embodiments of any of Formulas (LVII)-(LXIV), L¹ is —O—; L² is —CH(OH)—; L³ is —CH₂—; R² and R³ are each H; and R⁴ is 1,3-oxazol-2-yl-methyl. In certain embodiments of any of Formulas (LVII)-(LXIV), L¹ is —O—; L² is —NH—; L³ is —CH₂—; R² and R³ are each H; and R⁴ is 1,3-oxazol-2-yl-methyl. In certain embodiments of any of Formulas (LVII)-(LXIV), L¹ is —O—; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —O—; L² is —O—; L³ is —CH₂—; R² and R³ are each H; R⁴ is oxelan-3-yl-methyl; and R⁵ is —COOH. In certain embodiments of any of Formulas (LVII)-(LXIV), L¹ is —O—; L² is —CF₂—; L³ is —CH₂—; R² and R³ are each H; R⁴ is oxelan-3-yl-methyl; and R⁵ is —COOH. In certain embodiments of any of Formulas (LVII)-(LXIV), L¹ is —O—; L² is —CHF—; L³ is —CH₂—; R² and R³ are each H; R⁴ is oxelan-3-yl-methyl; and R⁵ is —COOH. In certain embodiments of any of Formulas (LVII)-(LXIV), L¹ is —O—; L² is —CH(OH)—; L³ is —CH₂—; R² and R³ are each H; R⁴ is oxelan-3-yl-methyl; and R⁵ is —COOH. In certain embodiments of any of Formulas (LVII)-(LXIV), L¹ is —O—; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —O—; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —O—; L² is —O—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —O—; L² is —CF₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —O—; L² is —CHF—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —O—; L² is —CH(OH)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —O—; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —O—; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —O—; L² is —O—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —O—; L² is —CF₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —O—; L² is —CHF—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —O—; L² is —CH(OH)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —O—; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —O—; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —O—; L² is —O—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —CF₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —CHF—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —CH(OH)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —O—; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —CH₂O—; L² is —O—; L³ is —CH₂—; R² and R³ are each H; and R⁴ is oxelan-3-yl-methyl. In certain embodiments of any of Formulas (LVII)-(LXIV), L¹ is —CH₂O—; L² is —CF₂—; L³ is —CH₂—; R² and R³ are each H; and R⁴ is oxelan-3-yl-methyl. In certain embodiments of any of Formulas (LVII)-(LXIV), L¹ is —CH₂O—; L² is —CHF—; L³ is —CH₂—; R² and R³ are each H; and R⁴ is oxelan-3-yl-methyl. In certain embodiments of any of Formulas (LVII)-(LXIV), L¹ is —CH₂O—; L² is —CH(OH)—; L³ is —CH₂—; R² and R³ are each H; and R⁴ is oxelan-3-yl-methyl. In certain embodiments of any of Formulas (LVII)-(LXIV), L¹ is —CH₂O—; L² is —NH—; L³ is —CH₂—; R² and R³ are each H; and R⁴ is oxelan-3-yl-methyl. In certain embodiments of any of Formulas (LVII)-(LXIV), L¹ is —CH₂O—; L² is —N(Me)-; L³ is —CH₂—; R² and R³ are each H; and R⁴ is oxelan-3-yl-methyl. In certain embodiments of any of Formulas (LVII)-(LXIV), L¹ is —CH₂O—; L² is —O—; L³ is —CH₂—; R² and R³ are each H; and R⁴ is 1,3-oxazol-2-yl-methyl. In certain embodiments of any of Formulas (LVII)-(LXIV), L¹ is —CH₂O—; L² is —CF₂—; L³ is —CH₂—; R² and R³ are each H; and R⁴ is 1,3-oxazol-2-yl-methyl. In certain embodiments of any of Formulas (LVII)-(LXIV), L¹ is —CH₂O—; L² is —CHF—; L³ is —CH₂—; R² and R³ are each H; and R⁴ is 1,3-oxazol-2-yl-methyl. In certain embodiments of any of Formulas (LVII)-(LXIV), L¹ is —CH₂O—; L² is —CH(OH)—; L³ is —CH₂—; R² and R³ are each H; and R⁴ is 1,3-oxazol-2-yl-methyl. In certain embodiments of any of Formulas (LVII)-(LXIV), L¹ is —CH₂O—; L² is —NH—; L³ is —CH₂—; R² and R³ are each H; and R⁴ is 1,3-oxazol-2-yl-methyl. In certain embodiments of any of Formulas (LVII)-(LXIV), L¹ is —CH₂O—; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —CH₂O—; L² is —O—; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂O—; L² is —CF₂—; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂O—; L² is —CHF—; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂O—; L² is —CH(OH)—; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂O—; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂O—; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —CH₂O—; L² is —O—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂O—; L² is —CF₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂O—; L² is —CHF—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂O—; L² is —CH(OH)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂O—; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂O—; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —CH₂O—; L² is —O—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CF₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CHF—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CH(OH)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —O—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CF₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CHF—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —CH(OH)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —CH₂O—; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —OCH₂—; L² is —O—; L³ is —CH₂—; R² and R³ are each H; and R⁴ is oxelan-3-yl-methyl. In certain embodiments of any of Formulas (LVII)-(LXIV), L¹ is —OCH₂—; L² is —CF₂—; L³ is —CH₂—; R² and R³ are each H; and R⁴ is oxelan-3-yl-methyl. In certain embodiments of any of Formulas (LVII)-(LXIV), L¹ is —OCH₂—; L² is —CHF—; L³ is —CH₂—; R² and R³ are each H; and R⁴ is oxelan-3-yl-methyl. In certain embodiments of any of Formulas (LVII)-(LXIV), L¹ is —OCH₂—; L² is —CH(OH)—; L³ is —CH₂—; R² and R³ are each H; and R⁴ is oxelan-3-yl-methyl. In certain embodiments of any of Formulas (LVII)-(LXIV), L¹ is —OCH₂—; L² is —NH—; L³ is —CH₂—; R² and R³ are each H; and R⁴ is oxelan-3-yl-methyl. In certain embodiments of any of Formulas (LVII)-(LXIV), L¹ is —OCH₂—; L² is —N(Me)-; L³ is —CH₂—; R² and R³ are each H; and R⁴ is oxelan-3-yl-methyl. In certain embodiments of any of Formulas (LVII)-(LXIV), L¹ is —OCH₂—; L² is —O—; L³ is —CH₂—; R² and R³ are each H; and R⁴ is 1,3-oxazol-2-yl-methyl. In certain embodiments of any of Formulas (LVII)-(LXIV), L¹ is —OCH₂—; L² is —CF₂—; L³ is —CH₂—; R² and R³ are each H; and R⁴ is 1,3-oxazol-2-yl-methyl. In certain embodiments of any of Formulas (LVII)-(LXIV), L¹ is —OCH₂—; L² is —CHF—; L³ is —CH₂—; R² and R³ are each H; and R⁴ is 1,3-oxazol-2-yl-methyl. In certain embodiments of any of Formulas (LVII)-(LXIV), L¹ is —OCH₂—; L² is —CH(OH)—; L³ is —CH₂—; R² and R³ are each H; and R⁴ is 1,3-oxazol-2-yl-methyl. In certain embodiments of any of Formulas (LVII)-(LXIV), L¹ is —OCH₂—; L² is —NH—; L³ is —CH₂—; R² and R³ are each H; and R⁴ is 1,3-oxazol-2-yl-methyl. In certain embodiments of any of Formulas (LVII)-(LXIV), L¹ is —OCH₂—; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —OCH₂—; L² is —O—; L³ is —CH₂—; R² and R³ are each H; R⁴ is oxelan-3-yl-methyl; and R⁵ is —COOH. In certain embodiments of any of Formulas (LVII)-(LXIV), L¹ is —OCH₂—; L² is —CF₂—; L³ is —CH₂—; R² and R³ are each H; R⁴ is oxelan-3-yl-methyl; and R⁵ is —COOH. In certain embodiments of any of Formulas (LVII)-(LXIV), L¹ is —OCH₂—; L² is —CHF—; L³ is —CH₂—; R² and R³ are each H; R⁴ is oxelan-3-yl-methyl; and R⁵ is —COOH. In certain embodiments of any of Formulas (LVII)-(LXIV), L¹ is —OCH₂—; L² is —CH(OH)—; L³ is —CH₂—; R² and R³ are each H; R⁴ is oxelan-3-yl-methyl; and R⁵ is —COOH. In certain embodiments of any of Formulas (LVII)-(LXIV), L¹ is —OCH₂—; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —OCH₂—; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —OCH₂—; L² is —O—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OCH₂—; L² is —CF₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OCH₂—; L² is —CHF—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OCH₂—; L² is —CH(OH)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OCH₂—; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OCH₂—; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OCH₂—; L² is —O—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CF₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CHF—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CH(OH)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —O—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CF₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CHF—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —CH(OH)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OCH₂—; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —NH—; L² is —O—; L³ is —CH₂—; R² and R³ are each H; and R⁴ is oxelan-3-yl-methyl. In certain embodiments of any of Formulas (LVII)-(LXIV), L¹ is —NH—; L² is —CF₂—; L³ is —CH₂—; R² and R³ are each H; and R⁴ is oxelan-3-yl-methyl. In certain embodiments of any of Formulas (LVII)-(LXIV), L¹ is —NH—; L² is —CHF—; L³ is —CH₂—; R² and R³ are each H; and R⁴ is oxelan-3-yl-methyl. In certain embodiments of any of Formulas (LVII)-(LXIV), L¹ is —NH—; L² is —CH(OH)—; L³ is —CH₂—; R² and R³ are each H; and R⁴ is oxelan-3-yl-methyl. In certain embodiments of any of Formulas (LVII)-(LXIV), L¹ is —NH—; L² is —NH—; L³ is —CH₂—; R² and R³ are each H; and R⁴ is oxelan-3-yl-methyl. In certain embodiments of any of Formulas (LVII)-(LXIV), L¹ is —NH—; L² is —N(Me)-; L³ is —CH₂—; R² and R³ are each H; and R⁴ is oxelan-3-yl-methyl. In certain embodiments of any of Formulas (LVII)-(LXIV), L¹ is —NH—; L² is —O—; L³ is —CH₂—; R² and R³ are each H; and R⁴ is 1,3-oxazol-2-yl-methyl. In certain embodiments of any of Formulas (LVII)-(LXIV), L¹ is —NH—; L² is —CF₂—; L³ is —CH₂—; R² and R³ are each H; and R⁴ is 1,3-oxazol-2-yl-methyl. In certain embodiments of any of Formulas (LVII)-(LXIV), L¹ is —NH—; L² is —CHF—; L³ is —CH₂—; R² and R³ are each H; and R⁴ is 1,3-oxazol-2-yl-methyl. In certain embodiments of any of Formulas (LVII)-(LXIV), L¹ is —NH—; L² is —CH(OH)—; L³ is —CH₂—; R² and R³ are each H; and R⁴ is 1,3-oxazol-2-yl-methyl. In certain embodiments of any of Formulas (LVII)-(LXIV), L¹ is —NH—; L² is —NH—; L³ is —CH₂—; R² and R³ are each H; and R⁴ is 1,3-oxazol-2-yl-methyl. In certain embodiments of any of Formulas (LVII)-(LXIV), L¹ is —NH—; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —NH—; L² is —O—; L³ is —CH₂—; R² and R³ are each H; R⁴ is oxelan-3-yl-methyl; and R⁵ is —COOH. In certain embodiments of any of Formulas (LVII)-(LXIV), L¹ is —NH—; L² is —CF₂—; L³ is —CH₂—; R² and R³ are each H; R⁴ is oxelan-3-yl-methyl; and R⁵ is —COOH. In certain embodiments of any of Formulas (LVII)-(LXIV), L¹ is —NH—; L² is —CHF—; L³ is —CH₂—; R² and R³ are each H; R⁴ is oxelan-3-yl-methyl; and R⁵ is —COOH. In certain embodiments of any of Formulas (LVII)-(LXIV), L¹ is —NH—; L² is —CH(OH)—; L³ is —CH₂—; R² and R³ are each H; R⁴ is oxelan-3-yl-methyl; and R⁵ is —COOH. In certain embodiments of any of Formulas (LVII)-(LXIV), L¹ is —NH—; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —NH—; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —NH—; L² is —O—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NH—; L² is —CF₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NH—; L² is —CHF—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NH—; L² is —CH(OH)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NH—; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NH—; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NH—; L² is —O—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NH—; L² is —CF₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NH—; L² is —CHF—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NH—; L² is —CH(OH)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NH—; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NH—; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NH—; L² is —O—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —CF₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —CHF—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —CH(OH)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NH—; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments of any of Formulas (LVII)-(LXIV), R⁴ is (R)-1,3-oxazol-2-yl-methyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —N(Me)-; L² is —O—; L³ is —CH₂—; R² and R³ are each H; and R⁴ is oxelan-3-yl-methyl. In certain embodiments of any of Formulas (LVII)-(LXIV), L¹ is —N(Me)-; L² is —CF₂—; L³ is —CH₂—; R² and R³ are each H; and R⁴ is oxelan-3-yl-methyl. In certain embodiments of any of Formulas (LVII)-(LXIV), L¹ is —N(Me)-; L² is —CHF—; L³ is —CH₂—; R² and R³ are each H; and R⁴ is oxelan-3-yl-methyl. In certain embodiments of any of Formulas (LVII)-(LXIV), L¹ is —N(Me)-; L² is —CH(OH)—; L³ is —CH₂—; R² and R³ are each H; and R⁴ is oxelan-3-yl-methyl. In certain embodiments of any of Formulas (LVII)-(LXIV), L¹ is —N(Me)-; L² is —NH—; L³ is —CH₂—; R² and R³ are each H; and R⁴ is oxelan-3-yl-methyl. In certain embodiments of any of Formulas (LVII)-(LXIV), L¹ is —N(Me)-; L² is —N(Me)-; L³ is —CH₂—; R² and R³ are each H; and R⁴ is oxelan-3-yl-methyl. In certain embodiments of any of Formulas (LVII)-(LXIV), L¹ is —N(Me)-; L² is —O—; L³ is —CH₂—; R² and R³ are each H; and R⁴ is 1,3-oxazol-2-yl-methyl. In certain embodiments of any of Formulas (LVII)-(LXIV), L¹ is —N(Me)-; L² is —CF₂—; L³ is —CH₂—; R² and R³ are each H; and R⁴ is 1,3-oxazol-2-yl-methyl. In certain embodiments of any of Formulas (LVII)-(LXIV), L¹ is —N(Me)-; L² is —CHF—; L³ is —CH₂—; R² and R³ are each H; and R⁴ is 1,3-oxazol-2-yl-methyl. In certain embodiments of any of Formulas (LVII)-(LXIV), L¹ is —N(Me)-; L² is —CH(OH)—; L³ is —CH₂—; R² and R³ are each H; and R⁴ is 1,3-oxazol-2-yl-methyl. In certain embodiments of any of Formulas (LVII)-(LXIV), L¹ is —N(Me)-; L² is —NH—; L³ is —CH₂—; R² and R³ are each H; and R⁴ is 1,3-oxazol-2-yl-methyl. In certain embodiments of any of Formulas (LVII)-(LXIV), L¹ is —N(Me)-; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —N(Me)-; L² is —O—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)-; L² is —CF₂—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)-; L² is —CHF—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)-; L² is —CH(OH)—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)-; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)-; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)-; L² is —O—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)-; L² is —CF₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)-; L² is —CHF—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)-; L² is —CH(OH)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)-; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)-; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)-; L² is —O—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CF₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CHF—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CH(OH)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —O—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CF₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CHF—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —CH(OH)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)-; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —N(Et)-; L² is —O—; L³ is —CH₂—; R² and R³ are each H; and R⁴ is oxelan-3-yl-methyl. In certain embodiments of any of Formulas (LVII)-(LXIV), L¹ is —N(Et)-; L² is —CF₂—; L³ is —CH₂—; R² and R³ are each H; and R⁴ is oxelan-3-yl-methyl. In certain embodiments of any of Formulas (LVII)-(LXIV), L¹ is —N(Et)-; L² is —CHF—; L³ is —CH₂—; R² and R³ are each H; and R⁴ is oxelan-3-yl-methyl. In certain embodiments of any of Formulas (LVII)-(LXIV), L¹ is —N(Et)-; L² is —CH(OH)—; L³ is —CH₂—; R² and R³ are each H; and R⁴ is oxelan-3-yl-methyl. In certain embodiments of any of Formulas (LVII)-(LXIV), L¹ is —N(Et)-; L² is —NH—; L³ is —CH₂—; R² and R³ are each H; and R⁴ is oxelan-3-yl-methyl. In certain embodiments of any of Formulas (LVII)-(LXIV), L¹ is —N(Et)-; L² is —N(Me)-; L³ is —CH₂—; R² and R³ are each H; and R⁴ is oxelan-3-yl-methyl. In certain embodiments of any of Formulas (LVII)-(LXIV), L¹ is —N(Et)-; L² is —O—; L³ is —CH₂—; R² and R³ are each H; and R⁴ is 1,3-oxazol-2-yl-methyl. In certain embodiments of any of Formulas (LVII)-(LXIV), L¹ is —N(Et)-; L² is —CF₂—; L³ is —CH₂—; R² and R³ are each H; and R⁴ is 1,3-oxazol-2-yl-methyl. In certain embodiments of any of Formulas (LVII)-(LXIV), L¹ is —N(Et)-; L² is —CHF—; L³ is —CH₂—; R² and R³ are each H; and R⁴ is 1,3-oxazol-2-yl-methyl. In certain embodiments of any of Formulas (LVII)-(LXIV), L¹ is —N(Et)-; L² is —CH(OH)—; L³ is —CH₂—; R² and R³ are each H; and R⁴ is 1,3-oxazol-2-yl-methyl. In certain embodiments of any of Formulas (LVII)-(LXIV), L¹ is —N(Et)-; L² is —NH—; L³ is —CH₂—; R² and R³ are each H; and R⁴ is 1,3-oxazol-2-yl-methyl. In certain embodiments of any of Formulas (LVII)-(LXIV), L¹ is —N(Et)-; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —N(Et)-; L² is —O—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Et)-; L² is —CF₂—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Et)-; L² is —CHF—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Et)-; L² is —CH(OH)—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Et)-; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Et)-; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —N(Et)-; L² is —O—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Et)-; L² is —CF₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Et)-; L² is —CHF—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Et)-; L² is —CH(OH)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Et)-; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Et)-; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Et)-; L² is —O—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —CF₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —CHF—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —CH(OH)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —O—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —CF₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —CHF—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —CH(OH)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Et)-; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —NHCH₂—; L² is —O—; L³ is —CH₂—; R² and R³ are each H; and R⁴ is oxelan-3-yl-methyl. In certain embodiments of any of Formulas (LVII)-(LXIV), L¹ is —NHCH₂—; L² is —CF₂—; L³ is —CH₂—; R² and R³ are each H; and R⁴ is oxelan-3-yl-methyl. In certain embodiments of any of Formulas (LVII)-(LXIV), L¹ is —NHCH₂—; L² is —CHF—; L³ is —CH₂—; R² and R³ are each H; and R⁴ is oxelan-3-yl-methyl. In certain embodiments of any of Formulas (LVII)-(LXIV), L¹ is —NHCH₂—; L² is —CH(OH)—; L³ is —CH₂—; R² and R³ are each H; and R⁴ is oxelan-3-yl-methyl. In certain embodiments of any of Formulas (LVII)-(LXIV), L¹ is —NHCH₂—; L² is —NH—; L³ is —CH₂—; R² and R³ are each H; and R⁴ is oxelan-3-yl-methyl. In certain embodiments of any of Formulas (LVII)-(LXIV), L¹ is —NHCH₂—; L² is —N(Me)-; L³ is —CH₂—; R² and R³ are each H; and R⁴ is oxelan-3-yl-methyl. In certain embodiments of any of Formulas (LVII)-(LXIV), L¹ is —NHCH₂—; L² is —O—; L³ is —CH₂—; R² and R³ are each H; and R⁴ is 1,3-oxazol-2-yl-methyl. In certain embodiments of any of Formulas (LVII)-(LXIV), L¹ is —NHCH₂—; L² is —CF₂—; L³ is —CH₂—; R² and R³ are each H; and R⁴ is 1,3-oxazol-2-yl-methyl. In certain embodiments of any of Formulas (LVII)-(LXIV), L¹ is —NHCH₂—; L² is —CHF—; L³ is —CH₂—; R² and R³ are each H; and R⁴ is 1,3-oxazol-2-yl-methyl. In certain embodiments of any of Formulas (LVII)-(LXIV), L¹ is —NHCH₂—; L² is —CH(OH)—; L³ is —CH₂—; R² and R³ are each H; and R⁴ is 1,3-oxazol-2-yl-methyl. In certain embodiments of any of Formulas (LVII)-(LXIV), L¹ is —NHCH₂—; L² is —NH—; L³ is —CH₂—; R² and R³ are each H; and R⁴ is 1,3-oxazol-2-yl-methyl. In certain embodiments of any of Formulas (LVII)-(LXIV), L¹ is —NHCH₂—; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —NHCH₂—; L² is —O—; L³ is —CH₂—; R² and R³ are each H; R⁴ is oxelan-3-yl-methyl; and R$ is —COOH. In certain embodiments of any of Formulas (LVII)-(LXIV), L¹ is —NHCH₂—; L² is —CF₂—; L³ is —CH₂—; R² and R³ are each H; R⁴ is oxelan-3-yl-methyl; and R⁵ is —COOH. In certain embodiments of any of Formulas (LVII)-(LXIV), L¹ is —NHCH₂—; L² is —CHF—; L³ is —CH₂—; R² and R³ are each H; R⁴ is oxelan-3-yl-methyl; and R⁵ is —COOH. In certain embodiments of any of Formulas (LVII)-(LXIV), L¹ is —NHCH₂—; L² is —CH(OH)—; L³ is —CH₂—; R² and R³ are each H; R⁴ is oxelan-3-yl-methyl; and R⁵ is —COOH. In certain embodiments of any of Formulas (LVII)-(LXIV), L¹ is —NHCH₂—; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —NHCH₂—; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —NHCH₂—; L² is —O—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NHCH₂—; L² is —CF₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NHCH₂—; L² is —CHF—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NHCH₂—; L² is —CH(OH)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NHCH₂—; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NHCH₂—; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —NHCH₂—; L² is —O—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CF₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CHF—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CH(OH)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —O—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CF₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CHF—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —CH(OH)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —NHCH₂—; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is 3-fluorooxetan-3-yl-methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —N(Me)CH₂—; L² is —O—; L³ is —CH₂—; R² and R³ are each H; and R⁴ is oxelan-3-yl-methyl. In certain embodiments of any of Formulas (LVII)-(LXIV), L¹ is —N(Me)CH₂—; L² is —CF₂—; L³ is —CH₂—; R² and R³ are each H; and R⁴ is oxelan-3-yl-methyl. In certain embodiments of any of Formulas (LVII)-(LXIV), L¹ is —N(Me)CH₂—; L² is —CHF—; L³ is —CH₂—; R² and R³ are each H; and R⁴ is oxelan-3-yl-methyl. In certain embodiments of any of Formulas (LVII)-(LXIV), L¹ is —N(Me)CH₂—; L² is —CH(OH)—; L³ is —CH₂—; R² and R³ are each H; and R⁴ is oxelan-3-yl-methyl. In certain embodiments of any of Formulas (LVII)-(LXIV), L¹ is —N(Me)CH₂—; L² is —NH—; L³ is —CH₂—; R² and R³ are each H; and R⁴ is oxelan-3-yl-methyl. In certain embodiments of any of Formulas (LVII)-(LXIV), L¹ is —N(Me)CH₂—; L² is —N(Me)-; L³ is —CH₂—; R² and R³ are each H; and R⁴ is oxelan-3-yl-methyl. In certain embodiments of any of Formulas (LVII)-(LXIV), L¹ is —N(Me)CH₂—; L² is —O—; L³ is —CH₂—; R² and R³ are each H; and R⁴ is 1,3-oxazol-2-yl-methyl. In certain embodiments of any of Formulas (LVII)-(LXIV), L¹ is —N(Me)CH₂—; L² is —CF₂—; L³ is —CH₂—; R² and R³ are each H; and R⁴ is 1,3-oxazol-2-yl-methyl. In certain embodiments of any of Formulas (LVII)-(LXIV), L¹ is —N(Me)CH₂—; L² is —CHF—; L³ is —CH₂—; R² and R³ are each H; and R⁴ is 1,3-oxazol-2-yl-methyl. In certain embodiments of any of Formulas (LVII)-(LXIV), L¹ is —N(Me)CH₂—; L² is —CH(OH)—; L³ is —CH₂—; R² and R³ are each H; and R⁴ is 1,3-oxazol-2-yl-methyl. In certain embodiments of any of Formulas (LVII)-(LXIV), L¹ is —N(Me)CH₂—; L² is —NH—; L³ is —CH₂—; R² and R³ are each H; and R⁴ is 1,3-oxazol-2-yl-methyl. In certain embodiments of any of Formulas (LVII)-(LXIV), L¹ is —N(Me)CH₂—; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —N(Me)CH₂—; L² is —O—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)CH₂—; L² is —CF₂—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)CH₂—; L² is —CHF—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)CH₂—; L² is —CH(OH)—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)CH₂—; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)CH₂—; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —N(Me)CH₂—; L² is —O—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)CH₂—; L² is —CF₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)CH₂—; L² is —CHF—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)CH₂—; L² is —CH(OH)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)CH₂—; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)CH₂—; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —N(Me)CH₂—; L² is —O—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CF₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CHF—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CH(OH)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —O—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CF₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CHF—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —CH(OH)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —N(Me)CH₂—; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is 3-fluorooxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy) propyl. In certain embodiments of any of Formulas (LVII)-(LXIV), R⁴ is (2S)-2-(difluoromethoxy) propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy) propyl. In certain embodiments of any of Formulas (LVII)-(LXIV), R⁴ is (2S)-2-(2,2,2-trifluoroethoxy) propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —OC(H,Me)-; L² is —CF₂—; L³ is —CH₂—; R² and R³ are each H; and R⁴ is oxelan-3-yl-methyl. In certain embodiments of any of Formulas (LVII)-(LXIV), L¹ is —OC(H,Me)-; L² is —CHF—; L³ is —CH₂—; R² and R³ are each H; and R⁴ is oxelan-3-yl-methyl. In certain embodiments of any of Formulas (LVII)-(LXIV), L¹ is —OC(H,Me)-; L² is —CH(OH)—; L³ is —CH₂—; R² and R³ are each H; and R⁴ is oxelan-3-yl-methyl. In certain embodiments of any of Formulas (LVII)-(LXIV), L¹ is —OC(H,Me)-; L² is —NH—; L³ is —CH₂—; R² and R³ are each H; and R⁴ is oxelan-3-yl-methyl. In certain embodiments of any of Formulas (LVII)-(LXIV), L¹ is —OC(H,Me)-; L² is —N(Me)-; L³ is —CH₂—; R² and R³ are each H; and R⁴ is oxelan-3-yl-methyl. In certain embodiments of any of Formulas (LVII)-(LXIV), L¹ is —OC(H,Me)-; L² is —O—; L³ is —CH₂—; R² and R³ are each H; and R⁴ is 1,3-oxazol-2-yl-methyl. In certain embodiments of any of Formulas (LVII)-(LXIV), L¹ is —OC(H,Me)-; L² is —CF₂—; L³ is —CH₂—; R² and R³ are each H; and R⁴ is 1,3-oxazol-2-yl-methyl. In certain embodiments of any of Formulas (LVII)-(LXIV), L¹ is —OC(H,Me)-; L² is —CHF—; L³ is —CH₂—; R² and R³ are each H; and R⁴ is 1,3-oxazol-2-yl-methyl. In certain embodiments of any of Formulas (LVII)-(LXIV), L¹ is —OC(H,Me)-; L² is —CH(OH)—; L³ is —CH₂—; R² and R³ are each H; and R⁴ is 1,3-oxazol-2-yl-methyl. In certain embodiments of any of Formulas (LVII)-(LXIV), L¹ is —OC(H,Me)-; L² is —NH—; L³ is —CH₂—; R² and R³ are each H; and R⁴ is 1,3-oxazol-2-yl-methyl. In certain embodiments of any of Formulas (LVII)-(LXIV), L¹ is —OC(H,Me)-; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —OC(H,Me)-; L² is —O—; L³ is —CH₂—; R² and R³ are each H; R⁴ is oxelan-3-yl-methyl; and R⁵ is —COOH. In certain embodiments of any of Formulas (LVII)-(LXIV), L¹ is —OC(H,Me)-; L² is —CF₂—; L³ is —CH₂—; R² and R³ are each H; R⁴ is oxelan-3-yl-methyl; and R⁵ is —COOH. In certain embodiments of any of Formulas (LVII)-(LXIV), L¹ is —OC(H,Me)-; L² is —CHF—; L³ is —CH₂—; R² and R³ are each H; R⁴ is oxelan-3-yl-methyl; and R⁵ is —COOH. In certain embodiments of any of Formulas (LVII)-(LXIV), L¹ is —OC(H,Me)-; L² is —CH(OH)—; L³ is —CH₂—; R² and R³ are each H; R⁴ is oxelan-3-yl-methyl; and R⁵ is —COOH. In certain embodiments of any of Formulas (LVII)-(LXIV), L¹ is —OC(H,Me)-; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —OC(H,Me)-; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —OC(H,Me)-; L² is —O—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OC(H,Me)-; L² is —CF₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OC(H,Me)-; L² is —CHF—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OC(H,Me)-; L² is —CH(OH)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OC(H,Me)-; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OC(H,Me)-; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —OC(H,Me)-; L² is —O—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CF₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CHF—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CH(OH)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —O—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CF₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CHF—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —CH(OH)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —OC(H,Me)-; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is 3-fluorooxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is in certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy)propyl. In certain embodiments of any of Formulas (LVII)-(LXIV), R⁴ is (2S)-2-(difluoromethoxy)propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy)propyl. In certain embodiments of any of Formulas (LVII)-(LXIV), R⁴ is (2S)-2-(2,2,2-trifluoroethoxy)propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —SO₂NH—; L² is —O—; L³ is —CH₂—; R² and R³ are each H; and R⁴ is oxelan-3-yl-methyl. In certain embodiments of any of Formulas (LVII)-(LXIV), L¹ is —SO₂NH—; L² is —CF₂—; L³ is —CH₂—; R² and R³ are each H; and R⁴ is oxelan-3-yl-methyl. In certain embodiments of any of Formulas (LVII)-(LXIV), L¹ is —SO₂NH—; L² is —CHF—; L³ is —CH₂—; R² and R³ are each H; and R⁴ is oxelan-3-yl-methyl. In certain embodiments of any of Formulas (LVII)-(LXIV), L¹ is —SO₂NH—; L² is —CH(OH)—; L³ is —CH₂—; R² and R³ are each H; and R⁴ is oxelan-3-yl-methyl. In certain embodiments of any of Formulas (LVII)-(LXIV), L¹ is —SO₂NH—; L² is —NH—; L³ is —CH₂—; R² and R³ are each H; and R⁴ is oxelan-3-yl-methyl. In certain embodiments of any of Formulas (LVII)-(LXIV), L¹ is —SO₂NH—; L² is —O—; L³ is —CH₂—; R² and R³ are each H; and R⁴ is 1,3-oxazol-2-yl-methyl. In certain embodiments of any of Formulas (LVII)-(LXIV), L¹ is —SO₂NH—; L² is —CF₂—; L³ is —CH₂—; R² and R³ are each H; and R⁴ is 1,3-oxazol-2-yl-methyl. In certain embodiments of any of Formulas (LVII)-(LXIV), L¹ is —SO₂NH—; L² is —CHF—; L³ is —CH₂—; R² and R³ are each H; and R⁴ is 1,3-oxazol-2-yl-methyl. In certain embodiments of any of Formulas (LVII)-(LXIV), L¹ is —SO₂NH—; L² is —CH(OH)—; L³ is —CH₂—; R² and R³ are each H; and R⁴ is 1,3-oxazol-2-yl-methyl. In certain embodiments of any of Formulas (LVII)-(LXIV), L¹ is —SO₂NH—; L² is —NH—; L³ is —CH₂—; R² and R³ are each H; and R⁴ is 1,3-oxazol-2-yl-methyl. In certain embodiments of any of Formulas (LVII)-(LXIV), L¹ is —SO₂NH—; L² is —N(Me)-; L³ is —CH₂—; R² and R³ are each H; and R⁴ is 1,3-oxazol-2-yl-methyl. In certain embodiments of any of Formulas (LVII)-(LXIV), L¹ is —SO₂NH—; L² is —O—; L³ is —CH₂—; R² and R³ are each H; R⁴ is oxelan-3-yl-methyl; and R⁵ is —COOH. In certain embodiments of any of Formulas (LVII)-(LXIV), L¹ is —SO₂NH—; L² is —CF₂—; L³ is —CH₂—; R² and R³ are each H; R⁴ is oxelan-3-yl-methyl; and R⁵ is —COOH. In certain embodiments of any of Formulas (LVII)-(LXIV), L¹ is —SO₂NH—; L² is —CHF—; L³ is —CH₂—; R² and R³ are each H; R⁴ is oxelan-3-yl-methyl; and R⁵ is —COOH. In certain embodiments of any of Formulas (LVII)-(LXIV), L¹ is —SO₂NH—; L² is —CH(OH)—; L³ is —CH₂—; R² and R³ are each H; R⁴ is oxelan-3-yl-methyl; and R⁵ is —COOH. In certain embodiments of any of Formulas (LVII)-(LXIV), L¹ is —SO₂NH—; L² is —NH—; L³ is —CH₂—; R² and R³ are each H; R⁴ is oxelan-3-yl-methyl; and R⁵ is —COOH. In certain embodiments of any of Formulas (LVII)-(LXIV), L¹ is —SO₂NH—; L² is —N(Me)-; L³ is —CH₂—; R² and R³ are each H; R⁴ is oxelan-3-yl-methyl; and R⁵ is —COOH. In certain embodiments of any of Formulas (LVII)-(LXIV), L¹ is —SO₂NH—; L² is —O—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —SO₂NH—; L² is —CF₂—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —SO₂NH—; L² is —CHF—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —SO₂NH—; L² is —CH(OH)—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —SO₂NH—; L² is —NH—; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —SO₂NH—; L² is —N(Me)-; L³ is —CH₂—; R² and R³ are each H; R⁴ is 1,3-oxazol-2-yl-methyl; and R⁵ is —COOH. In certain embodiments of any of Formulas (LVII)-(LXIV), L¹ is —SO₂NH—; L² is —O—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CF₂—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CHF—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CH(OH)—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —NH—; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —N(Me)-; L³ is —CH₂—; R² and R³ are each H; R⁴ is oxelan-3-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —O—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CF₂—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CHF—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —CH(OH)—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —NH—; L³ is —CH₂—; R² and R³ are each H; R⁴ is 1,3-oxazol-2-yl-methyl; and R⁵ is tetrazolyl, for instance 1H-1,2,3,4-tetrazol-5-yl. In certain embodiments of any of Formulas (LVII)-(LXIV), L¹ is —SO₂NH—; L² is —N(Me)-; L³ is —CH₂—; R² and R³ are each H; R⁴ is 1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In certain embodiments of any of Formulas (LVII)-(LXIV), R⁴ is azetidine-3-yl-methyl. In certain embodiments of any of Formulas (LVII)-(LXIV), R⁴ is (3R)-azetidine-3-yl-methyl. In certain embodiments of any of Formulas (LVII)-(LXIV), R⁴ is (3S)-azetidine-3-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is butan-1-yl-one. In certain embodiments of any of Formulas (LVII)-(LXIV), R⁴ is 1-(cyanomethyl)-cycloprop-1-yl-methyl. In certain embodiments of any of Formulas (LVII)-(LXIV), R⁴ is 1-(Fluoromethyl)-cycloprop-1-yl-methyl. In certain embodiments of any of Formulas (LVII)-(LXIV), R⁴ is 1-hydroxyl-cycloprop-1-yl-methyl. In certain embodiments of any of Formulas (LVII)-(LXIV), R⁴ is isoxazol-5-yl-methyl. In certain embodiments of any of Formulas (LVII)-(LXIV), R⁴ is (1-methyl-1H-imidazol-5-yl)-methyl. In certain embodiments of any of Formulas (LVII)-(LXIV), R⁴ is 1-methoxy-cycloprop-1yl-methyl. In certain embodiments of any of Formulas (LVII)-(LXIV), R⁴ is methoxyethan-2-yl. In certain embodiments of any of Formulas (LVII)-(LXIV), R⁴ is 1-(2-methoxyethyl)-cycloprop-1-yl-methyl. In certain embodiments of any of Formulas (LVII)-(LXIV), R⁴ is {[1-(1,2-oxazole-3-carbonyl)azetidin-2-yl]methyl}. In certain embodiments of any of Formulas (LVII)-(LXIV), R⁴ is {[(2R)-1-(1,2-oxazole-3-carbonyl)azetidin-2-yl]methyl}. In certain embodiments of any of Formulas (LVII)-(LXIV), R⁴ is {[(2S)-1-(1,2-oxazole-3-carbonyl)azetidin-2-yl]methyl. In certain embodiments of any of Formulas (LVII)-(LXIV), R⁴ is 2-oxabicyclo[2.1.1]hexa-1-yl-methyl. In certain embodiments of any of Formulas (LVII)-(LXIV), R⁴ is (S)-buta-4-yl-1,3-diol. In certain embodiments of any of Formulas (LVII)-(LXIV), R⁴ is 3-(difluoromethyl)oxetan-3-yl-methyl. In certain embodiments of any of Formulas (LVII)-(LXIV), R⁴ is 3-fluorooxetan-3-yl-methyl. In certain embodiments of any of Formulas (LVII)-(LXIV), R⁴ is ((R)-1,1-dioxidothietan-2-yl)methyl. In certain embodiments of any of Formulas (LVII)-(LXIV), R⁴ is ((S)-1,1-dioxidothietan-2-yl)methyl. In certain embodiments of any of Formulas (LVII)-(LXIV), R⁴ is (3R,4R)-4-methoxytetrahydrofuran-3-yl. In certain embodiments of any of Formulas (LVII)-(LXIV), R⁴ is (S)-1,3-oxazol-2-yl-methyl. In certain embodiments of any of Formulas (LVII)-(LXIV), R⁴ is (R)-1,3-oxazol-2-yl-methyl. In certain embodiments of any of Formulas (LVII)-(LXIV), R⁴ is 1-cyclobutylmethyl. In certain embodiments of any of Formulas (LVII)-(LXIV), R⁴ is (R)-2-cyclopropoxypropyl. In certain embodiments of any of Formulas (LVII)-(LXIV), R⁴ is (S)-2-cyclopropoxypropyl. In certain embodiments of any of Formulas (LVII)-(LXIV), R⁴ is (2R)-2-(difluoromethoxy)propyl. In certain embodiments of any of Formulas (LVII)-(LXIV), R⁴ is (2S)-2-(difluoromethoxy)propyl. In certain embodiments of any of Formulas (LVII)-(LXIV), R⁴ is (2S)-oxolan-2-yl-methyl. In certain embodiments of any of Formulas (LVII)-(LXIV), R⁴ is (1-methoxycyclobutyl)methyl. In certain embodiments of any of Formulas (LVII)-(LXIV), R⁴ is (2R)-2-(2,2,2-trifluoroethoxy)propyl. In certain embodiments of any of Formulas (LVII)-(LXIV), R⁴ is (2S)-2-(2,2,2-trifluoroethoxy)propyl. In certain embodiments of any of Formulas (LVII)-(LXIV), R⁴ is 2-(2,2,2-trifluoroethoxy)ethyl. In certain embodiments, provided is a compound of any of Formulas (LVII)-(LXIV), or a pharmaceutically salt, tautomer, stereoisomer, and/or mixture of stereoisomers thereof, wherein L² is —NH—. In certain embodiments, provided is a compound of any of Formulas (LVII)-(LXIV), or a pharmaceutically salt, tautomer, stereoisomer, and/or mixture of stereoisomers thereof, wherein L² is —N(Me)-.

In certain embodiments of any of Formulas (LVII)-(LXIV), L¹ is —OCH₂—; L² is —O—; L³ is —CH₂—; R² and R³ are each H; R⁴ is (2S)-oxetan-2-yl-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxetan-2-yl-methyl; and R⁵ is —CH₂COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxetan-2-yl-methyl; and R⁵ is —(CH₂)₂COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxetan-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxetan-2-yl-methyl; and R⁵ is

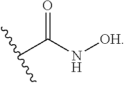 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxetan-2-yl-methyl; and R⁵ is

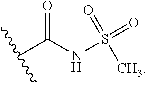 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxetan-2-yl-methyl; and R⁵ is

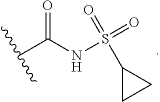 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-buta-4-yl-1,3-diol; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-buta-4-yl-1,3-diol; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-buta-4-yl-1,3-diol; and R⁵ is

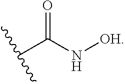 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃. In certain embodiments of any of Formulas (LVII)-(LXIV), L¹ is —OCH₂—; L² is —O—; L³ is —CH₂—; R² and R³ are each H; R⁴ is (S)-buta-4-yl-1,3-diol; and R⁵ is

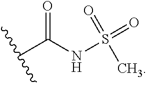 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-buta-4-yl-1,3-diol; and R⁵ is

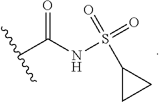 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-(difluoromethyl)oxetan-3-yl-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-(difluoromethyl)oxetan-3-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-(difluoromethyl)oxetan-3-yl-methyl; and R⁵ is

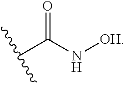 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-(difluoromethyl)oxetan-3-yl-methyl; and R⁵ is

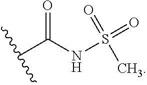 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-(difluoromethyl)oxetan-3-yl-methyl; and R⁵ is

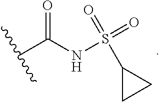 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-ethyl-1H-imidazol-5-yl)-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-ethyl-1H-imidazol-5-yl)-methyl; and R⁵ is

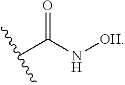 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-ethyl-1H-imidazol-5-yl)-methyl; and R⁵ is

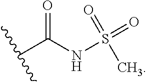 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-ethyl-1H-imidazol-5-yl)-methyl; and R⁵ is

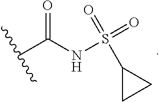 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-fluorooxetan-3-yl-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-fluorooxetan-3-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-fluorooxetan-3-yl-methyl; and R⁵ is

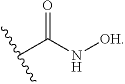 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-fluorooxetan-3-yl-methyl; and R⁵ is

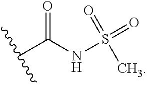 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-fluorooxetan-3-yl-methyl; and R⁵ is

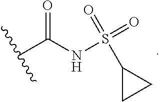 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (3R,4R)-4-methoxytetrahydrofuran-3-yl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (3R,4R)-4-methoxytetrahydrofuran-3-yl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (3R,4R)-4-methoxytetrahydrofuran-3-yl; and R⁵ is

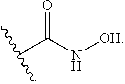 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (3R,4R)-4-methoxytetrahydrofuran-3-yl; and R⁵ is

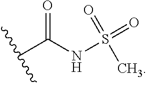 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (3R,4R)-4-methoxytetrahydrofuran-3-yl; and R⁵ is

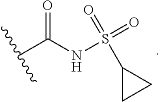 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-1,3-oxazol-2-yl-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-1,3-oxazol-2-yl-methyl; and R⁵ is

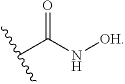 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-1,3-oxazol-2-yl-methyl; and R⁵ is

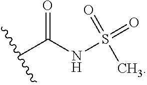 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-1,3-oxazol-2-yl-methyl; and R⁵ is

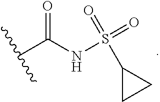 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-1,3-oxazol-2-yl-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-1,3-oxazol-2-yl-methyl; and R⁵ is

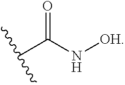 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-1,3-oxazol-2-yl-methyl; and R⁵ is

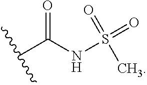 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-1,3-oxazol-2-yl-methyl; and R⁵ is

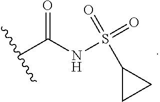 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-oxabicyclo[2.1.1]hexa-1-yl-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-oxabicyclo[2.1.1]hexa-1-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-oxabicyclo[2.1.1]hexa-1-yl-methyl; and R⁵ is

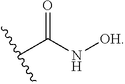 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-oxabicyclo[2.1.1]hexa-1-yl-methyl; and R⁵ is

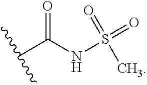 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-oxabicyclo[2.1.1]hexa-1-yl-methyl; and R⁵ is

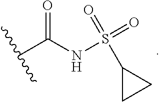 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (2S)-oxetan-2-yl-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (2S)-oxetan-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (2S)-oxetan-2-yl-methyl; and R⁵ is

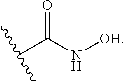 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (2S)-oxetan-2-yl-methyl; and R⁵ is

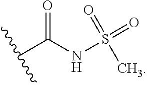 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (2S)-oxetan-2-yl-methyl; and R⁵ is

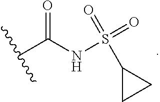 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (S)-buta-4-yl-1,3-diol; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (S)-buta-4-yl-1,3-diol; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (S)-buta-4-yl-1,3-diol; and R⁵ is

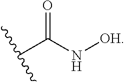 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (S)-buta-4-yl-1,3-diol; and R⁵ is

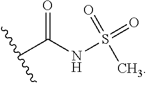 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (S)-buta-4-yl-1,3-diol; and R⁵ is

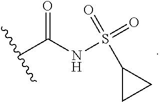 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is 3-(difluoromethyl)oxetan-3-yl-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is 3-(difluoromethyl)oxetan-3-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is 3-(difluoromethyl)oxetan-3-yl-methyl; and R⁵ is

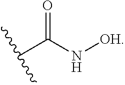 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is 3-(difluoromethyl)oxetan-3-yl-methyl; and R⁵ is

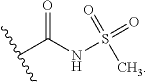 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is 3-(difluoromethyl)oxetan-3-yl-methyl; and R⁵ is

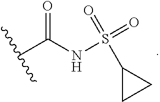 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (1-ethyl-1H-imidazol-5-yl)-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (1-ethyl-1H-imidazol-5-yl)-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (1-ethyl-1H-imidazol-5-yl)-methyl; and R⁵ is

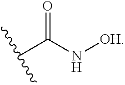 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (1-ethyl-1H-imidazol-5-yl)-methyl; and R⁵ is

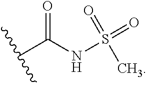 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (1-ethyl-1H-imidazol-5-yl)-methyl; and R⁵ is

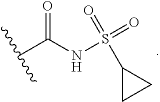 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is 3-fluorooxetan-3-yl-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is 3-fluorooxetan-3-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is 3-fluorooxetan-3-yl-methyl; and R⁵ is

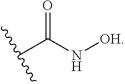 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is 3-fluorooxetan-3-yl-methyl; and R⁵ is

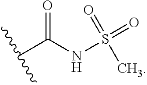 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is 3-fluorooxetan-3-yl-methyl; and R⁵ is

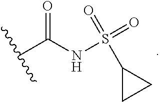 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (3R,4R)-4-methoxytetrahydrofuran-3-yl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (3R,4R)-4-methoxytetrahydrofuran-3-yl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (3R,4R)-4-methoxytetrahydrofuran-3-yl; and R⁵ is

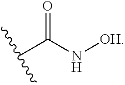 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (3R,4R)-4-methoxytetrahydrofuran-3-yl; and R⁵ is

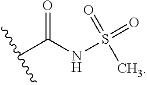 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (3R,4R)-4-methoxytetrahydrofuran-3-yl; and R⁵ is

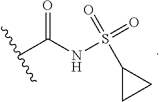 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (S)-1,3-oxazol-2-yl-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (S)-1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (S)-1,3-oxazol-2-yl-methyl; and R⁵ is

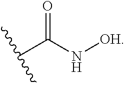 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (S)-1,3-oxazol-2-yl-methyl; and R⁵ is

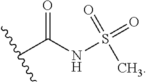 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (S)-1,3-oxazol-2-yl-methyl; and R⁵ is

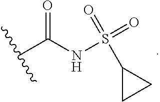 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (R)-1,3-oxazol-2-yl-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (R)-1,3-oxazol-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (R)-1,3-oxazol-2-yl-methyl; and R⁵ is

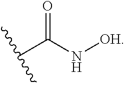 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (R)-1,3-oxazol-2-yl-methyl; and R⁵ is

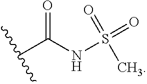 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is (R)-1,3-oxazol-2-yl-methyl; and R⁵ is

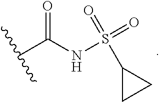 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is 2-oxabicyclo[2.1.1]hexa-1-yl-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is 2-oxabicyclo[2.1.1]hexa-1-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is 2-oxabicyclo[2.1.1]hexa-1-yl-methyl; and R⁵ is

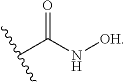 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is 2-oxabicyclo[2.1.1]hexa-1-yl-methyl; and R⁵ is

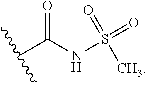 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH(CH₃)—; R² and R³ are each H; R⁴ is 2-oxabicyclo[2.1.1]hexa-1-yl-methyl; and R⁵ is

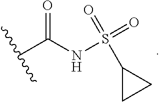 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —CH₂NH—; L² is —O—; L³ is —CH(CH₃)—; R² and R³ are each H; R⁴ is (2S)-oxetan-2-yl-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —CH₂NH—; L² is —O—; L³ is —CH(CH₃)—; R² and R³ are each H; R⁴ is (2S)-oxetan-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —CH₂NH—; L² is —O—; L³ is —CH(CH₃)—; R² and R³ are each H; R⁴ is (2S)-oxetan-2-yl-methyl; and R⁵ is

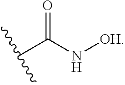 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —CH₂NH—; L² is —O—; L³ is —CH(CH₃)—; R² and R³ are each H; R⁴ is (2S)-oxetan-2-yl-methyl; and R⁵ is

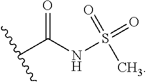 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —CH₂NH—; L² is —O—; L³ is —CH(CH₃)—; R² and R³ are each H; R⁴ is (2S)-oxetan-2-yl-methyl; and R⁵ is

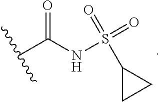 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —NHCH₂—; L² is —O—; L³ is —CH(CH₃)—; R² and R³ are each H; R⁴ is (2S)-oxetan-2-yl-methyl; and R⁵ is —COOH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —NHCH₂—; L² is —O—; L³ is —CH(CH₃)—; R² and R³ are each H; R⁴ is (2S)-oxetan-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —NHCH₂—; L² is —O—; L³ is —CH(CH₃)—; R² and R³ are each H; R⁴ is (2S)-oxetan-2-yl-methyl; and R⁵ is

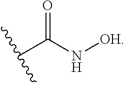 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —NHCH₂—; L² is —O—; L³ is —CH(CH₃)—; R² and R³ are each H; R⁴ is 2-oxabicyclo[2.1.1]hexa-1-yl-methyl; and R⁵ is

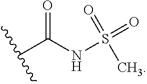 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —NHCH₂—; L² is —O—; L³ is —CH(CH₃)—; R² and R³ are each H; R⁴ is 2-oxabicyclo[2.1.1]hexa-1-yl-methyl; and R⁵ is

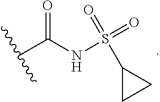 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((R)-1,1-dioxidothietan-2-yl)methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((R)-1,1-dioxidothietan-2-yl)methyl; and R⁵ is —CH₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((R)-1,1-dioxidothietan-2-yl)methyl; and R⁵ is —(CH₂)₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((R)-1,1-dioxidothietan-2-yl)methyl; and R⁵ is

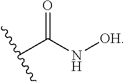 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((R)-1,1-dioxidothietan-2-yl)methyl; and R⁵ is

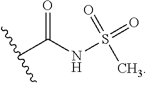 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((R)-1,1-dioxidothietan-2-yl)methyl; and R⁵ is

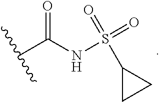 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((S)-1,1-dioxidothietan-2-yl)methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((S)-1,1-dioxidothietan-2-yl)methyl; and R⁵ is —CH₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((S)-1,1-dioxidothietan-2-yl)methyl; and R⁵ is —(CH₂)₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((S)-1,1-dioxidothietan-2-yl)methyl; and R⁵ is

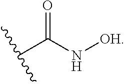 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((S)-1,1-dioxidothietan-2-yl)methyl; and R⁵ is

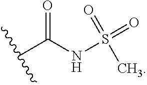 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((S)-1,1-dioxidothietan-2-yl)methyl; and R⁵ is

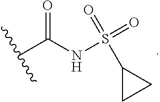 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 1-cyclobutylmethyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 1-cyclobutylmethyl; and R⁵ is —CH₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 1-cyclobutylmethyl; and R⁵ is —(CH₂)₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 1-cyclobutylmethyl; and R⁵ is

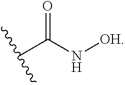 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 1-cyclobutylmethyl; and R⁵ is

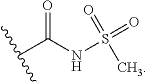 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 1-cyclobutylmethyl; and R⁵ is

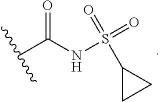 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 1-cyclobutylmethyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 1-cyclobutylmethyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 1-cyclobutylmethyl; and R⁵ is

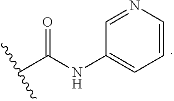 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-2-cyclopropoxypropyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-2-cyclopropoxypropyl; and R⁵ is —CH₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-2-cyclopropoxypropyl; and R⁵ is —(CH₂)₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-2-cyclopropoxypropyl; and R⁵ is

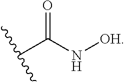 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-2-cyclopropoxypropyl; and R⁵ is

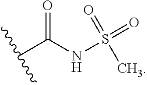 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-2-cyclopropoxypropyl; and R⁵ is

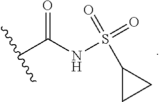 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-2-cyclopropoxypropyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-2-cyclopropoxypropyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-2-cyclopropoxypropyl; and R⁵ is

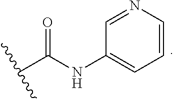 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-2-cyclopropoxypropyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-2-cyclopropoxypropyl; and R⁵ is —CH₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-2-cyclopropoxypropyl; and R⁵ is —(CH₂)₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-2-cyclopropoxypropyl; and R⁵ is

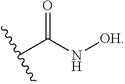 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-2-cyclopropoxypropyl; and R⁵ is

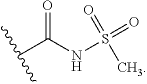 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-2-cyclopropoxypropyl; and R⁵ is

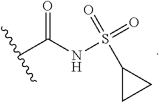 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-2-cyclopropoxypropyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-2-cyclopropoxypropyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-2-cyclopropoxypropyl; and R⁵ is

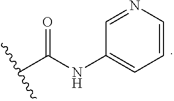 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(difluoromethoxy)propyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(difluoromethoxy)propyl; and R⁵ is —CH₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(difluoromethoxy)propyl; and R⁵ is —(CH₂)₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(difluoromethoxy)propyl; and R⁵ is

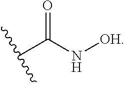 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(difluoromethoxy)propyl; and R⁵ is

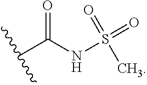 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(difluoromethoxy)propyl; and R⁵ is

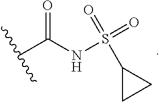 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(difluoromethoxy)propyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(difluoromethoxy)propyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(difluoromethoxy)propyl; and R⁵ is

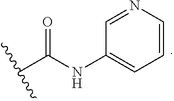 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(difluoromethoxy)propyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(difluoromethoxy)propyl; and R⁵ is —CH₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(difluoromethoxy)propyl; and R⁵ is —(CH₂)₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(difluoromethoxy)propyl; and R⁵ is

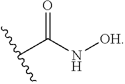 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(difluoromethoxy)propyl; and R⁵ is

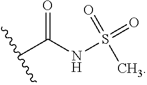 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(difluoromethoxy)propyl; and R⁵ is

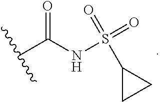 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(difluoromethoxy)propyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(difluoromethoxy)propyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(difluoromethoxy)propyl; and R⁵ is

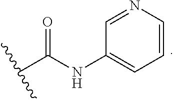 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxolan-2-yl-methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxolan-2-yl-methyl; and R⁵ is —CH₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxolan-2-yl-methyl; and R⁵ is —(CH₂)₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxolan-2-yl-methyl; and R⁵ is

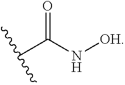 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxolan-2-yl-methyl; and R⁵ is

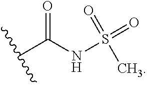 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxolan-2-yl-methyl; and R⁵ is

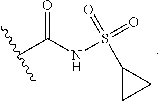 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxolan-2-yl-methyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxolan-2-yl-methyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxolan-2-yl-methyl; and R⁵ is

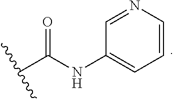 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-methoxycyclobutyl)methyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-methoxycyclobutyl)methyl; and R⁵ is —CH₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-methoxycyclobutyl)methyl; and R⁵ is —(CH₂)₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-methoxycyclobutyl)methyl; and R⁵ is

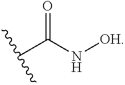 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-methoxycyclobutyl)methyl; and R⁵ is

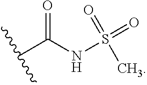 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-methoxycyclobutyl)methyl; and R⁵ is

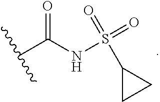 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-methoxycyclobutyl)methyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-methoxycyclobutyl)methyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-methoxycyclobutyl)methyl; and R⁵ is

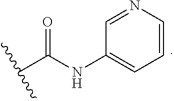 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(2,2,2-trifluoroethoxy)propyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(2,2,2-trifluoroethoxy)propyl; and R⁵ is —CH₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(2,2,2-trifluoroethoxy)propyl; and R⁵ is —(CH₂)₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(2,2,2-trifluoroethoxy)propyl; and R⁵ is

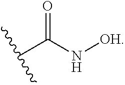 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(2,2,2-trifluoroethoxy)propyl; and R⁵ is

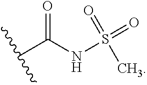 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(2,2,2-trifluoroethoxy)propyl; and R⁵ is

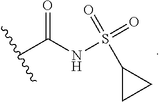 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(2,2,2-trifluoroethoxy)propyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(2,2,2-trifluoroethoxy)propyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2R)-2-(2,2,2-trifluoroethoxy)propyl; and R⁵ is

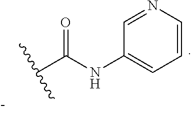 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(2,2,2-trifluoroethoxy)propyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(2,2,2-trifluoroethoxy)propyl; and R⁵ is —CH₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(2,2,2-trifluoroethoxy)propyl; and R⁵ is —(CH₂)₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(2,2,2-trifluoroethoxy)propyl; and R⁵ is

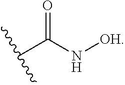 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(2,2,2-trifluoroethoxy)propyl; and R⁵ is

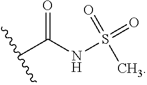 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(2,2,2-trifluoroethoxy)propyl; and R⁵ is

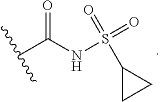 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(2,2,2-trifluoroethoxy)propyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(2,2,2-trifluoroethoxy)propyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-2-(2,2,2-trifluoroethoxy)propyl; and R⁵ is

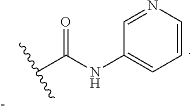 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-(2,2,2-trifluoroethoxy)ethyl; and R⁵ is tetrazolyl, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-(2,2,2-trifluoroethoxy)ethyl; and R⁵ is —CH₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-(2,2,2-trifluoroethoxy)ethyl; and R⁵ is —(CH₂)₂COOH, for instance 1H-1,2,3,4-tetrazol-5-yl. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-(2,2,2-trifluoroethoxy)ethyl; and R⁵ is

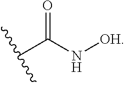 [SMILES: *C(=O)NO]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-(2,2,2-trifluoroethoxy)ethyl; and R⁵ is

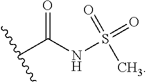 [SMILES: *C(=O)NS(C)(=O)=O]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-(2,2,2-trifluoroethoxy)ethyl; and R⁵ is

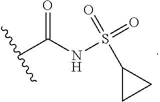 [SMILES: *C(=O)NS(=O)(=O)C1CC1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-(2,2,2-trifluoroethoxy)ethyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-(2,2,2-trifluoroethoxy)ethyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-(2,2,2-trifluoroethoxy)ethyl; and R⁵ is

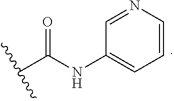 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-buta-4-yl-1,3-diol; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-buta-4-yl-1,3-diol; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-buta-4-yl-1,3-diol; and R⁵ is

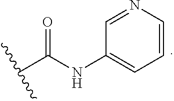 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-(difluoromethyl)oxetan-3-yl-methyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-(difluoromethyl)oxetan-3-yl-methyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-(difluoromethyl)oxetan-3-yl-methyl; and R⁵ is

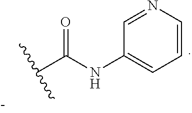 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1,1-dioxidothietan-2-yl)methyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1,1-dioxidothietan-2-yl)methyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1,1-dioxidothietan-2-yl)methyl; and R⁵ is

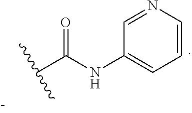 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((R)-1,1-dioxidothietan-2-yl)methyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((R)-1,1-dioxidothietan-2-yl)methyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((R)-1,1-dioxidothietan-2-yl)methyl and R⁵ is

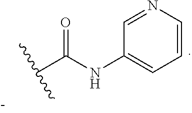 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((S)-1,1-dioxidothietan-2-yl)methyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((S)-1,1-dioxidothietan-2-yl)methyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is ((S)-1,1-dioxidothietan-2-yl)methyl; and R⁵ is

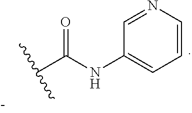 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-ethyl-1H-imidazol-5-yl)-methyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-ethyl-1H-imidazol-5-yl)-methyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (1-ethyl-1H-imidazol-5-yl)-methyl; and R⁵ is

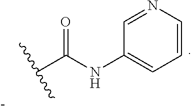 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-fluorooxetan-3-yl-methyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-fluorooxetan-3-yl-methyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 3-fluorooxetan-3-yl-methyl; and R⁵ is

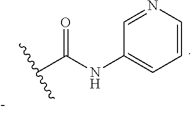 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (3R,4R)-4-methoxytetrahydrofuran-3-yl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (3R,4R)-4-methoxytetrahydrofuran-3-yl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (3R,4R)-4-methoxytetrahydrofuran-3-yl; and R⁵ is

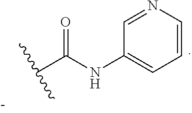 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is oxetan-2-yl-methyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is oxetan-2-yl-methyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is oxetan-2-yl-methyl; and R⁵ is

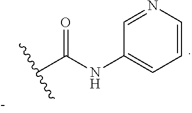 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxetan-2-yl-methyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxetan-2-yl-methyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (2S)-oxetan-2-yl-methyl; and R⁵ is

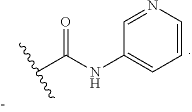 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 1,3-oxazol-2-yl-methyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 1,3-oxazol-2-yl-methyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 1,3-oxazol-2-yl-methyl; and R⁵ is

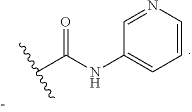 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-1,3-oxazol-2-yl-methyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-1,3-oxazol-2-yl-methyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (S)-1,3-oxazol-2-yl-methyl; and R⁵ is

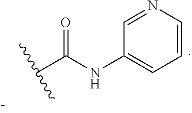 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-1,3-oxazol-2-yl-methyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-1,3-oxazol-2-yl-methyl; and R⁵ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is (R)-1,3-oxazol-2-yl-methyl; and R⁵ is

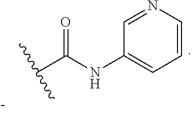 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-oxabicyclo[2.1.1]hexa-1-yl-methyl; and R⁵ is —H. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-oxabicyclo[2.1.1]hexa-1-yl-methyl; and R$ is —CH₂OH. In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments of any of Formulas (LVII)-(LXIV), L¹ is —OCH₂—; L² is —O—; L³ is —CH₂—; R² and R³ are each H; R⁴ is 2-oxabicyclo[2.1.1]hexa-1-yl-methyl; and R⁵ is

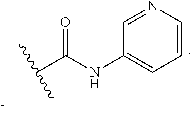 [SMILES: *C(=O)Nc1cccnc1]

In any embodiment according to this paragraph, R¹ can be selected from H, Cl, F, Me, —CF₃, —OMe, —CN, —C(O)NMe₂, alkyl, propyl, isopropyl, and cyclopropyl. In any embodiment according to this paragraph, R² can be selected from H and —OMe. In any embodiment according to this paragraph, R³ can be selected from H, F, Me, —CH₂CH₃, —CH₂CN, and —CF₃.

In certain embodiments, provided is a compound of Table 1 below, or a pharmaceutically salt, tautomer, stereoisomer, and/or mixture of stereoisomers thereof.

TABLE 1

| Com- | | |
|---|---|---|
| pound | | |
| No. | Structure | IUPAC Name |
| 552 | 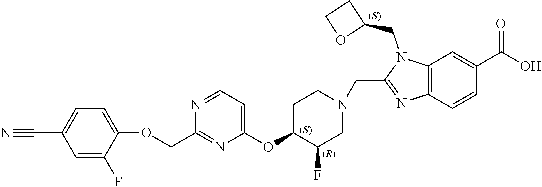 | 2-{[(3R,4S)-4-({2-[(4-cyano-2- fluorophenoxy)methyl]pyrimidin- 4-yl}oxy)-3-fluoropiperidin-1- yl]methyl}-1-{[(2S)-oxetan-2- yl]methyl}-1H-1,3-benzodiazole- 6-carboxylic acid |
| 553 | 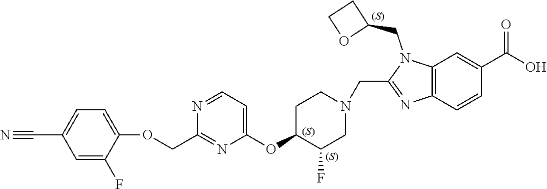 | 2-{[(3S,4S)-4-({2-[(4-cyano-2- fluorophenoxy)methyl]pyrimidin- 4-yl}oxy)-3-fluoropiperidin-1- yl]methyl}-1-{[(2S)-oxetan-2- yl]methyl}-1H-1,3-benzodiazole- 6-carboxylic acid |
| 558 | 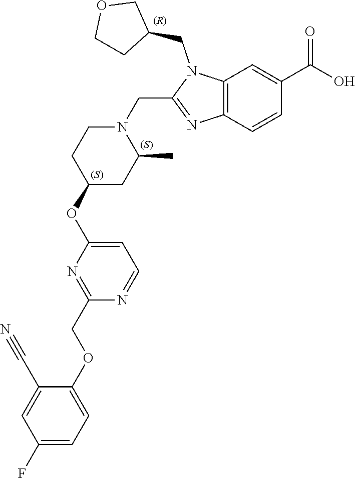 | 2-{[(2S,4S)-4-({2-[(2-cyano-4- fluorophenoxy)methyl]pyrimidin- 4-yl}oxy)-2-methylpiperidin-1- yl]methyl}-1-{[(3R)-oxolan-3- yl]methyl}-1H-1,3-benzodiazole- 6-carboxylic acid |
| 561 | 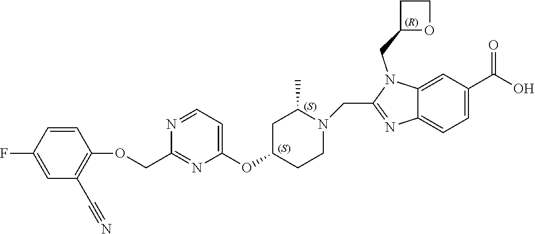 | 2-{[(2S,4S)-4-({2-[(2-cyano-4- fluorophenoxy)methyl]pyrimidin- 4-yl}oxy)-2-methylpiperidin-1- yl]methyl}-1-{[(2R)-oxetan-2- yl]methyl}-1H-1,3-benzodiazole- 6-carboxylic acid |
| 562 | 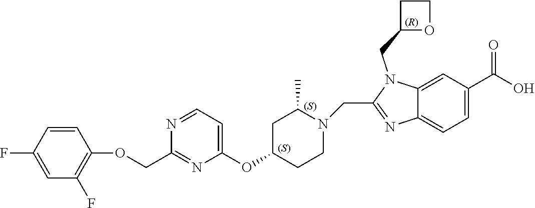 | 2-{[(2S,4S)-4-({2-[(2,4- difluorophenoxy)methyl]pyrimi- din-4-yl}oxy)-2-methylpiperi- din-1-yl]methyl}-1-{[(2R)-oxe- tan-2-yl]methyl}-1H-1,3-benzo- diazole-6-carboxylic acid |
| 572 | 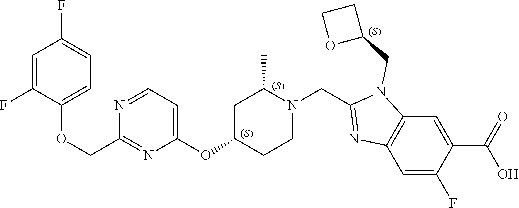 | 2-{[(2S,4S)-4-({2-[(2,4-di- fluorophenoxy)methyl]pyrimidin- 4-yl}oxy)-2-methylpiperidin-1- yl]methyl}-5-fluoro-1-{[(2S)- oxetan-2-yl]methyl}-1H-1,3- benzodiazole-6-carboxylic acid |
| 573 | 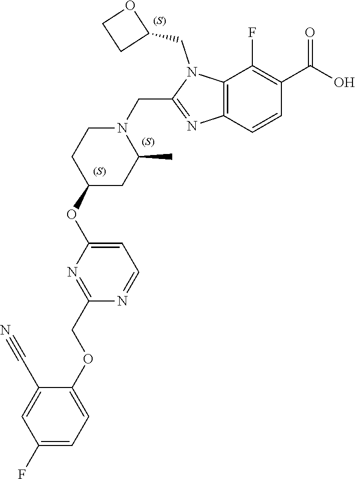 | 2-{[(2S,4S)-4-({2-[(2-cyano-4- fluorophenoxy)methyl]pyrimidin- 4-yl}oxy)-2-methylpiperidin-1- yl]methyl}-7-fluoro-1-{[(2S)- oxetan-2-yl]methyl}-1H-1,3- benzodiazole-6-carboxylic acid |
| 576 | 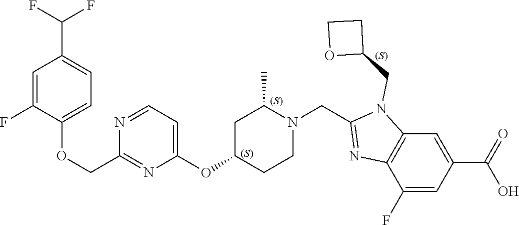 | 2-{[(2S,4S)-4-({2-[(2,4-di- fluorophenoxy)methyl]pyrimidin- 4-yl}oxy)-2-methylpiperidin-1- yl]methyl}-4-fluoro-1-{[(2S)- oxetan-2-yl]methyl}-1H-1,3- benzodiazole-6-carboxylic acid |
| 578 | 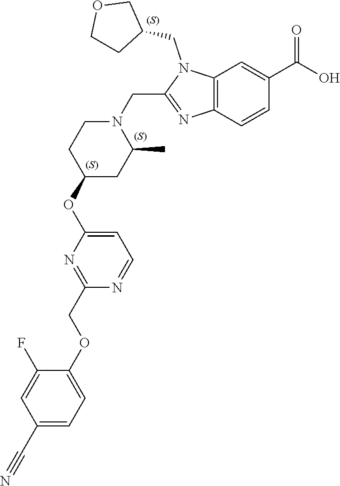 | 2-{[(2S,4S)-4-({2-[(4-cyano-2- fluorophenoxy)methyl]pyrimidin- 4-yl}oxy)-2-methylpiperidin-1- yl]methyl}-1-{[(3S)-oxolan-3- yl]methyl}-1H-1,3-benzodiazole- 6-carboxylic acid |
| 581 | 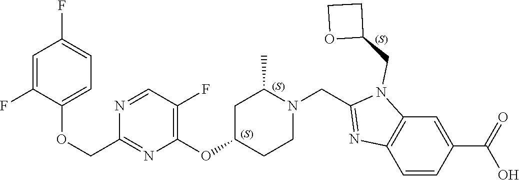 | 2-{[(2S,4S)-4-({2-[(2,4- difluorophenoxy)methyl]-5- fluoropyrimidin-4-yl}oxy)-2- methylpiperidin-1-yl]methyl}-1- {[(2S)-oxetan-2-yl]methyl}-1H- 1,3-benzodiazole-6-carboxylic acid |
| 640 | 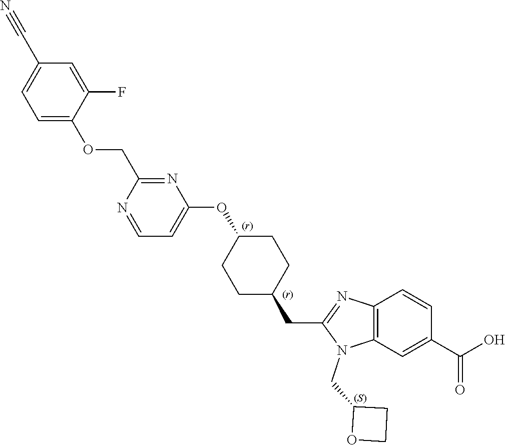 | 1-{[(2S)-oxetan-2-yl]methyl}-2- {[(1r,4r)-4-({2-[(4-cyano-2- fluorophenoxy)methyl]pyrimidin- 4-yl}oxy)cyclohexyl]methyl}-1H- 1,3-benzodiazole-6-carboxylic acid |
| 641 | 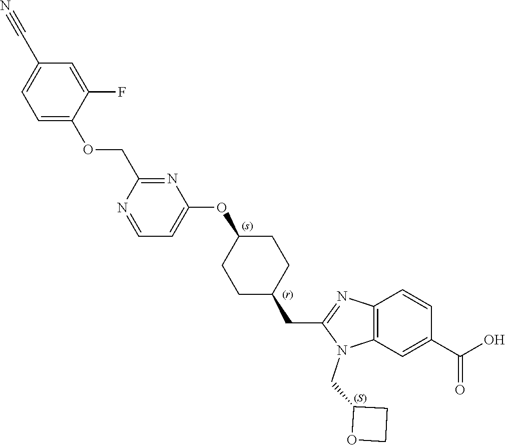 | 1-{[(2S)-oxetan-2-yl]methyl}-2- {[(1s,4s)-4-({2-[(4-cyano-2- fluorophenoxy)methyl]pyrimidin- 4-yl}oxy)cyclohcxyl]methyl}-1H- 1,3-benzodiazole-6-carboxylic acid |

In certain embodiments, provided is a compound of Table 2 below, or a pharmaceutically salt, tautomer, stereoisomer, and/or mixture of stereoisomers thereof.

TABLE 2

| Com- | | |
|---|---|---|
| pound | | |
| No. | Structure | IUPAC Name |
| 554 | 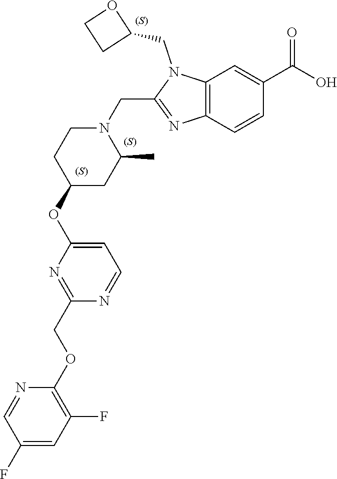 | 2-{[(2S,4S)-4-[(2-{[(5-chloro-3- fluoropyridin-2- yl)oxy]methyl}pyrimidin-4- yl)oxy]-2-methylpiperidin-1- yl]methyl}-1-{[(2S)-oxetan-2- yl]methyl}-1H-1,3- benzodiazole-6-carboxylic acid |
| 555 | 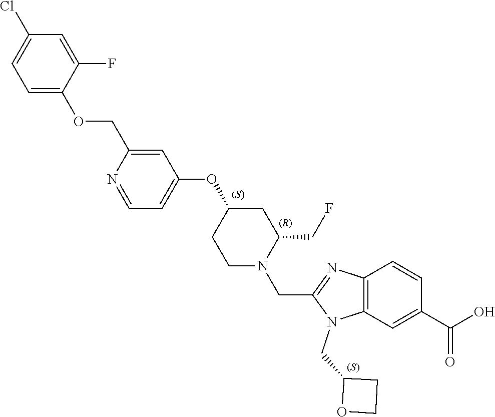 | 2-{[(2R,4S)-4-({2-[(4-chloro-2- fluorophenoxy)methyl]pyridin- 4-yl}oxy)-2- (fluoromethyl)piperidin-1- yl]methyl}-1-{[(2S)-oxetan-2- yl]methyl}-1H-1,3- benzodiazole-6-carboxylic acid |
| 556 | 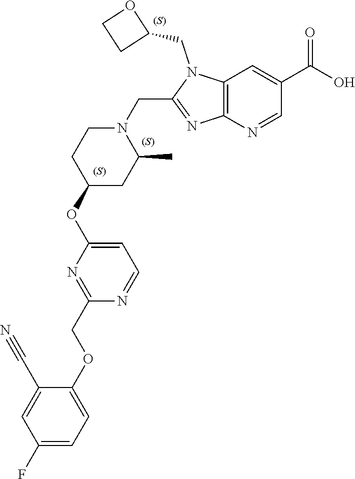 | 2-{[(2S,4S)-4-({2-[(2-cyano-4- fluorophenoxy)methyl]pyrimidin- 4-yl}oxy)-2-methylpiperidin-1- yl]methyl}-1-{[(2S)-oxetan-2- yl]methyl}-1H-imidazo[4,5- b]pyridine-6-carboxylic acid |
| 557 | 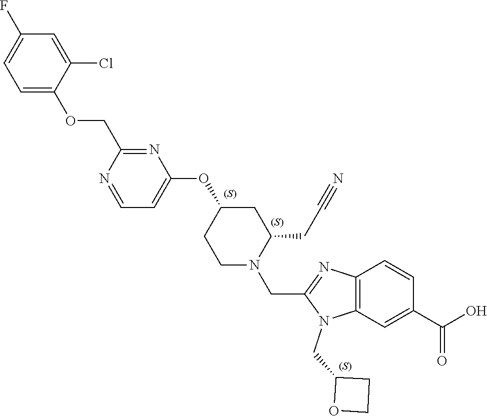 | 2-{[(2S,4S)-4-({2-[(2-chloro-4- fluorophenoxy)methyl]pyrimidin- 4-yl}oxy)-2- (cyanomethyl)piperidin-1- yl]methyl}-1-{[(2S)-oxetan-2- yl]methyl}-1H-1,3- benzodiazole-6-carboxylic acid |
| 559 | 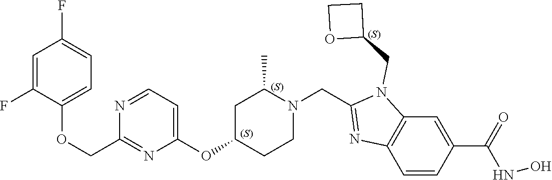 | 2-{[(2S,4S)-4-({2-[(2,4-di- fluorophenoxy)methyl]pyrimidin- 4-yl}oxy)-2-methylpiperidin- 1-yl]methyl}-N-hydroxy-1- {[(2S)-oxetan-2-yl]methyl}-1H- 1,3-benzodiazole-6-carboxamide |
| 560 | 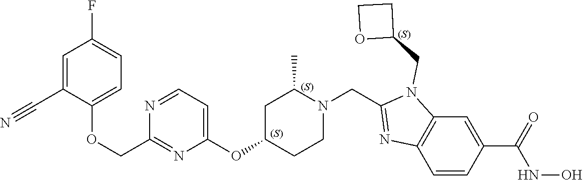 | 2-{[(2S,4S)-4-({2-[(2-cyano-4- fluorophenoxy)methyl]pyrimidin- 4-yl}oxy)-2-methylpiperidin-1- yl]methyl}-N-hydroxy-1-{[(2S)- oxetan-2-yl]methyl}-1H-1,3- benzodiazole-6-carboxamide |
| 563 | 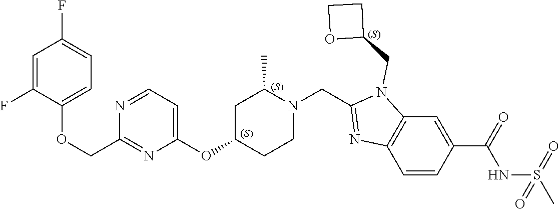 | 2-{[(2S,4S)-4-({2-[(2,4-di- fluorophenoxy)methyl]pyrimidin- 4-yl}oxy)-2-methylpiperidin- 1-yl]methyl}-N- methanesulfonyl-1-{[(2S)- oxetan-2-yl]methyl}-1H-1,3- benzodiazole-6-carboxamide |
| 564 | 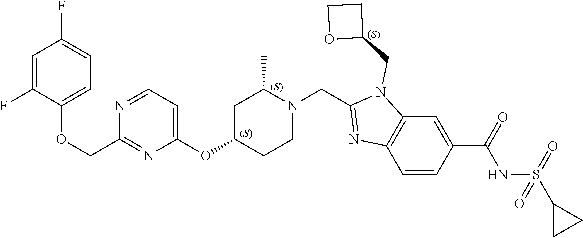 | N-(cyclopropanesulfonyl)-2- {[(2S,4S)-4-({2-[(2,4-di- fluorophenoxy )methyl]pyrimidin- 4-yl}oxy)-2-methylpiperidin- 1-yl]methyl}-1-{[(2S)-oxetan-2- yl]methyl}-1H-1,3- benzodiazole-6-carboxamide |
| 565 | 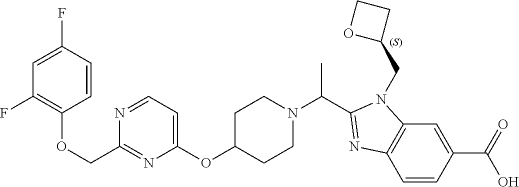 | 2-{1-14-({2-[(2,4-difluoro- phenoxy)methyl]pyrimidin- 4-yl}oxy)piperidin-1- yl]ethyl}-1-{[(2S)-oxetan-2- yl]methyl}-1H-1,3- benzodiazole-6-carboxylic acid |
| 566 | 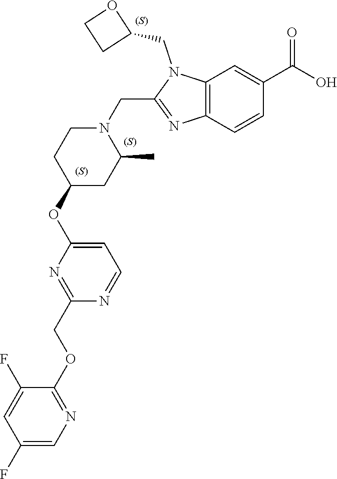 | 2-{[(2S,4S)-4-[(2-{[(3,5- difluoropyridin-2- yl)oxy]methyl}pyrimidin-4- yl)oxy]-2-methylpiperidin-1- yl]methyl}-1-{[(2S)-oxetan-2- yl]methyl}-1H-1,3- benzodiazole-6-carboxylic acid |
| 567 | 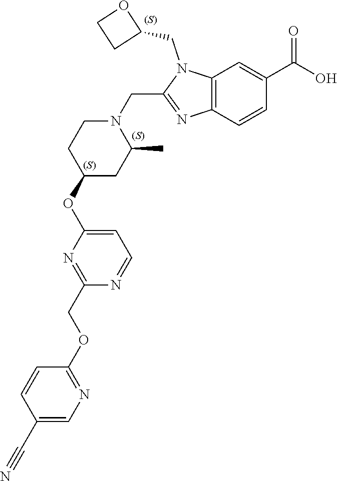 | 2-{[(2S,4S)-4-[(2-{[(5- cyanopyridin-2- yl)oxy]methyl}pyrimidin-4- yl)oxy]-2-methylpiperidin-1- yl]methyl}-1-{[(2S)-oxetan-2- yl]methyl}-1H-1,3- benzodiazole-6-carboxylic acid |
| 568 | 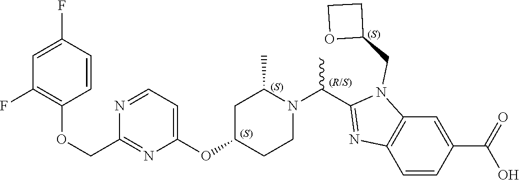 | 2-{1-[(2S,4S)-4-({2-[(2,4-di- fluorophenoxy)methyl]pyrimidin- 4-yl}oxy)-2-methylpiperidin- 1-yl]ethyl}-1-{[(2S)-oxetan-2- yl]methyl}-1H-1,3- benzodiazole-6-carboxylic acid |
| 569 | 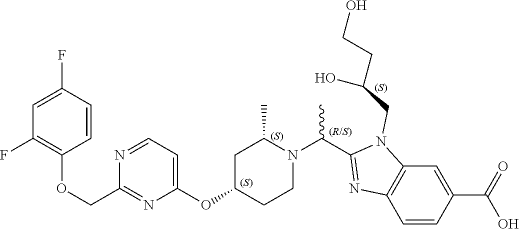 | 2-{1-[(2S,4S)-4-({2-[(2,4-di- fluorophenoxy)methyl]pyrimidin- 4-yl}oxy)-2-methylpiperidin- 1-yl]ethyl}-1-[(2S)-2,4- dihydroxybutyl]-1H-1,3- benzodiazole-6-carboxylic acid |
| 570 | 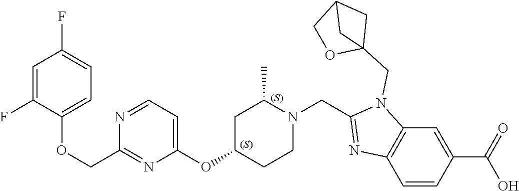 | 2-{[(2S,4S)-4-({2-[(2,4-di- fluorophenoxy)methyl]pyrimidin- 4-yl}oxy)-2-methylpiperidin- 1-yl]methyl}-1-({2- oxabicyclo[2.1.1]hexan-1- yl}methyl)-1H-1,3- benzodiazole-6-carboxylic acid |
| 571 | 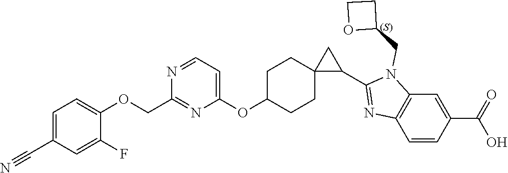 | 2-[6-({2-[(4-cyano-2- fluorophenoxy)methyl]pyrimidin- 4-yl}oxy)spiro[2.5]octan-1-yl]- 1-{[(2S)-oxetan-2-yl]methyl}- 1H-1,3-benzodiazole-6- carboxylic acid |
| 574 | 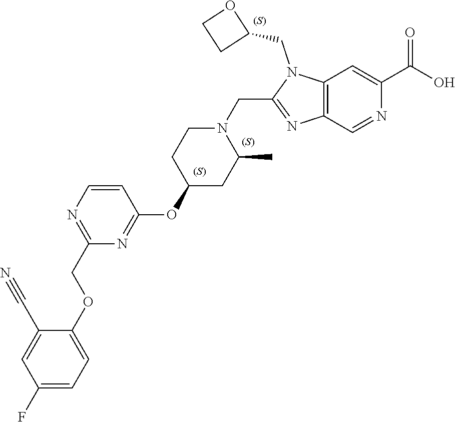 | 2-{[(2S,4S)-4-({2-[(2-cyano-4- fluorophenoxy)methyl]pyrimidin- 4-yl}oxy)-2-methylpiperidin-1- yl]methyl}-1-{[(2S)-oxetan-2- yl]methyl}-1H-imidazo[4,5- c]pyridine-6-carboxylic acid |
| 575 | 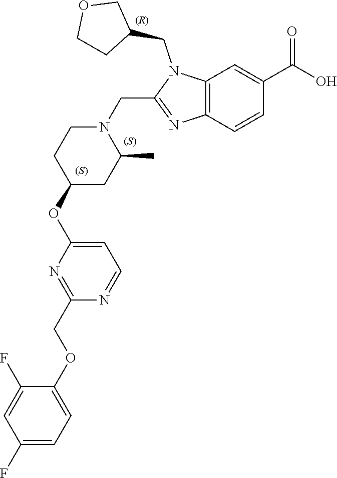 | 2-{[(2S,4S)-4-({2-[(2,4-di- fluorophenoxy)methyl]pyrimidin- 4-yl}oxy)-2-methylpiperidin- 1-yl]methyl}-1-{[(3R)-oxolan-3- yl]methyl}-1H-1,3- benzodiazole-6-carboxylic acid |
| 577 | 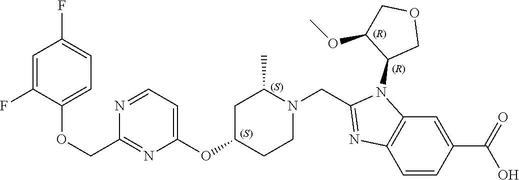 | 2-{[(2S,4S)-4-({2-[(2,4-di- fluorophenoxy)methyl]pyrimidin- 4-yl}oxy)-2-methylpiperidin- 1-yl]methyl}-1-[(3R,4R)-4- methoxyoxolan-3-yl]-1H-1,3- benzodiazole-6-carboxylic acid |
| 579 | 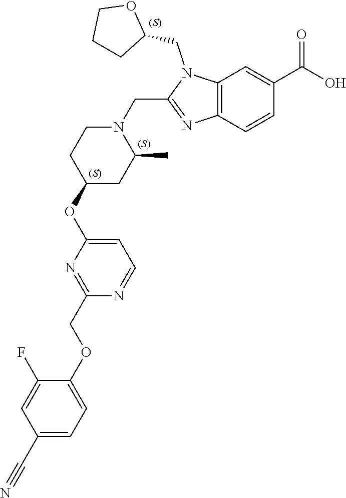 | 2-{[(2S,4S)-4-({2-[(4-cyano-2- fluorophenoxy)methyl]pyrimidin- 4-yl}oxy)-2-methylpiperidin-1- yl]methyl}-1-{[(2S)-oxolan-2- yl]methyl}-1H-1,3- benzodiazole-6-carboxylic acid |
| 580 | 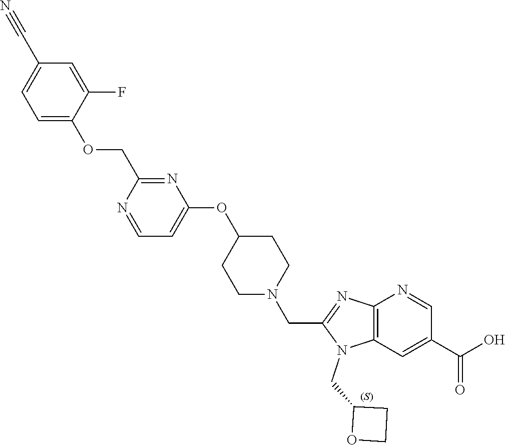 | 2-{[4-({2-[(4-cyano-2- fluorophenoxy)methyl]pyrimidin- 4-yl}oxy)piperidin-1- yl]methyl}-1-{[(2S)-oxetan-2- yl]methyl}-1H-imidazo[4,5- c]pyridine-6-carboxylic acid |
| 582 | 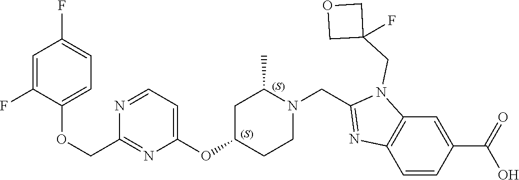 | 2-{[(2S,4S)-4-({2-[(2,4-di- fluorophenoxy)methyl]pyrimidin- 4-yl}oxy)-2-methylpiperidin- 1-yl]methyl}-1-[(3-fluorooxetan- 3-yl)methyl]-1H-1,3- benzodiazole-6-carboxylic acid |
| 583 | 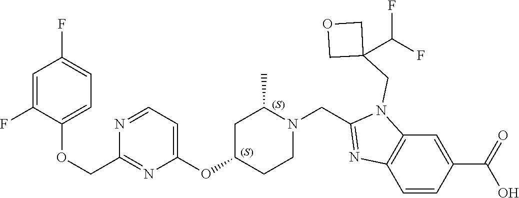 | 1-{[3-(difluoromethyl)oxetan-3- yl]methyl}-2-{[(2S,4S)-4-({2- [(2,4-difluoro- phenoxy)methyl]pyrimidin- 4-yl}oxy)-2-methylpiperidin- 1-yl]methyl}-1H-1,3- benzodiazole-6-carboxylic acid |
| 584 | 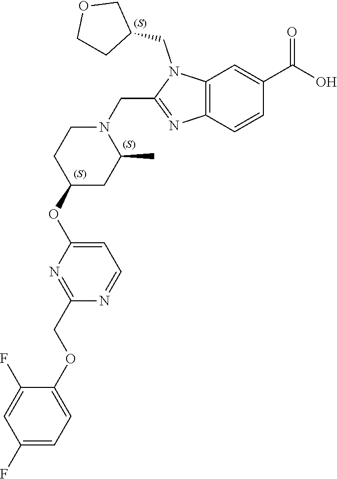 | 2-{[(2S,4S)-4-({2-[(2,4-di- fluorophenoxy)methyl]pyrimidin- 4-yl}oxy)-2-methylpiperidin- 1-yl]methyl}-1-{[(3S)-oxolan-3- yl]methyl}-1H-1,3- benzodiazole-6-carboxylic acid |
| 585 | 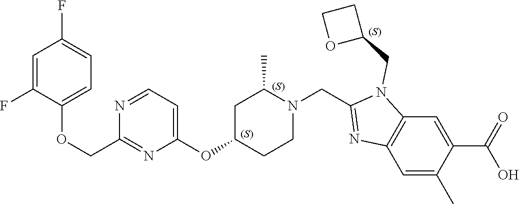 | 2-{[(2S,4S)-4-({2-[(2,4-di- fluorophenoxy)methyl]pyrimidin- 4-yl}oxy)-2-methylpiperidin- 1-yl]methyl}-5-methyl-1-{[(2S)- oxetan-2-yl]methyl}-1H-1,3- benzodiazole-6-carboxylic acid |
| 586 | 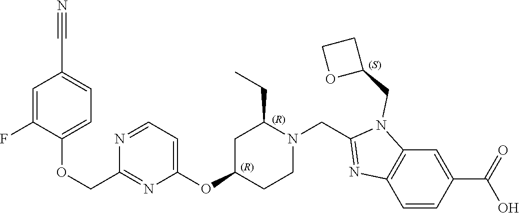 | 2-{[(2R,4R)-4-({2-[(4-cyano-2- fluorophenoxy)methyl]pyrimidin- 4-yl}oxy)-2-ethylpiperidin-1- yl]methyl}-1-{[(2S)-oxetan-2- yl]methyl}-1H-1,3- benzodiazole-6-carboxylic acid |
| 587 | 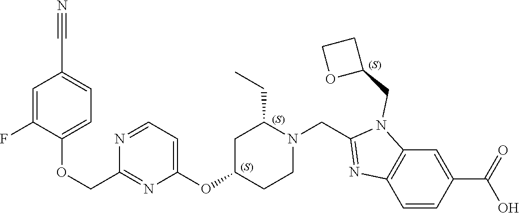 | 2-{[(2S,4S)-4-({2-[(4-cyano-2- fluorophenoxy)methyl]pyrimidin- 4-yl}oxy)-2-ethylpiperidin-1- yl]methyl}-1-{[(2S)-oxetan-2- yl]methyl}-1H-1,3- benzodiazole-6-carboxylic acid |
| 588 | 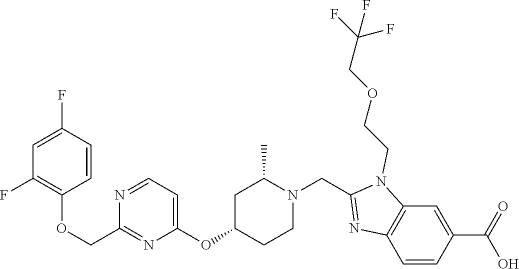 [SMILES: C[C@H]1C[C@@H](Oc2ccnc(COc3ccc(F)cc3F)n2)CCN1Cc1nc2ccc(C(=O)O)cc2n1CCOCC(F)(F)F] | 2-{[(2S,4S)-4-({2-[(2,4-di- fluorophenoxy)methyl]pyrimidin- 4-yl}oxy)-2-methylpiperidin- 1-yl]methyl}-1-[2-(2,2,2- trifluoroethoxy)ethyl]-1H-1,3- benzodiazole-6-carboxylic acid |
| 589 | 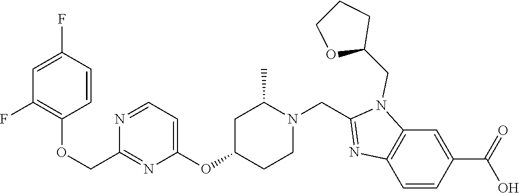 [SMILES: C[C@H]1C[C@@H](Oc2ccnc(COc3ccc(F)cc3F)n2)CCN1Cc1nc2ccc(C(=O)O)cc2n1C[C@@H]1CCCO1] | 2-{[(2,4S)-4-({2-[(2,4-di- fluorophenoxy)methyl]pyrimidin- 4-yl}oxy)-2-methylpiperidin- 1-yl]methyl}-1-{[(2S)-oxolan-2- yl]methyl}-1H-1,3- benzodiazole-6-carboxylic acid |
| 590 | 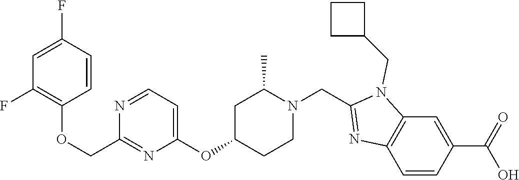 [SMILES: C[C@H]1C[C@@H](Oc2ccnc(COc3ccc(F)cc3F)n2)CCN1Cc1nc2ccc(C(=O)O)cc2n1CC1CCC1] | 1-(cyclobutylmethyl)-2- {[(2S,4S)-4-({2-[(2,4-di- fluorophenoxy)methyl]pyrimidin- 4-yl}oxy)-2-methylpiperidin- 1-yl]methyl}-1H-1,3- benzodiazole-6-carboxylic acid |
| 591 | 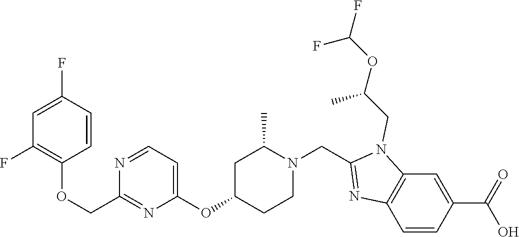 [SMILES: C[C@@H](Cn1c(CN2CC[C@H](Oc3ccnc(COc4ccc(F)cc4F)n3)C[C@@H]2C)nc2ccc(C(=O)O)cc21)OC(F)F] | 1-[(2S)-2- (difluoromethoxy)propyl]-2- {[(2S,4S)-4-({2-[(2,4-di- fluorophenoxy)methyl]pyrimidin- 4-yl}oxy)-2-methylpiperidin- 1-yl]methyl}-1H-1,3- benzodiazole-6-carboxylic acid |
| 592 | 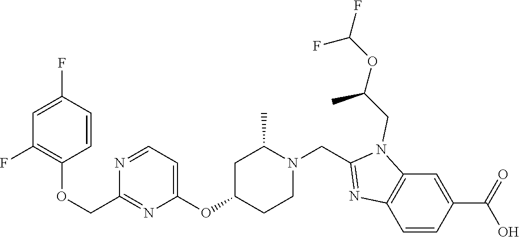 [SMILES: C[C@H](Cn1c(CN2CC[C@H](Oc3ccnc(COc4ccc(F)cc4F)n3)C[C@@H]2C)nc2ccc(C(=O)O)cc21)OC(F)F] | 1-[(2R)-2- (difluoromethoxy)propyl]-2- {[(2S,4S)-4-({2-[(2,4-di- fluorophenoxy)methyl]pyrimidin- 4-yl}oxy)-2-methylpiperidin- 1-yl]methyl}-1H-1,3- benzodiazole-6-carboxylic acid |
| 594 | 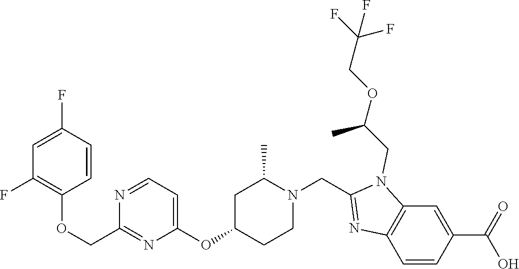 [SMILES: C[C@H](Cn1c(CN2CC[C@H](Oc3ccnc(COc4ccc(F)cc4F)n3)C[C@@H]2C)nc2ccc(C(=O)O)cc21)OCC(F)(F)F] | 2-{[(2S,4S)-4-({2-[(2,4-di- fluorophenoxy)methyl]pyrimidin- 4-yl}oxy)-2-methylpiperidin- 1-yl]methyl}-1-[(2R)-2-(2,2,2- trifluoroethoxy)propyl]-1H-1,3- benzodiazole-6-carboxylic acid |
| 595 | 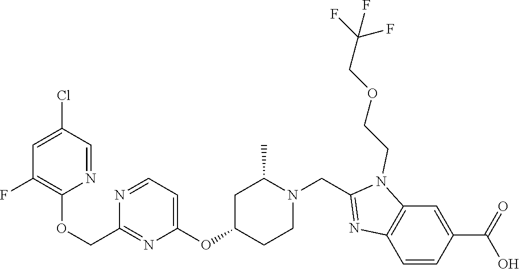 [SMILES: C[C@H]1C[C@@H](Oc2ccnc(COc3ncc(Cl)cc3F)n2)CCN1Cc1nc2ccc(C(=O)O)cc2n1CCOCC(F)(F)F] | 2-{[(2S,4S)-4-[(2-{[(5-chloro-3- fluoropyridin-2- yl)oxy]methyl}pyrimidin-4- yl)oxy]-2-methylpiperidin-1- yl]methyl}-1-[2-(2,2,2- trifluoroethoxy)ethyl]-1H-1,3- benzodiazole-6-carboxylic acid |
| 596 | 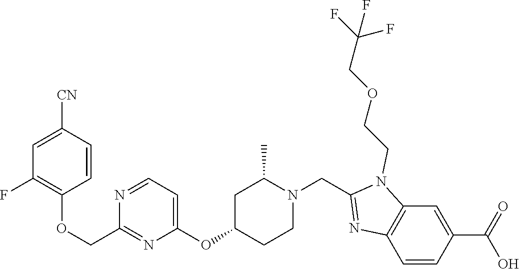 [SMILES: C[C@H]1C[C@@H](Oc2ccnc(COc3ccc(C#N)cc3F)n2)CCN1Cc1nc2ccc(C(=O)O)cc2n1CCOCC(F)(F)F] | 2-{[(2S,4S)-4-({2-[(4-cyano-2- fluorophenoxy)methyl]pyrimidin- 4-yl}oxy)-2-methylpiperidin-1- yl]methyl}-1-[2-(2,2,2- trifluoroethoxy)ethyl]-1H-1,3- benzodiazole-6-carboxylic acid |
| 597 | 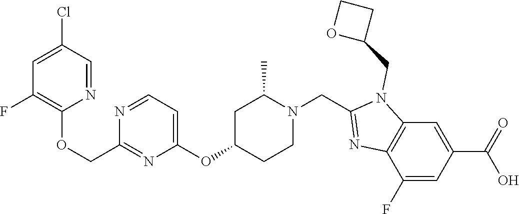 [SMILES: C[C@H]1C[C@@H](Oc2ccnc(COc3ncc(Cl)cc3F)n2)CCN1Cc1nc2c(F)cc(C(=O)O)cc2n1C[C@@H]1CCO1] | 2-{[(2S,4S)-4-[(2-{[(5-chloro-3- fluoropyridin-2- yl)oxy]methyl}pyrimidin-4- yl)oxy]-2-methylpiperidin-1- yl]methyl}-4-fluoro-1-{[(2S)- oxetan-2-yl]methyl}-1H-1,3- benzodiazole-6-carboxylic acid |
| 598 | 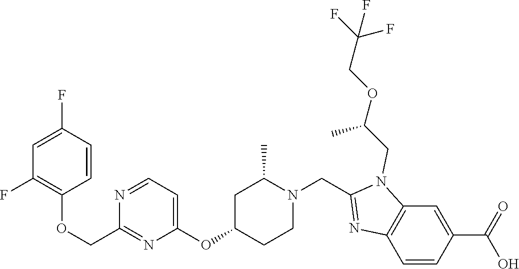 [SMILES: C[C@@H](Cn1c(CN2CC[C@H](Oc3ccnc(COc4ccc(F)cc4F)n3)C[C@@H]2C)nc2ccc(C(=O)O)cc21)OCC(F)(F)F] | 2-{[(2S,4S)-4-({2-[(2,4-di- fluorophenoxy)methyl]pyrimidin- 4-yl}oxy)-2-methylpiperidin- 1-yl]methyl}-1-[(2S)-2-(2,2,2- trifluoroethoxy)propyl]-1H-1,3- benzodiazole-6-carboxylic acid |
| 599 | 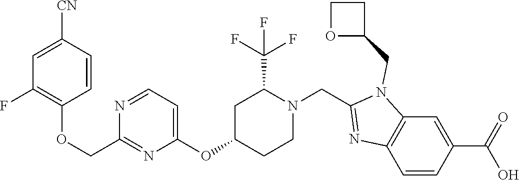 [SMILES: N#Cc1ccc(OCc2nccc(O[C@H]3CCN(Cc4nc5ccc(C(=O)O)cc5n4C[C@@H]4CCO4)[C@@H](C(F)(F)F)C3)n2)c(F)c1] | 2-{[(2R,4S)-4-({2-[(4-cyano-2- fluorophenoxy)methyl]pyrimidin- 4-yl}oxy)-2- (trifluoromethyl)piperidin-1- yl]methyl}-1-{[(2S)-oxetan-2- yl]methyl}-1H-1,3- benzodiazole-6-carboxylic acid |
| 600 | 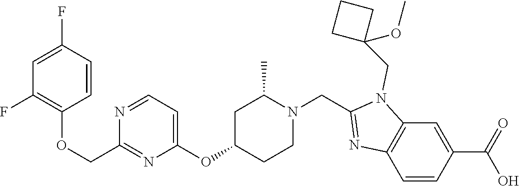 [SMILES: COC1(Cn2c(CN3CC[C@H](Oc4ccnc(COc5ccc(F)cc5F)n4)C[C@@H]3C)nc3ccc(C(=O)O)cc32)CCC1] | 2-{[(2S,4S)-4-({2-[(2,4-di- fluorophenoxy)methyl]pyrimidin- 4-yl}oxy)-2-methylpiperidin- 1-yl]methyl}-1-[(1- methoxycyclobutyl)methyl]-1H- 1,3-benzodiazole-6-carboxylic acid |
| 601 | 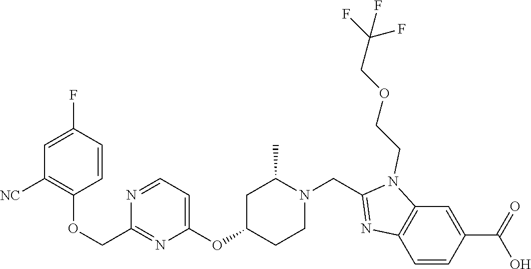 [SMILES: C[C@H]1C[C@@H](Oc2ccnc(COc3ccc(F)cc3C#N)n2)CCN1Cc1nc2ccc(C(=O)O)cc2n1CCOCC(F)(F)F] | 2-{[(2S,4S)-4-({2-[(2-cyano-4- fluorophenoxy)methyl]pyrimidin- 4-yl}oxy)-2-methylpiperidin-1- yl]methyl}-1-[2-(2,2,2- trifluoroethoxy)ethyl]-1H-1,3- benzodiazole-6-carboxylic acid |
| 602 | 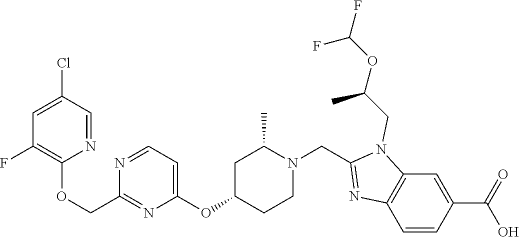 [SMILES: C[C@H](Cn1c(CN2CC[C@H](Oc3ccnc(COc4ncc(Cl)cc4F)n3)C[C@@H]2C)nc2ccc(C(=O)O)cc21)OC(F)F] | 2-{[(2S,4S)-4-[(2-{[(5-chloro-3- fluoropyridin-2- yl)oxy]methyl}pyrimidin-4- yl)oxy]-2-methylpiperidin-1- yl]methyl}-1-[(2R)-2- (difluoromethoxy)propyl]-1H- 1,3-benzodiazole-6-carboxylic acid |
| 603 | 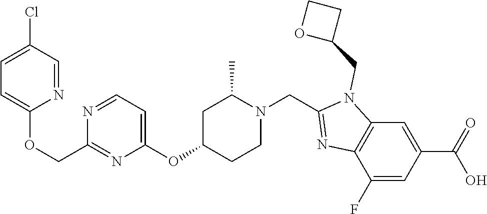 [SMILES: C[C@H]1C[C@@H](Oc2ccnc(COc3ccc(Cl)cn3)n2)CCN1Cc1nc2c(F)cc(C(=O)O)cc2n1C[C@@H]1CCO1] | 2-{[(2S,4S)-4-[(2-{[(5- chloropyridin-2- yl)oxy]methyl}pyrimidin-4- yl)oxy]-2-methylpiperidin-1- yl]methyl}-4-fluoro-1-{[(2S)- oxetan-2-yl]methyl}-1H-1,3- benzodiazole-6-carboxylic acid |
| 604 | 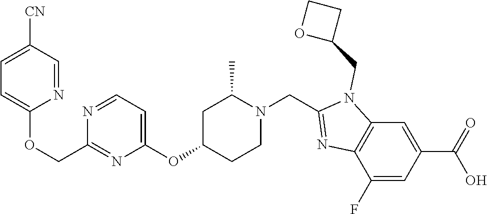 [SMILES: C[C@H]1C[C@@H](Oc2ccnc(COc3ccc(C#N)cn3)n2)CCN1Cc1nc2c(F)cc(C(=O)O)cc2n1C[C@@H]1CCO1] | 2-{[(2S,4S)-4-[(2-{[(5- cyanopyridin-2- yl)oxy]methyl}pyrimidin-4- yl)oxy]-2-methylpiperidin-1- yl]methyl}-4-fluoro-1-{[(2S)- oxetan-2-yl]methyl}-1H-1,3- benzodiazole-6-carboxylic acid |
| 605 | 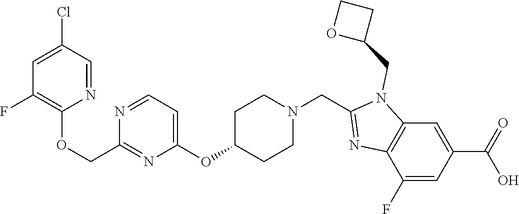 | 2-({4-[(2-{[(5-chloro-3- fluoropyridin-2- yl)oxy]methyl}pyrimidin-4- yl)oxy]piperidin-1-yl}methyl)-4- fluoro-1-{[(2S)-oxetan-2- yl]methyl}-1H-1,3- benzodiazole-6-carboxylic acid |
| 606 | 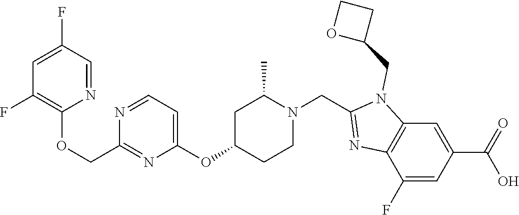 [SMILES: C[C@H]1C[C@@H](Oc2ccnc(COc3ncc(F)cc3F)n2)CCN1Cc1nc2c(F)cc(C(=O)O)cc2n1C[C@@H]1CCO1] | 2-{[(2S,4S)-4-[(2-{[(3,5- difluoropyridin-2- yl)oxy]methyl}pyrimidin-4- yl)oxy]-2-methylpiperidin-1- yl]methyl}-4-fluoro-1-[(oxetan- 2-yl)methyl]-1H-1,3- benzodiazole-6-carboxylic acid |
| 607 | 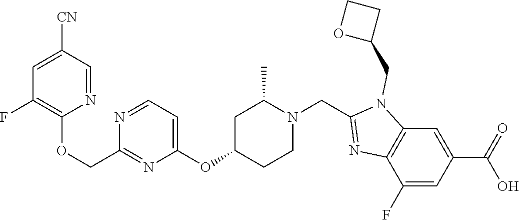 [SMILES: C[C@H]1C[C@@H](Oc2ccnc(COc3ncc(C#N)cc3F)n2)CCN1Cc1nc2c(F)cc(C(=O)O)cc2n1C[C@@H]1CCO1] | 2-{[(2S,4S)-4-[(2-{[(5-cyano-3- fluoropyridin-2- yl)oxy]methyl}pyrimidin-4- yl)oxy]-2-methylpiperidin-1- yl]methyl}-4-fluoro-1-[(oxetan- 2-yl)methyl]-1H-1,3- benzodiazole-6-carboxylic acid |
| 608 | 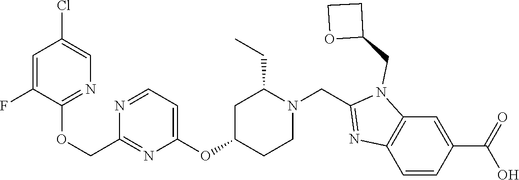 [SMILES: CC[C@H]1C[C@@H](Oc2ccnc(COc3ncc(Cl)cc3F)n2)CCN1Cc1nc2ccc(C(=O)O)cc2n1C[C@@H]1CCO1] | 2-{[(2S,4S)-4-[(2-{[(5-chloro-3- fluoropyridin-2- yl)oxy]methyl}pyrimidin-4- yl)oxy]-2-ethylpiperidin-1- yl]methyl}-1-[(oxetan-2- yl)methyl]-1H-1,3-benzodiazole- 6-carboxylic acid |
| 609 | 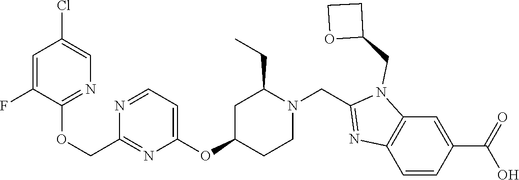 [SMILES: CC[C@@H]1C[C@H](Oc2ccnc(COc3ncc(Cl)cc3F)n2)CCN1Cc1nc2ccc(C(=O)O)cc2n1C[C@@H]1CCO1] | 2-{[(2R,4R)-4-[(2-{[(5-chloro-3- fluoropyridin-2- yl)oxy]methyl}pyrimidin-4- yl)oxy]-2-ethylpiperidin-1- yl]methyl}-1-[(oxetan-2- yl)methyl]-1H-1,3-benzodiazole- 6-carboxylic acid |
| 610 | 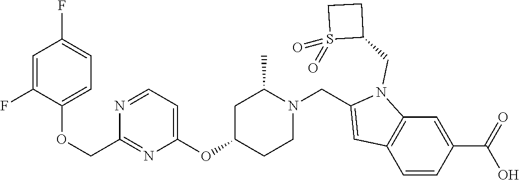 [SMILES: C[C@H]1C[C@@H](Oc2ccnc(COc3ccc(F)cc3F)n2)CCN1Cc1cc2ccc(C(=O)O)cc2n1C[C@H]1CCS1(=O)=O] | 2-(((2S,4S)-4-((2-((2,4-di- fluorophenoxy)methyl)pyrimidin- 4-yl)oxy)-2-methylpiperidin- 1-yl)methyl)-1-(((R)-1,1- dioxidothietan-2-yl)methyl)-1H- benzo[d]imidazole-6-carboxylic acid |
| 611 | 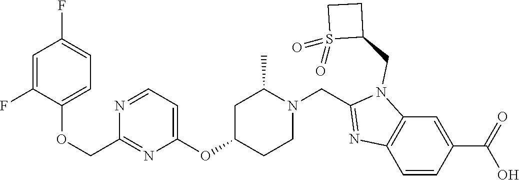 [SMILES: C[C@H]1C[C@@H](Oc2ccnc(COc3ccc(F)cc3F)n2)CCN1Cc1nc2ccc(C(=O)O)cc2n1C[C@@H]1CCS1(=O)=O] | 2-(((2S,4S)-4-((2-((2,4-di- fluorophenoxy)methyl)pyrimidin- 4-yl)oxy)-2-methylpiperidin- 1-yl)methyl)-1-(((S)-1,1- dioxidothietan-2-yl)methyl)-1H- benzo[d]imidazole-6-carboxylic acid |
| 612 | 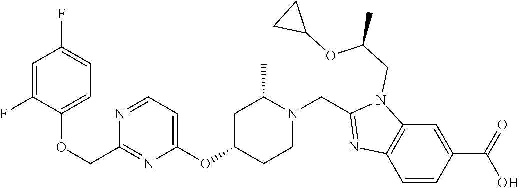 [SMILES: C[C@@H](Cn1c(CN2CC[C@H](Oc3ccnc(COc4ccc(F)cc4F)n3)C[C@@H]2C)nc2ccc(C(=O)O)cc21)OC1CC1] | 1-((S)-2-cyclopropoxypropyl)- 2-(((2S,4S)-4-((2-((2,4-di- fluorophenoxy)methyl)pyrimidin- 4-yl)oxy)-2-methylpiperidin- 1-yl)methyl)-1H- benzo[d]imidazole-6-carboxylic acid |
| 613 | 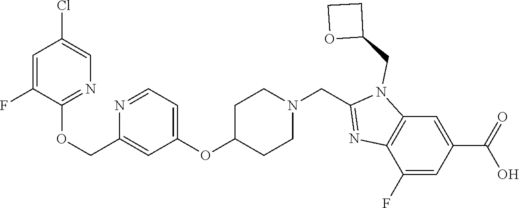 [SMILES: O=C(O)c1cc(F)c2nc(CN3CCC(Oc4ccnc(COc5ncc(Cl)cc5F)c4)CC3)n(C[C@@H]3CCO3)c2c1] | (S)-2-((4-((2-(((5-chloro-3- fluoropyridin-2- yl)oxy)methyl)pyridin-4- yl)oxy)piperidin-1-yl)methyl)-4- fluoro-1-(oxetan-2-ylmethyl)- 1H-benzo[d]imidazole-6- carboxylic acid |
| 614 | 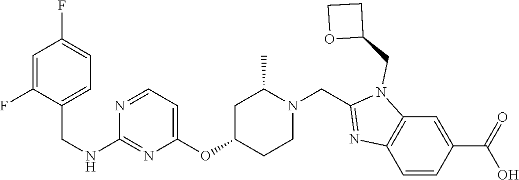 [SMILES: C[C@H]1C[C@@H](Oc2ccnc(NCc3ccc(F)cc3F)n2)CCN1Cc1nc2ccc(C(=O)O)cc2n1C[C@@H]1CCO1] | 2-(((2S,4S)-4-((2-((2,4- difluorobenzyl)amino)pyrimidin- 4-yl)oxy)-2-methylpiperidin-1- yl)methyl)-1-(((S)-oxetan-2- yl)methyl)-1H- benzo[d]imidazole-6-carboxylic acid |
| 615 | 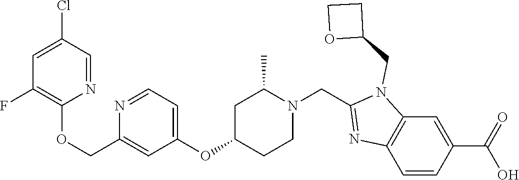 [SMILES: C[C@H]1C[C@@H](Oc2ccnc(COc3ncc(Cl)cc3F)c2)CCN1Cc1nc2ccc(C(=O)O)cc2n1C[C@@H]1CCO1] | 2-(((2S,4S)-4-((2-(((5-chloro-3- fluoropyridin-2- yl)oxy)methyl)pyridin-4- yl)oxy)-2-methylpiperidin-1- yl)methyl)-1-(((S)-oxetan-2- yl)methyl)-1H- benzo[d]imidazole-6-carboxylic acid |
| 616 | 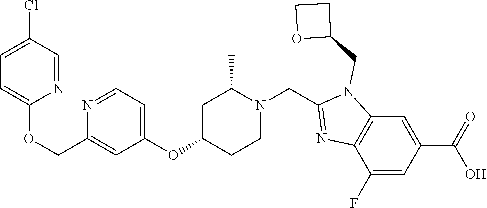 [SMILES: C[C@H]1C[C@@H](Oc2ccnc(COc3ccc(Cl)cn3)c2)CCN1Cc1nc2c(F)cc(C(=O)O)cc2n1C[C@@H]1CCO1] | 2-(((2S,4S)-4-((2-(((5- chloropyridin-2- yl)oxy)methyl)pyridin-4- yl)oxy)-2-methylpiperidin-1- yl)methyl)-4-fluoro-1-(((S)- oxetan-2-yl)methyl)-1H- benzo[d]imidazole-6-carboxylic acid |
| 617 | 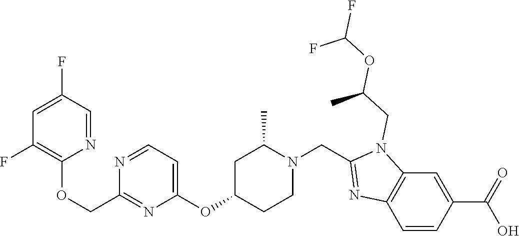 [SMILES: C[C@H](Cn1c(CN2CC[C@H](Oc3ccnc(COc4ncc(F)cc4F)n3)C[C@@H]2C)nc2ccc(C(=O)O)cc21)OC(F)F] | 1-((R)-2- (difluoromethoxy)propyl)-2- (((2S,4S)-4-((2-(((3,5- difluoropyridin-2- yl)oxy)methyl)pyrimidin-4- yl)oxy)-2-methylpiperidin-1- yl)methyl)-1H- benzo[d]imidazole-6-carboxylic acid |
| 618 | 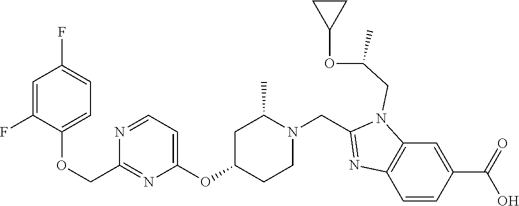 [SMILES: C[C@H](Cn1c(CN2CC[C@H](Oc3ccnc(COc4ccc(F)cc4F)n3)C[C@@H]2C)nc2ccc(C(=O)O)cc21)OC1CC1] | 1-((R)-2-cyclopropoxypropyl)-2- (((2S,4S)-4-((2-((2,4-di- fluorophenoxy)methyl)pyrimidin- 4-yl)oxy)-2-methylpiperidin- 1-yl)methyl)-1H- benzo[d]imidazole-6-carboxylic acid |
| 619 | 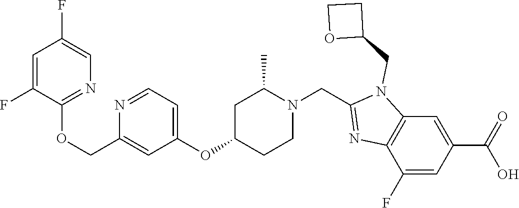 [SMILES: C[C@H]1C[C@@H](Oc2ccnc(COc3ncc(F)cc3F)c2)CCN1Cc1nc2c(F)cc(C(=O)O)cc2n1C[C@@H]1CCO1] | 2-(((2S,4S)-4-((2-(((3,5- difluoropyridin-2- yl)oxy)methyl)pyridin-4- yl)oxy)-2-methylpiperidin-1- yl)methyl)-4-fluoro-1-(((S)- oxetan-2-yl)methyl)-1H- benzo[d]imidazole-6-carboxylic acid |
| 620 | 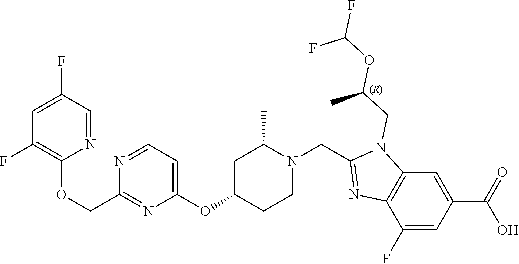 | 1-((R)-2- (difluoromethoxy)propyl)-2- (((2S,4S)-4-((2-(((3,5- difluoropyridin-2- yl)oxy)methyl)pyrimidin-4- yl)oxy)-2-methylpiperidin-1- yl)methyl)-4-fluoro-1H- benzo[d]imidazole-6-carboxylic acid |
| 621 | 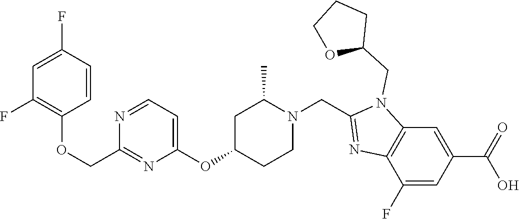 [SMILES: C[C@H]1C[C@@H](Oc2ccnc(COc3ccc(F)cc3F)n2)CCN1Cc1nc2c(F)cc(C(=O)O)cc2n1C[C@@H]1CCCO1] | 2-(((2S,4S)-4-((2-((2,4-di- fluorophenoxy)methyl)pyrimidin- 4-yl)oxy)-2-methylpiperidin- 1-yl)methyl)-4-fluoro-1-(((S)- tetrahydrofuran-2-yl)methyl)- 1H-benzo[d]imidazole-6- carboxylic acid |
| 622 | 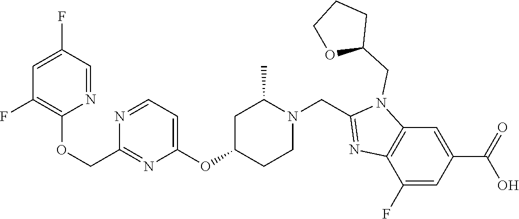 [SMILES: C[C@H]1C[C@@H](Oc2ccnc(COc3ncc(F)cc3F)n2)CCN1Cc1nc2c(F)cc(C(=O)O)cc2n1C[C@@H]1CCCO1] | 2-(((2S,4S)-4-((2-(((3,5- difluoropyridin-2- yl)oxy)methyl)pyrimidin-4- yl)oxy)-2-methylpiperidin-1- yl)methyl)-4-fluoro-1-(((S)- tetrahydrofuran-2-yl)methyl)- 1H-benzo[d]imidazole-6- carboxylic acid |
| 623 | 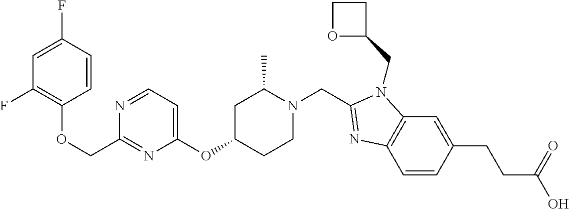 [SMILES: C[C@H]1C[C@@H](Oc2ccnc(COc3ccc(F)cc3F)n2)CCN1Cc1nc2ccc(CCC(=O)O)cc2n1C[C@@H]1CCO1] | 3-(2-(((2S,4S)-4-((2-((2,4-di- fluorophenoxy)methyl)pyrimidin- 4-yl)oxy)-2-methylpiperidin- 1-yl)methyl)-1-(((S)-oxetan-2- yl)methyl)-1H- benzo[d]imidazol-6-yl)propanoic acid |
| 624 | 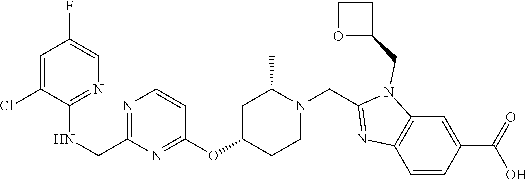 [SMILES: C[C@H]1C[C@@H](Oc2ccnc(CNc3ncc(F)cc3Cl)n2)CCN1Cc1nc2ccc(C(=O)O)cc2n1C[C@@H]1CCO1] | 2-(((2S,4S)-4-((2-(((3-chloro-5- fluoropyridin-2- yl)amino)methyl)pyrimidin-4- yl)oxy)-2-methylpiperidin-1- yl)methyl)-1-(((S)-oxetan-2- yl)methyl)-1H- benzo[d]imidazole-6-carboxylic acid |
| 625 | 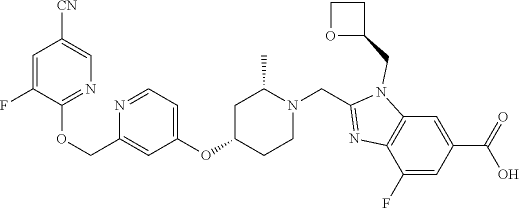 [SMILES: C[C@H]1C[C@@H](Oc2ccnc(COc3ncc(C#N)cc3F)c2)CCN1Cc1nc2c(F)cc(C(=O)O)cc2n1C[C@@H]1CCO1] | 2-(((2S,4S)-4-((2-(((5-cyano-3- fluoropyridin-2- yl)oxy)methyl)pyridin-4- yl)oxy)-2-methylpiperidin-1- yl)methyl)-4-fluoro-1-(((S)- oxetan-2-yl)methyl)-1H- benzo[d]imidazole-6-carboxylic acid |
| 626 | 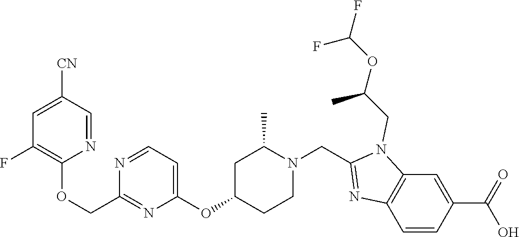 [SMILES: C[C@H](Cn1c(CN2CC[C@H](Oc3ccnc(COc4ncc(C#N)cc4F)n3)C[C@@H]2C)nc2ccc(C(=O)O)cc21)OC(F)F] | 2-(((2S,4S)-4-((2-(((5-cyano-3- fluoropyridin-2- yl)oxy)methyl)pyrimidin-4- yl)oxy)-2-methylpiperidin-1- yl)methyl)-1-((R)-2- (difluoromethoxy)propyl)-1H- benzo[d]imidazole-6-carboxylic acid |
| 627 | 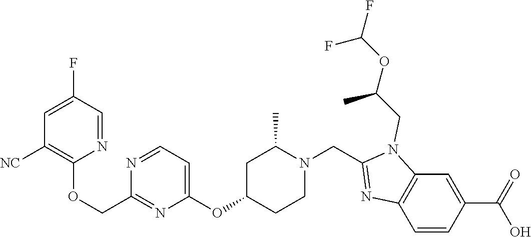 [SMILES: C[C@H](Cn1c(CN2CC[C@H](Oc3ccnc(COc4ncc(F)cc4C#N)n3)C[C@@H]2C)nc2ccc(C(=O)O)cc21)OC(F)F] | 2-(((2S,4S)-4-((2-(((3-cyano-5- fluoropyridin-2- yl)oxy)methyl)pyrimidin-4- yl)oxy)-2-methylpiperidin-1- yl)methyl)-1-((R)-2- (difluoromethoxy)propyl)-1H- benzo[d]imidazole-6-carboxylic acid |
| 628 | 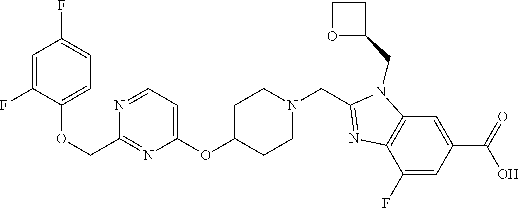 [SMILES: O=C(O)c1cc(F)c2nc(CN3CCC(Oc4ccnc(COc5ccc(F)cc5F)n4)CC3)n(C[C@@H]3CCO3)c2c1] | (S)-2-((4-((2-(((3,5- difluoropyridin-2- yl)oxy)methyl)pyrimidin-4- yl)oxy)piperidin-1-yl)methyl)-4- fluoro-1-(oxetan-2-ylmethyl)- 1H-benzo[d]imidazole-6- carboxylic acid |
| 629 | 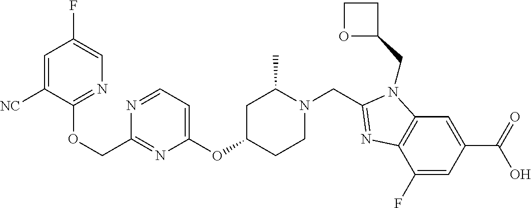 [SMILES: C[C@H]1C[C@@H](Oc2ccnc(COc3ncc(F)cc3C#N)n2)CCN1Cc1nc2c(F)cc(C(=O)O)cc2n1C[C@@H]1CCO1] | 2-(((2S,4S)-4-((2-(((3-cyano-5- fluoropyridin-2- yl)oxy)methyl)pyrimidin-4- yl)oxy)-2-methylpiperidin-1- yl)methyl)-4-fluoro-1 -(((S)- oxetan-2-yl)methyl)-1H- benzo[d]imidazole-6-carboxylic acid |
| 630 | 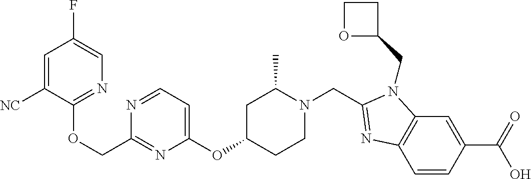 [SMILES: C[C@H]1C[C@@H](Oc2ccnc(COc3ncc(F)cc3C#N)n2)CCN1Cc1nc2ccc(C(=O)O)cc2n1C[C@@H]1CCO1] | 2-(((2S,4S)-4-((2-(((3-cyano-5- fluoropyridin-2- yl)oxy)methyl)pyrimidin-4- yl)oxy)-2-methylpiperidin-1- yl)methyl)-1-(((S)-oxetan-2- yl)methyl)-1H- benzo[d]imidazole-6-carboxylic acid |
| 631 | 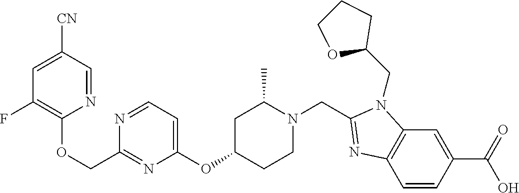 [SMILES: C[C@H]1C[C@@H](Oc2ccnc(COc3ncc(C#N)cc3F)n2)CCN1Cc1nc2ccc(C(=O)O)cc2n1C[C@@H]1CCCO1] | 2-(((2S,4S)-4-((2-(((5-cyano-3- fluoropyridin-2- yl)oxy)methyl)pyrimidin-4- yl)oxy)-2-methylpiperidin-1- yl)methyl)-1-(((S)- tetrahydrofuran-2-yl)methyl)- 1H-benzo[d]imidazole-6- carboxylic acid |
| 632 | 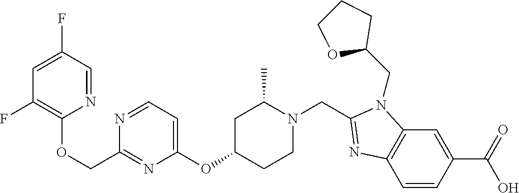 [SMILES: C[C@H]1C[C@@H](Oc2ccnc(COc3ncc(F)cc3F)n2)CCN1Cc1nc2ccc(C(=O)O)cc2n1C[C@@H]1CCCO1] | 2-(((2S,4S)-4-((2-(((3,5- difluoropyridin-2- yl)oxy)methyl)pyrimidin-4- yl)oxy)-2-methylpiperidin-1- yl)methyl)-1-(((S)- tetrahydrofuran-2-yl)methyl)- 1H-benzo[d]imidazole-6- carboxylic acid |
| 633 | 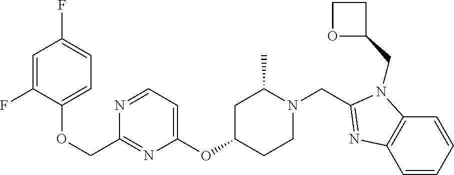 [SMILES: C[C@H]1C[C@@H](Oc2ccnc(COc3ccc(F)cc3F)n2)CCN1Cc1nc2ccccc2n1C[C@@H]1CCO1] | 2-(((2S,4S)-4-((2-((2,4-di- fluorophenoxy)methyl)pyrimidin- 4-yl)oxy)-2-methylpiperidin- 1-yl)methyl)-1-(((S)-oxetan-2- yl)methyl)-1H- benzo[d]imidazole |
| 634 | 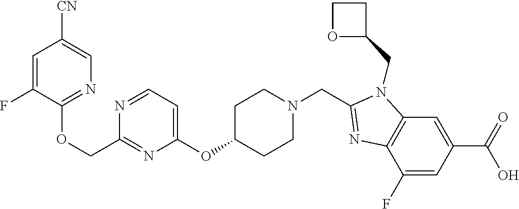 | (S)-2-((4-((2-(((5-cyano-3- fluoropyridin-2- yl)oxy)methyl)pyrimidin-4- yl)oxy)piperidin-1-yl)methyl)-1- (oxetan-2-ylmethyl)-1H- benzo[d]imidazole-6-carboxylic acid |
| 635 | 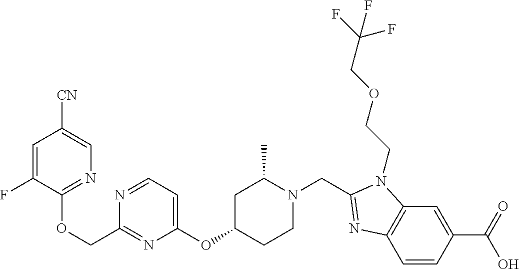 [SMILES: C[C@H]1C[C@@H](Oc2ccnc(COc3ncc(C#N)cc3F)n2)CCN1Cc1nc2ccc(C(=O)O)cc2n1CCOCC(F)(F)F] | 2-(((2S,4S)-4-((2-(((5-cyano-3- fluoropyridin-2- yl)oxy)methyl)pyrimidin-4- yl)oxy)-2-methylpiperidin-1- yl)methyl)-1-(2-(2,2,2- trifluoroethoxy)ethyl)-1H- benzo[d]imidazole-6-carboxylic acid |
| 636 | 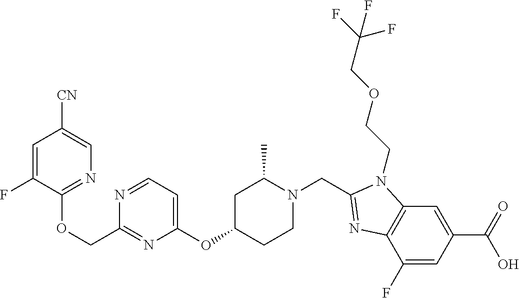 [SMILES: C[C@H]1C[C@@H](Oc2ccnc(COc3ncc(C#N)cc3F)n2)CCN1Cc1nc2c(F)cc(C(=O)O)cc2n1CCOCC(F)(F)F] | 2-(((2S,4S)-4-((2-(((5-cyano-3- fluoropyridin-2- yl)oxy)methyl)pyrimidin-4- yl)oxy)-2-methylpiperidin-1- yl)methyl)-4-fluoro-1-(2-(2,2,2- trifluoroethoxy)ethyl)-1H- benzo[d]imidazole-6-carboxylic acid |
| 637 | 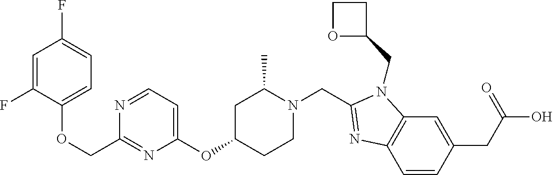 [SMILES: C[C@H]1C[C@@H](Oc2ccnc(COc3ccc(F)cc3F)n2)CCN1Cc1nc2ccc(CC(=O)O)cc2n1C[C@@H]1CCO1] | 2-(2-(((2S,4S)-4-((2-((2,4-di- fluorophenoxy)methyl)pyrimidin- 4-yl)oxy)-2-methylpiperidin- 1-yl)methyl)-1-(((S)-oxetan-2- yl)methyl)-1H- benzo[d]imidazol-6-yl)acetic acid |
| 638 | 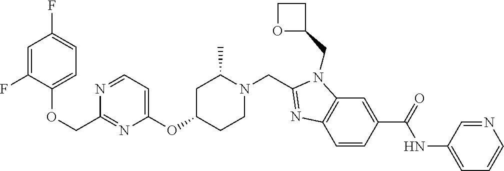 [SMILES: C[C@H]1C[C@@H](Oc2ccnc(COc3ccc(F)cc3F)n2)CCN1Cc1nc2ccc(C(=O)Nc3cccnc3)cc2n1C[C@@H]1CCO1] | 2-(((2S,4S)-4-((2-((2,4-di fluorophenoxy)methyl)pyrimidin- 4-yl)oxy)-2-methylpiperidin- 1-yl)methyl)-1-(((S)-oxetan-2- yl)methyl)-N-(pyridin-3-yl)-1H- benzo[d]imidazole-6- carboxamide |
| 639 | 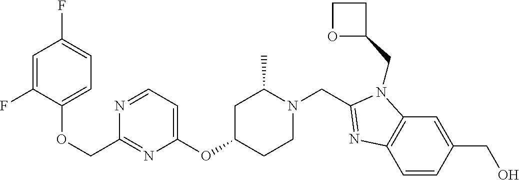 [SMILES: C[C@H]1C[C@@H](Oc2ccnc(COc3ccc(F)cc3F)n2)CCN1Cc1nc2ccc(CO)cc2n1C[C@@H]1CCO1] | (2-(((2S,4S)-4-((2-((2,4-di- fluorophenoxy)methyl)pyrimidin- 4-yl)oxy)-2-methylpiperidin- 1-yl)methyl)-1-(((S)-oxetan-2- yl)methyl)-1H- benzo[d]imidazol-6-yl)methanol |
| 640 | 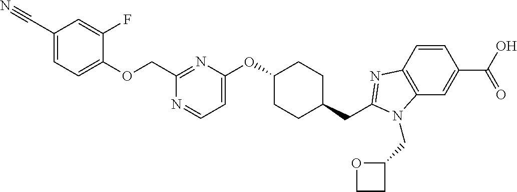 [SMILES: N#Cc1ccc(OCc2nccc(O[C@H]3CC[C@H](Cc4nc5ccc(C(=O)O)cc5n4C[C@@H]4CCO4)CC3)n2)c(F)c1] | 1-{[(2S)-oxetan-2-y1]methyl}- 2-{[(1r,4r)-4-({2-[(4-cyano-2- fluorophenoxy)methyl] pyrimi din-4-yl}oxy)cyclohexyl]meth- yl}-1H-1,3-benzodiazole- 6-carboxylic acid |
| 641 | 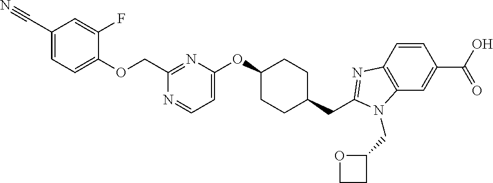 [SMILES: N#Cc1ccc(OCc2nccc(O[C@H]3CC[C@@H](Cc4nc5ccc(C(=O)O)cc5n4C[C@@H]4CCO4)CC3)n2)c(F)c1] | 1-{[(2S)-oxetan-2-yl]methyl}-2- {[(1s,4s)-4-({2-[(4-cyano-2- fluorophenoxy)methyl]pyrimi din-4-yl}oxy)cyclohexyl]meth- yl}-1H-1,3-benzodiazole- 6-carboxylic acid |

### Optically Active Compounds

In certain embodiments, compounds provided herein may have several chiral centers and may exist in and be isolated in optically active and racemic forms. In certain embodiments, some compounds may exhibit polymorphism. A person of skill in the art will appreciate that compounds provided herein can exist in any racemic, optically-active, diastereomeric, polymorphic, or stereoisomeric form, and/or mixtures thereof. A person of skill in the art will also appreciate that such compounds described herein that possess the useful properties also described herein is within the scope of this disclosure. A person of skill in the art will further appreciate how to prepare optically active forms of the compounds described herein, for example, by resolution of racemic forms via recrystallization techniques, by synthesis from optically-active starting materials, by chiral synthesis, or by chromatographic separation using a chiral stationary phase. In addition, most amino acids are chiral (i.e., designated as L- or D-, wherein the L-enantiomer is the naturally occurring configuration) and can exist as separate enantiomers.

Examples of methods to obtain optically active materials are known in the art, and include at least the following:
i) physical separation of crystals—a technique whereby macroscopic crystals of the individual enantiomers are manually separated. This technique can be used if crystals of the separate enantiomers exist (i.e., the material is a conglomerate, and the crystals are visually distinct);
ii) simultaneous crystallization—a technique whereby the individual enantiomers are separately crystallized from a solution of the racemate, only if the latter is a conglomerate in the solid state;
iii) enzymatic resolutions—a technique wherein partial, or complete separation of a racemate is accomplished by virtue of different rates of reaction of the enantiomers in the presence of an enzyme;
iv) enzymatic asymmetric synthesis—a synthetic technique wherein at least one step of the synthesis uses an enzymatic reaction to obtain an enantiomerically pure, or enriched synthetic precursor of the desired enantiomer;
v) chemical asymmetric synthesis—a synthetic technique wherein the desired enantiomer is synthesized from an achiral precursor using chiral catalysts, or chiral auxiliaries to produce asymmetry (i.e., chirality) in the product;
vi) diastereomer separations—a technique wherein a racemic compound is treated with an enantiomerically pure reagent (a chiral auxiliary) that converts the individual enantiomers to diastereomers. The resulting diastereomers are then separated by chromatography, or crystallization by virtue of their now more distinct diastereomeric differences, and then the chiral auxiliary is removed to obtain each enantiomer;
vii) first- and second-order asymmetric transformations—a technique wherein diastereomers of the racemate equilibrate in solution to yield a preponderance of a diastereomer of the desired enantiomer, or where kinetic, or thermodynamic crystallization of the diastereomer of the desired enantiomer perturbs the equilibrium such that eventually in principle all the material is converted to the crystalline diastereomer of the desired enantiomer. The desired enantiomer is then derived from the diastereomer;
viii) kinetic resolutions—this technique refers to the achievement of partial or complete resolution of a racemate (or of a further resolution of a partially resolved compound) by virtue of unequal reaction rates of the enantiomers with a chiral, or non-racemic reagent, or catalyst under kinetic conditions;
ix) enantiospecific synthesis from non-racemic precursors—a synthetic technique wherein the desired enantiomer is obtained from chiral starting materials, and where the stereochemical integrity is not or is only minimally compromised over the course of the synthesis;
x) chiral liquid chromatography—a technique wherein the enantiomers of a racemate are separated in a liquid mobile phase by virtue of their different interactions with a stationary phase. The stationary phase can be made of chiral material, or the mobile phase can contain an additional chiral material to provoke the different interactions;
xi) chiral gas chromatography—a technique wherein the racemate is volatilized and enantiomers are separated by virtue of their different interactions in the gaseous mobile phase with a column containing a fixed non-racemic adsorbent phase;
xii) extraction with chiral solvents—a technique wherein the enantiomers are separated by virtue of kinetic or thermodynamic dissolution of one enantiomer into a particular chiral solvent;
xiii) transport across chiral membranes—a technique wherein a racemate is placed in contact with a thin membrane barrier. The barrier typically separates two miscible fluids, one containing the racemate, and a driving force such as a concentration, or pressure differential causes preferential transport across the membrane barrier. Separation occurs as a result of the non-racemic nature of the membrane which allows only one enantiomer of the racemate to pass through.

In some embodiments, provided herein are compositions of compounds of any of Formulas (LVII)-(LXIV), that are substantially free of a designated stereoisomer of that compound. In certain embodiments, in the methods and compounds of this disclosure, the compounds are substantially free of other stereoisomers. In some embodiments, the composition includes a compound that is at least 85%, 90%, 95%, 98%, or 99% to 100% by weight of the compound, the remainder comprising other chemical species, or enantiomers. In some embodiments, provided herein are compositions of compounds of any of Formulas (LVII)-(LXIV).

That are substantially free of a designated enantiomer of that compound. In certain embodiments, in the methods and compounds of this disclosure, the compounds are substantially free of other enantiomers. In some embodiments, the composition includes a compound that is at least 85%, 90%, 95%, 98%, or 99% to 100% by weight of the compound, the remainder comprising other chemical species or enantiomers.

### Isotopically Enriched Compounds

Also provided herein are isotopically enriched compounds including, but not limited to, isotopically enriched compounds of any of Formulas (LVII)-(LXIV).

Isotopic enrichment (for example, deuteration) of pharmaceuticals to improve pharmacokinetics (“PK”), pharmacodynamics (“PD”), and/or toxicity profiles, has been previously demonstrated within some classes of drugs. See, for example, Lijinsky et al., Food Cosmet. Toxicol., 20:393 (1982); Lijinsky et al., J. Nat. Cancer Inst., 69:1127 (1982); Mangold et al., Mutation Res. 308:33 (1994); Gordon et al., Drug Metab. Dispos., 15:589 (1987); Zello et al., Metabolism, 43:487 (1994); Gately et al., J. Nucl. Med., 27:388 (1986); Wade D, Chem. Biol. Interact. 117:191 (1999).

Isotopic enrichment of a drug can be used, for example, to (1) reduce or eliminate unwanted metabolites; (2) increase the half-life of the parent drug; (3) decrease the number of doses needed to achieve a desired effect; (4) decrease the amount of a dose necessary to achieve a desired effect; (5) increase the formation of active metabolites if any are formed; and/or (6) decrease the production of deleterious metabolites in specific tissues. Isotopic enrichment of a drug can also be used to create a more effective and/or safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e., the step with the highest transition state energy), substitution of a (heavier) isotope for that reactive hydrogen will cause a decrease in the reaction rate. The Deuterium Kinetic Isotope Effect (“DKIE”) is the most common form of KIE. (See, e.g., Foster et al., Adv. Drug Res., vol. 14, pp. 1-36 (1985); Kushner et al., Can. J. Physiol. Pharmacol., vol. 77, pp. 79-88 (1999)).

The magnitude of the DKIE can be expressed as the ratio between the rates of a given reaction in which a C—H bond is broken, and the same reaction where deuterium is substituted for hydrogen and the C-D bond is broken. The DKIE can range from about one (no isotope effect) to very large numbers, such as 50, or more, meaning that the reaction can be fifty, or more, times slower when deuterium has been substituted for hydrogen.

Substitution of tritium (“T”) for hydrogen results in yet a stronger bond than deuterium and gives numerically larger isotope effects. Similarly, substitution of isotopes for other elements including, but not limited to, ¹³C, or ¹⁴C for carbon; ³³S, ³⁴S, or ³⁶S for sulfur; ¹⁵N for nitrogen; and ¹⁷O, or ¹⁸O for oxygen may lead to a similar kinetic isotope effect.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases to react with and convert these foreign substances to more polar intermediates, or metabolites for renal excretion. Some of the most common metabolic reactions of pharmaceutical compounds involve the oxidation of a carbon-hydrogen (C—H) bond to either a carbon-oxygen (C—O), or carbon-carbon (C═C) pi-bond. The resultant metabolites may be stable, or unstable under physiological conditions, and can have substantially different PK/PD, and acute, and long-term toxicity profiles relative to the parent compounds. For many drugs, such oxidations are rapid. Therefore, these drugs often require the administration of multiple or high daily doses.

Therefore, isotopic enrichment at certain positions of a compound provided herein will produce a detectable KIE that will affect the pharmacologic, PK, PD, and/or toxicological profiles of a compound provided herein in comparison with a similar compound having a natural isotopic composition.

### Pharmaceutical Compositions and Methods of Administration

The compounds provided herein can be formulated into pharmaceutical compositions using methods available in the art and those disclosed herein. Any of the compounds provided herein can be provided in the appropriate pharmaceutical composition and be administered by a suitable route of administration.

The methods provided herein encompass administering pharmaceutical compositions comprising at least one compound provided herein and one or more compatible and pharmaceutically acceptable carriers. In this context, the term “pharmaceutically acceptable” means approved by a regulatory agency of the Federal, or state government, or listed in the U.S. Pharmacopeia, or other generally recognized pharmacopeia for use in animals, and in certain embodiments in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils including petroleum, animal, vegetable, or oils of synthetic origin, such as peanut oil, soybean oil, mineral oil, sesame oil, and the like. Water can be used as a carrier when the pharmaceutical composition is administered intravenously. Saline solutions, and aqueous dextrose, and glycerol solutions can also be employed as liquid carriers, particularly for injectable solutions. Examples of suitable pharmaceutical carriers are described in Martin, E. W., Remington's Pharmaceutical Sciences.

In clinical practice the pharmaceutical compositions, or compounds provided herein may be administered by any route known in the art. Exemplary routes of administration include, but are not limited to, inhalation, intraarterial, intradermal, intramuscular, intraperitoneal, intravenous, nasal, parenteral, pulmonary, and subcutaneous routes. In some embodiments, a pharmaceutical composition or compound provided herein is administered parenterally.

The compositions for parenteral administration can be emulsions or sterile solutions. Parenteral compositions may include, for example, propylene glycol, polyethylene glycol, vegetable oils, and injectable organic esters (e.g., ethyl oleate). These compositions can also contain wetting, isotonizing, emulsifying, dispersing, and stabilizing agents. Sterilization can be carried out in several ways, for example, using a bacteriological filter, via radiation, or via heating. Parenteral compositions can also be prepared in the form of sterile solid compositions which can be dissolved at the time of use in sterile water, or any other injectable sterile medium.

In some embodiments, a composition provided herein is a pharmaceutical composition, or a single unit dosage form. Pharmaceutical compositions, and single unit dosage forms provided herein comprise a prophylactically, or therapeutically effective amount of one, or more prophylactic, or therapeutic compounds.

The pharmaceutical composition may comprise one, or more pharmaceutical excipients. Any suitable pharmaceutical excipient may be used, wherein a person of ordinary skill in the art is capable of selecting suitable pharmaceutical excipients. Non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. Whether a particular excipient is suitable for incorporation into a pharmaceutical composition, or dosage form depends on a variety of factors well known in the art including, but not limited to, the way in which the dosage form will be administered to a subject and the specific compound in the dosage form. The composition, or single unit dosage form, if desired, can also contain minor amounts of wetting, or emulsifying agents, or pH buffering agents. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference herein in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long-chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a co-solvent. Illustrative examples of co-solvents include, ethanol, poly(ethylene) glycol, butylene glycol, dimethylacetamide, glycerin, and propylene glycol.

In some embodiments, the pharmaceutical composition comprises a buffer. Illustrative examples of buffers include, acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier, or filler. Illustrative examples of carriers, or fillers include, lactose, maltodextrin, mannitol, sorbitol, chitosan, stearic acid, xanthan gum, and guar gum.

In some embodiments, the pharmaceutical composition comprises a surfactant. Illustrative examples of surfactants, include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene (glycol) succinate.

In some embodiments, the pharmaceutical composition comprises an anti-caking agent. Illustrative examples of anti-caking agents include, calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference herein in its entirety.

In some embodiments, the pharmaceutical composition comprises a solvent. In some aspects, the solvent is saline solution, such as a sterile isotonic saline solution, or dextrose solution. In some aspects, the solvent is water for injection.

In some embodiments, the pharmaceutical compositions are in a particulate form, such as a microparticle or a nanoparticle. Microparticles, and nanoparticles may be formed from any suitable material, such as a polymer, or a lipid. In some aspects, the microparticles, or nanoparticles are micelles, liposomes, or polymersomes.

Further provided herein are anhydrous pharmaceutical compositions, and dosage forms comprising a compound, since, in some embodiments, water can facilitate the degradation of some compounds.

Anhydrous pharmaceutical compositions, and dosage forms provided herein can be prepared using anhydrous, or low moisture containing ingredients, and low moisture, or low humidity conditions. Pharmaceutical compositions, and dosage forms that comprise lactose, and at least one active ingredient that comprises a primary, or secondary amine can be anhydrous if substantial contact with moisture, and/or humidity during manufacturing, packaging, and/or storage is expected.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Lactose-free compositions provided herein can comprise excipients that are well known in the art and are listed, for example, in the U.S. Pharmocopeia (USP) SP (XXI)/NF (XVI). In general, lactose-free compositions comprise an active ingredient, a binder/filler, and a lubricant in pharmaceutically compatible, and pharmaceutically acceptable amounts. Exemplary lactose-free dosage forms comprise an active ingredient, microcrystalline cellulose, pre gelatinized starch, and magnesium stearate.

Also provided are pharmaceutical compositions, and dosage forms that comprise one, or more excipients that reduce the rate by which a compound will decompose. Such excipients, which are referred to herein as “stabilizers,” include, but are not limited to, antioxidants such as ascorbic acid, pH buffers, or salt buffers.

### Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically sterile, or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to Water for Injection USP; aqueous vehicles such as, but not limited to, Sodium Chloride Injection, Ringer's Injection, Dextrose Injection, Dextrose, and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one, or more of the antibodies disclosed herein can also be incorporated into the parenteral dosage forms.

### Dosage and Unit Dosage Forms

In human therapeutics, the doctor will determine the posology which he considers most appropriate according to a preventive, or curative treatment, and according to the age, weight, condition, and other factors specific to the subject to be treated.

In certain embodiments, a composition provided herein is a pharmaceutical composition, or a single unit dosage form. Pharmaceutical compositions, and single unit dosage forms provided herein comprise a prophylactically, or therapeutically effective amount of one, or more prophylactic, or therapeutic antibodies, or antigen binding fragments thereof.

The amount of the compound, or composition which will be effective in the prevention, or treatment of a disorder, or one, or more symptoms thereof will vary with the nature, and severity of the disease, or condition, and the route by which the compound is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro, or animal model test systems.

In certain embodiments, exemplary doses of a composition include milligram, or microgram amounts of the compound per kilogram of subject, or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). In certain embodiments, the dosage of the compound provided herein, based on weight of the compound, administered to prevent, treat, manage, or ameliorate a disorder, or one, or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition, or a composition provided herein administered to prevent, treat, manage, or ameliorate a disorder, or one, or more symptoms thereof in a subject is 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 to 15 mg, 0.5 to 12 mg, 0.5 to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

The dose can be administered according to a suitable schedule, for example, once, two times, three times, or four times weekly. It may be necessary to use dosages of the compound outside the ranges disclosed herein in some cases, as will be apparent to those of ordinary skill in the art. Furthermore, it is noted that the clinician, or treating physician will know how, and when to interrupt, adjust, or terminate therapy in conjunction with subject response.

Different therapeutically effective amounts may be applicable for different diseases, and conditions, as will be readily known by those of ordinary skill in the art. Similarly, amounts sufficient to prevent, manage, treat, or ameliorate such disorders, but insufficient to cause, or sufficient to reduce, adverse effects associated with the antibodies, or antigen binding fragments thereof provided herein are also encompassed by the described dosage amounts, and dose frequency schedules herein. Further, when a subject is administered multiple dosages of a composition provided herein, not all of the dosages need be the same. For example, the dosage administered to the subject may be increased to improve the prophylactic, or therapeutic effect of the composition, or it may be decreased to reduce one, or more side effects that a particular subject is experiencing.

In certain embodiments, treatment, or prevention can be initiated with one, or more loading doses of a compound, or composition provided herein followed by one, or more maintenance doses.

In certain embodiments, a dose of a compound, or composition provided herein can be administered to achieve a steady-state concentration of the compound in blood, or serum of the subject. The steady-state concentration can be determined by measurement according to techniques available to those of skill or can be based on the physical characteristics of the subject such as height, weight, and age.

In certain embodiments, administration of the same composition may be repeated and the administrations may be separated by at least one day, two days, three days, five days, ten days, fifteen days, thirty days, forty-five days, two months, seventy-five days, three months, or six months. In other embodiments, administration of the same prophylactic, or therapeutic agent may be repeated, and the administration may be separated by at least one day, two days, three days, five days, ten days, fifteen days, thirty days, forty-five days, two months, seventy-five days, three months, or six months.

### Therapeutic Applications

For therapeutic applications, the compounds are administered to a mammal, in certain embodiments, a human, in a pharmaceutically acceptable dosage suitable for administration form such as those known in the art, and those discussed herein, intravenously as a bolus or by continuous infusion over a period of time, by intramuscular, intraperitoneal, intra-cerebrospinal, subcutaneous, intra-articular, intrasynovial, intrathecal, or intratumoral routes. The compounds also are suitably administered by peritumoral, intralesional, or perilesional routes, to exert local as well as systemic therapeutic effects. In certain embodiments, the compounds are administered to a mammal, in certain embodiments, a human, in a pharmaceutically acceptable dosage suitable for oral administration form such as those known in the art, and those discussed herein. For example, the compounds of this disclosure may be administered orally to a human as a liquid, or solid form. Solid dosage forms include, capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

The compounds provided herein may be useful for the treatment of any disease, or condition described herein (e.g., a metabolic disease or condition). In certain embodiments, the disease, or condition is any disease, or condition that benefits from modulation of GLP-1 receptor activity. In certain embodiments, the disease, or condition is any disease, or condition that benefits from agonizing GLP-1 receptor activity. In certain embodiments, the methods reduce blood glucose levels. In certain embodiments, the methods promote insulin synthesis, stimulate insulin secretion, increase the mass of B-cells, modulate gastric acid secretion, modulate gastric emptying, and/or decrease glucagon production. In certain embodiments, the disease, or condition is type 2 diabetes.

In certain embodiments, the disease, or condition is obesity, or one, or more diseases, or conditions associated with obesity. Non-limiting examples of obesity, and obesity related conditions include, symptomatic obesity, simple obesity, childhood obesity, morbid obesity, and abdominal obesity (central obesity characterized by abdominal adiposity). Non-limiting examples of symptomatic obesity include, endocrine obesity (e.g., Cushing syndrome, hypothyroidism, insulinoma, obese type II diabetes, pseudohypoparathyroidism, hypogonadism), hypothalamic obesity, hereditary obesity (e.g., Prader-Willi syndrome, Laurence-Moon-Biedl syndrome), and drug-induced obesity (e.g., steroid, phenothiazine, insulin, sulfonylurea agent, or β-blocker-induced obesity).

Examples of such diseases, and conditions associated with obesity include, without limitation, glucose tolerance disorders, diabetes (e.g., type 2 diabetes, obese diabetes), lipid metabolism abnormality, hyperlipidemia, hypertension, cardiac failure, hyperuricemia, gout, fatty liver (including non-alcoholic steatohepatitis (NASH)), coronary heart disease (e.g., myocardial infarction, angina pectoris), cerebral infarction (e.g., brain thrombosis, transient cerebral ischemic attack), bone, or articular disease (e.g., knee osteoarthritis, hip osteoarthritis, spondylitis deformans, lumbago), sleep apnea syndrome, obesity hypoventilation syndrome (Pickwickian syndrome), menstrual disorder (e.g., abnormal menstrual cycle, abnormality of menstrual flow and cycle, amenorrhea, abnormal catamenial symptom), visceral obesity syndrome, and metabolic syndrome. In certain embodiments, the compounds described herein can be used to treat subjects exhibiting symptoms of both obesity, and insulin deficiency.

In some embodiments, the disease, or condition is diabetes. Non-limiting examples of diabetes include, type 1 diabetes, type 2 diabetes (e.g., diet-treated type 2-diabetes, sulfonylurea-treated type 2-diabetes, a far-advanced stage type 2-diabetes, long-term insulin-treated type 2-diabetes), diabetes mellitus (e.g., non-insulin-dependent diabetes mellitus, insulin-dependent diabetes mellitus), gestational diabetes, obese diabetes, autoimmune diabetes, and borderline type diabetes.

In some embodiments, the disease, or condition is associated with diabetes (e.g., a complication of diabetes). Non-limiting examples of disorders associated with diabetes include, obesity, obesity-related disorders, metabolic syndrome, neuropathy, nephropathy (e.g., diabetic nephropathy), retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, diabetic cachexia, delayed wound healing, diabetic dyslipidemia peripheral blood circulation disorder, cardiovascular risk factors, (e.g., coronary artery disease, peripheral artery disease, cerebrovascular disease, hypertension, and risk factors related to unmanaged cholesterol, and/or lipid levels, and/or inflammation), NASH, bone fracture, and cognitive dysfunction.

Other non-limiting examples of diseases, or conditions related to diabetes include, pre-diabetes, hyperlipidemia (e.g., hypertriglyceridemia, hypercholesterolemia, high LDL-cholesterolemia, low HDL-cholesterolemia, postprandial hyperlipemia), metabolic syndrome (e.g., metabolic disorder where activation of GLP-1R is beneficial, metabolic syndrome X), hypertension, impaired glucose tolerance (IGT), insulin resistance, and sarcopenia.

In some embodiments, the disease, or condition is diabetes, and obesity (diabesity). In certain embodiments, the compounds described herein are useful in improving the therapeutic effectiveness of metformin.

In some embodiments, the disease or condition is a disorder of a metabolically important tissue.

In some embodiments, the disease, or condition is a fatty liver disease. Fatty liver diseases include, but are not limited to, non-alcoholic fatty acid liver disease (NAFLD), steatohepatitis, non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, glycogen storage diseases, Weber-Christian disease, Wolmans disease, acute fatty liver of pregnancy, and lipodystrophy.

Non-alcoholic fatty liver disease (NAFLD) represents a spectrum of disease occurring in the absence of alcohol abuse, and is typically characterized by the presence of steatosis (fat in the liver). NAFLD is believed to be linked to a variety of conditions, e.g., metabolic syndrome (including obesity, diabetes, and hypertriglyceridemia), and insulin resistance. It can cause liver disease in adults and children, and may ultimately lead to cirrhosis (Skelly et al., J Hepatol 2001; 35:195-9; Chitturi et al., Hepatology 2002; 35 (2): 373-9). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol 2002; 17 SuppkS 186-90). In certain embodiments, the subject is a pediatric subject (e.g., 6-16 years old; or 6-12 years old; or 6-10 years old). In certain embodiments, the subject is an adult subject.

Other non-limiting examples of diseases, or conditions in metabolically important tissues include, joint disorders (e.g., osteoarthritis, secondary osteoarthritis), steatosis (e.g. in the liver); gall stones; gallbladder disorders; gastroesophageal reflux; sleep apnea; hepatitis; fatty liver; bone disorder characterized by altered bone metabolism, such as osteoporosis, including post-menopausal osteoporosis, poor bone strength, osteopenia, Paget's disease, osteolytic metastasis in cancer patients, osteodistrophy in liver disease, and the altered bone metabolism caused by renal failure, or haemodialysis, bone fracture, bone surgery, aging, pregnancy, protection against bone fractures, and malnutritionpolycystic ovary syndrome; renal disease (e.g., chronic renal failure, glomerulonephritis, glomerulosclerosis, nephrotic syndrome, hypertensive nephrosclerosis, end-stage renal disease); muscular dystrophy, angina pectoris, acute, or chronic diarrhea, testicular dysfunction, respiratory dysfunction, frailty, sexual dysfunction (e.g., erectile dysfunction), and geriatric syndrome. In certain embodiments, the chemical entities described herein can be used for treating surgical trauma by improving recovery after surgery, and/or by preventing the catabolic reaction caused by surgical trauma.

In some embodiments, the disease, or condition is a cardiovascular disease. Non-limiting examples of cardiovascular disease include, congestive heart failure, atherosclerosis, arteriosclerosis, coronary heart disease, or peripheral artery disease, stroke, coronary artery disease, congestive heart failure, coronary heart disease, hypertension, cardiac failure, cerebrovascular disorder (e.g., cerebral infarction), vascular dysfunction, myocardial infarction, elevated blood pressure (e.g., 130/85 mm Hg or higher), and prothrombotic state (exemplified by high fibrinogen, or plasminogen activator inhibitor in the blood).

In some embodiments, the disease, or condition is a neurological disorder (e.g., neurodegenerative disorder), or a psychiatric disorder. Non-limiting examples of neurological disorders include, brain insulin resistance, mild cognitive impairment (MCI), Alzheimer's disease (AD), Parkinson's disease (PD), anxiety, dementia (e.g., senile dementia), traumatic brain injury, Huntington's chores, tardive dyskinesia, hyperkinesia, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis (ALS), glaucoma, and apoptosis-mediated degenerative diseases of the central nervous system (e.g., Creutzfeld-Jakob Disease, bovine spongiform encephalopathy (mad cow disease), chronic wasting syndrome). See, e.g., US20060275288A1.

Non-limiting examples of psychiatric disorders include, drug dependence/addiction (narcotics, amphetamines, and attention deficit/hyperactivity disorder (ADHD). The chemical entities described herein can be useful in improving behavioral response to addictive drugs, decreasing drug dependence, prevention drug abuse relapse, and relieving anxiety caused by the absence of a given addictive substance. See, e.g., US20120021979A1.

In certain embodiments, the chemical entities described herein are useful in improving learning, and memory by enhancing neuronal plasticity, and facilitation of cellular differentiation, and also in preserving dopamine neurons, and motor function in Morbus Parkinson.

In some embodiments, the disease, or condition is impaired fasting glucose (IFG), impaired fasting glycemia (IFG), hyperglycemia, insulin resistance (impaired glucose homeostasis), hyperinsulinemia, elevated blood levels of fatty acids, or glycerol, a hypoglycemic condition, insulin resistant syndrome, paresthesia caused by hyperinsulinemia, hyperlipidaemia, hypercholesteremia, impaired wound healing, leptin resistance, glucose intolerance, increased fasting glucose, dyslipidemia (e.g., hyperlipidemia, atherogenic dyslipidemia characterized by high triglycerides and low HDL cholesterol), glucagonoma, hyperuricacidemia, hypoglycemia (e.g., nighttime hypoglycemia), and concomitant comatose endpoint associated with insulin.

In certain embodiments, the compounds described herein can reduce, or slow down the progression of borderline type, impaired fasting glucose, or impaired fasting glycemia into diabetes.

In some embodiments, the disease, or condition is an autoimmune disorder. Non-limiting examples of autoimmune disorders include, multiple sclerosis, experimental autoimmune encephalomyelitis, autoimmune disorder is associated with immune rejection, graft versus host disease, uveitis, optic neuropathies, optic neuritis, transverse myelitis, inflammatory bowel disease, rheumatoid arthritis, ankylosing spondylitis, systemic lupus erythematosus, myasthenia gravis, and Graves disease. See, e.g., US20120148586A1.

In some embodiments, the disease, or condition is a stomach, or intestine related disorder. Non-limiting examples of these disorders include, ulcers of any etiology (e.g. peptic ulcers, Zollinger-Ellison syndrome, drug-induced ulcers, ulcers related to infections, or other pathogens), digestion disorders, malabsorption, short bowel syndrome, cul-de-sac syndrome, inflammatory bowel diseases (Crohn's disease, and ulcerative colitis), celiac sprue, hypogammaglobulinemic sprue, chemotherapy, and/or radiation therapy-induced mucositis, and diarrhea, gastrointestinal inflammation, short bowel syndrome, colitis ulcerosa, gastric mucosal injury (e.g., gastric mucosal injury caused by aspirin), small intestinal mucosal injury, and cachexia (e.g., cancerous cachexia, tuberculous cachexia, cachexia associated with blood disease, cachexia associated with endocrine disease, cachexia associated with infectious disease, cachexia caused by acquired immunodeficiency syndrome).

In some embodiments, the compounds described herein can be used to reduce body weight (e.g., excess body weight), prevent body weight gain, induce weight loss, decrease body fat, or reduce food intake in a subject (e.g., a subject in need thereof). In certain embodiments, the weight increase in a subject may be attributed to excessive ingestion of food, or unbalanced diets, or may be weight increase derived from a concomitant drug (e.g., insulin sensitizers having a PPARγ agonist-like action, such as troglitazone, rosiglitazone, englitazone, ciglitazone, pioglitazone, and the like). Alternatively, the weight increase may be weight increase before reaching obesity, or may be weight increase in an obese subject. The weight increase may also be medication-induced weight gain, or weight gain subsequent to cessation of smoking.

In some embodiments, the condition, disease, or disorder is an eating disorder, such as hyperphagia, binge eating, bulimia, or compulsive eating.

In some embodiments, the disease, or condition is an inflammatory disorder. Non-limiting examples of inflammatory disorders include, chronic rheumatoid arthritis, spondylitis deformans, arthritis deformans, lumbago, gout, post-operational or post-traumatic inflammation, bloating, neuralgia, laryngopharyngitis, cystitis, pneumonia, pancreatitis, enteritis, inflammatory bowel disease (including inflammatory large bowel disease), inflammation in metabolically important tissues including liver, fat, pancreas, kidney, and gut, and a proinflammatory state (e.g., elevated levels of proinflammatory cytokines or, markers of inflammation-like C-reactive protein in the blood).

In some embodiments, the disease, or condition is cancer. Suitable examples of cancer include, breast cancer (e.g., invasive ductal breast cancer, noninvasive ductal breast cancer, inflammatory breast cancer), prostate cancer (e.g., hormone-dependent prostate cancer, hormone-independent prostate cancer), pancreatic cancer (e.g., ductal pancreatic cancer), gastric cancer (e.g., papillary adenocarcinoma, mucous adenocarcinoma, adenosquamous carcinoma), lung cancer (e.g., non-small cell lung cancer, small-cell lung cancer, malignant mesothelioma), colon cancer (e.g., gastrointestinal stromal tumor), rectal cancer (e.g., gastrointestinal stromal tumor), colorectal cancer (e.g., familial colorectal cancer, hereditary non-polyposis colorectal cancer, gastrointestinal stromal tumor), small intestinal cancer (e.g., non-Hodgkin's lymphoma, gastrointestinal stromal tumor), esophageal cancer, duodenal cancer, tongue cancer, pharyngeal cancer (e.g., nasopharyngeal cancer, oropharynx cancer, hypopharyngeal cancer), salivary gland cancer, brain tumor (e.g., pineal astrocytoma, pilocytic astrocytoma, diffuse astrocytoma, anaplastic astrocytoma), neurilemmoma, liver cancer (e.g., primary liver cancer, extrahepatic bile duct cancer), renal cancer (e.g., renal cell cancer, transitional cell cancer of the renal pelvis and ureter), bile duct cancer, endometrial cancer, uterine cervical cancer, ovarian cancer (e.g., epithelial ovarian cancer, extragonadal germ cell tumor, ovarian germ cell tumor, ovarian tumor of low malignant potential), bladder cancer, urethral cancer, skin cancer (e.g., intraocular (ocular) melanoma, Merkel cell carcinoma), hemangioma, malignant lymphoma, malignant melanoma, thyroid cancer (e.g., medullary thyroid cancer), parathyroid cancer, nasal cavity cancer, sinus cancer, bone tumor (e.g., osteosarcoma, Ewing tumor, uterine sarcoma, soft tissue sarcoma), angiofibroma, sarcoma of the retina, penis cancer, testicular tumor, pediatric solid tumor (e.g., Wilms' tumor, childhood kidney tumor), Kaposi's sarcoma, Kaposi's sarcoma caused by AIDS, tumor of maxillary sinus, fibrous histiocytoma, leiomyosarcoma, rhabdomyosarcoma, and leukemia (e.g., acute myeloid leukemia, acute lymphoblastic leukemia).

In certain embodiments, provided herein are methods for the treatment that include, the administration of an effective amount of compounds provided herein, or a pharmaceutically acceptable salt thereof. In certain embodiments, the methods encompass the step of administering to the subject in need thereof an amount of a compound described herein effective for the treatment of disease, or condition in combination with a second agent effective for the treatment, or prevention of the disease, or condition. In certain embodiments, the compound is in the form of a pharmaceutical composition, or dosage form, as described elsewhere herein.

In certain embodiments, the subject is a treatment naïve subject. In further embodiments, the subject has previously received therapy. For instance, in certain embodiments, the subject has not responded to a single agent treatment regimen.

In certain embodiments, the subject is a subject that discontinued some other therapy because of one or more adverse events associated with the other therapy. In certain embodiments, the subject has received some other therapy and discontinued that therapy prior to administration of a method provided herein. In further embodiments, the subject has received therapy and continues to receive that therapy along with administration of a compound provided herein. The compounds described herein can be co-administered with other therapy for treatment of the disease or condition according to the judgment of one of skill in the art. In certain embodiments, the methods or compositions provided herein can be co-administered with a reduced dose of the other therapy for the treatment of the disease or condition.

### Diagnostic Applications

In some embodiments, the compounds provided herein are used in diagnostic applications. These applications may be useful, for example, in making a diagnosis, and/or prognosis for a disease, or condition, such as a metabolic disease, or condition.

In some diagnostic and prognostic applications or embodiments, the compound may be labeled with a detectable moiety. Suitable detectable moieties include, but are not limited to, radioisotopes, fluorescent labels, and enzyme-substrate labels. In another embodiment, the compound need not be labeled, and the presence of the compound can be detected using a labeled antibody, or antigen binding fragment thereof which specifically binds to the compound.

### Kits

In some embodiments, a compound provided herein is provided in the form of a kit (i.e., a packaged combination of reagents in predetermined amounts with instructions for performing a procedure). In some embodiments, the procedure is a diagnostic assay. In certain embodiments, the procedure is a therapeutic procedure.

In some embodiments, the kit further comprises a solvent for the reconstitution of the compound. In some embodiments, the compound is provided in the form of a pharmaceutical composition.

In some embodiments, the kits can include a compound, or composition provided herein, an optional second agent, or composition, and instructions providing information to a health care provider regarding usage for treating the disorder. Instructions may be provided in printed form, or in the form of an electronic medium such as a floppy disc, CD, or DVD, or in the form of a website address where such instructions may be obtained. A unit dose of a compound, or a composition provided herein, or a second agent, or composition, can include a dosage such that when administered to a subject, a therapeutically, or prophylactically effective plasma level of the compound, or composition can be maintained in the subject for at least one day. In some embodiments, a compound, or composition can be included as a sterile aqueous pharmaceutical composition, or dry powder (e.g., lyophilized) composition.

In some embodiments, suitable packaging is provided. As used herein, “packaging” includes a solid matrix, or material customarily used in a system, and capable of holding within fixed limits a compound provided herein, and/or a second agent suitable for administration to a subject. Such materials include, glass, and plastic (e.g., polyethylene, polypropylene, and polycarbonate) bottles, vials, paper, plastic, plastic-foil laminated envelopes, and the like. If e-beam sterilization techniques are employed, the packaging should have sufficiently low density to permit sterilization of the contents.

### Preparation and Synthetic Procedures

In some embodiments, the compounds described herein are prepared as outlined in Schemes 1-4. The synthesis of the compounds in this application is not limited to these general reaction schemes illustrated here. For detailed synthesis of each individual compound, please check the Examples section.

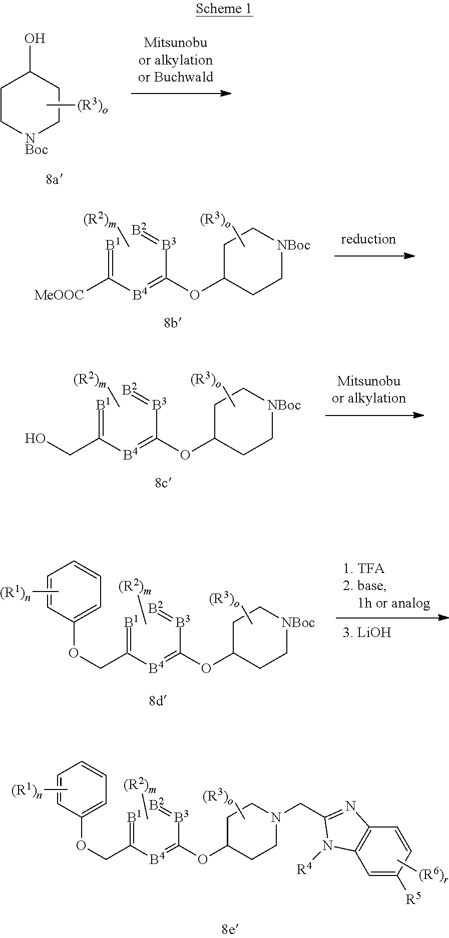

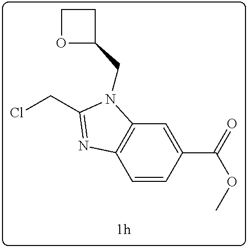 [SMILES: COC(=O)c1ccc2nc(CCl)n(C[C@@H]3CCO3)c2c1]

Scheme 1. (zero, one, or two of B¹, B², B³, and B⁴ are N, and the rest are CH). Alcohol 8a′ can be used to synthesize 8b′ through Mitsunobu reaction with an alcohol (e.g., phenol), or converted to a mesylate followed by alkylation with a phenol, or coupling with aryl halide through Buchwald reaction to form 8b′. Reduction of the ester group in 8b′ to afford alcohol 8c′. Then through Mitsunobu reaction with a phenol or mesylate formation of 8c′ followed by alkylation with a phenol to provide 8d′. Deprotection of the Boc group with TFA, followed by coupling with 1h (methyl 2-(chloromethyl)-3-[[(2S)-oxetan-2-yl]methyl]benzimidazole-5-carboxylate) and hydrolysis to afford 8e′.

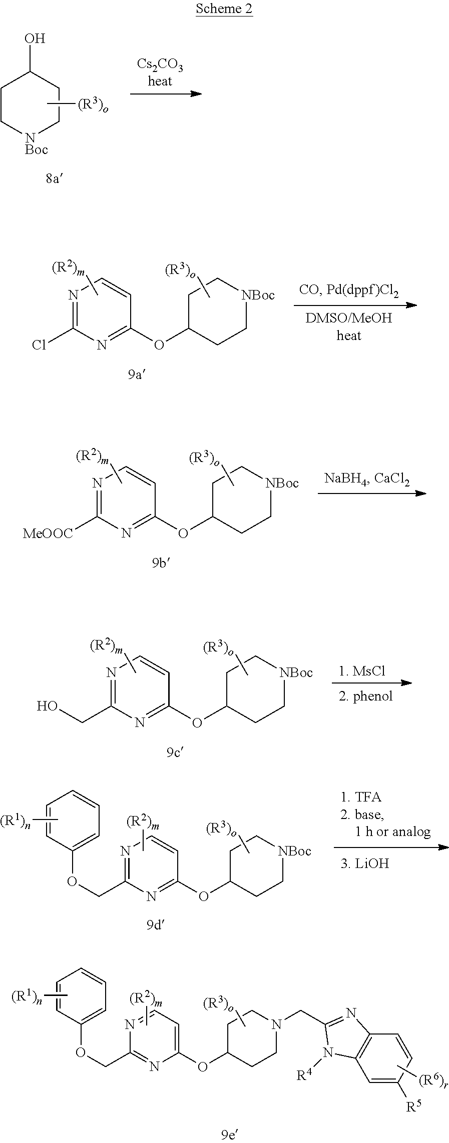

Scheme 2. 9a′ can be synthesized from 8a′ through alkylation. Palladium-catalyzed carbonylation would afford ester 9b′, which can be reduced to provide alcohol 9c′. Mesylation formation of alcohol 9c′ followed by alkylation with a phenol can provide 9d′. Deprotection of the Boc group with TFA, followed by coupling with 1 h and hydrolysis to afford 9e′.

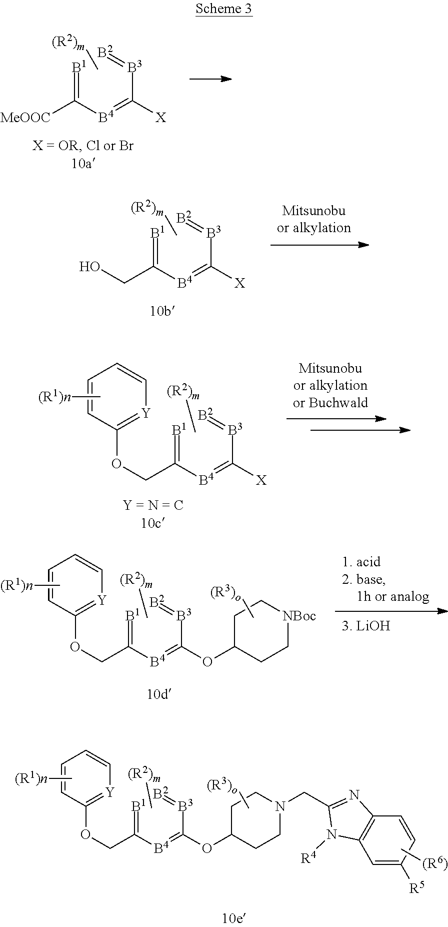

Scheme 3. (zero, one, or two of B¹, B², B³, and B⁴ are N, and the rest are CH). Reduction of compound 10a′ resulted in an alcohol 10b′, which can be coupled with a phenol through alkylation of a mesylate intermediate of 10b′, a Mitusnobu reaction or alkylation of 10b′ with an alpha-halopyridine to generate 10c′. Then 10d′ can be synthesized from 10c′ through a Buchwald reaction of Boc-protected piperidine alcohol (when X═Cl or Br) or alkylation through a mesylate intermediate of the alcohol or a Mitsunobu reaction (X═OR, after deprotection). After removal of the Boc group in 10d′ with TFA or HCl, coupling with ester 1 h or its analogs followed by hydrolysis led to final product 10e′.

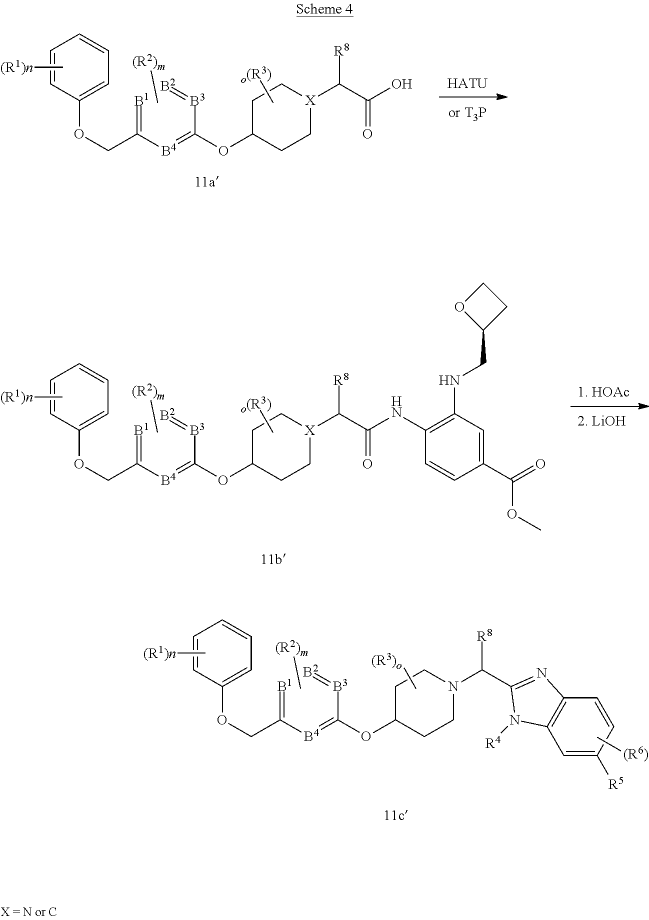

Scheme 4. (zero, one, or two of B¹, B², B³, and B⁴ are N, and the rest are CH). Sometimes compounds can be made from 11a′. HATU or T₃P-mediated coupling of 11a′ with 1g or its analogs results in 11b′. Heating of 11b′ in acetic acid cyclizes 11b′ into a benzimidazole ester intermediate which was hydrolyzed with LiOH to afford 11c′.

### EXAMPLES

### Preparation of the Compounds

The compounds used in the reactions described herein are made according to organic synthesis techniques known to those skilled in this art, starting from commercially available chemicals, and/or from compounds described in the chemical literature. “Commercially available chemicals” are obtained from standard commercial sources such as Acros Organics (Pittsburgh, PA), Advanced ChemBlocks, Inc (Burlingame, CA), Aldrich Chemical (Milwaukee, WI, including Sigma Chemical and Fluka), AK Scientific (Union City, CA), AstaTech, Inc. (Bristol, PA), Aurum Pharmatech LLC (Franklin Park, NJ), Combi-Blocks, Inc. (San Diego, CA), Enamine (Monmouth Jct., NJ), Fisher Scientific Co. (Pittsburgh, PA), Frontier Scientific (Logan, UT), TCI America (Portland, OR), and VWR (Radnor, PA). Specific and analogous reactants are optionally identified through the indices of known chemicals prepared by the Chemical Abstract Service of the American Chemical Society, which are available in most public and university libraries, as well as through on-line databases.

Suitable reference books that detail the synthesis of reactants useful in the preparation of compounds described herein, or provide references to articles that describe their preparation, include for example, “Synthetic Organic Chemistry”, John Wiley & Sons, Inc., New York; S. R. Sandler et al., “Organic Functional Group Preparations,” 2nd Ed., Academic Press, New York, 1983; “T. L. Gilchrist, “Heterocyclic Chemistry”, 2nd Ed., John Wiley & Sons, New York, 1992; and J. March, “Advanced Organic Chemistry: Reactions, Mechanisms and Structure”, 4th Ed., Wiley-Interscience, New York, 1992; R. C. Larock “Comprehensive Organic Transformations: A Guide to Functional Group Preparations” 2nd Edition (1999) Wiley-VCH, ISBN: 0-471-19031-4; “Organic Reactions” (1942-2000) John Wiley & Sons, in over 55 volumes; and “Chemistry of Functional Groups” John Wiley & Sons, in 73 volumes. Some compounds require the application of protecting groups. The need for such protection is within the skill in the art. For a general description of protecting groups and their use, see T.W. Greene, P.G.M. Nuts, and Protective Groups in Organic Synthesis, John Wiley & Sons, New York, 1999.

### Analytical Methods and Instrumentation

Proton nuclear magnetic resonance (NMR) spectra were obtained on either Bruker, or Varian spectrometers at 400, or 600 MHz. NMR spectra are reported relative to residual solvent signals as follows: chemical shift δ (ppm), multiplicity, coupling constant J (Hz), and integration. Tetramethylsilane (TMS) was used as an internal standard in some of the cases. Mass spectral data were measured using one of the two following systems: System A: Waters Acquity i-class ultra-performance liquid chromatography (UPLC) system with Acquity Photo Diode Array Detector, Acquity Evaporative Light Scattering Detector (ELSD) and Waters ZQ Mass Spectrometer. Data was acquired using Waters MassLynx 4.1 software and purity characterized by UV wavelength 220 nm, evaporative light scattering detection (ELSD) and electrospray positive ion (ESI) (column: Acquity UPLC BEH C18 1.7μ t 2.1×50 mm). System B: Agilent LC/MS consisting of a 1200 series LC and 6140 Quadrupole MS detector (column: Agilent USGYL01131, HPH—C18 2.7 μM, 2.1×50 mm). Solvents used: acetonitrile/water, containing 0.1% formic acid; flow rate 0.7 mL/min. Preparatory HPLC purifications were conducted with a flow rate of 15 mL/min and detection by UV wavelength 214 nm and 254 nm (Column: Jupiter® 10 μM Proteo 90 Å, 250×21.2 mm A, solvent: acetonitrile/water, containing modifier such as 0.1% trifluoroacetic acid, formic acid or acetic acid). Compound purity was checked on an analytical HPLC (Waters Acquity UPLC H-Class instrument), with a flow rate of 0.5 mL/min (Acquity BEH C18, 50×2.1 mm column).

### Abbreviations Used in the Examples Include

| Abbreviation | Name |
|---|---|
| CH₃CN | acetonitrile |
| aq. | aqueous |
| atm | atmospheres |
| BINAP | (1,1′-binaphthalene-2,2′-diyl)bis(diphenylphosphine) |
| Boc | t-butoxycarbonyl |
| CCl₄ | carbon tetrachloride |
| CDCl₃ | deuterated chloroform |
| CO | carbon monoxide gas |
| CO₂ | carbon dioxide |
| Cs₂CO₃ | cesium carbonate |
| CuBr | copper(I) bromide |
| Cu(OAc)₂ | copper(II) acetate |
| DCM | dichloromethane |
| DEAD | diethyl azodicarboxylate |
| DIPEA | diisopropylethylamine |
| DMF | N,N-dimethylformamide |
| DMSO | dimethylsulfoxide |
| ESI | electrospray ionization |
| Et₃N | triethylamine |
| EtOAc | ethyl acetate |
| EtOH | ethanol |
| h | hours |
| H₂O | water |
| HATU | 1-[bis(dimethylamino)methylene]-1H-1,2,3- |
| | triazolo[4,5-b]pyridinium |
| | 3-oxid hexafluorophosphate |
| HCl | hydrochloric acid |
| HOAc | acetic acid |
| K₂CO₃ | potassium carbonate |
| KI | potassium iodide |
| KOH | potassium hydroxide |
| KOtBu | potassium tert-butoxide |
| LiAlH₄ | lithium aluminum hydride |
| LiOH | lithium hydroxide |
| MeOH | methanol |
| min | minutes |
| N₂ | nitrogen |
| Na₂CO₃ | sodium carbonate |
| Na₂SO₄ | sodium sulfate |
| NaH | sodium hydride |
| NaHCO₃ | sodium bicarbonate |
| NaN₃ | sodium azide |
| NaOH | sodium hydroxide |
| NBS | N-bromosuccinimide |
| NH₄Cl | ammonium chloride |
| NH₄HCO₃ | ammonium bicarbonate |
| NMR | nuclear magnetic resonance |
| Pd₂(dba)₃ | tris(dibenzylideneacetone)dipalladium(0) |
| Pd(dppf)Cl₂ | [1,1′- |
| | bis(diphenylphosphino)ferrocene]dichloropalladium(II) |
| Pd/C | palladium on carbon |
| Pd(OAc)₂ | palladium(II) acetate |
| Prep-TLC | preparatory thin layer chromatography |
| RuPhos-Pd-G2 | chloro(2-dicyclohexylphosphino-2′,6′-diisopropoxy- |
| | 1,1′-biphenyl)[2-(2′-amino-1,1′- |
| | biphenyl)]palladium(II) |
| sat. | saturated |
| t-BuOH | tert-butanol |
| T₃P | propylphosphonic anhydride |
| TBD | 1,5,7-triazabicyclo[4.4.0]dec-5-ene |
| TFA | trifluoracetic acid |
| THF | tetrahydrofuran |

Unless otherwise noted, reagents, and solvents were used as received from commercial suppliers. Anhydrous solvents and oven-dried glassware were used for synthetic transformations sensitive to moisture, and/or oxygen. Reaction times, and yields were not optimized. Example numbers and compound numbers are the same.

### Examples

### Example 552. 2-{[(3R,4S)-4-({2-[(4-Cyano-2-fluorophenoxy)methyl]pyrimidin-4-yl}oxy)-3-fluoropiperidin-1-yl]methyl}-1-{[(2S)-oxetan-2-yl]methyl}-1H-1,3-benzodiazole-6-carboxylic acid

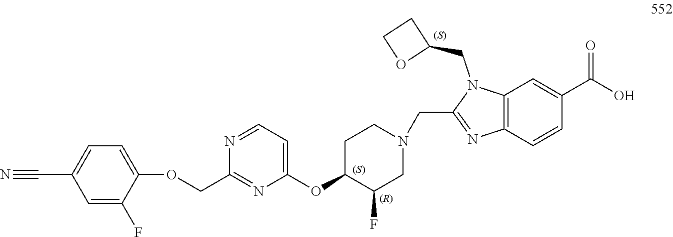

Example 552 was synthesized from tert-butyl (3R,4S)-3-fluoro-4-hydroxypiperidine-1-carboxylate as described for Example 599.

### Example 553. 2-{[(3S,4S)-4-({2-[(4-Cyano-2-fluorophenoxy)methyl]pyrimidin-4-yl}oxy)-3-fluoropiperidin-1-yl]methyl}-1-{[(2S)-oxetan-2-yl]methyl}-1H-1,3-benzodiazole-6-carboxylic acid

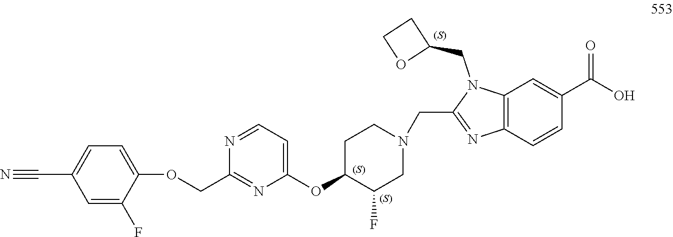

Example 553 was synthesized according to Example 599.

### Example 554. 2-(((2S,4S)-4-((2-(((5-Chloro-3-fluoropyridin-2-yl)oxy)methyl)pyrimidin-4-yl)oxy)-2-methylpiperidin-1-yl)methyl)-1-(((S)-oxetan-2-yl)methyl)-1H-benzo[d]imidazole-6-carboxylic acid (554)

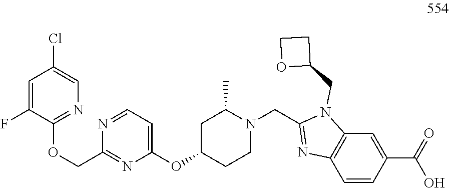 [SMILES: C[C@H]1C[C@@H](Oc2ccnc(COc3ncc(Cl)cc3F)n2)CCN1Cc1nc2ccc(C(=O)O)cc2n1C[C@@H]1CCO1]

### Step A. tert-Butyl (2S,4S)-4-((2-(((5-chloro-3-fluoropyridin-2-yl)oxy)methyl)pyrimidin-4-yl)oxy)-2-methylpiperidine-1-carboxylate (554a)

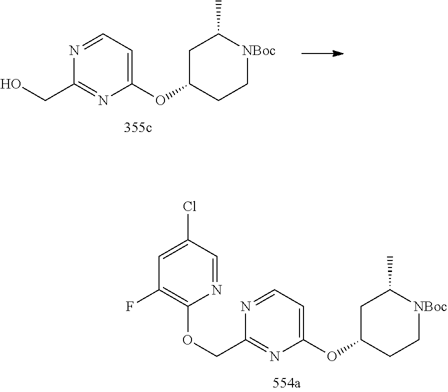 [SMILES: C[C@H]1C[C@@H](Oc2ccnc(CO)n2)CCN1C(=O)OC(C)(C)C ; C[C@H]1C[C@@H](Oc2ccnc(COc3ncc(Cl)cc3F)n2)CCN1C(=O)OC(C)(C)C]

To a stirred solution of 606e (50 mg, 0.15 mmol) in THF (2 mL) under nitrogen at 0° C. was added NaH (25 mg, 0.62 mmol, 60% dispersion in mineral oil). The reaction was stirred at 0° C. for 1H and 5-chloro-2,3-difluoropyridine (35 mg, 0.23 mmol) was added. After heating at 50° C. for 2H, the reaction was quenched with ice-water (10 mL) and extracted with EtOAc (30 mL×3). The organic layer was washed with water (15 mL), brine (15 mL), dried over Na₂SO₄, filtered and concentrated in vacuo to provide a crude residue, which was purified by silica gel column chromatography (EtOAc:petroleum ether=1:4) to afford the title product (554a) (56 mg, 76% yield). m/z (ESI, +ve ion)=453.1 [M+H]⁺.

### Step B. 2-(((5-Chloro-3-fluoropyridin-2-yl)oxy)methyl)-4-(((2S,4S)-2-methylpiperidin-4-yl)oxy)pyrimidine (554b)

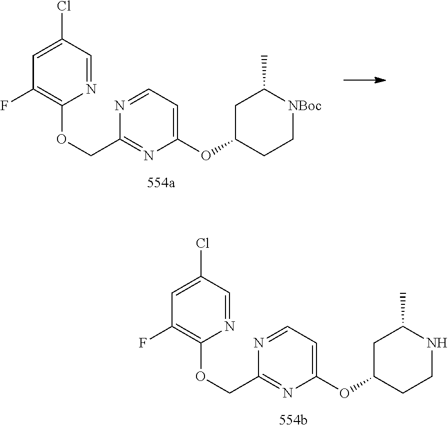 [SMILES: C[C@H]1C[C@@H](Oc2ccnc(COc3ncc(Cl)cc3F)n2)CCN1C(=O)OC(C)(C)C ; C[C@H]1C[C@@H](Oc2ccnc(COc3ncc(Cl)cc3F)n2)CCN1]

A solution of 554a (35 mg, 0.07 mmol) in 4 N HCl in dioxane (2 mL) was stirred at 20° C. for 2H. After completion, the reaction was diluted with H₂O (15 mL) and extracted with EtOAc (20 mL×3). The organic layer was dried, filtered, and concentrated to afford the title product (554b) (crude, 27 mg), which was used in the next step without further purification. m/z (ESI, +ve ion)=353.1 [M+H]⁺.

### Step C. Methyl 2-(((2S,4S)-4-((2-(((5-chloro-3-fluoropyridin-2-yl)oxy)methyl)pyrimidin-4-yl)oxy)-2-methylpiperidin-1-yl)methyl)-1-(((S)-oxetan-2-yl)methyl)-1H-benzo[d]imidazole-6-carboxylate (554c)

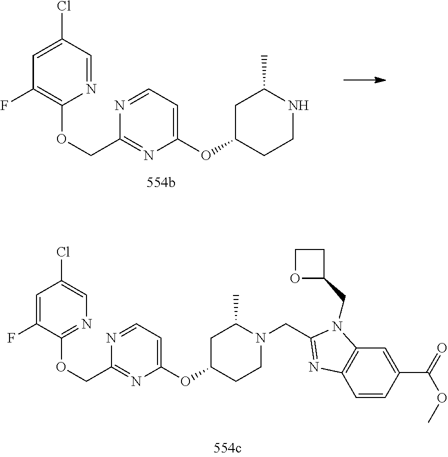 [SMILES: C[C@H]1C[C@@H](Oc2ccnc(COc3ncc(Cl)cc3F)n2)CCN1 ; COC(=O)c1ccc2nc(CN3CC[C@H](Oc4ccnc(COc5ncc(Cl)cc5F)n4)C[C@@H]3C)n(C[C@@H]3CCO3)c2c1]

A solution of 554b (50 mg, 0.14 mmol), methyl 2-(chloromethyl)-3-[[(2S)-oxetan-2-yl]methyl]benzimidazole-5-carboxylate (50 mg, 0.17 mmol), K₂CO₃ (59 mg, 0.43 mmol) and potassium iodide (24 mg, 0.14 mmol) in DMF (2 mL) was stirred at 50° C. for 4 h. The reaction was quenched with ice-water (10 mL) and extracted with EtOAc (30 mL×3). The combined organic layer was washed with water (15 mL), brine (15 mL), dried over Na₂SO₄, filtered and concentrated in vacuo to provide a crude residue, which was purified by silica gel column chromatography (MeOH:DCM=1:20) to afford the title product (554c) (65 mg, 90% yield). m/z (ESI, +ve ion)=611.2 [M+H]⁺.

### Step D. 2-(((2S,4S)-4-((2-(((5-Chloro-3-fluoropyridin-2-yl)oxy)methyl)pyrimidin-4-yl)oxy)-2-methylpiperidin-1-yl)methyl)-1-(((S)-oxetan-2-yl)methyl)-1H-benzo[d]imidazole-6-carboxylic acid (554)

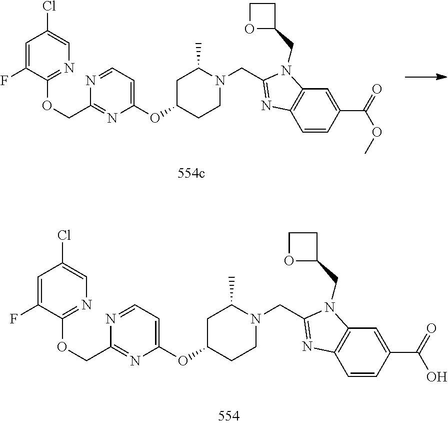 [SMILES: COC(=O)c1ccc2nc(CN3CC[C@H](Oc4ccnc(COc5ncc(Cl)cc5F)n4)C[C@@H]3C)n(C[C@@H]3CCO3)c2c1 ; C[C@H]1C[C@@H](Oc2ccnc(COc3ncc(Cl)cc3F)n2)CCN1Cc1nc2ccc(C(=O)O)cc2n1C[C@@H]1CCO1]

To a solution of 554c (45 mg, 0.07 mmol) in MeOH (1 mL) stirred at 20° C. was added a solution of LiOH (35 mg, 1.47 mmol) in H₂O (1 mL). The reaction was stirred at 20° C. for 5 h. After completion, 1 N HCl (about 2.0 mL) was added to the mixture to adjust to pH=6 in an ice bath. The reaction mixture was then extracted with EtOAc (15 mL×3). The combined organic layer was dried, filtered, concentrated and purified by silica gel column chromatography (MeOH:DCM=1:10) afford the title product (554) (36 mg, 78% yield). m/z (ESI, +ve ion)=597.2 [M+H]⁺. ¹H NMR (400 MHz, CD₃OD) δ ppm 8.37 (d, 1H), 8.29 (s, 1H), 7.98 (brd, 1H), 7.82 (d, 1H), 7.73-7.76 (m, 1H), 7.68 (d, 1H), 6.65 (d, 1H), 5.57 (s, 2H), 5.22-5.34 (m, 1H), 4.89-4.95 (m, 1H), 4.80-4.83 (m, 1H), 4.71 (dd, 1H), 4.57-4.64 (m, 2H), 4.30 (dt, 1H), 3.56 (d, 1H), 2.66-2.78 (m, 2H), 2.32-2.49 (m, 2H), 2.08-2.17 (m, 1H), 1.89-1.98 (m, 1H), 1.71-1.78 (m, 1H), 1.37-1.53 (m, 2H), 1.20 (d, 3H).

### Examples 566. 2-{[(2S,4S)-4-[(2-{[(3,5-Difluoropyridin-2-yl)oxy]methyl}pyrimidin-4-yl)oxy]-2-methylpiperidin-1-yl]methyl}-1-{[(2S)-oxetan-2-yl]methyl}-1H-1,3-benzodiazole-6-carboxylic acid

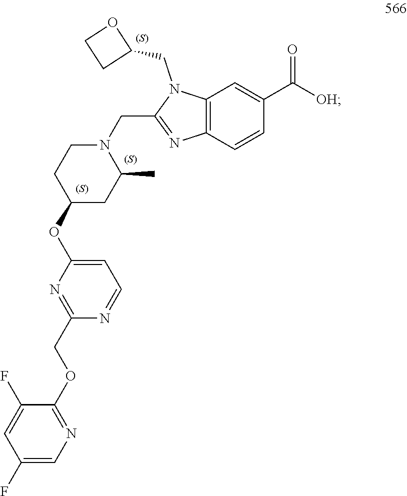

and

### Example 567. 2-{[(2S,4S)-4-[(2-{[(5-Cyanopyridin-2-yl)oxy]methyl}pyrimidin-4-yl)oxy]-2-methylpiperidin-1-yl]methyl}-1-{[(2S)-oxetan-2-yl]methyl}-1H-1,3-benzodiazole-6-carboxylic acid

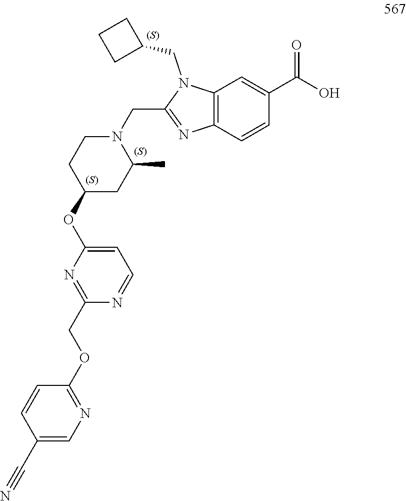

Example 566 and 567 were synthesized in similar procedures as described in Example 554.

### Examples 573. 2-{[(2S,4S)-4-({2-[(2-Cyano-4-fluorophenoxy)methyl]pyrimidin-4-yl}oxy)-2-methylpiperidin-1-yl]methyl}-7-fluoro-1-{[(2S)-oxetan-2-yl]methyl}-1H-1,3-benzodiazole-6-carboxylic acid

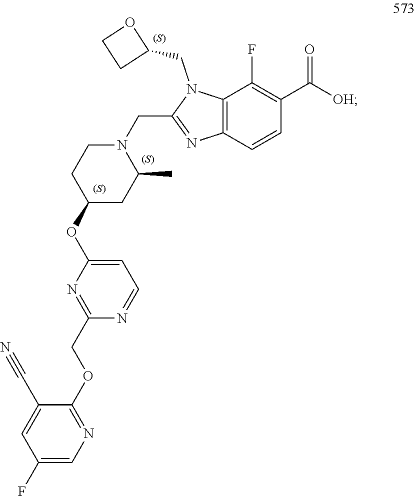

and

### Example 574. 2-{[(2S,4S)-4-({2-[(2-cyano-4-fluorophenoxy)methyl]pyrimidin-4-yl}oxy)-2-methylpiperidin-1-yl]methyl}-1-{[(2S)-oxetan-2-yl]methyl}-1H-imidazo[4,5-c]pyridine-6-carboxylic acid

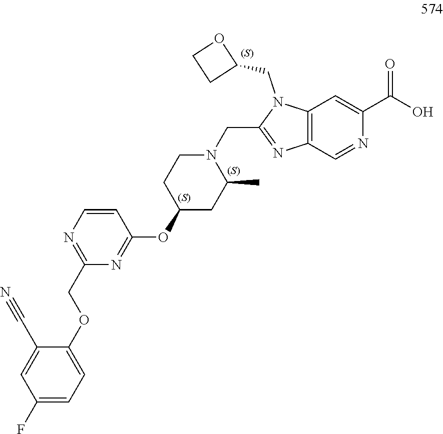

Examples 573 and 574 were prepared similarly to Example 606.

### Example 555. 2-(((2R,4S)-4-((2-((4-Chloro-2-fluorophenoxy)methyl)pyridin-4-yl)oxy)-2-(fluoromethyl)piperidin-1-yl)methyl)-1-(((S)-oxetan-2-yl)methyl)-1H-benzo[d]imidazole-6-carboxylic acid (555)

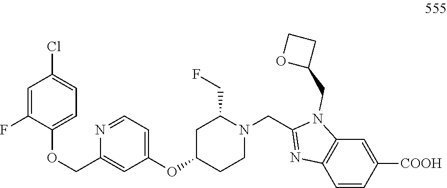 [SMILES: O=C(O)c1ccc2nc(CN3CC[C@H](Oc4ccnc(COc5ccc(Cl)cc5F)c4)C[C@@H]3CF)n(C[C@@H]3CCO3)c2c1]

### Step A. 4-(Benzyloxy)-2-((4-chloro-2-fluorophenoxy)methyl)pyridine (555a)

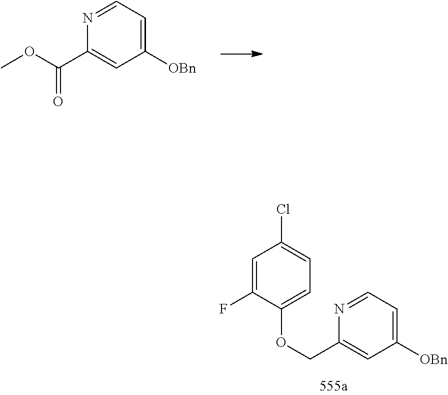 [SMILES: COC(=O)c1cc(OCc2ccccc2)ccn1 ; Fc1cc(Cl)ccc1OCc1cc(OCc2ccccc2)ccn1]

Methyl 4-(benzyloxy) picolinate was first reduced to (4-(benzyloxy)pyridin-2-yl)methanol with sodium borohydride. The title compound 555a was synthesized from the mesylate of (4-(benzyloxy)pyridin-2-yl)methanol and 4-chloro-2-fluorophenol using K₂CO₃ in the second step. m/z (ESI, +ve ion)=344.1 [M+H]⁺.

### Step B. 2-((4-Chloro-2-fluorophenoxy)methyl)pyridin-4-ol (555b)

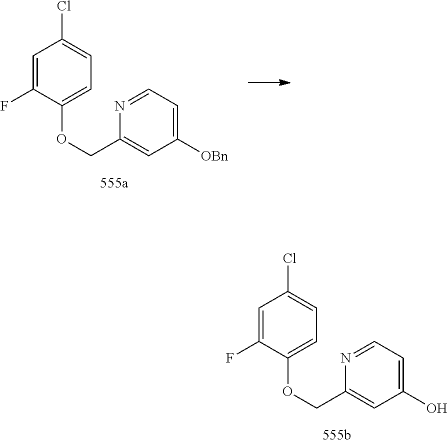 [SMILES: Fc1cc(Cl)ccc1OCc1cc(OCc2ccccc2)ccn1 ; Oc1ccnc(COc2ccc(Cl)cc2F)c1]

A mixture of 555a (300 mg, 0.87 mmol) in TFA (10 mL) was stirred at 80° C. for 16 h under N₂. After completion, the mixture was concentrated in vacuo. The residue was diluted with EtOAc (30 mL), washed with aq. NaHCO₃ (10 mL), H₂O (10 mL), and brine (10 mL). The organic layer was dried over anhydrous Na₂SO₄, filtered and concentrated in vacuo to afford the title product (555b) (200 mg, 86% yield). m/z (ESI, +ve ion)=254.2 [M+H]⁺.

### Step C. 1-(tert-Butyl) 2-methyl(2R,4S)-4-((2-((4-chloro-2-fluorophenoxy)methyl)pyridin-4-yl)oxy)piperidine-1,2-dicarboxylate (555c)

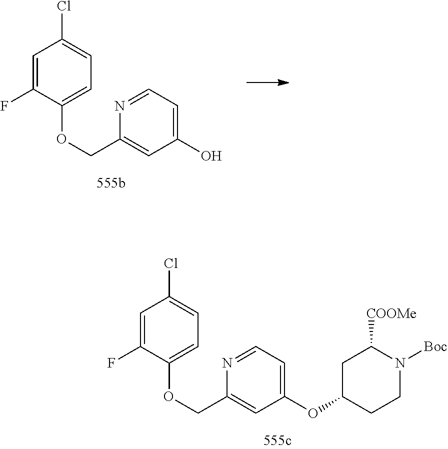 [SMILES: Oc1ccnc(COc2ccc(Cl)cc2F)c1 ; COC(=O)[C@H]1C[C@@H](Oc2ccnc(COc3ccc(Cl)cc3F)c2)CCN1C(=O)OC(C)(C)C]

The title compound 555c was synthesized from the mesylate of 1-tert-butyl 2-methyl(2R,4R)-4-hydroxypiperidine-1,2-dicarboxylate and 555b using K₂CO₃ in the second step. m/z (ESI, +ve ion)=495.1 [M+H]⁺.

### Step D. tert-Butyl(2R,4S)-4-((2-((4-chloro-2-fluorophenoxy)methyl)pyridin-4-yl)oxy)-2-(hydroxymethyl)piperidine-1-carboxylate (555d)

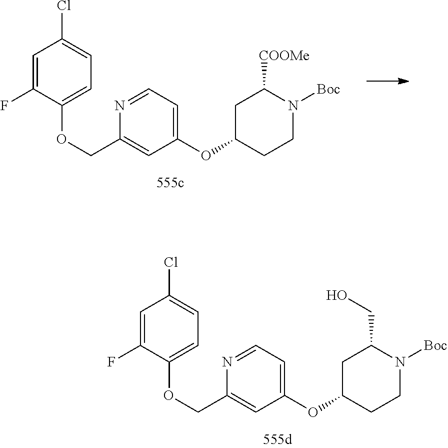 [SMILES: COC(=O)[C@H]1C[C@@H](Oc2ccnc(COc3ccc(Cl)cc3F)c2)CCN1C(=O)OC(C)(C)C ; CC(C)(C)OC(=O)N1CC[C@H](Oc2ccnc(COc3ccc(Cl)cc3F)c2)C[C@@H]1CO]

To a solution of 555c (240 mg, 0.48 mmol) in THF (10 mL) was added DIBAL-H (2.4 mL, 2.4 mmol, 1 M in hexanes) at 0° C. The reaction was stirred at 20° C. for 3H under N₂. After completion, it was quenched with Na₂SO₄·10H₂O (1 g), filtered and concentrated in vacuo to afford the title product (555d) (200 mg, 80% yield) as a colorless oil. m/z (ESI, +ve ion)=467.2 [M+H]⁺.

### Step E. ((2R,4S)-4-((2-((4-Chloro-2-fluorophenoxy)methyl)pyridin-4-yl)oxy)piperidin-2-yl)methanol (555e)

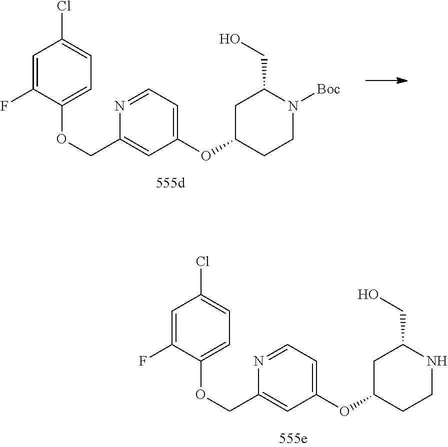 [SMILES: CC(C)(C)OC(=O)N1CC[C@H](Oc2ccnc(COc3ccc(Cl)cc3F)c2)C[C@@H]1CO ; OC[C@H]1C[C@@H](Oc2ccnc(COc3ccc(Cl)cc3F)c2)CCN1]

To a solution of 555d (200 mg, 0.43 mmol) in DCM (8 mL) was added TFA (2 mL) at 0° C. The reaction was stirred at 20° C. for 2H under N₂. After completion, the reaction mixture was concentrated in vacuo to afford the title product (555e) (160 mg, 92% yield) as a colorless oil. m/z (ESI, +ve ion)=367.1 [M+H]⁺.

### Step F. Methyl 2-(((2R,4S)-4-((2-((4-chloro-2-fluorophenoxy)methyl)pyridin-4-yl)oxy)-2-(hydroxymethyl)piperidin-1-yl)methyl)-1-(((S)-oxetan-2-yl)methyl)-1H-benzo[d]imidazole-6-carboxylate (555f)

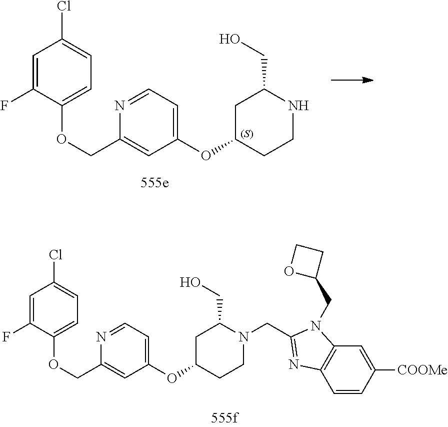

To a solution of 555e (180 mg, 0.49 mmol) and 1h (144 mg, 0.49 mmol) in CH₃CN (10 mL) was added K₂CO₃ (203 mg, 1.5 mmol) at 20° C. The mixture was stirred at 50° C. for 16 h under N₂. After completion, the reaction was quenched with H₂O (10 mL) and extracted with EtOAc (10 mL×3). The combined organic layer was washed with H₂O (10 mL), brine (10 mL), dried over anhydrous Na₂SO₄, filtered and concentrated in vacuo to give a crude residue, which was purified by silica gel column chromatography (MeOH:DCM=1:20) to afford the title product (555f) (100 mg, 31% yield) as a yellow oil. m/z (ESI, +ve ion)=625.2 [M+H]⁺.

### Step G. Methyl 2-(((2R,4S)-4-((2-((4-Chloro-2-fluorophenoxy)methyl)pyridin-4-yl)oxy)-2-(fluoromethyl)piperidin-1-yl)methyl)-1-(((S)-oxetan-2-yl)methyl)-1H-benzo[d]imidazole-6-carboxylate (555g)

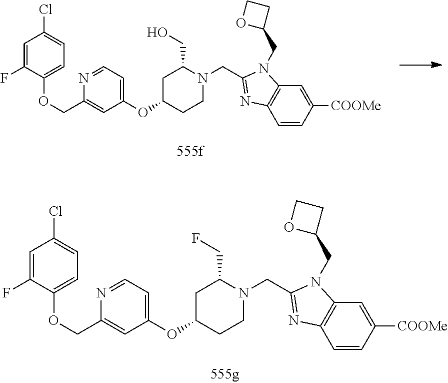 [SMILES: COC(=O)c1ccc2nc(CN3CC[C@H](Oc4ccnc(COc5ccc(Cl)cc5F)c4)C[C@@H]3CO)n(C[C@@H]3CCO3)c2c1 ; COC(=O)c1ccc2nc(CN3CC[C@H](Oc4ccnc(COc5ccc(Cl)cc5F)c4)C[C@@H]3CF)n(C[C@@H]3CCO3)c2c1]

To a solution of 555f (80 mg, 0.13 mmol) in DCM (5 mL) was added DAST (41 mg, 0.26 mmol) at 0° C. The reaction was stirred at 20° C. for 4 h under N₂. After completion, the mixture was quenched with H₂O (5 mL) and extracted with DCM (5 mL×3). The combined organic layer was washed with H₂O (5 mL), brine (5 mL), dried over anhydrous Na₂SO₄, filtered and concentrated in vacuo to give a crude residue, which was purified by silica gel column chromatography (DCM:MeOH=20:1) to afford the title product (555g) (50 mg, 56% yield) as a colorless oil. m/z (ESI, +ve ion)=627.2 [M+H]⁺.

### Step H. 2-(((2R,4S)-4-((2-((4-Chloro-2-fluorophenoxy)methyl)pyridin-4-yl)oxy)-2-(fluoromethyl)piperidin-1-yl)methyl)-1-(((S)-oxetan-2-yl)methyl)-1H-benzo[d]imidazole-6-carboxylic acid (555)

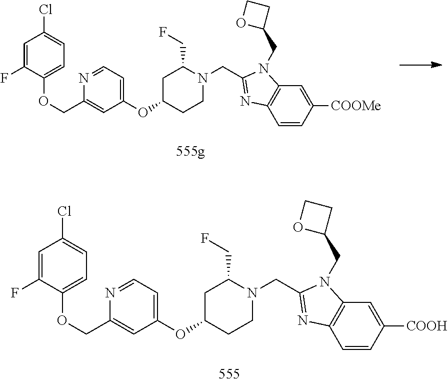 [SMILES: COC(=O)c1ccc2nc(CN3CC[C@H](Oc4ccnc(COc5ccc(Cl)cc5F)c4)C[C@@H]3CF)n(C[C@@H]3CCO3)c2c1 ; O=C(O)c1ccc2nc(CN3CC[C@H](Oc4ccnc(COc5ccc(Cl)cc5F)c4)C[C@@H]3CF)n(C[C@@H]3CCO3)c2c1]

To a mixture of 555g (40 mg, 0.064 mmol) in THF/H₂O (1:1, 4 mL) was added LiOH (7.6 mg, 0.32 mmol) at 20° C. After stirring at 20° C. for 16 h under N₂, the mixture was adjusted to pH=7 with 1 N HCl and extracted with EtOAc (5 mL×3). The combined organic layer was washed with H₂O (5 mL), brine (5 mL), dried over anhydrous Na₂SO₄, filtered and concentrated in vacuo to give a crude residue, which was purified by reverse phase HPLC (30% CH₃CN in H₂O, with 0.05% NH₃·H₂O as a modifier) to afford the title product (555) (10 mg, 25% yield). m/z (ESI, +ve ion)=613.2 [M+H]⁺. ¹H NMR (400 MHz, CD₃OD) δ ppm 8.33 (d, 1H), 8.16 (s, 1H) 7.94 (d, 1H) 7.58 (d, 1H), 7.19-7.27 (m, 1H), 7.07-7.18 (m, 3H), 6.96 (dd, 1H), 5.24-5.33 (m, 1H), 5.16 (s, 2H), 4.89-4.96 (m, 1H), 4.56-4.74 (m, 4H), 4.32 (dt, 1H), 3.78 (brd, 1H), 2.80-2.94 (m, 3H), 2.67-2.80 (m, 2H), 2.36-2.53 (m, 2H), 2.11-2.23 (m, 1H), 1.97-2.05 (m, 1H), 1.72-1.84 (m, 1H), 1.56-1.70 (m, 1H).

### Examples 575. 2-{[(2S,4S)-4-({2-[(2,4-Difluorophenoxy)methyl]pyrimidin-4-yl}oxy)-2-methylpiperidin-1-yl]methyl}-1-{[(3R)-oxolan-3-yl]methyl}-1H-1,3-benzodiazole-6-carboxylic acid

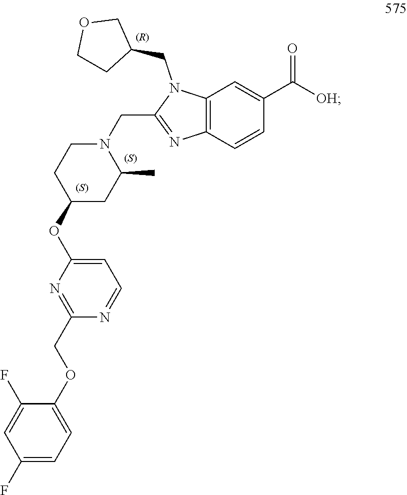

and

### Example 584. 2-{[(2S,4S)-4-({2-[(2,4-Difluorophenoxy)methyl]pyrimidin-4-yl}oxy)-2-methylpiperidin-1-yl]methyl}-1-{[(3S)-oxolan-3-yl]methyl}-1H-1,3-benzodiazole-6-carboxylic acid

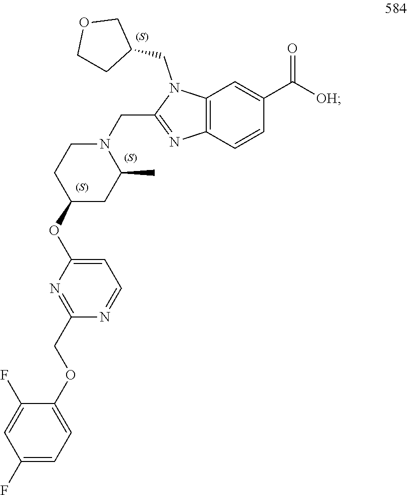

Examples 575 and 584 were prepared in a manner similar to Example 577.

### Example 580. 2-{[4-({2-[(4-Cyano-2-fluorophenoxy)methyl]pyrimidin-4-yl}oxy)piperidin-1-yl]methyl}-1-{[(2S)-oxetan-2-yl]methyl}-1H-imidazo[4,5-c]pyridine-6-carboxylic acid

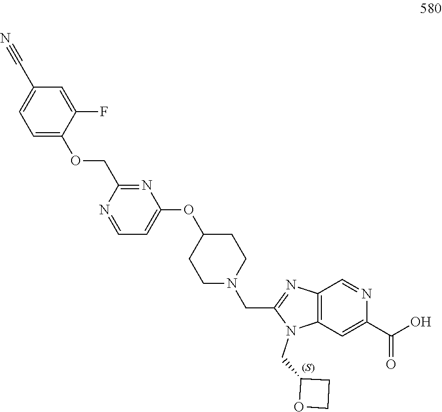

Example 580 was synthesized in a manner similar to Example 606.

### Examples 578. 2-{[(2S,4S)-4-({2-[(4-Cyano-2-fluorophenoxy)methyl]pyrimidin-4-yl}oxy)-2-methylpiperidin-1-yl]methyl}-1-{[(3S)-oxolan-3-yl]methyl}-1H-1,3-benzodiazole-6-carboxylic acid

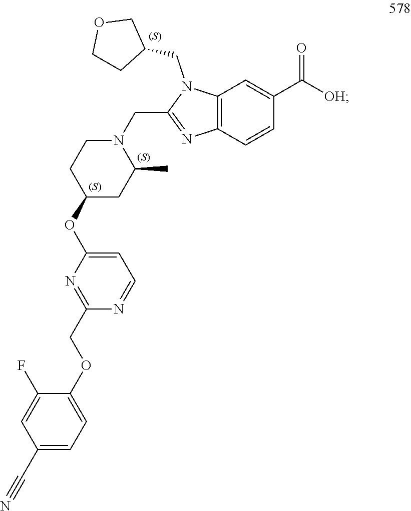

and

### Example 579. 2-{[(2S,4S)-4-({2-[(4-cyano-2-fluorophenoxy)methyl]pyrimidin-4-yl}oxy)-2-methylpiperidin-1-yl]methyl}-1-{[(2S)-oxolan-2-yl]methyl}-1H-1,3-benzodiazole-6-carboxylic acid

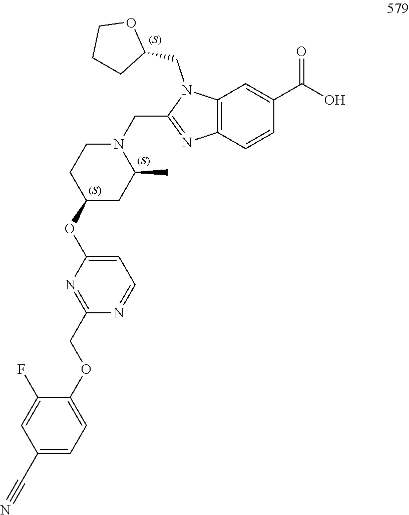

Example 578 and 579 were synthesized in similar procedures as described in Example 555, with NaI added at the second to last step.

### Example 556. 2-(((2S,4S)-4-((2-((2-Cyano-4-fluorophenoxy)methyl)pyrimidin-4-yl)oxy)-2-methylpiperidin-1-yl)methyl)-1-(((S)-oxetan-2-yl)methyl)-1H-imidazo[4,5-b]pyridine-6-carboxylic acid (556)

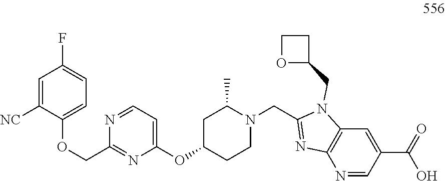 [SMILES: C[C@H]1C[C@@H](Oc2ccnc(COc3ccc(F)cc3C#N)n2)CCN1Cc1nc2ncc(C(=O)O)cc2n1C[C@@H]1CCO1]

### Step A. Methyl 2-(((2S,4S)-4-((2-((2-cyano-4-fluorophenoxy)methyl)pyrimidin-4-yl)oxy)-2-methylpiperidin-1-yl)methyl)-1-(((S)-oxetan-2-yl)methyl)-1H-imidazo[4,5-b]pyridine-6-carboxylate (556b)

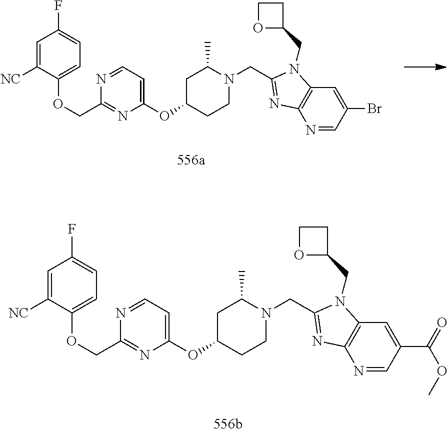 [SMILES: C[C@H]1C[C@@H](Oc2ccnc(COc3ccc(F)cc3C#N)n2)CCN1Cc1nc2ncc(Br)cc2n1C[C@@H]1CCO1 ; COC(=O)c1cnc2nc(CN3CC[C@H](Oc4ccnc(COc5ccc(F)cc5C#N)n4)C[C@@H]3C)n(C[C@@H]3CCO3)c2c1]

To a mixture of 556a (synthesized similarly as Example 599 (18 mg, 0.029 mmol) in DMSO/MeOH (1:1, 3 mL) was added Pd(dppf)Cl₂ (2 mg, 0.003 mmol) and triethylamine (9 mg, 0.087 mmol) at 20° C. The reaction was heated at 80° C. for 12H under CO. After completion, the reaction was quenched with H₂O (10 mL) and extracted with EtOAc (10 mL×3). The combined organic layer was washed with H₂O (10 mL), brine (10 mL), dried over Na₂SO₄, filtered and concentrated in vacuo to afford the title product (556b) (21 mg, 84% yield) as a brown oil. m/z (ESI, +ve ion)=602.2 [M+H]⁺.

### Step B. 2-(((2S,4S)-4-((2-((2-Cyano-4-fluorophenoxy)methyl)pyrimidin-4-yl)oxy)-2-methylpiperidin-1-yl)methyl)-1-(((S)-oxetan-2-yl)methyl)-1H-imidazo[4,5-b]pyridine-6-carboxylic acid (556)

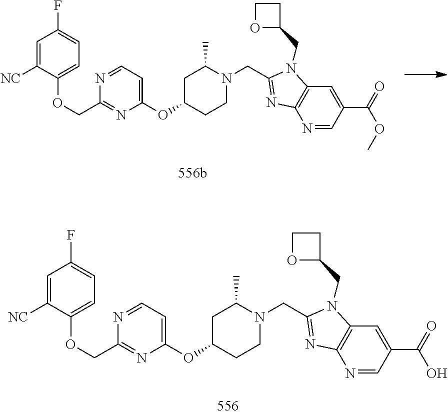 [SMILES: COC(=O)c1cnc2nc(CN3CC[C@H](Oc4ccnc(COc5ccc(F)cc5C#N)n4)C[C@@H]3C)n(C[C@@H]3CCO3)c2c1 ; C[C@H]1C[C@@H](Oc2ccnc(COc3ccc(F)cc3C#N)n2)CCN1Cc1nc2ncc(C(=O)O)cc2n1C[C@@H]1CCO1]

To a mixture of 556b (20 mg, 0.033 mmol) in THF/H₂O (1:1, 2 mL) was added lithium hydroxide (8 mg, 0.332 mmol) at 20° C. After stirring for 5 h, the mixture was quenched by 1 N HCl (5 mL), adjusted to pH=6 and extracted with EtOAc (10 mL×3). The combined organic layer was dried over anhydrous Na₂SO₄, filtered and concentrated to afford a crude residue which was purified by reverse phase HPLC (15% CH₃CN in H₂O with 0.1% NH₃H₂O as a modifier) to afford the title product (556) (7.7 mg, 39% yield). m/z (ESI, +ve ion)=588.2 [M+H]⁺. ¹H NMR (400 MHz, CD₃OD) δ ppm 9.06 (d, 1H), 8.49-8.58 (m, 1H), 8.41 (d, 1H), 7.52 (dd, 1H), 7.27-7.39 (m, 1H), 7.14 (dd, 1H), 6.70 (d, 1H), 5.38 (s, 2H), 5.23-5.33 (m, 1H), 4.90-5.03 (m, 2H), 4.71-4.81 (m, 1H), 4.52-4.66 (m, 2H), 4.34 (dt, 1H), 3.66 (d, 1H), 2.69-2.84 (m, 2H), 2.39-2.53 (m, 2H), 2.20-2.31 (m, 1H), 1.93-2.02 (m, 1H), 1.84-1.93 (m, 1H), 1.52-1.65 (m, 1H), 1.39-1.52 (m, 1H), 1.20 (d, 3H).

### Example 557. 2-{[(2S,4S)-4-({2-[(2-Chloro-4-fluorophenoxy)methyl]pyrimidin-4-yl}oxy)-2-(cyanomethyl)piperidin-1-yl]methyl}-1-{[(2S)-oxetan-2-yl]methyl}-1H-1,3-benzodiazole-6-carboxylic acid

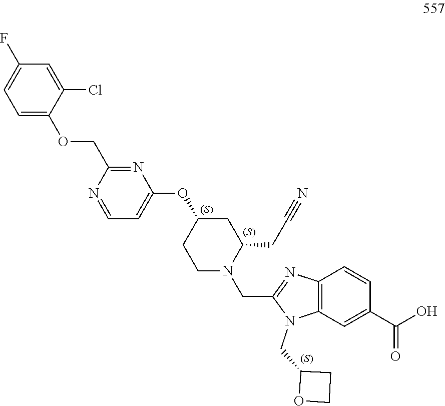

Example 557 was prepared in a manner similar to Example 555f, followed by fluoromethyl to cyanomethyl conversion with NaCN, DMF, 50° C. for 8 h, then Step B in Example 556.

### Example 558. 2-{[(2S,4S)-4-({2-[(2-Cyano-4-fluorophenoxy)methyl]pyrimidin-4-yl}oxy)-2-methylpiperidin-1-yl]methyl}-1-{[(3R)-oxolan-3-yl]methyl}-1H-1,3-benzodiazole-6-carboxylic acid

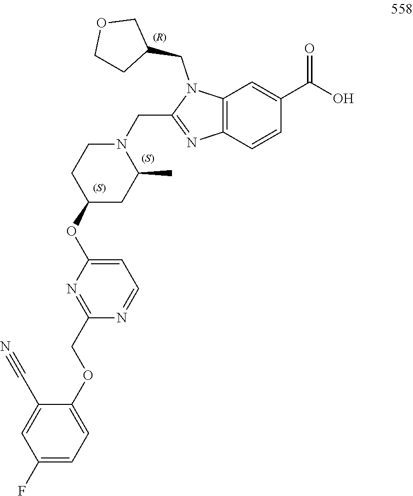

Example 558 was prepared in a manner similar to Example 599.

### Example 559. 2-(((2S,4S)-4-((2-((2,4-Difluorophenoxy)methyl)pyrimidin-4-yl)oxy)-2-methylpiperidin-1-yl)methyl)-N-hydroxy-1-(((S)-oxetan-2-yl)methyl)-1H-benzo[d]imidazole-6-carboxamide (559)

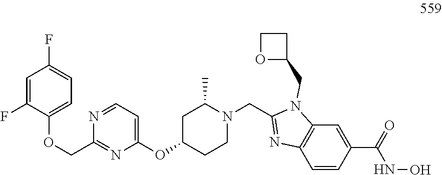 [SMILES: C[C@H]1C[C@@H](Oc2ccnc(COc3ccc(F)cc3F)n2)CCN1Cc1nc2ccc(C(=O)NO)cc2n1C[C@@H]1CCO1]

### Step A. 2-(((2S,4S)-4-((2-((2,4-Difluorophenoxy)methyl)pyrimidin-4-yl)oxy)-2-methylpiperidin-1-yl)methyl)-N-hydroxy-1-(((S)-oxetan-2-yl)methyl)-1H-benzo[d]imidazole-6-carboxamide (559)

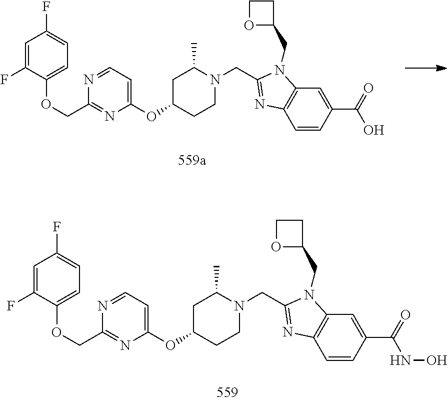 [SMILES: C[C@H]1C[C@@H](Oc2ccnc(COc3ccc(F)cc3F)n2)CCN1Cc1nc2ccc(C(=O)O)cc2n1C[C@@H]1CCO1 ; C[C@H]1C[C@@H](Oc2ccnc(COc3ccc(F)cc3F)n2)CCN1Cc1nc2ccc(C(=O)NO)cc2n1C[C@@H]1CCO1]

To a THF (3 mL) solution of 559a (prepared in a manner similar to Example 599) (30 mg, 0.052 mmol), was added CDI at 0° C. and stirred for 30 min at room temperature. After cooling to 0° C., hydroxyl amine hydrochloride salt (5.4 mg, 0.052 mmol) was added and the reaction was stirred at room temperature for 3H. 1 mL water was added, followed by addition of 15 mg DMAP and more hydroxyl amine hydrochloride. The reaction was stirred overnight, quenched with brine and extracted with ethyl acetate (3×). The combined organic layer was dried over anhydrous Na₂SO₄, filtered and concentrated under reduced pressure. The crude residue was purified by silica gel column chromatography with methanol in DCM to provide the title product (559) (7 mg, 22.7% yield). m/z (ESI, +ve ion)=595.2 [M+H]⁺. ¹H NMR (400 MHz, CDCl₃) δ ppm 8.41 (d, 1H), 8.01 (s, 1H), 7.69 (d, 1H), 7.54-7.56 (m, 1H), 6.85-6.96 (m, 2H), 6.71-6.75 (m, 1H), 6.56 (d, 1H), 5.20-5.25 (m, 3H), 5.01-5.08 (m, 1H), 4.91 (d, 1H), 4.50-4.61 (m, 3H), 4.28-4.31 (m, 1H), 3.58 (d, 1H), 2.77-2.80 (m, 1H), 2.67-2.69 (m, 1H), 2.38-2.45 (m, 2H), 2.25 (t, 1H), 2.02-2.05 (m, 1H), 1.89-1.92 (m, 1H), 1.55-1.59 (m, 1H), 1.42-1.51 (m, 1H), 1.20 (d, 3H).

### Example 560. 2-{[(2S,4S)-4-({2-[(2-Cyano-4-fluorophenoxy)methyl]pyrimidin-4-yl}oxy)-2-methylpiperidin-1-yl]methyl}-N-hydroxy-1-{[(2S)-oxetan-2-yl]methyl}-1H-1,3-benzodiazole-6-carboxamide

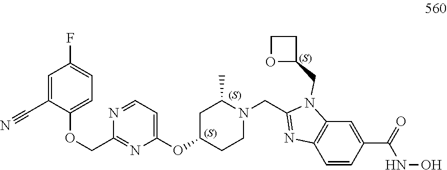

Example 560 was synthesized in similar procedures as Example 559, with DIPEA as the base.

### Example 561 2-{[(2S,4S)-4-({2-[(2-Cyano-4-fluorophenoxy)methyl]pyrimidin-4-yl}oxy)-2-methylpiperidin-1-yl]methyl}-1-{[(2R)-oxetan-2-yl]methyl}-1H-1,3-benzodiazole-6-carboxylic acid

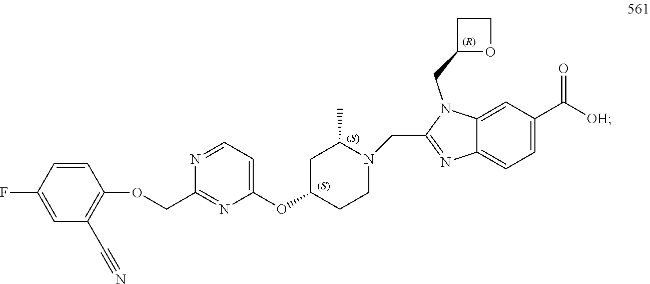

and

### Example 562. 2-{[(2S,4S)-4-({2-[(2,4-Difluorophenoxy)methyl]pyrimidin-4-yl}oxy)-2-methylpiperidin-1-yl]methyl}-1-{[(2R)-oxetan-2-yl]methyl}-1H-1,3-benzodiazole-6-carboxylic acid

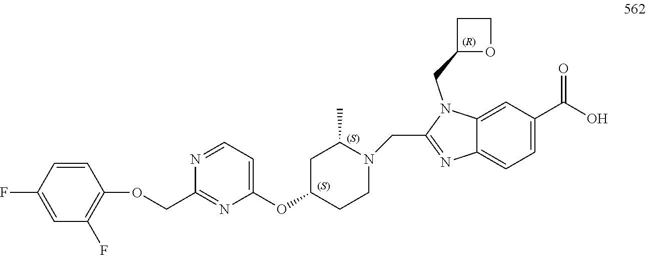

Examples 561 and 562 were prepared in a manner similar to Example 599.

### Example 563. 2-(((2S,4S)-4-((2-((2,4-Difluorophenoxy)methyl)pyrimidin-4-yl)oxy)-2-methylpiperidin-1-yl)methyl)-N-(methylsulfonyl)-1-(((S)-oxetan-2-yl)methyl)-1H-benzo[d]imidazole-6-carboxamide (563)

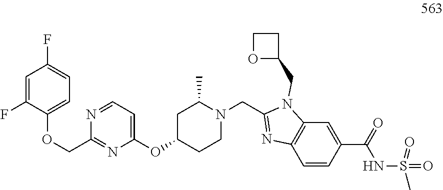 [SMILES: C[C@H]1C[C@@H](Oc2ccnc(COc3ccc(F)cc3F)n2)CCN1Cc1nc2ccc(C(=O)NS(C)(=O)=O)cc2n1C[C@@H]1CCO1]

### Step A. 2-(((2S,4S)-4-((2-((2,4-Difluorophenoxy)methyl)pyrimidin-4-yl)oxy)-2-methylpiperidin-1-yl)methyl)-N-(methylsulfonyl)-1-(((S)-oxetan-2-yl)methyl)-1H-benzo[d]imidazole-6-carboxamide (563)

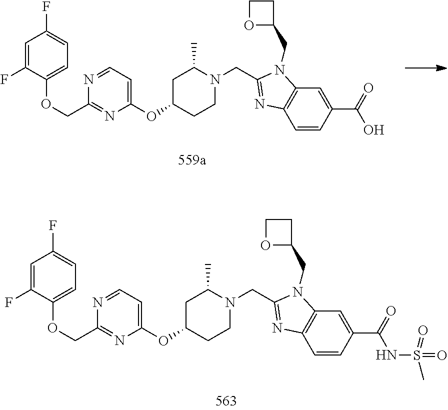 [SMILES: C[C@H]1C[C@@H](Oc2ccnc(COc3ccc(F)cc3F)n2)CCN1Cc1nc2ccc(C(=O)O)cc2n1C[C@@H]1CCO1 ; C[C@H]1C[C@@H](Oc2ccnc(COc3ccc(F)cc3F)n2)CCN1Cc1nc2ccc(C(=O)NS(C)(=O)=O)cc2n1C[C@@H]1CCO1]

A solution of Compound 559a (prepared as described in Example 599) (30 mg, 0.052 mmol) was dissolved in anhydrous DCM. CH₃SO₂NH₂ (14.8 mg, 0.16 mmol), DMAP (19.1 mg, 0.16 mmol) and DCC (32 mg, 0.16 mmol) were added to the reaction mixture. After stirring at room temperature overnight, the reaction was quenched with brine and extracted with DCM (3×). The combined organic layer was dried over anhydrous Na₂SO₄, filtered and concentrated under reduced pressure. The crude residue was purified by silica gel column chromatography with methanol in DCM to provide the title compound (563) (23 mg, 68% yield). m/z (ESI, +ve ion)=657.2 [M+H]⁺. ¹H NMR (400 MHz, CDCl₃) δ ppm 8.41 (d, 1H), 8.06 (s, 1H), 7.74 (s, 2H), 6.85-6.96 (m, 2H), 6.71-6.76 (m, 1H), 6.56 (d, 1H), 5.22-5.25 (m, 3H), 5.03-5.08 (m, 1H), 4.89-4.93 (m, 1H), 4.48-4.65 (m, 3H), 4.30-4.35 (m, 1H), 3.62 (d, 1H), 3.43 (s, 3H), 2.82-2.83 (m, 1H), 2.67-2.74 (m, 1H), 2.42-2.64 (m, 2H), 2.28-2.34 (m, 1H), 2.04-2.07 (m, 1H), 1.93-1.95 (m, 1H), 1.57-1.61 (m, 1H), 1.43-1.52 (m, 1H), 1.20 (d, 3H).

### Example 564. N-(Cyclopropanesulfonyl)-2-{[(2S,4S)-4-({2-[(2,4-difluorophenoxy)methyl]pyrimidin-4-yl}oxy)-2-methylpiperidin-1-yl]methyl}-1-{[(2S)-oxetan-2-yl]methyl}-1H-1,3-benzodiazole-6-carboxamide

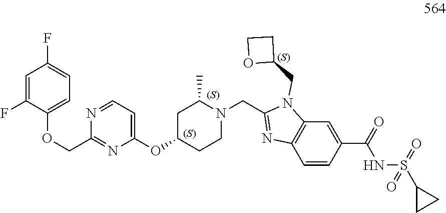

Example 564 was synthesized in similar procedures as Example 563.

### Example 565. 2-(1-(4-((2-((2,4-Difluorophenoxy)methyl)pyrimidin-4-yl)oxy)piperidin-1-yl)ethyl)-1-(((S)-oxetan-2-yl)methyl)-1H-benzo[d]imidazole-6-carboxylic acid (565)

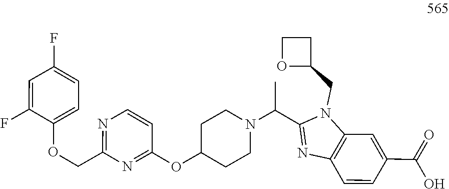 [SMILES: CC(c1nc2ccc(C(=O)O)cc2n1C[C@@H]1CCO1)N1CCC(Oc2ccnc(COc3ccc(F)cc3F)n2)CC1]

### Step A. tert-Butyl 2-(4-((2-((2,4-difluorophenoxy)methyl)pyrimidin-4-yl)oxy)piperidin-1-yl) propanoate (565b)

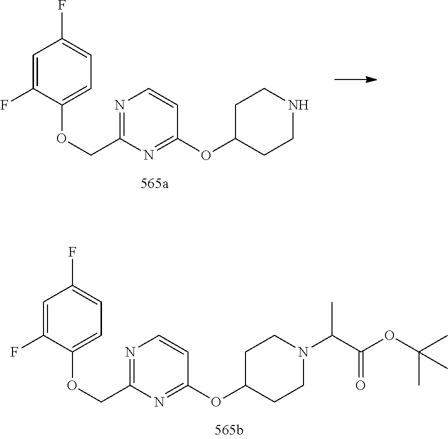 [SMILES: Fc1ccc(OCc2nccc(OC3CCNCC3)n2)c(F)c1 ; CC(C(=O)OC(C)(C)C)N1CCC(Oc2ccnc(COc3ccc(F)cc3F)n2)CC1]

Example 565a (22 mg, 0.069 mmol) and tert-butyl 2-bromopropanate (21.5 mg, 0.069 mmol) were dissolved in anhydrous DMF. DIPEA (0.036 mL, 0.069 mmol) was added to the reaction mixture. The reaction was heated at 60° C. for 2H, quenched with brine and extracted with EtOAc (3×). The combined organic layer was dried over anhydrous Na₂SO₄, filtered and concentrated under reduced pressure. The crude reaction mixture was purified by silica gel column chromatography with EtOAc in hexanes to provide the title product (565b) (20 mg, 65% yield). m/z (ESI, +ve ion)=450.3 [M+H]⁺.

### Step B. 2-(4-((2-((2,4-Difluorophenoxy)methyl)pyrimidin-4-yl)oxy)piperidin-1-yl) propanoic acid (565c)

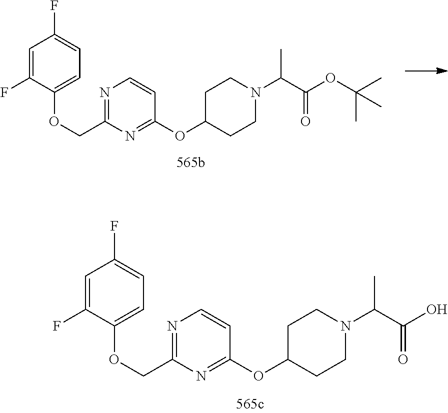 [SMILES: CC(C(=O)OC(C)(C)C)N1CCC(Oc2ccnc(COc3ccc(F)cc3F)n2)CC1 ; CC(C(=O)O)N1CCC(Oc2ccnc(COc3ccc(F)cc3F)n2)CC1]

The title compound 565c was synthesized as Step E in Example 555. m/z (ESI, +ve ion)=394.4 [M+H]⁺.

### Step C. Methyl 4-(2-(4-((2-((2,4-difluorophenoxy)methyl)pyrimidin-4-yl)oxy)piperidin-1-yl) propanamido)-3-((((S)-oxetan-2-yl)methyl)amino)benzoate (565d)

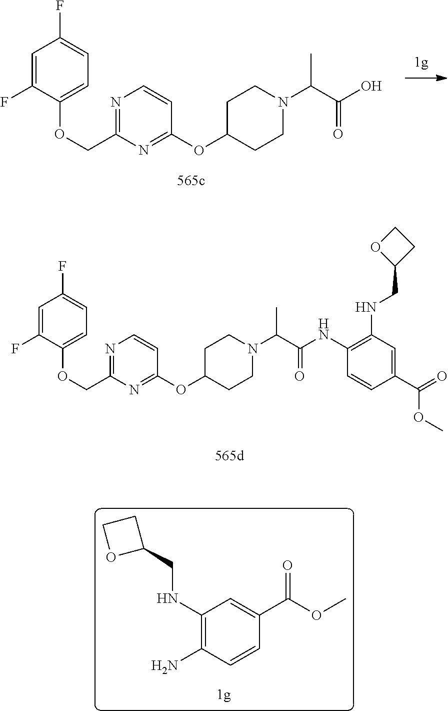 [SMILES: CC(C(=O)O)N1CCC(Oc2ccnc(COc3ccc(F)cc3F)n2)CC1 ; COC(=O)c1ccc(NC(=O)C(C)N2CCC(Oc3ccnc(COc4ccc(F)cc4F)n3)CC2)c(NC[C@@H]2CCO2)c1 ; COC(=O)c1ccc(N)c(NC[C@@H]2CCO2)c1]

565c (19 mg, 0.048 mmol) was dissolved in anhydrous DMF and added DIPEA (0.025 mL, 0.15 mmol). After 5 min, HATU (36.7 mg, 0.097 mmol) was added, followed by dropwise addition of a DMF solution of 1g (12.6 mg, 0.053 mmol). After stirring at room temperature overnight, the reaction was quenched with water and extracted with EtOAc (5 mL×3). The combined organic layer was dried over anhydrous Na₂SO₄, filtered, concentrated and purified by silica gel column chromatography to afford the title product (565d) (14 mg, 47% yield). m/z (ESI, +ve ion)=612.3 [M+H]⁺.

### Step D. Methyl 2-(1-(4-((2-((2,4-difluorophenoxy)methyl)pyrimidin-4-yl)oxy)piperidin-1-yl)ethyl)-1-(((S)-oxetan-2-yl)methyl)-1H-benzo[d]imidazole-6-carboxylate (565f)

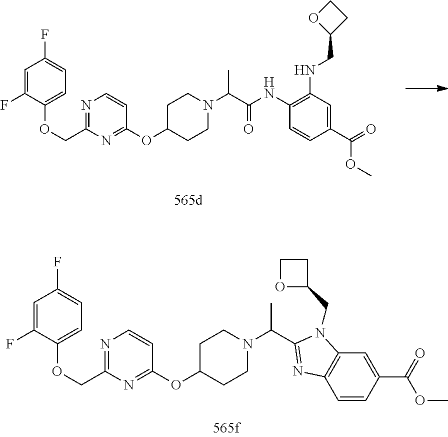 [SMILES: COC(=O)c1ccc(NC(=O)C(C)N2CCC(Oc3ccnc(COc4ccc(F)cc4F)n3)CC2)c(NC[C@@H]2CCO2)c1 ; COC(=O)c1ccc2nc(C(C)N3CCC(Oc4ccnc(COc5ccc(F)cc5F)n4)CC3)n(C[C@@H]3CCO3)c2c1]

565d (14 mg, 0.023 mmol) was dissolved in acetic acid (1 mL) and heated at 60° C. overnight. The acetic acid in the reaction was removed under reduced pressure. Then the reaction was quenched with sat NaHCO₃ and extracted with EtOAc (5 mL×3). The combined organic layer was dried over anhydrous Na₂SO₄, filtered and concentrated to provide the title product (565f), which was used in the next step without further purification. m/z (ESI, +ve ion)=594.3 [M+H]⁺.

### Step E. 2-(1-(4-((2-((2,4-Difluorophenoxy)methyl)pyrimidin-4-yl)oxy)piperidin-1-yl)ethyl)-1-(((S)-oxetan-2-yl)methyl)-1H-benzo[d]imidazole-6-carboxylic acid (565)

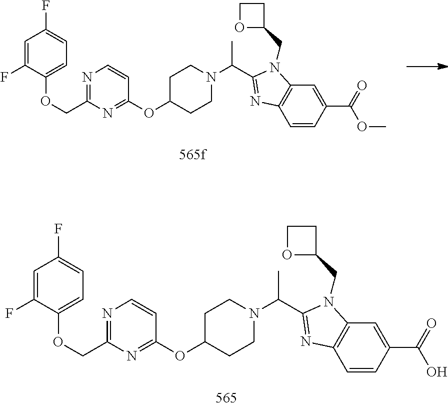 [SMILES: COC(=O)c1ccc2nc(C(C)N3CCC(Oc4ccnc(COc5ccc(F)cc5F)n4)CC3)n(C[C@@H]3CCO3)c2c1 ; CC(c1nc2ccc(C(=O)O)cc2n1C[C@@H]1CCO1)N1CCC(Oc2ccnc(COc3ccc(F)cc3F)n2)CC1]

The title compound (565) was synthesized in similar procedures as described in Example 554, Step D, with THE/MeOH/H₂O (6:1:1) as solvent. m/z (ESI, +ve ion)=580.3 [M+H]⁺.

### Example 568. 2-{1-[(2S,4S)-4-({2-[(2,4-difluorophenoxy)methyl]pyrimidin-4-yl}oxy)-2-methylpiperidin-1-yl]ethyl}-1-{[(2S)-oxetan-2-yl]methyl}-1H-1,3-benzodiazole-6-carboxylic acid

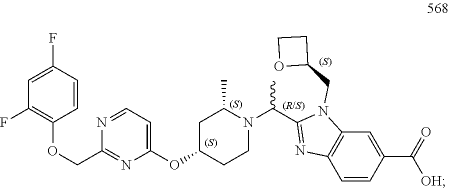

and

### Example 569. 2-{1-[(2S,4S)-4-({2-[(2,4-difluorophenoxy)methyl]pyrimidin-4-yl}oxy)-2-methylpiperidin-1-yl]ethyl}-1-[(2S)-2,4-dihydroxybutyl]-1H-1,3-benzodiazole-6-carboxylic acid

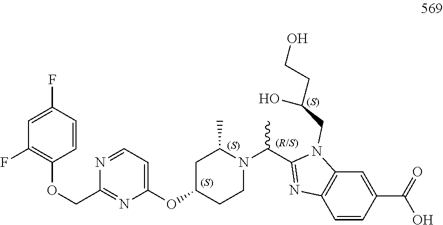

Example 568 was synthesized in similar procedures as described in Example 565. Example 569 was the side product isolated from the last step of making 568.

### Example 570. 2-{[(2S,4S)-4-({2-[(2,4-Difluorophenoxy)methyl]pyrimidin-4-yl}oxy)-2-methylpiperidin-1-yl]methyl}-1-({2-oxabicyclo[2.1.1]hexan-1-yl}methyl)-1H-1,3-benzodiazole-6-carboxylic acid

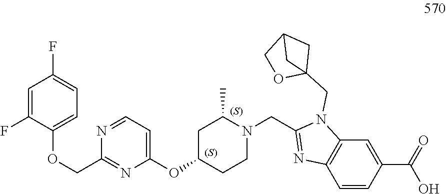

Example 570 was prepared in a manner similar to Example 577.

### Example 571. 2-(6-((2-((4-Cyano-2-fluorophenoxy)methyl)pyrimidin-4-yl)oxy)spiro[2.5]octan-1-yl)-1-(((S)-oxetan-2-yl)methyl)-1H-benzo[d]imidazole-6-carboxylic acid (571)

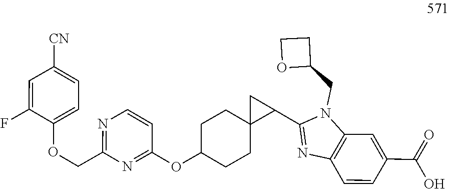 [SMILES: N#Cc1ccc(OCc2nccc(OC3CCC4(CC3)CC4c3nc4ccc(C(=O)O)cc4n3C[C@@H]3CCO3)n2)c(F)c1]

### Step A. Methyl 2-(4-((tert-butyldiphenylsilyl)oxy)cyclohexylidene)acetate (571a)

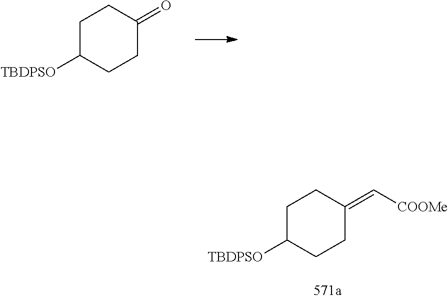

t-BuOK (2.27 g, 20.2 mmol) was added to a solution of methyl 2-(diethoxyphosphoryl)acetate (4.26 g, 20.2 mmol) in THF (30 mL) at 0° C. After stirring at 0° C. for 1H, the reaction was cooled to −50° C. A solution of 4-[(tert-butyldiphenylsilyl)oxy]cyclohexan-1-one (5.5 g, 15.6 mmol) in THF (30 mL) was added dropwise at −50° C., and the reaction mixture was allowed to slowly warm to 20° C., and stirred for 16 h. After completion, the reaction was quenched with H₂O (50 mL) and extracted with EtOAc (50 mL×3). The combined organic layer was washed with H₂O (50 mL), brine (50 mL), dried over anhydrous Na₂SO₄, filtered and concentrated in vacuo to give a crude residue, which was purified by silica gel column chromatography (petroleum ether:EtOAc=20:1) to afford the title product (571a) (4.1 g, 60.9% yield) as a yellow oil. m/z (ESI, +ve ion)=431.1 [M+Na]⁺.

### Step B. Methyl 6-((tert-butyldiphenylsilyl)oxy)spiro[2.5]octane-1-carboxylate (571b)

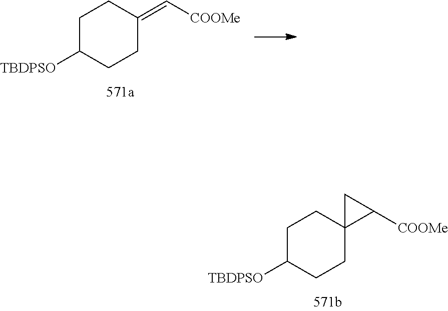

A solution of t-BuOK (0.48 g, 4.3 mmol) was added to a solution of trimethylsulfoxonium iodide (0.95 g, 4.3 mmol) in DMSO (5 mL). The mixture was stirred at 20° C. for 1H. A solution of 571a (1.0 g, 2.4 mmol) in DMSO (5 mL) was added at 20° C., and the reaction was stirred for 23H. After completion, the reaction was diluted with H₂O (30 mL) and extracted with EtOAc (30 mL×3). The combined organic layer was washed with H₂O (30 mL), brine (30 mL), dried over anhydrous Na₂SO₄, filtered and concentrated in vacuo to provide a crude residue, which was purified by reverse phase HPLC (CH₃CN in H₂O, with 0.1% TFA as a modifier) to afford the title product (571b) (200 mg, 16.7% yield) as a yellow oil. m/z (ESI, +ve ion)=445.1 [M+Na]⁺.

### Step C. Methyl 6-hydroxyspiro[2.5]octane-1-carboxylate (571c)

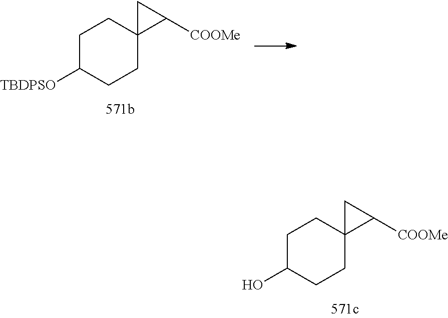

A solution of 571b (1.2 g, 2.8 mmol) and NH₄F (1.04 g, 28.0 mmol) in MeOH (10 mL) was heated at 70° C. for 16 h. After completion, the reaction was quenched with H₂O (5 mL) and extracted with EtOAc (10 mL×3). The organic layer was washed with H₂O (10 mL), brine (10 mL), dried over anhydrous Na₂SO₄, filtered and concentrated in vacuo to give a crude residue, which was purified by reverse phase HPLC (CH₃CN in H₂O, with 0.1% TFA as a modifier) to afford the title product (571c) (52.3 mg) with some impurity. m/z (ESI, +ve ion)=185.1 [M+H]⁺.

### Step D. 2-(6-((2-((4-Cyano-2-fluorophenoxy)methyl)pyrimidin-4-yl)oxy)spiro[2.5]octan-1-yl)-1-(((S)-oxetan-2-yl)methyl)-1H-benzo[d]imidazole-6-carboxylic acid (571)

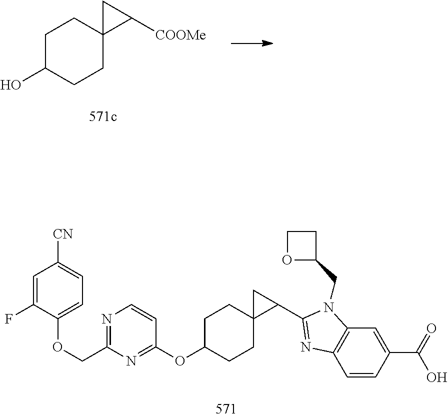 [SMILES: COC(=O)C1CC12CCC(O)CC2 ; N#Cc1ccc(OCc2nccc(OC3CCC4(CC3)CC4c3nc4ccc(C(=O)O)cc4n3C[C@@H]3CCO3)n2)c(F)c1]

The title compound 571 was prepared as a pair of diastereomers from 571c as described in Example 565, m/z (ESI, +ve ion)=584.3 [M+H]⁺.

### Example 590. 1-(Cyclobutylmethyl)-2-{[(2S,4S)-4-({2-[(2,4-difluorophenoxy)methyl]pyrimidin-4-yl}oxy)-2-methylpiperidin-1-yl]methyl}-1H-1,3-benzodiazole-6-carboxylic acid

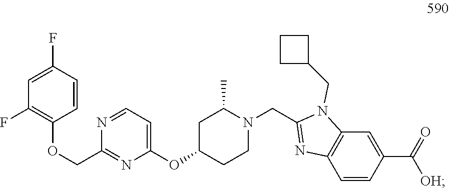 [SMILES: C[C@H]1C[C@@H](Oc2ccnc(COc3ccc(F)cc3F)n2)CCN1Cc1nc2ccc(C(=O)O)cc2n1CC1CCC1]

and

### Example 591. 1-[(2S)-2-(Difluoromethoxy)propyl]-2-{[(2S,4S)-4-({2-[(2,4-difluorophenoxy)methyl]pyrimidin-4-yl}oxy)-2-methylpiperidin-1-yl]methyl}-1H-1,3-benzodiazole-6-carboxylic acid

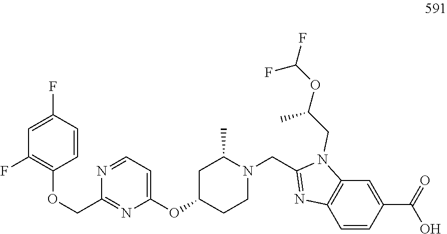 [SMILES: C[C@@H](Cn1c(CN2CC[C@H](Oc3ccnc(COc4ccc(F)cc4F)n3)C[C@@H]2C)nc2ccc(C(=O)O)cc21)OC(F)F]

Examples 590 and 591 were synthesized from methyl 2-(4-hydroxycyclohexyl)acetate in similar procedures as Example 571 and were separated by reverse phase HPLC at the last step.

### Examples 572. 2-{[(2S,4S)-4-({2-[(2,4-Difluorophenoxy)methyl]pyrimidin-4-yl}oxy)-2-methylpiperidin-1-yl]methyl}-5-fluoro-1-{[(2S)-oxetan-2-yl]methyl}-1H-1,3-benzodiazole-6-carboxylic acid

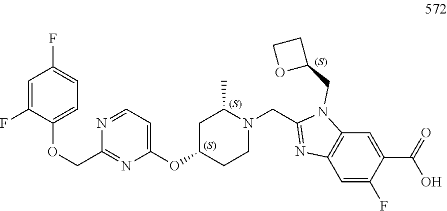

### Example 576. 2-{[(2S,4S)-4-({2-[(2,4-Difluorophenoxy)methyl]pyrimidin-4-yl}oxy)-2-methylpiperidin-1-yl]methyl}-4-fluoro-1-{[(2S)-oxetan-2-yl]methyl}-1H-1,3-benzodiazole-6-carboxylic acid

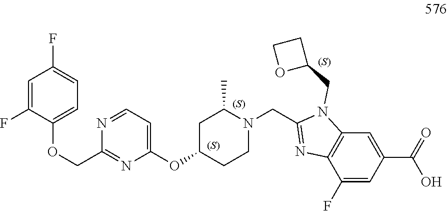

### Example 582. 2-{[(2S,4S)-4-({2-[(2,4-Difluorophenoxy)methyl]pyrimidin-4-yl}oxy)-2-methylpiperidin-1-yl]methyl}-1-[(3-fluorooxetan-3-yl)methyl]-1H-1,3-benzodiazole-6-carboxylic acid

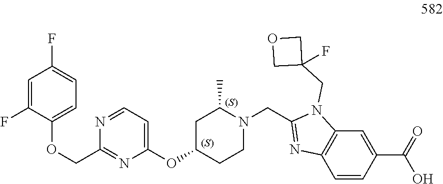

### Example 583. 1-{[3-(difluoromethyl)oxetan-3-yl]methyl}-2-{[(2S,4S)-4-({2-[(2,4-difluorophenoxy)methyl]pyrimidin-4-yl}oxy)-2-methylpiperidin-1-yl]methyl}-1H-1,3-benzodiazole-6-carboxylic acid

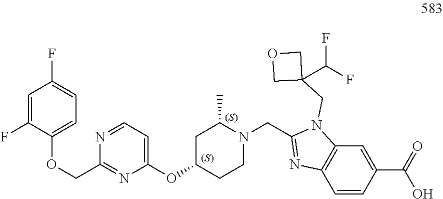

### Example 585. 2-{[(2S,4S)-4-({2-[(2,4-Difluorophenoxy)methyl]pyrimidin-4-yl}oxy)-2-methylpiperidin-1-yl]methyl}-5-methyl-1-{[(2S)-oxetan-2-yl]methyl}-1H-1,3-benzodiazole-6-carboxylic acid

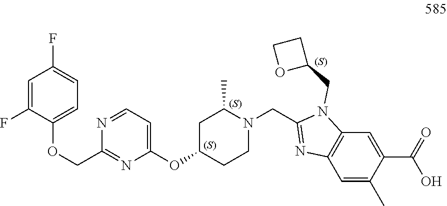

Examples 572-576, 582-583, and 585 were synthesized from 2-((2,4-difluorophenoxy)methyl)-4-(((2S,4S)-2-methylpiperidin-4-yl)oxy)pyrimidine as described for Example 606f, Step F, and corresponding imidazole core was prepared by following synthetic procedure of 577d.

### Example 581 2-{[(2S,4S)-4-({2-[(2,4-Difluorophenoxy)methyl]-5-fluoropyrimidin-4-yl}oxy)-2-methylpiperidin-1-yl]methyl}-1-{[(2S)-oxetan-2-yl]methyl}-1H-1,3-benzodiazole-6-carboxylic acid

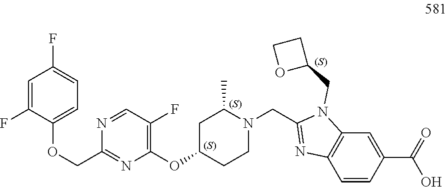

### Example 586. 2-{[(2R,4R)-4-({2-[(4-Cyano-2-fluorophenoxy)methyl]pyrimidin-4-yl}oxy)-2-ethylpiperidin-1-yl]methyl}-1-{[(2S)-oxetan-2-yl]methyl}-1H-1,3-benzodiazole-6-carboxylic acid

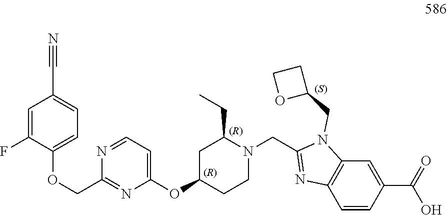

### 2-{[(2S,4S)-4-({2-[(4-Cyano-2-fluorophenoxy)methyl]pyrimidin-4-yl}oxy)-2-ethylpiperidin-1-yl]methyl}-1-{[(2S)-oxetan-2-yl]methyl}-1H-1,3-benzodiazole-6-carboxylic acid

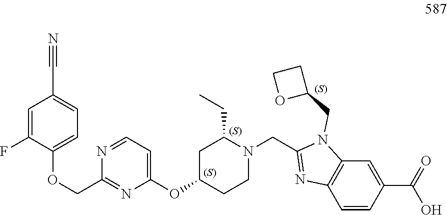

Example 581, Example 586, and Example 587 were synthesized in similar procedures as described in Example 599. Examples 586 and 587 were chirally separated at the second to last step through SFC chiral separation.

### Example 577. 2-(((2S,4S)-4-((2-((2,4-Difluorophenoxy)methyl)pyrimidin-4-yl)oxy)-2-methylpiperidin-1-yl)methyl)-1-((3R,4R)-4-methoxytetrahydrofuran-3-yl)-1H-benzo[d]imidazole-6-carboxylic acid (577)

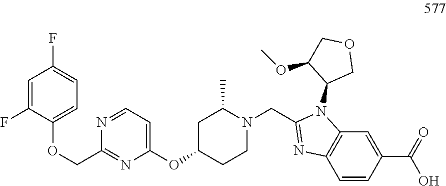 [SMILES: CO[C@H]1COC[C@H]1n1c(CN2CC[C@H](Oc3ccnc(COc4ccc(F)cc4F)n3)C[C@@H]2C)nc2ccc(C(=O)O)cc21]

### Step A. Methyl 3-(((3R,4R)-4-methoxytetrahydrofuran-3-yl)amino)-4-nitrobenzoate (577b)

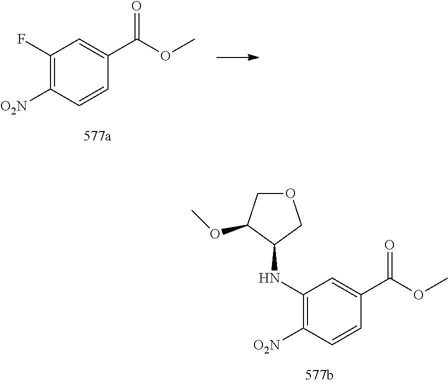

To a solution of 577a (300 mg, 1.51 mmol) in THF (5.0 mL) was added [cis-4-methoxytetrahydro-3-furanyl]amine hydrochloride (231.0 mg, 1.51 mmol) and triethylamine (1.05 mL, 7.53 mmol) at room temperature. Reaction mixture was allowed to stir at 60° C. for 12H. Upon completion, it was cooled down to room temperature and diluted with EtOAc (20 mL) and water (10 mL). Organic layer was separated. The aqueous layer was extracted with EtOAc (20 mL×3). The combined organic layer was washed with brine (5 mL), dried over Na₂SO₄, filtered and concentrated and the resulting crude residue was purified by silica gel column chromatography (gradient elution, 0-60% EtOAc/hexanes) to afford the product 577b (363.0 mg, 81% yield) as a yellow oil. m/z (ESI, +ve ion)=297.3 [M+H]⁺.

### Step B. Methyl 4-amino-3-(((3R,4R)-4-methoxytetrahydrofuran-3-yl)amino)benzoate (577c)

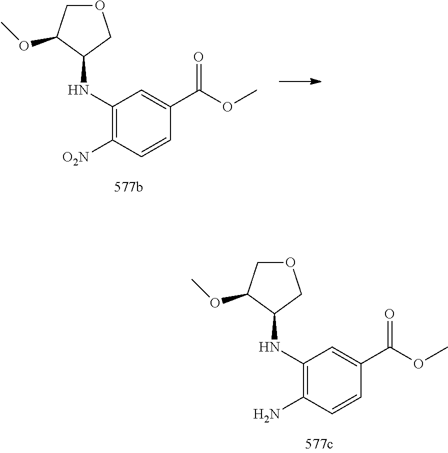

To a solution of 577b (363.0 mg, 1.23 mmol) in methanol (12.3 mL) was added 10% Pd/C (261.0 mg). Reaction mixture was stirred at room temperature under a balloon of hydrogen for 12H. After completion, Pd/C was removed by filtration. The filtrate was concentrated to afford the crude product (577c) (310.0 mg) without further purification. m/z (ESI, +ve ion)=267.3 [M+H]⁺.

### Step C. Methyl 2-(chloromethyl)-1-((3R,4R)-4-methoxytetrahydrofuran-3-yl)-1H-benzo[d]imidazole-6-carboxylate (577d)

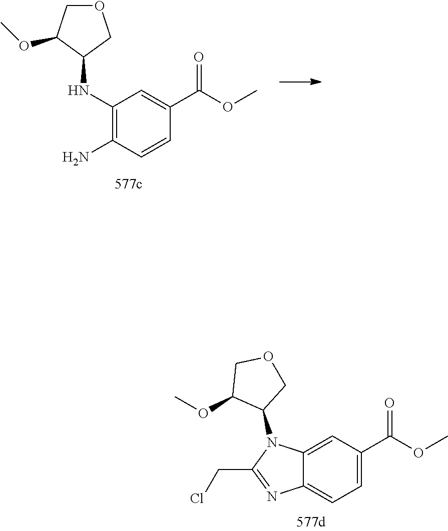

A suspension of 577c (310.0 mg, 1.16 mmol), 2-chloro-1,1,1-trimethoxyethane (172 μL, 1.28 mmol) and TsOH (22.1 mg, 0.12 mmol) in acetonitrile (3.9 mL) was stirred at 60° C. for 2H. After completion, the reaction solvent was removed under reduced pressure and the resulting residue was purified by silica gel column chromatography (gradient elution, 40-80% EtOAc/hexanes) to afford product 577d (300.0 mg, 79% yield). m/z (ESI, +ve ion)=325.3 [M+H]⁺.

### Step D. Methyl 2-(((2S,4S)-4-((2-((2,4-difluorophenoxy)methyl)pyrimidin-4-yl)oxy)-2-methylpiperidin-1-yl)methyl)-1-((3R,4R)-4-methoxytetrahydrofuran-3-yl)-1H-benzo[d]imidazole-6-carboxylate (577e)

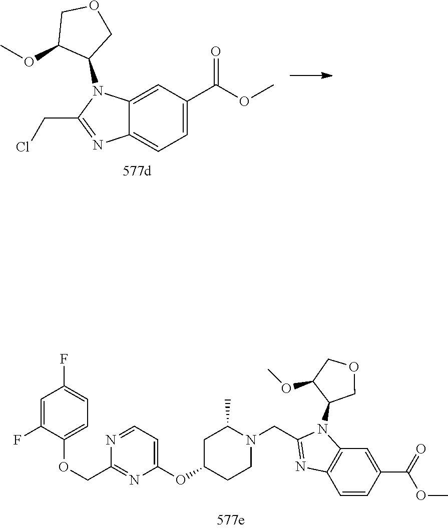

A solution of 577d (24.5 mg, 0.075 mmol), 2-[(2,4-difluorophenoxy)methyl]-4-{[(2S,4S)-2-methylpiperidin-4-yl]oxy}pyrimidine (25.3 mg, 0.075 mmol) and DIPEA (39.4 μL, 0.23 mmol) in DMF (0.3 mL) was stirred at 60° C. for 2H. After completion, the reaction mixture was diluted with EtOAc (5 mL). The organic layer was washed with water (3 mL×2), brine (3 mL), dried over Na₂SO₄, filtered and concentrated and the resulting crude residue was purified by silica gel column chromatography (gradient elution, 0-10% MeOH/DCM) to afford the title product (577e) (41.2 mg, 88% yield) as a gummy oil. m/z (ESI, +ve ion)=624.4 [M+H]⁺.

### Step E. 2-(((2S,4S)-4-((2-((2,4-Difluorophenoxy)methyl)pyrimidin-4-yl)oxy)-2-methylpiperidin-1-yl)methyl)-1-((3R,4R)-4-methoxytetrahydrofuran-3-yl)-1H-benzo[d]imidazole-6-carboxylic acid (577)

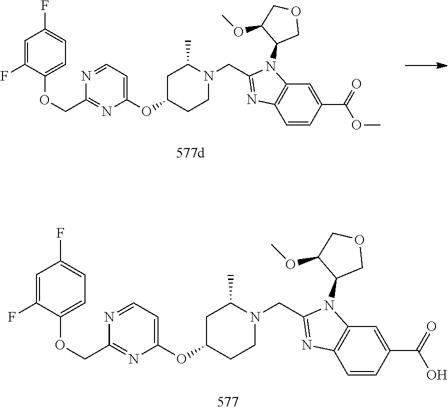 [SMILES: COC(=O)c1ccc2nc(CN3CC[C@H](Oc4ccnc(COc5ccc(F)cc5F)n4)C[C@@H]3C)n([C@@H]3COC[C@@H]3OC)c2c1 ; CO[C@H]1COC[C@H]1n1c(CN2CC[C@H](Oc3ccnc(COc4ccc(F)cc4F)n3)C[C@@H]2C)nc2ccc(C(=O)O)cc21]

A solution of 577d (41.2 mg, 0.066 mmol) and LiOH (7.9 mg, 0.33 mmol) in THF: ⁱPrOH:H₂O=1:1:1 (0.6 mL) was stirred at 30° C. for 2H. After completion, the reaction solvent was removed under reduced pressure and the resulting residue was diluted with water to 1 mL and purified by reverse phase HPLC (gradient elution, 20-60% CH₃CN in H₂O, with 20 mM NH₄HCO₃ as a modifier) to afford the title product (577) (29.4 mg, 73% yield). ¹H NMR (400 MHz, DMSO-d₆) δ ppm 12.71 (s, 1H), 8.49 (d, 1H), 8.36 (d, 1H), 7.76 (d, 1H), 7.60 (d, 1H), 7.24-7.34 (m, 1H), 7.14 (td, 1H), 6.94 (brt, 1H), 6.80 (d, 1H), 5.42-5.77 (m, 1H), 5.30 (s, 2H), 4.82-4.93 (m, 1H), 4.52-4.68 (m, 1H), 4.05-4.38 (m, 4H), 3.94-4.03 (m, 1H), 3.76-3.91 (m, 1H), 2.88-2.97 (m, 3H), 2.55-2.78 (m, 1H), 2.26-2.47 (m, 1H), 2.00-2.11 (m, 1H), 1.79-1.99 (m, 1H), 1.26-1.54 (m, 2H), 1.07-1.24 (m, 3H); m/z (ESI, +ve ion)=610.3 [M+H]⁺.

### Example 588. 2-{[(2S,4S)-4-({2-[(2,4-Difluorophenoxy)methyl]pyrimidin-4-yl}oxy)-2-methylpiperidin-1-yl]methyl}-1-[2-(2,2,2-trifluoroethoxy)ethyl]-1H-1,3-benzodiazole-6-carboxylic acid (588)

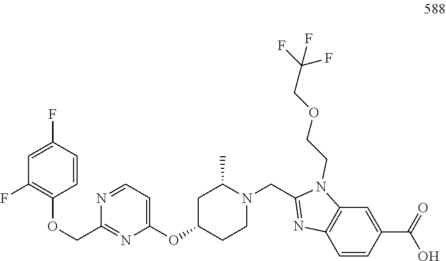 [SMILES: C[C@H]1C[C@@H](Oc2ccnc(COc3ccc(F)cc3F)n2)CCN1Cc1nc2ccc(C(=O)O)cc2n1CCOCC(F)(F)F]

### Step A. Methyl 4-nitro-3-((2-(2,2,2-trifluoroethoxy)ethyl)amino)benzoate (588c)

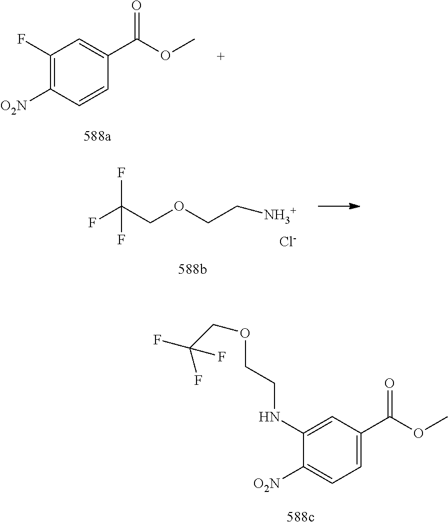 [SMILES: COC(=O)c1ccc([N+](=O)[O-])c(F)c1 ; [NH3+]CCOCC(F)(F)F ; [Cl-] ; COC(=O)c1ccc([N+](=O)[O-])c(NCCOCC(F)(F)F)c1]

To a stirred solution of compound 588a (100 mg, 0.502 mmol) in anhydrous DMF, compound 588b (108 mg, 0.603 mmol) followed by Et₃N (0.486 ml, 3.52 mmol) was added under argon atmosphere. The reaction mixture was stirred at room temperature for overnight. The reaction mixture was quenched by addition of water (2 ml), the aqueous layer was extracted by ethyl acetate (3×5 ml). The combined organic layer was dried over anhydrous Na₂SO₄, filtered and concentrated under reduced pressure. The crude solid was washed with 20% ethyl acetate in hexane three times. The yellow solid was air dried and carried forward for the next step without further purification (162 mg, 99% yield); m/z (ESI, +ve ion)=323.1 [M+H]⁺.

### Step B. Methyl 2-(chloromethyl)-1-(2-(2,2,2-trifluoroethoxy)ethyl)-1H-benzo[d]imidazole-6-carboxylate (588e)

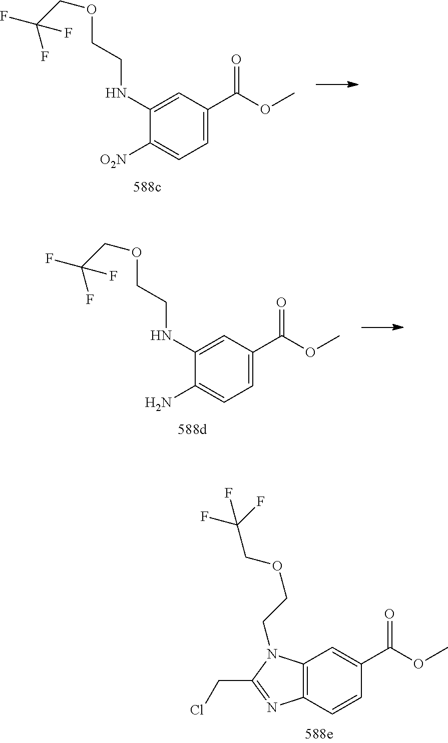 [SMILES: COC(=O)c1ccc([N+](=O)[O-])c(NCCOCC(F)(F)F)c1 ; COC(=O)c1ccc(N)c(NCCOCC(F)(F)F)c1 ; COC(=O)c1ccc2nc(CCl)n(CCOCC(F)(F)F)c2c1]

Compound 588c (160 mg, 0.497) was dissolved in a mixture of solvents, EtOH:THF (5 ml, 1:1) then catalytic amount of Pd/C was added and stirred at room temperature under hydrogen atmosphere for 1.5 h. The reaction mixture was filtered, and solvent was removed under reduced pressure. The crude 588d (145 mg, 91% yield) was pure enough for the next step; m/z (ESI, +ve ion)=293.3 [M+H]⁺. Crude 588d (130 mg, 0.445 mmol) was dissolved in the anhydrous acetonitrile, 2-chloro-1,1,1, trimethoxyethane (0.066 ml, 0.489 mmol) followed PTSA (3.8 mg, 0.022 mmol) was added to the reaction mixture. The reaction mixture was heated to 60° C. for 2H. The reaction mixture was concentrated under reduced pressure and crude reaction mixture was purified using silica gel column chromatography to get compound 588e (153 mg, 76% yield); m/z (ESI, +ve ion)=351.1 [M+H]⁺.

### Step C. Methyl 2-(((2S,4S)-4-((2-((2,4-difluorophenoxy)methyl)pyrimidin-4-yl)oxy)-2-methylpiperidin-1-yl)methyl)-1-(2-(2,2,2-trifluoroethoxy)ethyl)-1H-benzo[d]imidazole-6-carboxylate (588f)

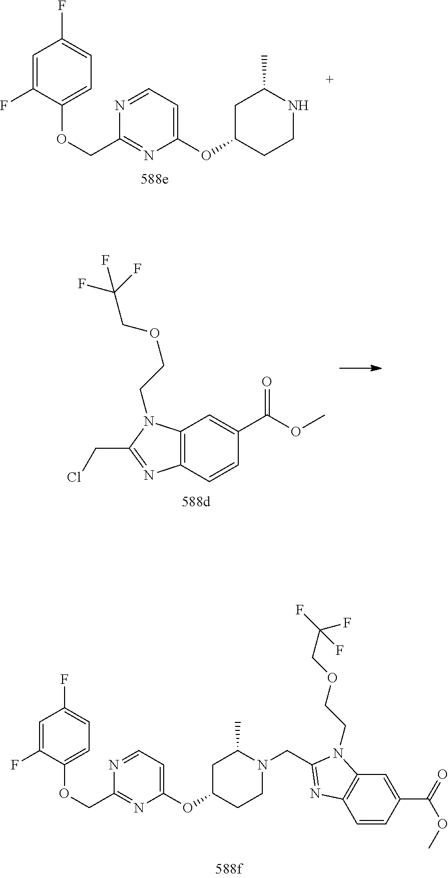 [SMILES: C[C@H]1C[C@@H](Oc2ccnc(COc3ccc(F)cc3F)n2)CCN1 ; COC(=O)c1ccc2nc(CCl)n(CCOCC(F)(F)F)c2c1 ; COC(=O)c1ccc2nc(CN3CC[C@H](Oc4ccnc(COc5ccc(F)cc5F)n4)C[C@@H]3C)n(CCOCC(F)(F)F)c2c1]

To a solution of compound 588e (prepared using a synthetic procedure as described for 599e) (27 mg, 0.080 mmol) and compound 588d (37 mg, 0.105) in anhydrous DMF, diisopropylethylamine (0.042 ml, 0.242 mmol) was added. The reaction mixture was heated to 60° C. for overnight. The reaction mixture was quenched with the addition of water, the aqueous layer was extracted with the ethyl acetate (3*5 ml). The combined organic layer was dried over anhydrous Na₂SO₄, filtered and concentrate under reduced pressure. The crude reaction mixture was purified by silica gel column chromatography to get pure compound 588f (52 mg, 40% yield); m/z (ESI, +ve ion)=650.3 [M+H]⁺.

### Step D. 2-{[(2S,4S)-4-({2-[(2,4-Difluorophenoxy)methyl]pyrimidin-4-yl}oxy)-2-methylpiperidin-1-yl]methyl}-1-[2-(2,2,2-trifluoroethoxy)ethyl]-1H-1,3-benzodiazole-6-carboxylic acid

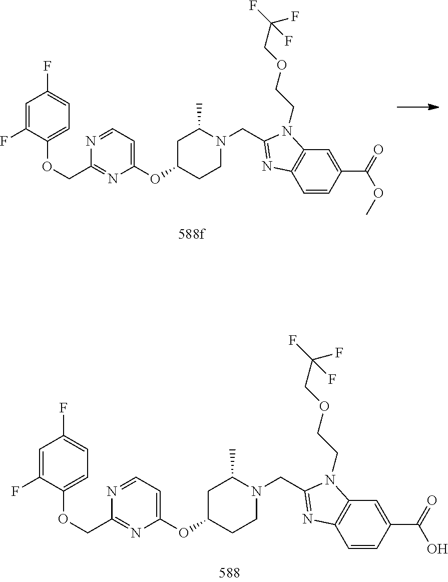 [SMILES: COC(=O)c1ccc2nc(CN3CC[C@H](Oc4ccnc(COc5ccc(F)cc5F)n4)C[C@@H]3C)n(CCOCC(F)(F)F)c2c1 ; C[C@H]1C[C@@H](Oc2ccnc(COc3ccc(F)cc3F)n2)CCN1Cc1nc2ccc(C(=O)O)cc2n1CCOCC(F)(F)F]

Compound 588f (21 mg, 0.032 mmol) was dissolved in a mixture of solvents THF:CH₃OH:H₂O (5 ml, 6:1:1) and LiOH·H₂O (4 mg, 0.162 mmol) was added and stirred for 12H at room temperature. The reaction mixture was quenched with the addition of acetic acid to adjust the pH around 7.0. The reaction mixture was concentrated under reduced pressure and diluted with water. The aqueous layer was extracted with ethyl acetate (4*5 ml), the combined organic layer was dried over anhydrous Na₂SO₄ and concentrated under vacuum. The crude reaction mixture was purified using silica gel column chromatography to get pure 588 (21 mg, 68% yield); ¹H NMR (400 MHz, CDCl₃) δ ppm 8.44 (d, 1H), 8.22 (s, 1H), 8.11 (d, 1H), 7.86 (d, 1H), 6.97-6.85 (m, 2H), 6.76-6.71 (m, 1H), 6.57 (d, 1H), 5.24 (s, 2H), 5.11-4.99 (m, 2H), 4.57-4.51 (m, 2H), 4.07-3.98 (m, 2H), 3.81-3.72 (m, 2H), 3.63 (d, 1H), 2.80-2.77 (m, 1H), 2.52-2.46 (m, 1H), 2.32-2.26 (m, 1H), 2.08 (d, 1H), 1.94 (d, 1H), 1.62-1.45 (m, 2H), 1.27 (d, 3H); m/z (ESI, +ve ion)=636.5 [M+H]⁺.

### Example 589. 2-{[(2S,4S)-4-({2-[(2,4-Difluorophenoxy)methyl]pyrimidin-4-yl}oxy)-2-methylpiperidin-1-yl]methyl}-1-{[(2S)-oxolan-2-yl]methyl}-1H-1,3-benzodiazole-6-carboxylic acid (589)

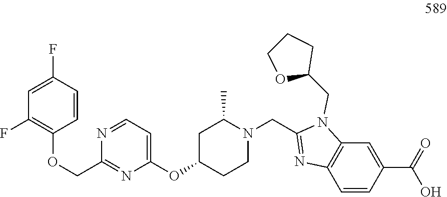 [SMILES: C[C@H]1C[C@@H](Oc2ccnc(COc3ccc(F)cc3F)n2)CCN1Cc1nc2ccc(C(=O)O)cc2n1C[C@@H]1CCCO1]

Example 589 was synthesized using the synthetic procedure of Example 579. ¹H NMR (400 MHz, MeOD) δ 8.41 (d, 1H), 8.16 (s, 1H), 7.93 (dd, 1H), 7.56 (d, 1H), 7.10-6.97 (m, 2H), 6.85-6.78 (m, 1H), 6.71 (d, 1H), 5.25 (s, 2H), 5.07-4.99 (m, 1H), 4.86-4.69 (m, 1H), 4.56-4.45 (m, 2H), 4.42-4.32 (m, 1H), 3.86 (dd, 1H), 3.72 (dd, 1H), 3.57 (d, 1H), 2.78 (d, 1H), 2.45 (s, 1H), 2.28-2.17 (m, 1H), 2.16-2.05 (m, 1H), 2.05-1.98 (m, 1H), 1.95-1.79 (m, 3H), 1.74-1.53 (m, 2H), 1.47 (dd, 1H), 1.23 (d, 3H); m/z (ESI, +ve ion)=594.2 [M+H]⁺.

### Example 590. 1-(Cyclobutylmethyl)-2-{[(2S,4S)-4-({2-[(2,4-difluorophenoxy)methyl]pyrimidin-4-yl}oxy)-2-methylpiperidin-1-yl]methyl}-1H-1,3-benzodiazole-6-carboxylic acid (590)

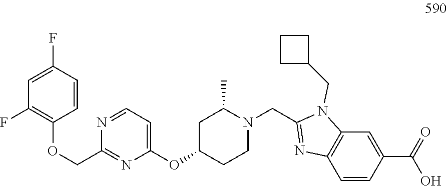 [SMILES: C[C@H]1C[C@@H](Oc2ccnc(COc3ccc(F)cc3F)n2)CCN1Cc1nc2ccc(C(=O)O)cc2n1CC1CCC1]

### Step A: Methyl 3-[(cyclobutylmethyl)amino]-4-nitrobenzoate (590c)

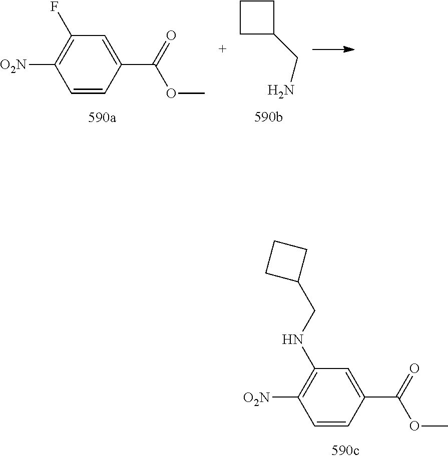 [SMILES: COC(=O)c1ccc([N+](=O)[O-])c(F)c1 ; NCC1CCC1 ; COC(=O)c1ccc([N+](=O)[O-])c(NCC2CCC2)c1]

To a solution of 590a (2.34 g, 11.8 mmol) in THF (20 mL) was added 590b (1.51g, 17.7 mmol) and TEA (3.58 g, 35.4 mmol). The reaction was stirred at 20° C. for 16 h. After completion, the mixture was diluted with H₂O (20 mL), extracted with ethyl acetate (50 mL*3). The combined organic layer was washed with brine (20 mL), dried over anhydrous Na₂SO₄ and filtered. The filtrate was concentrated under reduce pressure to give a residue. The residue was purified by flash column on silica gel (PE/EA=4:1) to afford 590c (2.8 g, 89.5% yield) as yellow solid; m/z (ESI, +ve ion)=265.1 [M+H]⁺.

### Step B: methyl 4-amino-3-[(cyclobutylmethyl)amino]benzoate (590d)

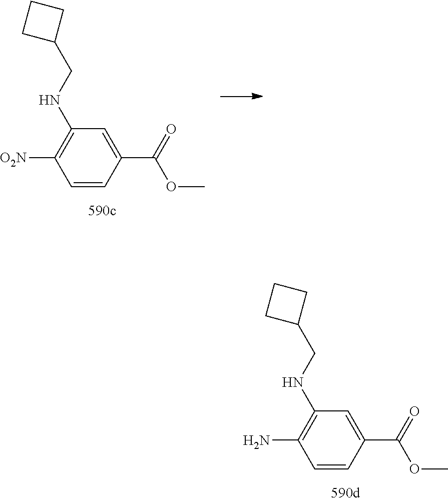 [SMILES: COC(=O)c1ccc([N+](=O)[O-])c(NCC2CCC2)c1 ; COC(=O)c1ccc(N)c(NCC2CCC2)c1]

To a solution of 590c (2.7 g, 10.2 mmol) in MeOH (30 mL) was added Pd/C (0.11 g, 1 mol, 0.1 eq). The reaction was stirred at 20° C. for 16 h under H₂ atmosphere. After completion, the mixture was filtered, and the filter cake was washed with methanol. The organic layers were concentrated under vacuum to give a residue. The residue was purified by flash column on silica gel (PE/EA=4:1) to afford 590d (2.07 g, 86.2% yield) as yellow solid; m/z (ESI, +ve ion)=235.3 [M+H]⁺.

### Step C: Methyl 2-(chloromethyl)-3-(cyclobutylmethyl)-1,3-benzodiazole-5-carboxylate (590e)

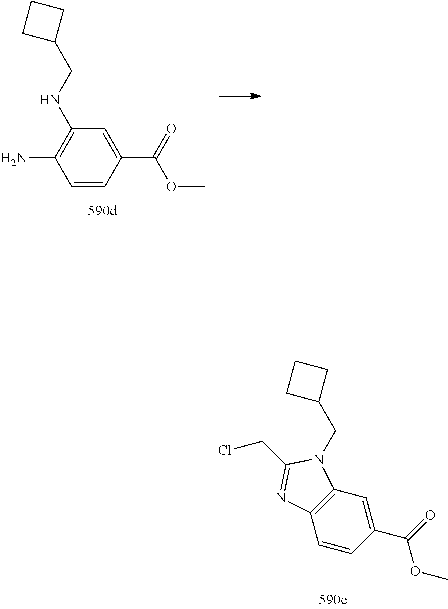 [SMILES: COC(=O)c1ccc(N)c(NCC2CCC2)c1 ; COC(=O)c1ccc2nc(CCl)n(CC3CCC3)c2c1]

To a solution of 590d (200 mg, 0.85 mmol) in ACN (2 mL) was added 2-chloro-1,1,1-trimethoxyethane (395.9 mg, 2.56 mmol) and PTSA (14.7 mg, 0.085 mmol). The mixture was stirred at 20° C. for 2H. After completion, the mixture was concentrated in vacuo to give a residue. The residue was purified by flash column on silica gel (PE/EA=2:1) to afford 590e (245 mg, 98.7% yield) as yellow solid; m/z (ESI, +ve ion)=293.3 [M+H]⁺.

### Step D: Methyl 1-(cyclobutylmethyl)-2-(((2S,4S)-4-((2-((2,4-difluorophenoxy)methyl)pyrimidin-4-yl)oxy)-2-methylpiperidin-1-yl)methyl)-1H-benzo[d]imidazole-6-carboxylate (590f)

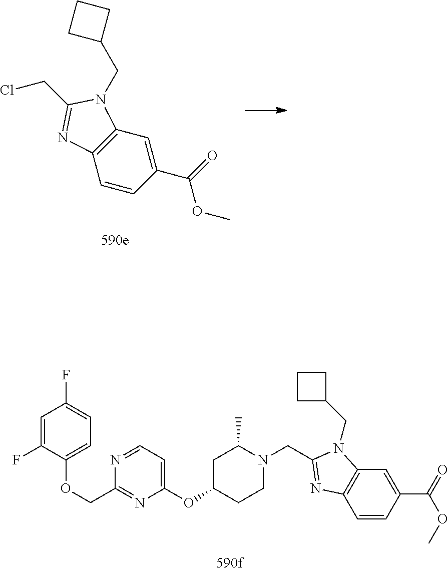 [SMILES: COC(=O)c1ccc2nc(CCl)n(CC3CCC3)c2c1 ; COC(=O)c1ccc2nc(CN3CC[C@H](Oc4ccnc(COc5ccc(F)cc5F)n4)C[C@@H]3C)n(CC3CCC3)c2c1]

To a solution of compound 588e (triflate salt which was prepared using the same procedure as 355f (27 mg, 0.08 mmol), 590e (24 mg, 0.08 mmol), K₂CO₃ (34 mg, 0.24 mmol) and KI (14 mg, 0.08 mmol) in CH₃CN (4 mL) was stirred at 20° C. for 16 h. After completion, the mixture was diluted with H₂O (10 mL), extracted with EtOAc (10 mL×3). The organic layer was washed with H₂O (10 mL), brine (10 mL), dried over anhydrous Na₂SO₄, filtrated and concentrated and purified by flash column on silica gel (DCM:MeOH=20:1) to afford product (30 mg, 60% yield) as yellow oil; m/z (ESI, +ve ion)=592.2 [M+H]⁺.

### Step E: 1-(Cyclobutylmethyl)-2-(((2S,4S)-4-((2-((2,4-difluorophenoxy)methyl)pyrimidin-4-yl)oxy)-2-methylpiperidin-1-yl)methyl)-1H-benzo[d]imidazole-6-carboxylic acid (590)

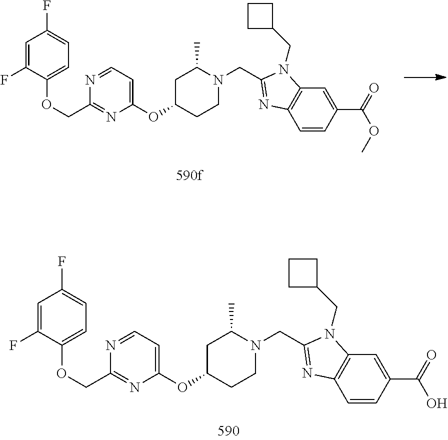 [SMILES: COC(=O)c1ccc2nc(CN3CC[C@H](Oc4ccnc(COc5ccc(F)cc5F)n4)C[C@@H]3C)n(CC3CCC3)c2c1 ; C[C@H]1C[C@@H](Oc2ccnc(COc3ccc(F)cc3F)n2)CCN1Cc1nc2ccc(C(=O)O)cc2n1CC1CCC1]

A solution of 590f (30 mg, 0.05 mmol, 1.0 eq) and LiOH (6 mg, 0.25 mmol, 5.0 eq) in THF/H₂O=1/1 (4 mL) was stirred at 20° C. for 16 h. After completion, 1N HCl was added to the mixture to adjust pH to 5 in an ice bath. The reaction mixture was extracted with EtOAc (10 mL×3). The organic layer was washed with H₂O (5 mL), brine (5 mL), dried over anhydrous Na₂SO₄, filtrated and concentrated in vacuo to give a residue. The residue was purified by Pre-HPLC (Mobile Phase: ACN-H₂O (0.1% NH₄OH)) to afford 590 (13.7 mg, 46% yield). ¹H NMR (400 MHz, DMSO) δ 8.49 (d, 1H), 8.05 (s, 1H), 7.77 (dd, 1H), 7.43 (d, 1H), 7.29 (ddd, 1H), 7.14 (td, 1H), 6.95 (dt, 1H), 6.79 (d, 1H), 5.31 (s, 2H), 4.91-4.82 (m, 1H), 4.45 (dd, 1H), 4.36-4.26 (m, 2H), 3.41 (s, 1H), 2.90 (dd, 1H), 2.73-2.67 (m, 1H), 2.36 (dd, 1H), 2.09 (t, 1H), 1.99-1.80 (m, 8H), 1.46-1.38 (m, 1H), 1.35-1.26 (m, 1H), 1.20 (d, 3H); m/z (ESI, +ve ion)=578.2 [M+H]⁺.

### Example 591. 1-[(2S)-2-(Difluoromethoxy)propyl]-2-{[(2S,4S)-4-({2-[(2,4-difluorophenoxy)methyl]pyrimidin-4-yl}oxy)-2-methylpiperidin-1-yl]methyl}-1H-1,3-benzodiazole-6-carboxylic acid (591)

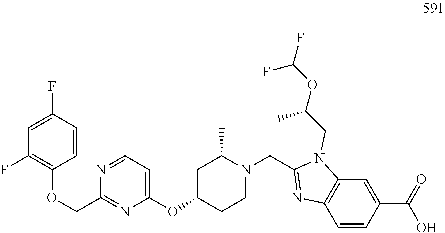 [SMILES: C[C@@H](Cn1c(CN2CC[C@H](Oc3ccnc(COc4ccc(F)cc4F)n3)C[C@@H]2C)nc2ccc(C(=O)O)cc21)OC(F)F]

### Step A: Benzyl(S)-(2-(difluoromethoxy)propyl)carbamate (591b)

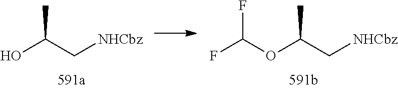 [SMILES: C[C@H](O)CNC(=O)OCc1ccccc1 ; C[C@@H](CNC(=O)OCc1ccccc1)OC(F)F]

A mixture of 591a (7 g, 33.45 mmol) and CuI (1.27 g, 6.69 mmol) in dry CH₃CN (70 mL) was degassed and purged with N₂ for 3 times. The mixture was heated to 65° C. 2,2-difluoro-2-fluorosulfonyl-acetic acid (8.94 g, 50.18 mmol, 5.20 mL) in CH₃CN (70 mL) was added dropwise over 50 min at 65° C. The mixture was stirred at 65° C. for another 10 min under N₂. After completion, the mixture was concentrated to give crude product. The crude product was diluted with ethyl acetate (20 mL), and then filtered through a Celite® pad, and the filtrate was concentrated to give crude product. The crude product was purified by flash silica gel chromatography (ISCO®; 40 g SepaFlash® Silica Flash Column, Petroleum ether:Ethyl acetate=1:0~82:18) to afford desired product 591b (5.4 g, 62.26% yield) as colorless oil. m/z (ESI, +ve ion)=260.2 [M+H]⁺.

### Step B. Methyl(S)-3-((2-(difluoromethoxy)propyl)amino)-4-nitrobenzoate (591c)

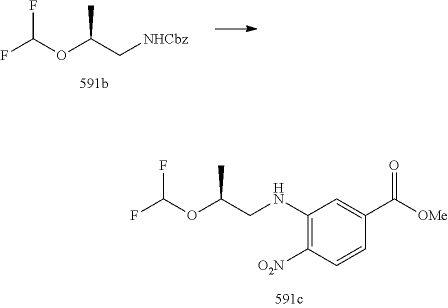 [SMILES: C[C@@H](CNC(=O)OCc1ccccc1)OC(F)F ; COC(=O)c1ccc([N+](=O)[O-])c(NC[C@H](C)OC(F)F)c1]

To a solution of 591b (5 g, 19.29 mmol) in THF (50 mL) was added Pd/C (5 g, 50% w/w) under Ar. The suspension was degassed under vacuum and purged with H₂ several times. The mixture was stirred under H₂ (50 psi) at 40° C. for 5 h. After completion, the mixture was filtered through a Celite® pad, the filter cake was washed with THF (30 mL×5). The filtrate was used for next step directly without work up. The crude amine was dissolved in THF (~200 mL) was obtained as a colorless liquid.

A mixture of amine (2.4 g, 19.18 mmol), methyl 3-fluoro-4-nitro-benzoate (3.82 g, 19.18 mmol), K₂CO₃ (7.95 g, 57.55 mmol) in THF (200 mL) was degassed and purged with N₂ for 3 times, and then the mixture was stirred at 45° C. for 16 h under N₂ atmosphere. After completion, the mixture was filtered, and the filtrate was concentrated to give crude product. The product was diluted with ethyl acetate (50 mL), H₂O (50 mL) was added. Separated, the organic phase was concentrated in vacuum. The residue was purified by flash silica gel chromatography (ISCO®; 80 g SepaFlash® Silica Flash Column, petroleum ether:ethyl acetate=1:0~5:1) to afford desired product 591c (3.8 g, 65.11% yield) as a yellow solid. m/7. (ESI, +ve ion)=305.1 [M+H]⁺.

### Step C: Methyl(S)-2-(chloromethyl)-1-(2-(difluoromethoxy)propyl)-1H-benzo[d]imidazole-6-carboxylate (591d)

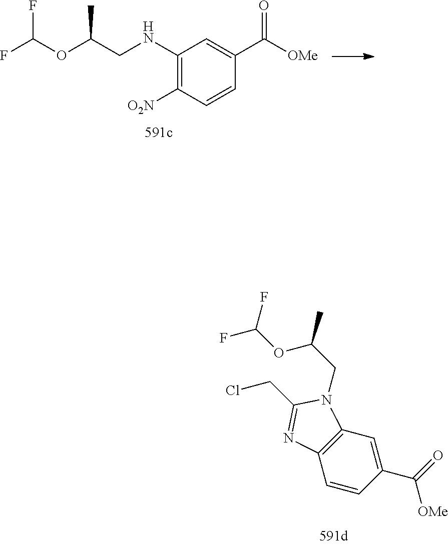 [SMILES: COC(=O)c1ccc([N+](=O)[O-])c(NC[C@H](C)OC(F)F)c1 ; COC(=O)c1ccc2nc(CCl)n(C[C@H](C)OC(F)F)c2c1]

MeOH (40 mL) was added Pd/C (3 g, 50% w/w) under N₂. The suspension was degassed under vacuum and purged with H₂ (19.88 mg, 9.86 mmol) several times. The mixture was stirred under H₂ (15 psi) at 30° C. for 1H. Compound 591c (3 g, 9.86 mmol) was added under N₂. The suspension was degassed under vacuum and purged with H₂ several times. The mixture was stirred under H₂ (15 psi) at 30° C. for 2H. After completion, the mixture was filtered through a Celite® pad, the filter cake was washed with MeOH (20 mL×4). The filtrate was concentrated to afford amine (2.7 g, 99.84% yield) as a colorless oil. m/z (ESI, +ve ion)=275.2 [M+H]⁺.

To a solution of amine (2.7 g, 9.84 mmol) in MeCN (30 mL) was added 4-methylbenzenesulfonic acid; hydrate (187.26 mg, 984.46 umol) and 2-chloro-1,1,1-trimethoxy-ethane (2.28 g, 14.77 mmol, 1.99 mL). The mixture was stirred at 60° C. for 30 min. After completion, the mixture was concentrated to give crude product. The reaction was quenched by sat. NaHCO₃ (30 mL) and then extracted with ethyl acetate (30 mL×2). The combined organic phase was dried over anhydrous Na₂SO₄, filtered and concentrated in vacuo. The residue was purified by flash silica gel chromatography (ISCOR; 40 g SepaFlash® Silica Flash Column, petroleum ether:ethyl acetate=1:0~3:1) to afford desired product 591d (2.5 g, 76.18% yield). m/z (ESI, +ve ion)=333.0 [M+H]⁺.

### Step D: Methyl 1-((S)-2-(difluoromethoxy)propyl)-2-(((2S,4S)-4-((2-((2,4-difluorophenoxy)methyl)pyrimidin-4-yl)oxy)-2-methylpiperidin-1-yl)methyl)-1H-benzo[d]imidazole-6-carboxylate (591e)

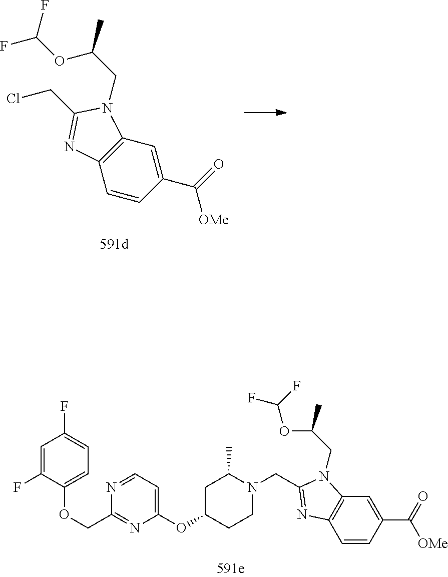 [SMILES: COC(=O)c1ccc2nc(CCl)n(C[C@H](C)OC(F)F)c2c1 ; COC(=O)c1ccc2nc(CN3CC[C@H](Oc4ccnc(COc5ccc(F)cc5F)n4)C[C@@H]3C)n(C[C@H](C)OC(F)F)c2c1]

To a solution of 588e (triflate salt which was prepared using the same procedure as 355f (0.1 g, 298.20 umol) and 591d (99.22 mg, 298.20 umol) in DMF (3 mL) was added DIEA (192.70 mg, 1.49 mmol, 259.70 μL). The mixture was stirred at 80° C. for 16 h under N₂. After completion, the reaction was quenched by H₂O (10 mL) and then extracted with ethyl acetate (10 mL×2). The combined organic phase was dried over anhydrous Na₂SO₄, filtered and concentrated in vacuo. The residue was purified by prep-TLC (SiO₂, Petroleum ether:Ethyl acetate=0:1, Rf=0.6) to afford 591e (100 mg, 53.09% yield). m/z (ESI, +ve ion)=632.2 [M+H]⁺.

### Step E: 1-((S)-2-(Difluoromethoxy)propyl)-2-(((2S,4S)-4-((2-((2,4-difluorophenoxy)methyl)pyrimidin-4-yl)oxy)-2-methylpiperidin-1-yl)methyl)-1H-benzo[d]imidazole-6-carboxylic acid (591)

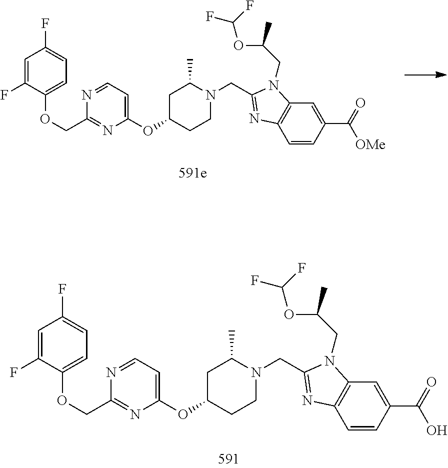 [SMILES: COC(=O)c1ccc2nc(CN3CC[C@H](Oc4ccnc(COc5ccc(F)cc5F)n4)C[C@@H]3C)n(C[C@H](C)OC(F)F)c2c1 ; C[C@@H](Cn1c(CN2CC[C@H](Oc3ccnc(COc4ccc(F)cc4F)n3)C[C@@H]2C)nc2ccc(C(=O)O)cc21)OC(F)F]

To a solution of 591e (80 mg, 126.66 umol) in THF (2 mL), MeOH (1 mL) and H₂O (1 mL) was added LiOH·H₂O (26.58 mg, 633.30 umol). The mixture was stirred at 15° C. for 16 hr. After completion, the mixture was concentrated to remove THF and MeOH. The mixture was adjusted to pH ~6 with HCl (1 M, ~0.6 mL) and extracted with DCM (10 mL×3). The combined organic layers were concentrated under reduced pressure. The residue was purified by prep-HPLC (column: Waters Xbridge Prep OBD C18 150*40 mm*10 μm; mobile phase: [water (NH₄HCO₃)-ACN]; B %: 15%-45%, 8 min) to afford compound 591 (60 mg, 76.70% yield). ¹H NMR (400 MHz, DMSO-d6) δ 8.49 (d, 1H), 8.21 (s, 1H), 7.79 (dd, 1H), 7.63 (d, 1H), 7.28 (ddd, 1H), 7.14 (dt, 1H), 6.98-6.89 (m, 1H), 6.79 (d, 1H), 6.74-6.31 (m, 1H), 5.30 (s, 2H), 4.94-4.84 (m, 1H), 4.83-4.75 (m, 1H), 4.66-4.52 (m, 2H), 4.26 (d, 1H), 3.67 (d, 1H), 2.80-2.68 (m, 1H), 2.46 (brd, 1H), 2.23 (brt, 1H), 1.97-1.80 (m, 2H), 1.48 (dq, 1H), 1.32 (d, 4H), 1.10 (d, 3H); m/z (ESI, +ve ion)=618.2 [M+H]⁺.

### Example 592. 1-[(2R)-2-(Difluoromethoxy)propyl]-2-{[(2S,4S)-4-({2-[(2,4-difluorophenoxy)methyl]pyrimidin-4-yl}oxy)-2-methylpiperidin-1-yl]methyl}-1H-1,3-benzodiazole-6-carboxylic acid (592)

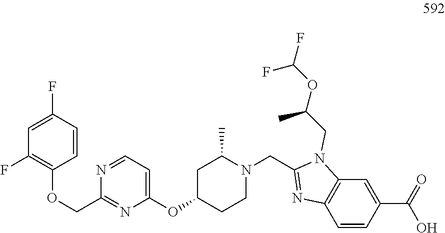 [SMILES: C[C@H](Cn1c(CN2CC[C@H](Oc3ccnc(COc4ccc(F)cc4F)n3)C[C@@H]2C)nc2ccc(C(=O)O)cc21)OC(F)F]

Example 592 was synthesized by following similar synthetic route starting from opposite enantiomer of 591a. ¹H NMR (400 MHz, DMSO-d6) δ 8.55-8.39 (m, 1H), 8.22 (d, 1H), 7.79 (dd, 1H), 7.63 (d, 1H), 7.29 (ddd, 1H), 7.14 (dt, 1H), 6.99-6.88 (m, 1H), 6.79 (d, 1H), 6.66-6.20 (m, 1H), 5.36-5.26 (m, 2H), 4.94-4.74 (m, 2H), 4.69-4.37 (m, 3H), 3.36 (s, 1H), 2.70-2.59 (m, 1H), 2.39-2.28 (m, 1H), 2.10-1.78 (m, 3H), 1.46-1.24 (m, 5H), 1.20 (d, 3H); m/z (ESI, +ve ion)=618.2 [M+H]⁺.

### Example 594. 2-{[(2S,4S)-4-({2-[(2,4-Difluorophenoxy)methyl]pyrimidin-4-yl}oxy)-2-methylpiperidin-1-yl]methyl}-1-[(2R)-2-(2,2,2-trifluoroethoxy)propyl]-1H-1,3-benzodiazole-6-carboxylic acid (594)

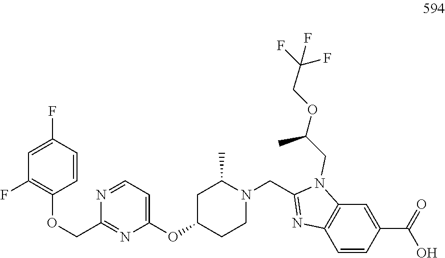 [SMILES: C[C@H](Cn1c(CN2CC[C@H](Oc3ccnc(COc4ccc(F)cc4F)n3)C[C@@H]2C)nc2ccc(C(=O)O)cc21)OCC(F)(F)F]

Example 594 was synthesized using similar procedure as compound 592. ¹H NMR (400 MHz, DMSO-d6) δ ppm 8.55 (d, 1H), 8.33 (s, 1H), 7.90 (brd, 1H), 7.79 (brd, 1H), 7.27 (ddd, 1H), 7.17 (td, 1H), 6.96 (brt, 1H), 6.88 (d, 1H), 5.30 (s, 2H), 5.03-5.19 (m, 2H), 4.74-4.88 (m, 1H), 4.01-4.16 (m, 4H), 3.38 (brs, 5H), 1.86-2.28 (m, 4H), 1.67 (d, 3H), 1.38 (brs, 3H), 1.23 (s, 1H); m/z (ESI, +ve ion)=650.2 [M+H]⁺.

### Example 595. 2-{[(2S,4S)-4-[(2-{[(5-chloro-3-fluoropyridin-2-yl)oxy]methyl}pyrimidin-4-yl)oxy]-2-methylpiperidin-1-yl]methyl}-1-[2-(2,2,2-trifluoroethoxy)ethyl]-1H-1,3-benzodiazole-6-carboxylic acid (595)

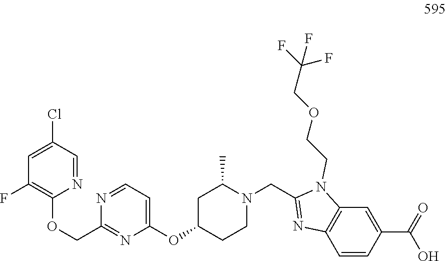 [SMILES: C[C@H]1C[C@@H](Oc2ccnc(COc3ncc(Cl)cc3F)n2)CCN1Cc1nc2ccc(C(=O)O)cc2n1CCOCC(F)(F)F]

Example 595 was synthesized by following the synthetic route A-B for Example 554 followed by C-D of 588. ¹H NMR (400 MHz, CDCl₃) δ ppm 8.39 (d, 1H), 8.19 (s, 1H), 8.08 (d, 1H), 7.84-7.82 (m, 2H), 7.44 (dd, 1H), 6.53 (d, 1H), 5.59 (s, 1H), 5.04-4.89 (m, 2H), 4.57-4.49 (m, 2H), 4.07-3.96 (m, 2H), 3.77 (q, 2H), 3.61-3.58 (m, 1H), 2.76-2.74 (m, 1H), 2.42 (bs, 1H), 2.20-2.18 (m, 1H), 2.03-2.00 (m, 2H), 1.87-1.85 (m, 2H), 1.54-1.43 (m, 2H), 1.26 (d, 3H); m/z (ESI, +ve ion)=654.3 [M+H]⁺.

### Example 596. 2-{[(2S,4S)-4-({2-[(4-Cyano-2-fluorophenoxy)methyl]pyrimidin-4-yl}oxy)-2-methylpiperidin-1-yl]methyl}-1-[2-(2,2,2-trifluoroethoxy)ethyl]-1H-1,3-benzodiazole-6-carboxylic acid

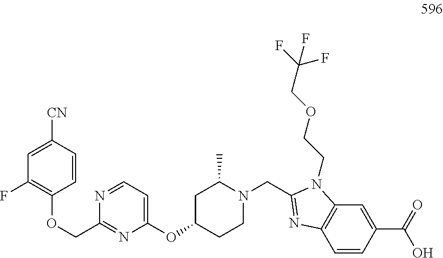 [SMILES: C[C@H]1C[C@@H](Oc2ccnc(COc3ccc(C#N)cc3F)n2)CCN1Cc1nc2ccc(C(=O)O)cc2n1CCOCC(F)(F)F]

Example 596 was synthesized using similar procedure as described in Example 588. Compound 596. ¹H NMR (400 MHz, CDCl₃) δ ppm 8.33 (d, 1H), 8.10 (s, 1H), 7.98 (d, 1H), 7.73 (d, 1H), 7.32 (d, 1H), 7.27 (d, 1H), 6.94 (t, 1H), 6.5 (d, 1H), 5.22 (s, 2H), 4.94-4.88 (m, 2H), 4.46-4.40 (m, 2H), 3.98-3.88 (m, 2H), 3.71-3.64 (m, 2H), 3.49 (d, 1H), 2.70-2.67 (m, 1H), 2.38-2.30 (m, 1H), 2.17-2.11 (m, 1H), 1.96-1.93 (m, 1H), 1.84-1.82 (m, 1H), 1.52-1.35 (m, 3H), 1.17 (d, 3H); m/z (ESI, +ve ion)=643.3 [M+H]⁺.

### Example 597. 2-{[(2S,4S)-4-[(2-{[(5-Chloro-3-fluoropyridin-2-yl)oxy]methyl}pyrimidin-4-yl)oxy]-2-methylpiperidin-1-yl]methyl}-4-fluoro-1-{[(2S)-oxetan-2-yl]methyl}-1H-1,3-benzodiazole-6-carboxylic acid (597)

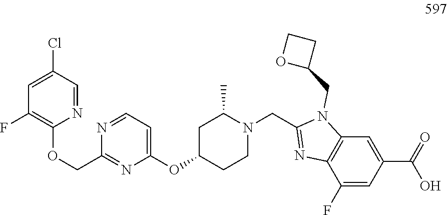 [SMILES: C[C@H]1C[C@@H](Oc2ccnc(COc3ncc(Cl)cc3F)n2)CCN1Cc1nc2c(F)cc(C(=O)O)cc2n1C[C@@H]1CCO1]

Example 597 was synthesized by following the synthetic route for Example 606. ¹H NMR (400 MHz, CDCl₃) δ ppm 8.30 (d, 1H), 7.95 (s, 1H), 7.73 (d, 1H), 7.63 (d, 1H), 7.37 (d, 1H), 6.44 (d, 1H), 5.5 (s, 2H), 5.18-5.16 (m, 1H), 4.94-4.89 (m, 1H), 4.56-4.42 (m, 3H), 4.23-4.18 (m, 1H), 3.56 (d, 1H), 2.68-2.61 (m, 2H), 2.36-2.27 (m, 2H), 2.16-2.10 (m, 1H), 1.91-1.88 (m, 1H), 1.77-1.75 (m, 1H), 1.49-1.31 (m, 3H), 1.12 (d, 3H); m/z (ESI, +ve ion)=615.3 [M+H]⁺.

### Example 598. 2-{[(2S,4S)-4-({2-[(2,4-Difluorophenoxy)methyl]pyrimidin-4-yl}oxy)-2-methylpiperidin-1-yl]methyl}-1-[(2S)-2-(2,2,2-trifluoroethoxy)propyl]-1H-1,3-benzodiazole-6-carboxylic acid (598)

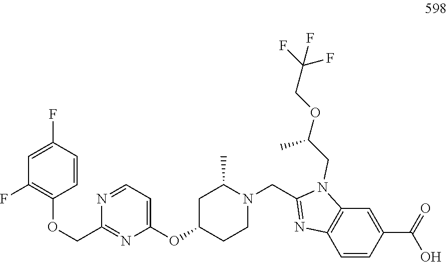 [SMILES: C[C@@H](Cn1c(CN2CC[C@H](Oc3ccnc(COc4ccc(F)cc4F)n3)C[C@@H]2C)nc2ccc(C(=O)O)cc21)OCC(F)(F)F]

Example 598 was synthesized using similar procedure as compound 592. ¹H NMR (400 MHz, DMSO-d6) δ ppm 8.48 (d, 1H), 8.20 (s, 1H), 7.80 (brd, 1H), 7.65 (brd, 1H), 7.28 (ddd, 1H), 7.10-7.19 (m, 1H), 6.89-6.98 (m, 1H), 6.77 (d, 1H), 5.32-5.39 (m, 1H), 5.30 (s, 2H), 4.83-4.92 (m, 1H), 4.38 (brd, 1H), 4.15 (brt, 1H), 3.96-4.10 (m, 3H), 3.39-3.45 (m, 3H), 2.33 (brd, 1H), 2.04-2.13 (m, 1H), 1.91-1.99 (m, 1H), 1.82-1.89 (m, 1H), 1.60 (brd, 3H), 1.38-1.49 (m, 1H), 1.26-1.36 (m, 1H), 1.17 (brd, 3H); m/z (ESI, +ve ion)=650.2 [M+H]⁺.

### Example 599. 2-{[(2R,4S)-4-({2-[(4-cyano-2-fluorophenoxy)methyl]pyrimidin-4-yl}oxy)-2-(trifluoromethyl)piperidin-1-yl]methyl}-1-{[(2S)-oxetan-2-yl]methyl}-1H-1,3-benzodiazole-6-carboxylic acid (599)

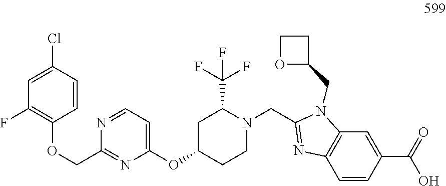 [SMILES: O=C(O)c1ccc2nc(CN3CC[C@H](Oc4ccnc(COc5ccc(Cl)cc5F)n4)C[C@@H]3C(F)(F)F)n(C[C@@H]3CCO3)c2c1]

### Step A: tert-Butyl(2R,4S)-4-((2-chloropyrimidin-4-yl)oxy)-2-(trifluoromethyl)piperidine-1-carboxylate (599b)

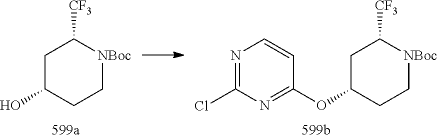 [SMILES: CC(C)(C)OC(=O)N1CC[C@H](O)C[C@@H]1C(F)(F)F ; CC(C)(C)OC(=O)N1CC[C@H](Oc2ccnc(Cl)n2)C[C@@H]1C(F)(F)F]

To a solution of 2,4-dichloropyrimidine (180 mg, 1.21 mmol) and 599a (195.20 mg, 724.94 umol) in DMF (3 mL) was added Cs₂CO₃ (1.18 g, 3.62 mmol). The mixture was stirred at 80° C. for 16 hr. After completion, the reaction mixture was diluted with NH₄Cl water solution (30 mL). The aqueous phase was extracted with ethyl acetate (10 mL*3). The combined organic phase was washed with brine (50 mL*2), dried with anhydrous Na₂SO₄, filtered and concentrated in vacuum. The residue was purified by prep-TLC (PE/EA=3:1, Rf=0.3) to afford 599b (178 mg, 38.59% yield) as a yellow oil; m/z (ESI, +ve ion)=382.3 [M+H]⁺.

### Step B: Methyl 4-(((2R,4S)-1-(tert-butoxycarbonyl)-2-(trifluoromethyl)piperidin-4-yl)oxy)pyrimidine-2-carboxylate (599c)

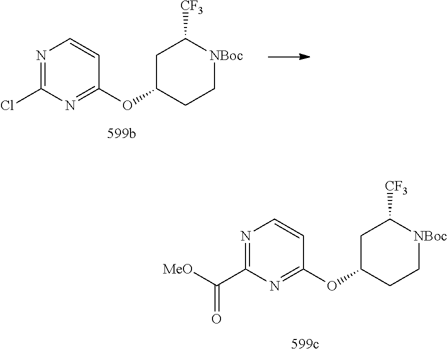 [SMILES: CC(C)(C)OC(=O)N1CC[C@H](Oc2ccnc(Cl)n2)C[C@@H]1C(F)(F)F ; COC(=O)c1nccc(O[C@H]2CCN(C(=O)OC(C)(C)C)[C@@H](C(F)(F)F)C2)n1]

A mixture of 599b (178 mg, 466.24 umol), TEA (47.18 mg, 466.24 umol, 64.89 uL) and Pd(dppf)Cl₂ (17.06 mg, 23.31 umol) in MeOH (2 mL) and DMSO (2 mL) was degassed and purged with CO for 3 times, and then the mixture was stirred at 65° C. for 12H under CO atmosphere. After completion, the residue was diluted with NH₄Cl water solution (50 mL). The aqueous phase was extracted with ethyl acetate (30 mL*3). The combined organic phase was washed with brine (100 mL*2), dried with anhydrous Na₂SO₄, filtered and concentrated in vacuum. The residue was purified by prep-TLC (PE/EA=0:1, Rf=0.3) to afford 599c (120 mg, 63.49% yield) as a yellow solid. m/z (ESI, +ve ion)=406.2 [M+H]⁺.

### Step C: tert-Butyl(2R,4S)-4-((2-(hydroxymethyl)pyrimidin-4-yl)oxy)-2-(trifluoromethyl)piperidine-1-carboxylate (599d)

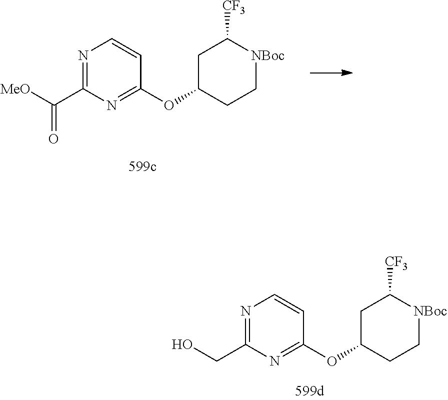 [SMILES: COC(=O)c1nccc(O[C@H]2CCN(C(=O)OC(C)(C)C)[C@@H](C(F)(F)F)C2)n1 ; CC(C)(C)OC(=O)N1CC[C@H](Oc2ccnc(CO)n2)C[C@@H]1C(F)(F)F]

A mixture of methyl 599c (120 mg, 296.03 umol) and CaCl₂) (19.71 mg, 177.62 umol) in EtOH (2 mL) was degassed and purged with N₂ for 3 times, then NaBH₄ (6.72 mg, 177.62 umol) was added, and then the mixture was stirred at 0-20° C. for 1H under N₂ atmosphere. After completion, the reaction mixture was diluted with water (30 mL). The aqueous phase was extracted with ethyl acetate (20 mL*3). The combined organic phase was dried with anhydrous Na₂SO₄, filtered and concentrated in vacuum. The residue was purified by prep-TLC (PE/EA=1:1, Rf=0.3) to afford 599d (80 mg, 71.62% yield). m/z (ESI, +ve ion)=378.3 [M+H]⁺.

### Step D: tert-Butyl(2R,4S)-4-((2-((4-cyano-2-fluorophenoxy)methyl)pyrimidin-4-yl)oxy)-2-(trifluoromethyl)piperidine-1-carboxylate (599e)

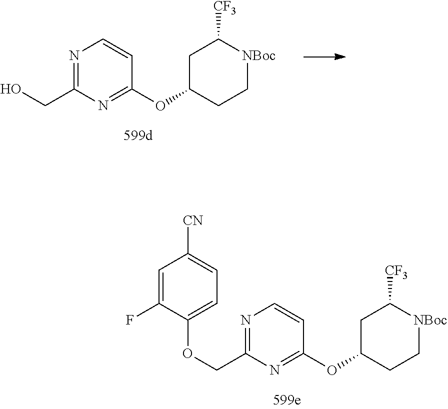 [SMILES: CC(C)(C)OC(=O)N1CC[C@H](Oc2ccnc(CO)n2)C[C@@H]1C(F)(F)F ; CC(C)(C)OC(=O)N1CC[C@H](Oc2ccnc(COc3ccc(C#N)cc3F)n2)C[C@@H]1C(F)(F)F]

A mixture of 599d (75 mg, 198.75 umol) and DIPEA (77.06 mg, 596.25 umol, 103.86 uL) in DCM (1 mL) was degassed and purged with N₂ for 3 times, then MsCl (34.15 mg, 298.13 umol, 23.07 uL) was added, and then the mixture was stirred at 0-20° C. for 2H under N₂ atmosphere. After completion, the reaction mixture was diluted with water (50 mL). The aqueous phase was extracted with DCM (30 mL*3). The combined organic phase was dried with anhydrous Na₂SO₄, filtered and concentrated in vacuum to afford the crude product (90 mg, 99.43% yield) as a yellow oil, which was used in the next step reaction without further purification.

A mixture of mesyl ester (90 mg, 197.61 umol), 3-fluoro-4-hydroxy-benzonitrile (32.51 mg, 237.13 umol) and K₂CO₃ (81.93 mg, 592.82 umol) in DMF (2 mL) was degassed and purged with N₂ for 3 times, and then the mixture was stirred at 35° C. for 14 h under N₂ atmosphere. After completion, the reaction mixture was diluted with NH₄Cl water solution (20 mL). The aqueous phase was extracted with ethyl acetate (10 mL*3). The combined organic phase was dried with anhydrous Na₂SO₄, filtered and concentrated in vacuum to afford the crude product (110 mg, 94.35% yield) as a yellow oil, which was used in the next step reaction without further purification. m/z (ESI, +ve ion)=497.2 [M+H]⁺.

### Step E: Methyl 2-(((2R,4S)-4-((2-((4-cyano-2-fluorophenoxy)methyl)pyrimidin-4-yl)oxy)-2-(trifluoromethyl)piperidin-1-yl)methyl)-1-(((S)-oxetan-2-yl)methyl)-1H-benzo[d]imidazole-6-carboxylate (599f)

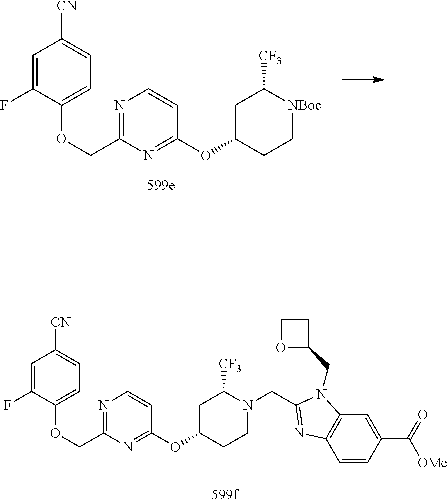 [SMILES: CC(C)(C)OC(=O)N1CC[C@H](Oc2ccnc(COc3ccc(C#N)cc3F)n2)C[C@@H]1C(F)(F)F ; COC(=O)c1ccc2nc(CN3CC[C@H](Oc4ccnc(COc5ccc(C#N)cc5F)n4)C[C@@H]3C(F)(F)F)n(C[C@@H]3CCO3)c2c1]

A mixture of 599e (120 mg, 241.71 umol) and TFA (145 mg, 1.27 mmol, 94.16 μL) in DCM (1 mL) was degassed and purged with N₂ for 3 times, and then the mixture was stirred at 15° C. for 3H under N₂ atmosphere. After completion, the reaction mixture was adjusted to pH=8 with NaHCO₃ water solution. The aqueous phase was extracted with DCM (30 mL*3). The combined organic phase was dried with anhydrous Na₂SO₄, filtered and concentrated in vacuum to afford the crude product (90 mg, 93.95% yield) as a yellow oil, which was used in the next step reaction without further purification. m/z (ESI, +ve ion)=397.3 [M+H]⁺.

To a solution of amine (70 mg, 176.62 umol) and methyl 2-(chloromethyl)-3-[[(2S)-oxetan-2-yl]methyl]benzimidazole-5-carboxylate (52.05 mg, 176.62 umol) in ACN (2 mL) was added K₂CO₃ (73.23 mg, 529.85 umol) and KI (29.32 mg, 176.62 umol). The mixture was stirred at 50° C. for 16 h. After completion, the reaction mixture was diluted with NH₄Cl water solution (30 mL). The aqueous phase was extracted with ethyl acetate (30 mL*3). The combined organic phase was washed with brine (100 mL*2), dried with anhydrous Na₂SO₄, filtered and concentrated in vacuum. The residue was purified by prep-TLC (PE/EA=0:1, Rf=0.3) to afford 599f (50 mg, 76.38 umol, 43.25% yield) as a yellow solid; m/z (ESI, +ve ion)=655.4 [M+H]⁺.

### Step F: 2-{[(2R,4S)-4-({2-[(4-cyano-2-fluorophenoxy)methyl]pyrimidin-4-yl}oxy)-2-(trifluoromethyl)piperidin-1-yl]methyl}-1-{[(2S)-oxetan-2-yl]methyl}-1H-1,3-benzodiazole-6-carboxylic acid

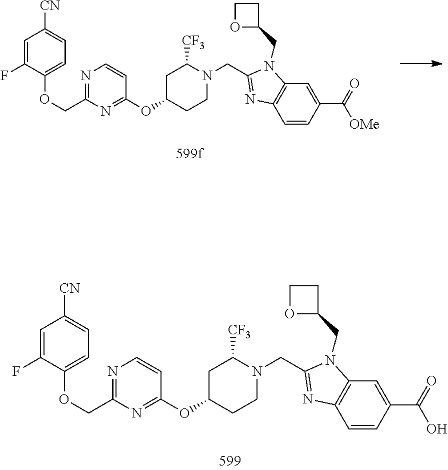 [SMILES: COC(=O)c1ccc2nc(CN3CC[C@H](Oc4ccnc(COc5ccc(C#N)cc5F)n4)C[C@@H]3C(F)(F)F)n(C[C@@H]3CCO3)c2c1 ; N#Cc1ccc(OCc2nccc(O[C@H]3CCN(Cc4nc5ccc(C(=O)O)cc5n4C[C@@H]4CCO4)[C@@H](C(F)(F)F)C3)n2)c(F)c1]

To a solution of 599f (40 mg, 61.11 umol) in THF (0.2 mL), MeOH (0.1 mL) and H₂O (0.1 mL) was added LiOH·H₂O (5.13 mg, 122.21 umol). The mixture was stirred at 15° C. for 16 h. After completion, the reaction mixture was adjusted to pH=5 with 1M HCl, extracted with DCM (10 mL*3), The combined organic phase was dried with anhydrous Na₂SO₄, filtered and concentrated in vacuum. The residue was purified by prep-HPLC (column: Phenomenex Luna C18 75*30 mm*3 μm; mobile phase: [water (FA)-ACN]; B %: 25%-55%, 8 min) to afford the desired product 599 (13.13 mg, 20.50 umol, 33.54% yield). 1H NMR (400 MHz, CD₃OD) δ=8.46 (d, 1H), 8.33-8.29 (m, 1H), 7.98 (d, 1H), 7.69 (dd, 1H), 7.59 (ddd, 1H), 7.47 (d, 1H), 7.24 (t, 1H), 6.76 (dd, 1H), 5.40 (s, 2H), 5.25-5.11 (m, 2H), 4.72-4.58 (m, 4H), 4.54-4.40 (m, 2H), 4.18-3.94 (m, 1H), 3.52-3.41 (m, 1H), 2.97-2.73 (m, 2H), 2.58-2.22 (m, 3H), 2.01-1.81 (m, 2H), 1.79-1.58 (m, 1H); m/z (ESI, +ve ion)=641.2 [M+H]⁺.

### Example 600. 2-{[(2S,4S)-4-({2-[(2,4-Difluorophenoxy)methyl]pyrimidin-4-yl}oxy)-2-methylpiperidin-1-yl]methyl}-1-[(1-methoxycyclobutyl)methyl]-1H-1,3-benzodiazole-6-carboxylic acid (600)

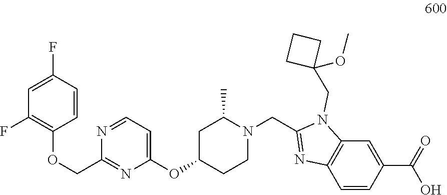 [SMILES: COC1(Cn2c(CN3CC[C@H](Oc4ccnc(COc5ccc(F)cc5F)n4)C[C@@H]3C)nc3ccc(C(=O)O)cc32)CCC1]

### Step A: (1-Methoxycyclobutyl) methanamine (600b)

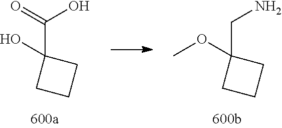

To a solution of 600a (2 g, 17.22 mmol) in DMF (30 mL) was added NaH (2.76 g, 68.90 mmol, 60% purity) at 0° C. The mixture was stirred at 15° C. for 2H. Mel (9.78 g, 68.90 mmol, 4.29 mL) was added. The mixture was stirred at 15° C. for 16 h. After completion. The mixture was dropwise added to sat. NH₄Cl (70 mL) and extracted with MTBE (50 mL×3). The organic layer was washed with brine (20 mL×2), dried over Na₂SO₄, filtered and concentrated in vacuo to afford desired ester (2.6 g, crude) as a yellow oil.

A solution of ester (2.6 g, 18.03 mmol) in THF (10 mL) saturated with NH₃·H₂O (31.55 g, 225.04 mmol, 34.67 mL, 25% purity) was stirred at 75° C. for 24 h in a 100 mL of sealed tube. After completion, the reaction was quenched by brine (30 mL) and then extracted with ethyl acetate (60 mL×3). The combined organic phase was dried over anhydrous Na₂SO₄, filtered and concentrated in vacuo to afford amide (2 g, 85.86% yield). The crude product was used for next step directly without purification.

To a solution of amide (2 g, 15.49 mmol) in THF (20 mL) was added BH₃·THF (1 M, 46.46 mL) at 15° C. under N₂. The mixture was stirred at 70° C. for 3 Hr. After completion, the reaction was quenched by MeOH (20 mL) and brine (30 mL) was added, then extracted with ethyl acetate (50 mL×5). The combined organic phase was washed with brine (20 mL), dried over anhydrous Na₂SO₄, filtered was diluted with 4 M HCl/dioxane (8 mL), and the mixture was concentrated to afford 600b (2 g, crude, HCl salt) as colorless oil.

Example 600 was synthesized using the similar procedure as compounds 590 from amine, 600b. ¹H NMR (400 MHz, DMSO-d6) δ 13.02-12.49 (m, 1H), 8.52-8.44 (m, 1H), 8.30 (s, 1H), 7.79 (brd, 1H), 7.64 (d, 1H), 7.30-7.20 (m, 1H), 7.13 (dt, 1H), 6.97-6.88 (m, 1H), 6.78 (d, 1H), 5.30 (s, 2H), 4.92-4.79 (m, 1H), 4.77-4.63 (m, 2H), 4.48 (brd, 1H), 3.58 (brd, 1H), 3.20 (s, 3H), 2.66 (brd, 2H), 2.36-2.23 (m, 2H), 2.17 (brt, 1H), 1.96-1.89 (m, 1H), 1.86-1.75 (m, 4H), 1.72 (brd, 1H), 1.50-1.37 (m, 1H), 1.30 (q, 1H), 1.14 (brd, 3H); m/z (ESI, +ve ion)=608.3 [M+H]⁺.

### Example 601. 2-{[(2S,4S)-4-({2-[(2-cyano-4-fluorophenoxy)methyl]pyrimidin-4-yl}oxy)-2-methylpiperidin-1-yl]methyl}-1-[2-(2,2,2-trifluoroethoxy)ethyl]-1H-1,3-benzodiazole-6-carboxylic acid (601)

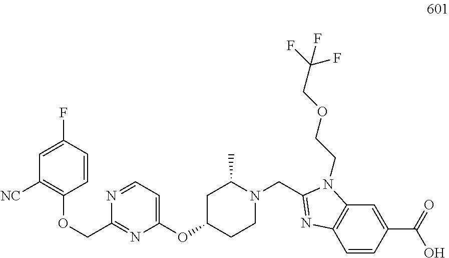 [SMILES: C[C@H]1C[C@@H](Oc2ccnc(COc3ccc(F)cc3C#N)n2)CCN1Cc1nc2ccc(C(=O)O)cc2n1CCOCC(F)(F)F]

Example 601 was synthesized using similar procedure as described in Example 588. ¹H NMR (400 MHz, CDCl₃) δ ppm 8.31 (d, 1H), 8.09 (s, 1H), 7.96 (d, 1H), 7.71 (d, 1H), 7.23-7.21 (m, 1H), 7.12-7.08 (m, 1H), 6.87-6.84 (m, 1H), 6.49 (d, 1H), 5.21 (s, 2H), 4.99-4.86 (m, 2H), 4.43-4.40 (m, 2H), 3.98-3.87 (m, 2H), 3.72-3.63 (m, 2H), 3.48 (d, 1H), 2.70-2.64 (m, 1H), 2.41-2.34 (m, 1H), 2.18-2.12 (m, 1H), 1.98-1.95 (m, 1H), 1.83-1.80 (m, 1H), 1.42-1.34 (m, 2H), 1.17 (d, 3H); m/z (ESI, +ve ion)=643.3 [M+H]⁺.

### Example 602. 2-{[(2S,4S)-4-[(2-{[(5-Chloro-3-fluoropyridin-2-yl)oxy]methyl}pyrimidin-4-yl)oxy]-2-methylpiperidin-1-yl]methyl}-1-[(2R)-2-(difluoromethoxy)propyl]-1H-1,3-benzodiazole-6-carboxylic acid (602)

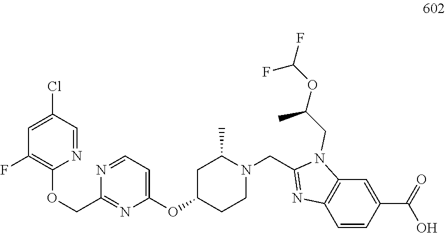 [SMILES: C[C@H](Cn1c(CN2CC[C@H](Oc3ccnc(COc4ncc(Cl)cc4F)n3)C[C@@H]2C)nc2ccc(C(=O)O)cc21)OC(F)F]

Example 602 was synthesized by following the synthetic route A-B for Example 554 followed by C-D of 588. ¹H NMR (400 MHz, CD₃OD) δ ppm 8.37 (d, 1H), 8.26 (s, 1H), 7.96 (dd, 1H), 7.82 (d, 1H), 7.76 (dd, 1H), 7.66 (d, 1H), 6.66 (d, 1H), 6.15 (t, 1H), 5.57 (s, 2H), 4.89-4.98 (m, 2H), 4.72 (dd, 1H), 4.63 (d, 1H), 4.42 (dd, 1H), 3.37 (brd, 2H), 2.57-2.67 (m, 1H), 2.26-2.34 (m, 1H), 1.93-2.06 (m, 2H), 1.69-1.78 (m, 1H), 1.47 (d, 3H), 1.42 (brd, 2H), 1.30 (d, 4H); m/z (ESI, +ve ion)=635.2 [M+H]⁺.

### Example 603. 2-{[(2S,4S)-4-[(2-{[(5-Chloropyridin-2-yl)oxy]methyl}pyrimidin-4-yl)oxy]-2-methylpiperidin-1-yl]methyl}-4-fluoro-1-{[(2S)-oxetan-2-yl]methyl}-1H-1,3-benzodiazole-6-carboxylic acid (603)

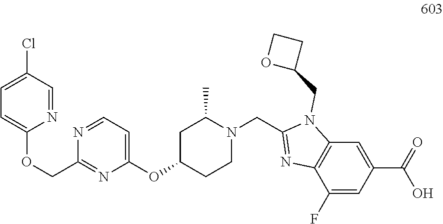 [SMILES: C[C@H]1C[C@@H](Oc2ccnc(COc3ccc(Cl)cn3)n2)CCN1Cc1nc2c(F)cc(C(=O)O)cc2n1C[C@@H]1CCO1]

Compound 603 was synthesized by following the synthetic route A-D for Example 606. ¹H NMR (400 MHz, DMSO-d6) δ 13.20-12.76 (m, 1H), 8.43 (d, 1H), 8.14 (d, 1H), 8.10-8.07 (m, 1H), 7.83 (dd, 1H), 7.51 (dd, 1H), 7.03 (d, 1H), 6.71 (d, 1H), 5.54-5.37 (m, 2H), 5.21-5.06 (m, 1H), 4.82-4.68 (m, 3H), 4.52-4.37 (m, 2H), 4.24 (td, 1H), 3.57 (d, 1H), 2.68-2.61 (m, 2H), 2.37-2.30 (m, 2H), 2.19-2.04 (m, 1H), 1.80-1.64 (m, 2H), 1.38 (dq, 1H), 1.29-1.18 (m, 1H), 1.07 (d, 3H); m/z (ESI, +ve ion)=597.2 [M+H]⁺.

### Example 604. 2-{[(2S,4S)-4-[(2-{[(5-cyanopyridin-2-yl)oxy]methyl}pyrimidin-4-yl)oxy]-2-methylpiperidin-1-yl]methyl}-4-fluoro-1-{[(2S)-oxetan-2-yl]methyl}-1H-1,3-benzodiazole-6-carboxylic acid (604)

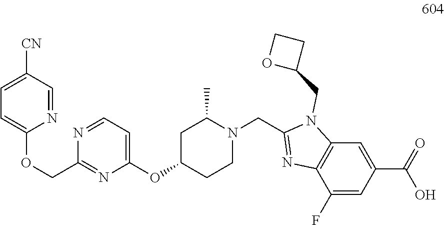 [SMILES: C[C@H]1C[C@@H](Oc2ccnc(COc3ccc(C#N)cn3)n2)CCN1Cc1nc2c(F)cc(C(=O)O)cc2n1C[C@@H]1CCO1]

Example 604 was synthesized by following the synthetic route A-D for Example 606. ¹H NMR (400 MHz, DMSO-d6) δ 8.60 (d, 1H), 8.44 (d, 1H), 8.22 (dd, 1H), 8.12 (d, 1H), 7.51 (dd, 1H), 7.19 (d, 1H), 6.73 (d, 1H), 5.64-5.46 (m, 2H), 5.20-5.08 (m, 1H), 4.81-4.68 (m, 3H), 4.51-4.37 (m, 2H), 4.28-4.19 (m, 1H), 3.58 (d, 1H), 2.73-2.60 (m, 2H), 2.40-2.27 (m, 2H), 2.14 (brt, 1H), 1.77-1.68 (m, 2H), 1.50-1.36 (m, 1H), 1.24 (q, 1H), 1.07 (d, 3H); m/z (ESI, +ve ion)=588.3 [M+H]⁺.

### Example 605. 2-({4-[(2-{[(5-Chloro-3-fluoropyridin-2-yl)oxy]methyl}pyrimidin-4-yl)oxy]piperidin-1-yl}methyl)-4-fluoro-1-{[(2S)-oxetan-2-yl]methyl}-1H-1,3-benzodiazole-6-carboxylic acid (605)

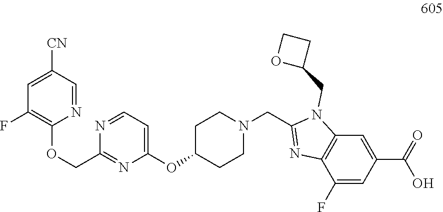

Example 605 was synthesized by following the synthetic route A-D for Example 606. ¹H NMR (400 MHz, DMSO-d6) δ 8.44 (d, 1H), 8.13-8.06 (m, 2H), 7.97 (d, 1H), 7.50 (d, 1H), 6.76 (d, 1H), 5.56 (s, 2H), 5.13-5.02 (m, 1H), 4.86-4.71 (m, 2H), 4.70-4.58 (m, 1H), 4.53-4.45 (m, 1H), 4.36 (td, 1H), 3.94 (d, 1H), 3.79 (d, 1H), 2.80-2.62 (m, 3H), 2.47-2.36 (m, 1H), 2.25-2.12 (m, 2H), 1.75 (brdd, 2H), 1.60-1.45 (m, 2H); m/z (ESI, +ve ion)=601.1 [M+H]⁺.

### Example 606. 2-{[(2S,4S)-4-[(2-{[(3,5-Difluoropyridin-2-yl)oxy]methyl}pyrimidin-4-yl)oxy]-2-methylpiperidin-1-yl]methyl}-4-fluoro-1-[(oxetan-2-yl)methyl]-1H-1,3-benzodiazole-6-carboxylic acid (606)

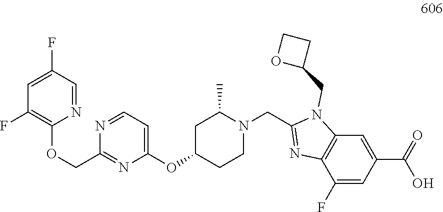 [SMILES: C[C@H]1C[C@@H](Oc2ccnc(COc3ncc(F)cc3F)n2)CCN1Cc1nc2c(F)cc(C(=O)O)cc2n1C[C@@H]1CCO1]

### Step A: Tert-butyl(2S,4S)-4-((2-chloropyrimidin-4-yl)oxy)-2-methylpiperidine-1-carboxylate (606c)

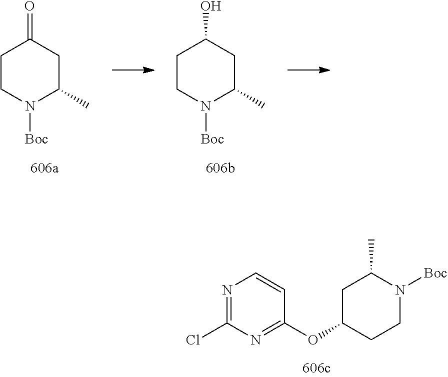 [SMILES: C[C@H]1CC(=O)CCN1C(=O)OC(C)(C)C ; C[C@H]1C[C@@H](O)CCN1C(=O)OC(C)(C)C ; C[C@H]1C[C@@H](Oc2ccnc(Cl)n2)CCN1C(=O)OC(C)(C)C]

To a mixture of TEA (117.19 g, 1.16 mol, 161.20 mL) was added dropwise HCOOH (51.36 g, 1.07 mol) at 25° C. After that N-[(1S,2S)-2-amino-1,2-diphenyl-ethyl]-4-methyl-benzenesulfonamide; chlororuthenium; 1-isopropyl-4-methyl-benzene (1.42 g, 2.23 mmol) was added to the mixture and stirred at 30° C. for 10 min under N₂. Then tert-butyl (2S)-2-methyl-4-oxo-piperidine-1-carboxylate, 606a (95 g, 445.44 mmol) was added to the mixture and stirred at 50° C. for 12H. After completion. The mixture was quenched by addition of H₂O (1500 mL) and citric acid (50%, aq. 500 mL), then extracted with EtOAc (600 mL*3). The combined organic layer was washed with NaHCO₃ (sat. 200 mL*2), dried with Na₂SO₄ and concentrated under reduce pressure to give a residue. The residue was triturated with n-heptane (20V) at 25° C. for 3H and the mixture was filtered to give (2S,4S)-tert-butyl 4-hydroxy-2-methylpiperidine-1-carboxylate, 606b (82 g, 380.88 mmol, 85% yield).

To a solution of 2,4-dichloropyrimidine (94.11 g, 631.71 mmol) and tert-butyl (2S,4S)-4-hydroxy-2-methyl-piperidine-1-carboxylate, 606b (80 g, 371.59 mmol) in DMF (900 mL) was added Cs₂CO₃ (363.22 g, 1.11 mol). The mixture was stirred at 80° C. for 16 hr. After completion. The reaction was quenched with ice H₂O (1500 mL), then extracted with PE (1000 mL*3), the organic layer was dried over Na₂SO₄, concentrated. The residue was purified by column chromatography (SiO₂, Petroleum ether/Ethyl acetate=50/1 to 10/1) to give (2S,4S)-tert-butyl 4-((2-chloropyrimidin-4-yl)oxy)-2-methylpiperidine-1-carboxylate, 606c (63.33 g, 49% yield). m/z (ESI, +ve ion)=328.2 [M+H]⁺.

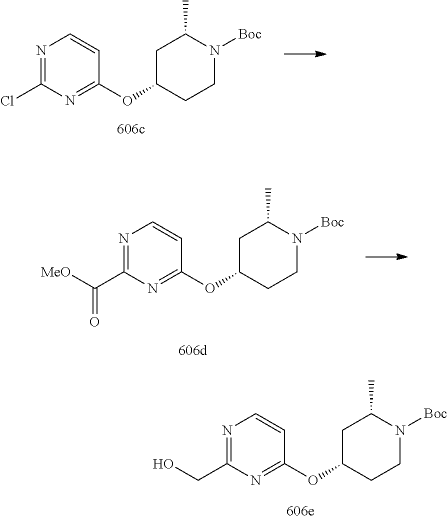 [SMILES: C[C@H]1C[C@@H](Oc2ccnc(Cl)n2)CCN1C(=O)OC(C)(C)C ; COC(=O)c1nccc(O[C@H]2CCN(C(=O)OC(C)(C)C)[C@@H](C)C2)n1 ; C[C@H]1C[C@@H](Oc2ccnc(CO)n2)CCN1C(=O)OC(C)(C)C]

### Step B: tert-butyl(2S,4S)-4-((2-(hydroxymethyl)pyrimidin-4-yl)oxy)-2-methylpiperidine-1-carboxylate (606e)

A mixture of tert-butyl(2S,4S)-4-(2-chloropyrimidin-4-yl)oxy-2-methyl-piperidine-1-carboxylate, 606c (63 g, 192.19 mmol), TEA (25.28 g, 249.84 mmol, 34.77 mL), Pd(dppf)Cl₂ (14.06 g, 19.22 mmol) in MeOH (400 mL) and DMSO (200 mL) was degassed and purged with N₂ for 3 times, and then the mixture was stirred at 60° C. for 16 h under CO (50 psi) atmosphere. After completion. The reaction was concentrated and added H₂O (800 mL), extracted with EtOAc (500 mL*3), the organic layer was dried over Na₂SO₄, concentrated. The residue was purified by column chromatography (SiO₂, Petroleum ether/Ethyl acetate=40/1 to 1/1) to give methyl 4-(((2S,4S)-1-(tert-butoxycarbonyl)-2-methylpiperidin-4-yl)oxy)pyrimidine-2-carboxylate, 606d (35.04 g, 52% yield) as yellow oil. m/z (ESI, +ve ion)=352.3 [M+H]⁺.

A mixture of 606d (25 g, 71.14 mmol), CaCl₂) (4.74 g, 42.69 mmol) in EtOH (300 mL) was degassed and purged with N₂ for 3 times, then NaBH₄ (2.15 g, 56.92 mmol) was added, and then the mixture was stirred at 20° C. for 1 hr under N₂ atmosphere. After completion, the reaction was quenched by addition of MeOH (300 mL) and concentrated under reduce pressure to give a residue. The residue was diluted with sat. aqueous NH₄Cl (100 mL) and EtOAc (300 mL*3). The combined organic layer was washed with brine (100 mL), concentrated under reduce pressure to give a residue. The residue was triturated with PE (10V) at 50° C. until dissolved and then cooled to 25° C. and precipitated to give (2S,4S)-tert-butyl 4-((2-(hydroxymethyl)pyrimidin-4-yl)oxy)-2-methylpiperidine-1-carboxylate, 606e (21.05 g, 85% yield). m/z (ESI, +ve ion)=324.2 [M+H]⁺.

### Step C: 2-(((3,5-difluoropyridin-2-yl)oxy)methyl)-4-(((2S,4S)-2-methylpiperidin-4-yl)oxy)pyrimidine (606f)

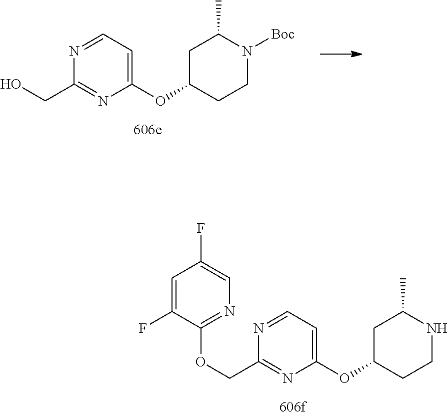 [SMILES: C[C@H]1C[C@@H](Oc2ccnc(CO)n2)CCN1C(=O)OC(C)(C)C ; C[C@H]1C[C@@H](Oc2ccnc(COc3ncc(F)cc3F)n2)CCN1]

To a solution of 606e (150 mg, 463.84 umol), 2,3,5-trifluoropyridine (94.48 mg, 695.76 umol, 98% purity), NaH (74.21 mg, 1.86 mmol, 60% purity) in THF (6 mL) at 0° C. was degassed and purged with N₂ for 3 times, and then the mixture was stirred at 0-50° C. for 3 Hr under N₂ atmosphere. After completion, reaction mixture was quenched with sat. NH₄Cl (5 mL), and then was extracted with EA (10 mL*3), the combined organic layer was washed with brine (10 mL), dried over anhydrous Na₂SO₄, filtered, concentrated and purified by prep-TLC (PE/EA=1:1, Rf=0.43) to afford the desired product (154 mg, 76% yield) as a brown oil. m/z (ESI, +ve ion)=437.3 [M+H]⁺.

To a solution of tert-butyl(2S,4S)-4-[2-[(3,5-difluoro-2-pyridyl)oxymethyl]pyrimidin-4-yl]oxy-2-methyl-piperidine-1-carboxylate (154 mg, 352.85 umol) in TFA (1 mL) and DCM (1 mL). The mixture was stirred at 15° C. for 1 Hr. After completion, reaction mixture was quenched with H₂O (5 mL) and Na₂CO₃, and then was extracted with EA (10 mL*3), the combined organic layer was washed with brine (10 mL), dried over anhydrous Na₂SO₄, filtered and concentrated, without further purification to afford the desired product, 606f (118.6 mg, 100% yield). m/z (ESI, +ve ion)=337.3 [M+H]⁺.

### Step D: Methyl(S)-3-fluoro-4-nitro-5-((oxetan-2-ylmethyl)amino)benzoate (606h)

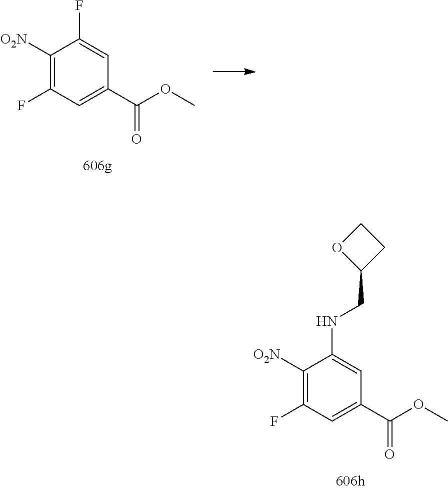 [SMILES: COC(=O)c1cc(F)c([N+](=O)[O-])c(F)c1 ; COC(=O)c1cc(F)c([N+](=O)[O-])c(NC[C@@H]2CCO2)c1]

A mixture of methyl 3,5-difluoro-4-nitro-benzoate, 606g (2 g, 9.21 mmol) [(2S)-oxetan-2-yl]methanamine (1.04 g, 11.97 mmol), DIEA (3.57 g, 27.63 mmol, 4.81 mL) and THF (39.7 mL) and DMF (39.7 mL) was stirred at 50° C. for 4 h. After completion, the mixture was then quenched with ammonium chloride aqueous solution (100 mL) and extracted with ethyl acetate (100 mL*2). The extract was washed with brine, (60 ml) dried over sodium sulfate, filtered, and concentrated. The residue was purified by column chromatography (SiO₂, Petroleum ether/Ethyl acetate=100/1 to 10/1) to afford the desired product, 606h (1.7 g, 65% yield) as a yellow solid. m/z (ESI, +ve ion)=285.0 [M+H]⁺.

### Step E: methyl(S)-2-(chloromethyl)-4-fluoro-1-(oxetan-2-ylmethyl)-1H-benzo[d]imidazole-6-carboxylate (606i)

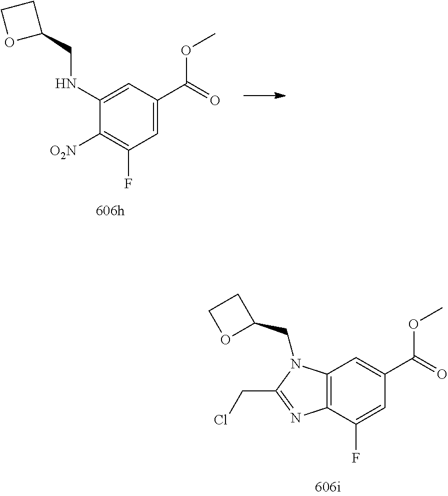 [SMILES: COC(=O)c1cc(F)c([N+](=O)[O-])c(NC[C@@H]2CCO2)c1 ; COC(=O)c1cc(F)c2nc(CCl)n(C[C@@H]3CCO3)c2c1]

To a solution of 606h (1.6 g, 5.63 mmol, 1 eq) in THF (100 mL) was added Pd/C (1.6 g, 10% purity) under Ar₂ atmosphere. The suspension was degassed and purged with H₂ for 3 times. The mixture was stirred under H₂ (15 Psi) at 25° C. for 2 Hr. After completion, the reaction mixture was filtered and concentrated to give the di-amine intermediate (1.5 g, crude). m/z (ESI, +ve ion)=255.0 [M+H]⁺.

A mixture of crude methyl 4-amino-3-fluoro-5-[[(2S)-oxetan-2-yl]methylamino]benzoate (2.6 g, 10.23 mmol), ACN (32 mL), 2-chloro-1,1,1-trimethoxy-ethane (1.58 g, 10.23 mmol, 1.37 mL) and PTSA (88.04 mg, 511.29 umol) was stirred at 60° C. for 3H. After completion, the reaction mixture was concentrated. The residue was diluted with H₂O (50 mL) and extracted with EA (50 mL×3). The combined organic layers were dried over anhydrous sodium sulfate and concentrated under reduced pressure to give crude product. The residue was purified by column chromatography (SiO₂, Petroleum ether/Ethyl acetate=20/1 to 5/1) to afford the desired product, 606i (1.8 g, 56% yield). 1H NMR (400 MHz, DMSO-d6) δ=8.25 (s, 1H), 7.61 (d, 1H), 5.20-5.11 (m, 3H), 4.79-4.77 (m, 1H), 4.70-4.69 (m, 1H), 4.48-4.47 (m, 1H), 4.34-4.32 (m, 1H), 3.90 (s, 3H), 2.72-2.68 (m, 1H), 2.40-2.35 (m, 1H). m/z (ESI, +ve ion)=172.2 [M+H]⁺.

### Step F: 2-(((2S,4S)-4-((2-(((3,5-difluoropyridin-2-yl)oxy)methyl)pyrimidin-4-yl)oxy)-2-methylpiperidin-1-yl)methyl)-4-fluoro-1-(((S)-oxetan-2-yl)methyl)-1H-benzo[d]imidazole-6-carboxylic acid (606)

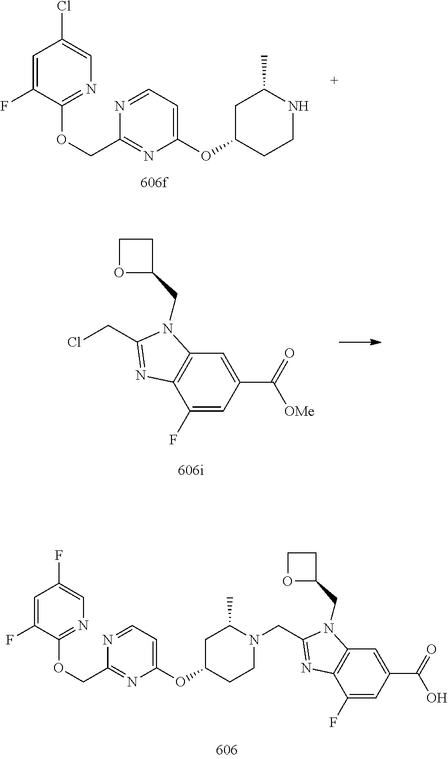 [SMILES: C[C@H]1C[C@@H](Oc2ccnc(COc3ncc(Cl)cc3F)n2)CCN1 ; COC(=O)c1cc(F)c2nc(CCl)n(C[C@@H]3CCO3)c2c1 ; C[C@H]1C[C@@H](Oc2ccnc(COc3ncc(F)cc3F)n2)CCN1Cc1nc2c(F)cc(C(=O)O)cc2n1C[C@@H]1CCO1]

To a solution of 2-[(3,5-difluoro-2-pyridyl)oxymethyl]-4-[[(2S,4S)-2-methyl-4-piperidyl]oxy]pyrimidine, 606f (118.6 mg, 352.62 umol), methyl 2-(chloromethyl)-7-fluoro-3-[[(2S)-oxetan-2-yl]methyl]benzimidazole-5-carboxylate, 606i (110.34 mg, 352.62 umol), DIEA (228.01 mg, 1.76 mmol) in DMF (5 mL) was degassed and purged with N₂ for 3 times, and then the mixture was stirred at 80° C. for 3 Hr under N₂ atmosphere. After completion, reaction mixture was quenched with H₂O (5 mL), and then was extracted with EA (10 mL*3), the combined organic layer was washed with brine (10 mL), dried over anhydrous Na₂SO₄, filtered, concentrated and purified by prep-TLC (PE/EA=0:1, Rf=0.35) to afford the corresponding ester (170 mg, 79% yield). m/z (ESI, +ve ion)=613.2 [M+H]⁺.

To a solution of methyl 2-[[(2S,4S)-4-[2-[(3,5-difluoro-2-pyridyl)oxymethyl]pyrimidin-4-yl]oxy-2-methyl-1-piperidyl]methyl]-7-fluoro-3-[[(2S)-oxetan-2-yl]methyl]benzimidazole-5-carboxylate (170 mg, 277.51 umol), LiOH·H₂O (1.16 mg, 27.75 umol) in THF (0.6 mL), MeOH (0.3 mL), H₂O (0.3 mL) was degassed and purged with N₂ for 3 times, and then the mixture was stirred at 15° C. for 16 hr under N₂ atmosphere. After completion, reaction mixture was adjusted to pH-5 with 1M HCl, extracted with EA (50 mL*3), the combined organic phase was washed with brine (100 mL*2), dried with anhydrous Na₂SO₄, filtered, concentrated in vacuum and purified by prep-TLC (PE/EA=0:1, Rf=0.40) and prep-HPLC to afford the desired product, 606 (48 mg, 29% yield). ¹H NMR (400 MHz, CDCl₃) δ 1.22 (brd, 3H) 1.40-1.64 (m, 2H) 1.89 (brd, 1H) 2.01 (brd, 1H) 2.26 (brt, 1H) 2.35-2.49 (m, 2H) 2.67-2.83 (m, 2H) 3.67 (brd, 1H) 4.30 (dt, 1H) 4.50-4.67 (m, 3H) 4.91-5.03 (m, 2H) 5.22-5.29 (m, 1H) 5.58 (s, 2H) 6.53 (d, 1H) 7.28-7.32 (m, 1H) 7.70-7.77 (m, 2H) 8.04 (s, 1H) 8.40 (d, 1H); m/z (ESI, +ve ion)=599.2 [M+H]⁺.

### Example 607. 2-{[(2S,4S)-4-[(2-{[(5-Cyano-3-fluoropyridin-2-yl)oxy]methyl}pyrimidin-4-yl)oxy]-2-methylpiperidin-1-yl]methyl}-4-fluoro-1-[(oxetan-2-yl)methyl]-1H-1,3-benzodiazole-6-carboxylic acid (607)

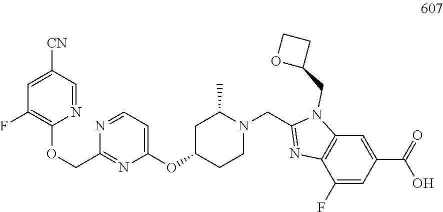 [SMILES: C[C@H]1C[C@@H](Oc2ccnc(COc3ncc(C#N)cc3F)n2)CCN1Cc1nc2c(F)cc(C(=O)O)cc2n1C[C@@H]1CCO1]

Example 607 was synthesized by following the synthetic route A-D for Example 606. ¹H NMR (400 MHz, CDCl₃) δ ppm 1.23 (brd, 3H) 1.47-1.58 (m, 3H) 1.90 (brd, 1H) 2.01 (brd, 1H) 2.23-2.32 (m, 1H) 2.36-2.50 (m, 2H) 2.67-2.83 (m, 2H) 3.68 (brd, 1H) 4.29 (dt, 1H) 4.48-4.67 (m, 3H) 4.90-5.04 (m, 2H) 5.20-5.31 (m, 1H) 5.64 (d, 2H) 6.55 (d, 1H) 7.65 (brd, 1H) 7.72 (d, 1H) 8.03 (s, 1H) 8.19 (d, 1H) 8.38 (d, 1H); m/z (ESI, +ve ion)=606.2 [M+H]⁺.

### Example 608. 2-{[(2S,4S)-4-[(2-{[(5-Chloro-3-fluoropyridin-2-yl)oxy]methyl}pyrimidin-4-yl)oxy]-2-ethylpiperidin-1-yl]methyl}-1-[(oxetan-2-yl)methyl]-1H-1,3-benzodiazole-6-carboxylic acid

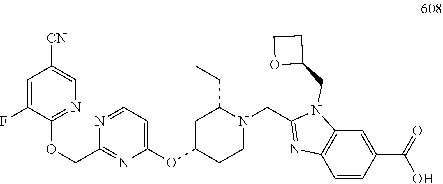

### Example 609. 2-{[(2R,4R)-4-[(2-{[(5-Chloro-3-fluoropyridin-2-yl)oxy]methyl}pyrimidin-4-yl)oxy]-2-ethylpiperidin-1-yl]methyl}-1-[(oxetan-2-yl)methyl]-1H-1,3-benzodiazole-6-carboxylic acid

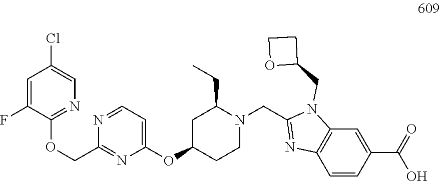 [SMILES: CC[C@@H]1C[C@H](Oc2ccnc(COc3ncc(Cl)cc3F)n2)CCN1Cc1nc2ccc(C(=O)O)cc2n1C[C@@H]1CCO1]

Examples 608 and 609 (stereoisomers) were synthesized using a similar procedure as Example 599 followed by chiral separation by SFC.

### Examples 610 and 611. 2-(((2S,4S)-4-((2-((2,4-difluorophenoxy)methyl)pyrimidin-4-yl)oxy)-2-methylpiperidin-1-yl)methyl)-1-(((R)-1,1-dioxidothietan-2-yl)methyl)-1H-benzo[d]imidazole-6-carboxylic acid (610) and 2-(((2S,4S)-4-((2-((2,4-difluorophenoxy)methyl)pyrimidin-4-yl)oxy)-2-methylpiperidin-1-yl)methyl)-1-(((S)-1,1-dioxidothietan-2-yl)methyl)-1H-benzo[d]imidazole-6-carboxylic acid (611)

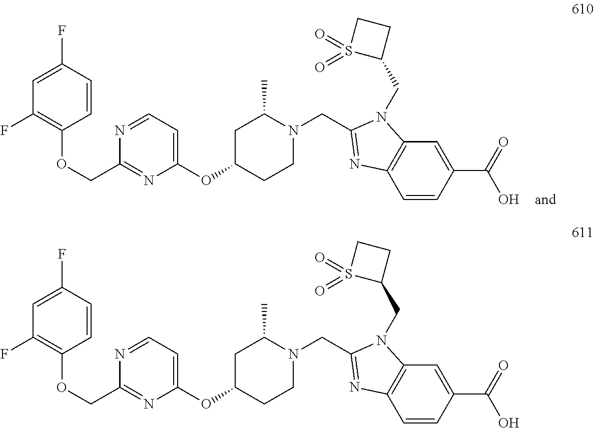 [SMILES: C[C@H]1C[C@@H](Oc2ccnc(COc3ccc(F)cc3F)n2)CCN1Cc1nc2ccc(C(=O)O)cc2n1C[C@H]1CCS1(=O)=O ; C[C@H]1C[C@@H](Oc2ccnc(COc3ccc(F)cc3F)n2)CCN1Cc1nc2ccc(C(=O)O)cc2n1C[C@@H]1CCS1(=O)=O]

### Step A: tert-Butyl (2,4-dihydroxybutyl)carbamate (610b)

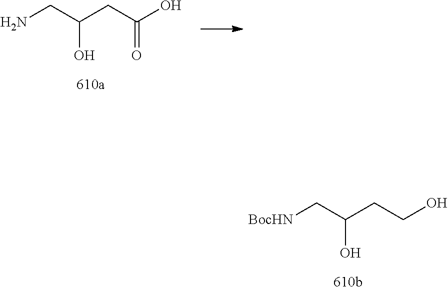 [SMILES: NCC(O)CC(=O)O ; CC(C)(C)OC(=O)NCC(O)CCO]

To a solution of 610a (1 g, 8.39 mmol) in H₂O (15 mL) was added NaOH (738.70 mg, 18.47 mmol, 35.09 mL) and (Boc) 20 (2.38 g, 10.91 mmol, 2.51 mL) and dioxane (2.5 mL). The mixture was stirred at 15° C. for 72H. After completion, the reaction was added HCl (1N) till pH=2-3, then extracted with DCM (80 mL*3), the organic layer was dried over Na₂SO₄, filtered and concentrated in vacuum. The residue was purified by column chromatography (SiO₂, Petroleum ether/Ethyl acetate=20/1 to 0/1) to afford Boc-protected compound (1.5 g, 81% yield) as a white oil.

To a solution of Boc-protected compound (12 g, 54.74 mmol) in THF (150 mL) was added BH₃·THF (1 M, 147.79 mL) at 0° C. under N₂ atmosphere. The mixture was stirred at 0° C. for 2H. After completion, the mixture was quenched with 10% AcOH in MeOH (100 ml). Then EtOAc (160 mL*3) was added, and the reaction mixture was washed with sat. NaHCO₃ (100 mL), the organic layer was dried over Na₂SO₄. The filtrate was concentrated to afford 610b (5.6 g, 49.8% yield) as a yellow oil.

### Step B: tert-butyl (thietan-2-ylmethyl)carbamate (610c)

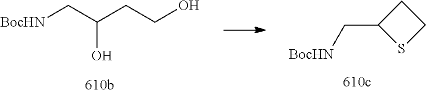 [SMILES: CC(C)(C)OC(=O)NCC(O)CCO ; CC(C)(C)OC(=O)NCC1CCS1]

To a solution of 610b (5.6 g, 27.28 mmol) in DCM (120 mL) was added TEA (16.56 g, 163.70 mmol) and MsCl (7.19 g, 62.75 mmol) at 0° C. under N₂ atmosphere. The mixture was stirred at 0° C. for 2H. After completion, the reaction mixture was quenched by the addition of the sat. NaHCO₃ (200 mL) at 15° C., and then extracted with DCM (200 mL*3). The combined organic layers were washed with sat. NH₄Cl (200 mL*2), dried over Na₂SO₄, filtered and concentrated under reduced pressure to give a residue. The filtrate was concentrated to afford di mesylated compound (9.17 g, 92.99% yield). To a solution of di mesylated compound (9.17 g, 25.37 mmol) in MeOH (900 mL) was added Na₂S·9H₂O (30.47 g, 126.86 mmol) under N₂ atmosphere. The mixture was stirred at 40° C. for 16 h. After completion, the mixture was extracted with DCM (20 mL*3), dried with anhydrous Na₂SO₄, filtered and concentrated in vacuum. The filtrate was concentrated to afford 610c (3.74 g, 72.5% yield) as a white oil.

### Step C: 2-(Aminomethyl)thietane 1,1-dioxide (610d)

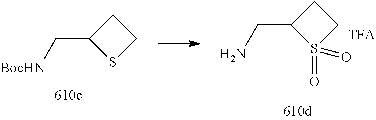 [SMILES: CC(C)(C)OC(=O)NCC1CCS1 ; NCC1CCS1(=O)=O]

To a solution of 610c (3.34 g, 16.42 mmol) in DCM (60 mL) was added m-CPBA (10.00 g, 49.28 mmol, 85% purity) under N₂ atmosphere. The mixture was stirred at 15° C. for 16 h. After completion, the mixture was washed with the sat. NaHCO₃ (60 ml) and extracted with EtOAc (60 mL×3). The combined organic layer was dried over Na₂SO₄ and filtered. The filtrate was concentrated under reduced pressure to give sulfone (2.1 g, 51.32% yield) as a white oil.

To a solution of sulfone (2.1 g, 8.92 mmol) in DCM (10 mL) was added TFA (15.40 g, 135.06 mmol) under N₂ atmosphere. The mixture was stirred at 15° C. for 4 hr. After completion, the reaction mixture was concentrated under reduced pressure to remove solvent. The filtrate was concentrated to afford amine (2.22 g, crude) as a white oil.

### Final Steps:

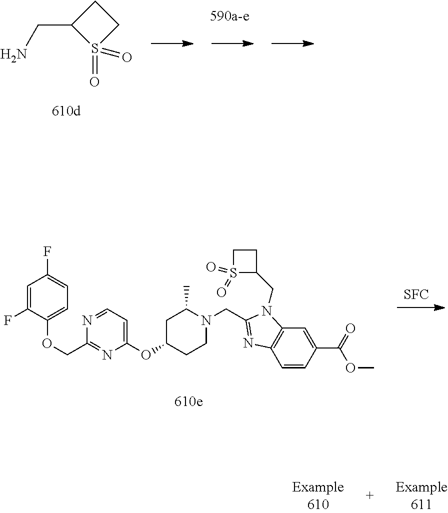 [SMILES: NCC1CCS1(=O)=O ; COC(=O)c1ccc2nc(CN3CC[C@H](Oc4ccnc(COc5ccc(F)cc5F)n4)C[C@@H]3C)n(CC3CCS3(=O)=O)c2c1 ; "SFC"]

Compound 610e was synthesized from amine 610d by following the similar procedure as 590a-e.

To a solution of 610e (0.09 g, 140.26 umol) in THF (2.5 mL) MeOH (1.25 mL) and H₂O (1.25 mL) was added LiOH·H₂O (29.43 mg, 701.30 umol). The mixture was stirred at 20° C. for 3H. After completion, the reaction solution was added 1N HCl (701.30 umol), diluted with H₂O (5 mL) and extracted with DCM/i-PrOH (3/1) (30 mL×3). The combine organic layer was washed with brine (30 mL×3), dried over Na₂SO₄, concentrated and the product was purified by SFC (column: DAICEL CHIRALCEL OD (250 mm*30 mm, 10 um); mobile phase: [0.1% NH₃/H₂O/IPA]; B %: 45%-45%, 17 min, P1: Rt=1.481 min, P2: Rt=1.656 min) to afford the desired products.

Example 610: ¹H NMR (400 MHz, CD₃OD) δ ppm 8.39-8.44 (m, 2H), 8.00 (dd, 1H), 7.69 (d, 1H), 6.99-7.11 (m, 2H), 6.79-6.86 (m, 1H), 6.71 (d, 1H), 5.25 (s, 2H), 5.14-5.23 (m, 2H), 4.98-5.10 (m, 1H), 4.65-4.81 (m, 2H), 4.10-4.22 (m, 1H), 3.97-4.08 (m, 1H), 3.45-3.52 (m, 1H), 2.65-2.73 (m, 1H), 2.38-2.50 (m, 2H), 2.15-2.26 (m, 1H), 2.04-2.13 (m, 2H), 1.89-2.00 (m, 1H), 1.46-1.63 (m, 2H), 1.31-1.36 (m, 3H); m/z (ESI, +ve ion)=628.2 [M+H]⁺.

Example 611: ¹H NMR (400 MHz, CD₃OD) δ ppm 8.43 (d, 1H), 8.39 (s, 1H), 8.01 (dd, 1H), 7.70 (d, 1H), 6.99-7.13 (m, 2H), 6.80-6.88 (m, 1H), 6.73 (d, 1H), 5.26 (s, 2H), 5.02-5.14 (m, 3H), 4.98 (t, 1H), 4.53 (d, 1H), 4.12-4.24 (m, 1H), 4.01-4.12 (m, 1H), 3.65 (d, 1H), 2.80 (brd, 1H), 2.40-2.56 (m, 2H), 2.27 (brt, 1H), 2.10-2.19 (m, 1H), 2.01-2.08 (m, 1H), 1.92-1.99 (m, 1H), 1.57-1.74 (m, 2H), 1.29 (d, 3H); m/z (ESI, +ve ion)=628.2 [M+H]⁺.

### Example 612. 1-((S)-2-cyclopropoxypropyl)-2-(((2S,4S)-4-((2-((2,4-difluorophenoxy)methyl)pyrimidin-4-yl)oxy)-2-methylpiperidin-1-yl)methyl)-1H-benzo[d]imidazole-6-carboxylic acid (612)

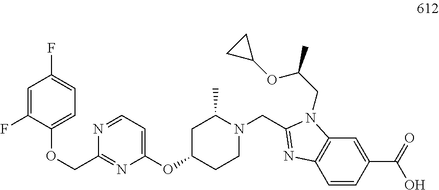 [SMILES: C[C@@H](Cn1c(CN2CC[C@H](Oc3ccnc(COc4ccc(F)cc4F)n3)C[C@@H]2C)nc2ccc(C(=O)O)cc21)OC1CC1]

### Step A: (S)—N,N-dibenzyl-2-(vinyloxy) propan-1-amine (612b)

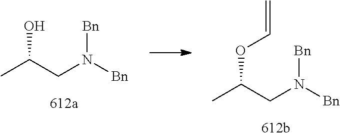 [SMILES: C[C@H](O)CN(Cc1ccccc1)Cc1ccccc1 ; C=CO[C@@H](C)CN(Cc1ccccc1)Cc1ccccc1]

To a solution of 612a (7 g, 27.41 mmol) in 1-vinyloxybutane (41.18 g, 411.19 mmol) was added 4,7-diphenyl-1,10-phenanthroline (455.60 mg, 1.37 mmol) and Pd(OAc) 2 (307.72 mg, 1.37 mmol) and TEA (1.11 g, 10.97 mmol). The mixture was degassed and purged with N₂ for 3 times, and then the mixture was stirred at 100° C. for 16 h under N₂ atmosphere. After completion, the reaction was filtered and concentrated. 20 mL of H₂O was added, and then was extracted with EtOAc (10 mL×3), the combined organic layer was washed with brine (10 mL), dried over anhydrous Na₂SO₄, filtered, concentrated and purified by silica gel column (PE:EA=10:1) to afford the desired product 612b (4.6 g, 59.63% yield) as a colorless oil. m/z (ESI, +ve ion)=282.4 [M+H]⁺.

### Step B: (S)—N,N-dibenzyl-2-cyclopropoxypropan-1-amine (612c)

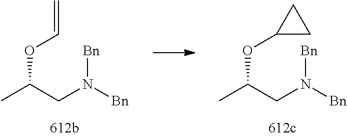 [SMILES: C=CO[C@@H](C)CN(Cc1ccccc1)Cc1ccccc1 ; C[C@@H](CN(Cc1ccccc1)Cc1ccccc1)OC1CC1]

To a solution of CH₂I₂ (71.39 g, 266.53 mmol) in DCM (60 mL) was cooled to −65° C., and ZnEt₂ (1 M, 266.53 mL) was added dropwise via syringe. The solution was degassed and purged with N₂ for 3 times. To this solution was added 612b (5 g, 17.77 mmol) in DCM (30 mL) dropwise via syringe, and the reaction mixture was stirred for 16 h at 15° C. under N₂ atmosphere. After completion, the reaction was quenched with saturated NH₄Cl solution (20 mL), and then was extracted with EtOAc (10 mL×3), the combined organic layer was washed with brine (10 mL), dried over anhydrous Na₂SO₄, filtered and concentrated and purified by prep-HPLC (column: Welch Xtimate C18 250*70 mm #10 μm; mobile phase: [water (NH₄HCO₃)-ACN]; B %: 65%-95%, 20 min) to afford the desired product 612c (1.35 g, 25.72% yield) as a colorless oil. m/z (ESI, +ve ion)=296.2 [M+H]⁺.

### Step C: (S)-2-cyclopropoxypropan-1-amine (612d)

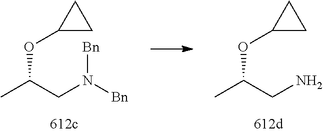

To a solution of Pd/C (3.23 g, 50% w/w) in MeOH (10 mL) was added Compound 612c (900 mg, 3.05 mmol), KOAc (298.99 mg, 3.05 mmol). The solution was degassed and purged with H₂ (15 Psi) for 3 times, and then the mixture was stirred at 30° C. for 16 h under H₂ atmosphere. After completion, the mixture was filtered and concentrated in vacuum, without further purification to afford the crude product 612d (350 mg, 99.75% yield) as a colorless oil.

### Step d: 1-((S)-2-cyclopropoxypropyl)-2-(((2S,4S)-4-((2-((2,4-difluorophenoxy)methyl)pyrimidin-4-yl)oxy)-2-methylpiperidin-1-yl)methyl)-1H-benzo[d]imidazole-6-carboxylic acid (612)

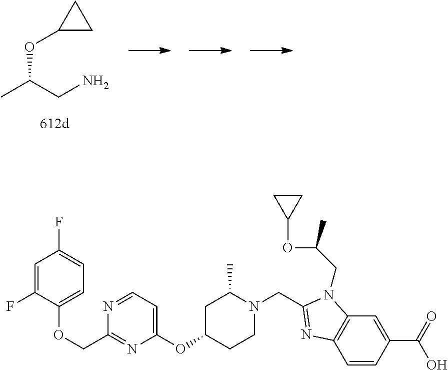

Example 612 was synthesized from 612d using the same synthetic procedure as Example 588. ¹H NMR (400 MHz, CDCl₃) δ ppm 8.43 (d, 1H), 8.21 (s, 1H), 8.03 (d, 1H), 7.78 (d, 1H), 6.98-6.85 (m, 2H), 6.77-6.70 (m, 1H), 6.58 (d, 1H), 5.23 (s, 2H), 5.13-5.00 (m, 1H), 4.59-4.52 (m, 1H), 4.44-4.30 (m, 2H), 4.04-3.96 (m, 1H), 3.69 (brd, 1H), 3.18 (brd, 1H), 2.87-2.79 (m, 1H), 2.57-2.48 (m, 1H), 2.38-2.24 (m, 1H), 2.07-1.90 (m, 3H), 1.32 (d, 3H), 1.26 (s, 1H), 1.24-1.18 (m, 3H), 0.46-0.38 (m, 2H), 0.27-0.19 (m, 1H), −0.03-−0.13 (m, 1H); m/z (ESI, +ve ion)=608.2 [M+H]⁺.

### Example 613. (S)-2-((4-((2-(((5-Chloro-3-fluoropyridin-2-yl)oxy)methyl)pyridin-4-yl)oxy)piperidin-1-yl)methyl)-4-fluoro-1-(oxetan-2-ylmethyl)-1H-benzo[d]imidazole-6-carboxylic acid (613)

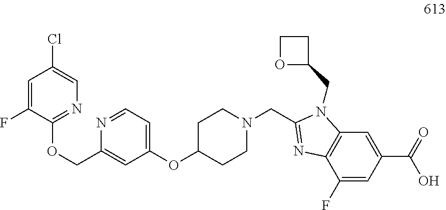 [SMILES: O=C(O)c1cc(F)c2nc(CN3CCC(Oc4ccnc(COc5ncc(Cl)cc5F)c4)CC3)n(C[C@@H]3CCO3)c2c1]

Example 613 was synthesized by following the synthetic route A-D for Example 554. ¹H NMR (400 MHz, DMSO-d6) δ 13.07 (brs, 1H), 8.34 (d, 1H), 8.16 (d, 1H), 8.12-8.04 (m, 2H), 7.50 (dd, 1H), 7.03-6.92 (m, 2H), 5.41 (s, 2H), 5.14-5.01 (m, 1H), 4.88-4.75 (m, 1H), 4.71-4.62 (m, 1H), 4.61-4.52 (m, 1H), 4.52-4.46 (m, 1H), 4.40-4.32 (m, 1H), 3.96 (d, 1H), 3.81 (d, 1H), 2.83-2.65 (m, 3H), 2.46-2.33 (m, 3H), 2.01-1.90 (m, 2H), 1.71-1.55 (m, 2H); m/z (ESI, +ve ion)=600.0 [M+H]⁺.

### Example 614. 2-(((2S,4S)-4-((2-((2,4-Difluorobenzyl)amino)pyrimidin-4-yl)oxy)-2-methylpiperidin-1-yl)methyl)-1-(((S)-oxetan-2-yl)methyl)-1H-benzo[d]imidazole-6-carboxylic acid (614)

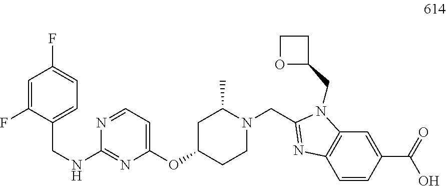 [SMILES: C[C@H]1C[C@@H](Oc2ccnc(NCc3ccc(F)cc3F)n2)CCN1Cc1nc2ccc(C(=O)O)cc2n1C[C@@H]1CCO1]

### Step A: tert-Butyl(2S,4S)-4-((2-chloropyrimidin-4-yl)oxy)-2-methylpiperidine-1-carboxylate (614b)

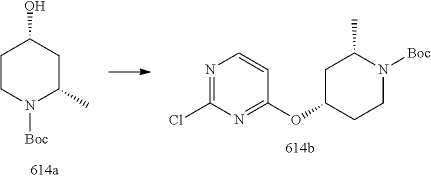 [SMILES: C[C@H]1C[C@@H](O)CCN1C(=O)OC(C)(C)C ; C[C@H]1C[C@@H](Oc2ccnc(Cl)n2)CCN1C(=O)OC(C)(C)C]

A mixture of 614a (1.5 g, 6.97 mmol) in DMF (2 mL) was added Cs₂CO₃ (6.81 g, 20.90 mmol) and 2,4-dichloropyrimidine (2.08 g, 13.93 mmol). The mixture was stirred at 80° C. for 12H. After completion, the residue was poured water (100 mL). The aqueous phase was extracted with ethyl acetate (100 mL*3). The combined organic phase was washed with brine (200 mL*2), dried with anhydrous Na₂SO₄, filtered and concentrated in vacuum. The resulting residue was purified by column chromatography (SiO₂, Petroleum ether/Ethyl acetate=40/1 to 0/1)) to afford the desired product 614b (1.05 g, 45.97% yield). m/z (ESI, +ve ion)=328.2 [M+H]⁺.

### Step B: tert-Butyl(2S,4S)-4-((2-((2,4-difluorobenzyl)amino)pyrimidin-4-yl)oxy)-2-methylpiperidine-1-carboxylate (614c)

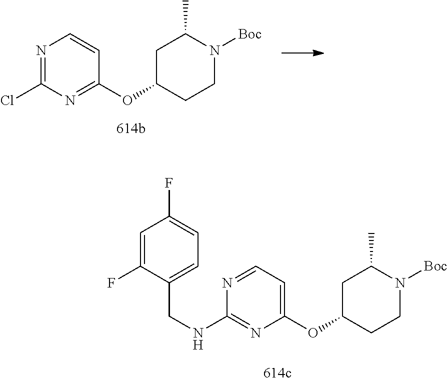 [SMILES: C[C@H]1C[C@@H](Oc2ccnc(Cl)n2)CCN1C(=O)OC(C)(C)C ; C[C@H]1C[C@@H](Oc2ccnc(NCc3ccc(F)cc3F)n2)CCN1C(=O)OC(C)(C)C]

A mixture of 614b (100 mg, 305.06 umol), (2,4-difluorophenyl) methanamine (218.32 mg, 1.53 mmol), BINAP (19.00 mg, 30.51 umol) and Cs₂CO₃ (198.79 mg, 610.12 umol) in toluene (2 mL) was degassed and purged with N₂ for 3 times, then Pd₂(dba)₃ (13.97 mg, 15.25 umol) was added and degassed and purged with N₂ for 3 times, BINAP (19.00 mg, 30.51 umol) and then the mixture was stirred at 90° C. for 16 h under N₂ atmosphere. After completion, the residue was poured into water (30 mL). The aqueous phase was extracted with ethyl acetate (50 mL*3). The organic layer was dried over Na₂SO₄ and concentrated to afford the crude product 614c (25 mg, 18.86% yield) as a colorless oil. m/z (ESI, +ve ion)=328.2 [M+H]⁺.

### Step D: Methyl 2-(((2S,4S)-4-((2-((2,4-difluorobenzyl)amino)pyrimidin-4-yl)oxy)-2-methylpiperidin-1-yl)methyl)-1-(((S)-oxetan-2-yl)methyl)-1H-benzo[d]imidazole-6-carboxylate (614d)

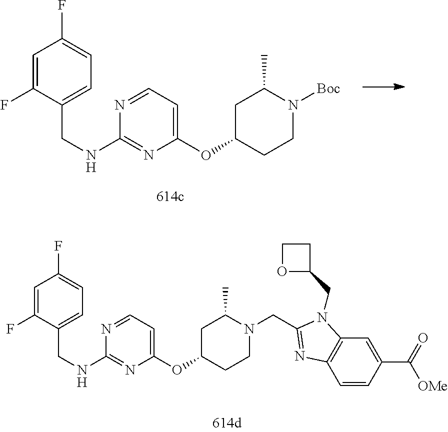 [SMILES: C[C@H]1C[C@@H](Oc2ccnc(NCc3ccc(F)cc3F)n2)CCN1C(=O)OC(C)(C)C ; COC(=O)c1ccc2nc(CN3CC[C@H](Oc4ccnc(NCc5ccc(F)cc5F)n4)C[C@@H]3C)n(C[C@@H]3CCO3)c2c1]

To a solution of 614c (100 mg, 230.16 umol) in TFA (0.4 mL) and DCM (1.2 mL). The mixture was stirred at 27° C. for 1H. The mixture was adjusted to pH=7 with saturated sodium bicarbonate solution water solution, extracted with DCM (20 mL*3), the organic layer was dried over Na₂SO₄ and concentrated to afford the crude amine (75 mg, 97.46% yield) as a colorless oil. m/z (ESI, +ve ion)=335.3 [M+H]⁺.

A mixture of amine (67.87 mg, 230.29 umol) and N-[(2,4-difluorophenyl)methyl]-4-[[(2S,4S)-2-methyl-4-piperidyl]oxy]pyrimidin-2-amine (77 mg, 230.29 umol), DIEA (148.82 mg, 1.15 mmol) in DMF (1 mL) was degassed and purged with N₂ for 3 times, and then the mixture was stirred at 85° C. for 5 h under N₂ atmosphere. After completion, the residue was poured into water (30 mL). The aqueous phase was extracted with ethyl acetate (20 mL*3). dried over Na₂SO₄, concentrated and purified by silica gel column (PE:EA=1:1) to afford the desired product 614d (110 mg, 80.60% yield) as a yellow solid, m/z (ESI, +ve ion)=593.4 [M+H]⁺.

### Step E: 2-(((2S,4S)-4-((2-((2,4-Difluorobenzyl)amino)pyrimidin-4-yl)oxy)-2-methylpiperidin-1-yl)methyl)-1-(((S)-oxetan-2-yl)methyl)-1H-benzo[d]imidazole-6-carboxylic acid (614)

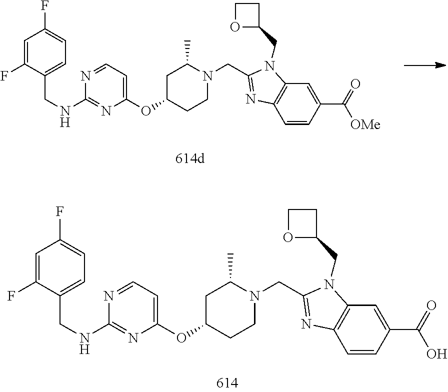 [SMILES: COC(=O)c1ccc2nc(CN3CC[C@H](Oc4ccnc(NCc5ccc(F)cc5F)n4)C[C@@H]3C)n(C[C@@H]3CCO3)c2c1 ; C[C@H]1C[C@@H](Oc2ccnc(NCc3ccc(F)cc3F)n2)CCN1Cc1nc2ccc(C(=O)O)cc2n1C[C@@H]1CCO1]

To a solution of 614d (110 mg, 185.61 umol) in THF (0.4 mL) MeOH (0.2 mL) and H₂O (0.2 mL) was added LiOH·H₂O (38.94 mg, 928.06 umol). The mixture was stirred at 20° C. for 16 h. After completion, the mixture was adjusted to pH=7 with HCl solution, extracted with DCM:IPA (3:1) (20ML*3), dried over Na₂SO₄, filtrated and concentrated in vacuum. The resulting residue was purified by prep-TLC (PE/EA=1:1) to afford the desired product 614 (24.75 mg, 23.05% yield). ¹H NMR (400 MHz, CD₃OD) δ ppm 1.19 (brd, 3H) 1.36-1.58 (m, 2H) 1.96 (brs, 2H) 2.23 (brt, 1H) 2.40-2.51 (m, 2H) 2.69-2.80 (m, 2H) 3.56 (d, 1H) 4.29 (dt, 1H) 4.47-4.64 (m, 4H) 4.49 (s, 1H) 4.71 (dd, 1H) 4.93 (brdd, 2H) 5.24-5.33 (m, 1H) 5.97 (d, 1H) 6.82-6.98 (m, 2H) 7.29-7.40 (m, 1H) 7.65 (d, 1H) 7.88-8.02 (m, 2H) 8.25 (s, 1H); m/z (ESI, +ve ion)=579.2 [M+H]⁺.

### Example 615. 2-(((2S,4S)-4-((2-(((5-Chloro-3-fluoropyridin-2-yl)oxy)methyl)pyridin-4-yl)oxy)-2-methylpiperidin-1-yl)methyl)-1-(((S)-oxetan-2-yl)methyl)-1H-benzo[d]imidazole-6-carboxylic acid (615)

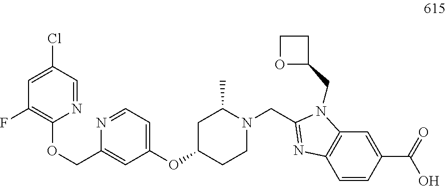 [SMILES: C[C@H]1C[C@@H](Oc2ccnc(COc3ncc(Cl)cc3F)c2)CCN1Cc1nc2ccc(C(=O)O)cc2n1C[C@@H]1CCO1]

Example 615 was synthesized by following the synthetic route A-D for Example 554. ¹H NMR (400 MHz, DMSO-d6) δ ppm 12.88-12.67 (m, 1H), 8.35 (brd, 1H), 8.27 (brs, 1H), 8.08 (dt, 2H), 7.85-7.78 (m, 1H), 7.70-7.62 (m, 1H), 7.04-6.95 (m, 2H), 5.41 (s, 2H), 5.16-5.08 (m, 1H), 4.80-4.70 (m, 2H), 4.61-4.35 (m, 3H), 4.30-4.21 (m, 1H), 3.68-3.52 (m, 1H), 2.81-2.61 (m, 2H), 2.41-2.27 (m, 2H), 2.17-1.80 (m, 3H), 1.52-1.30 (m, 2H), 1.26-1.11 (m, 3H); m/z (ESI, +ve ion)=596.2 [M+H]⁺.

### Example 616. 2-(((2S,4S)-4-((2-(((5-Chloropyridin-2-yl)oxy)methyl)pyridin-4-yl)oxy)-2-methylpiperidin-1-yl)methyl)-4-fluoro-1-(((S)-oxetan-2-yl)methyl)-1H-benzo[d]imidazole-6-carboxylic acid (616)

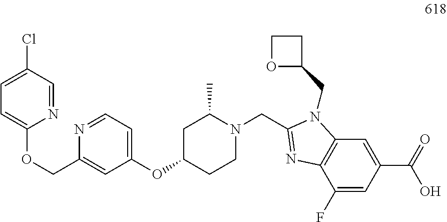 [SMILES: C[C@H]1C[C@@H](Oc2ccnc(COc3ccc(Cl)cn3)c2)CCN1Cc1nc2c(F)cc(C(=O)O)cc2n1C[C@@H]1CCO1]

Example 616 was synthesized by following the synthetic route A-D for Example 606. ¹H NMR (400 MHz, CD₃OD) δ=8.30 (d, 1H), 8.17 (s, 1H), 8.09 (d, 1H), 7.72 (dd, 1H), 7.64 (d, 1H), 7.05 (d, 1H), 6.97-6.90 (m, 2H), 5.38 (s, 2H), 5.32-5.24 (m, 1H), 4.96 (brd, 1H), 4.75 (dd, 1H), 4.66-4.51 (m, 3H), 4.32 (td, 1H), 3.67 (d, 1H), 2.90-2.81 (m, 1H), 2.80-2.70 (m, 1H), 2.69-2.59 (m, 1H), 2.51-2.35 (m, 2H), 2.11 (brd, 1H), 2.07-1.98 (m, 1H), 1.68-1.49 (m, 2H), 1.25 (d, 3H); m/z (ESI, +ve ion)=629.1 [M+H]⁺.

### Example 617. 1-((R)-2-(Difluoromethoxy)propyl)-2-(((2S,4S)-4-((2-(((3,5-difluoropyridin-2-yl)oxy)methyl)pyrimidin-4-yl)oxy)-2-methylpiperidin-1-yl)methyl)-1H-benzo[d]imidazole-6-carboxylic acid (617)

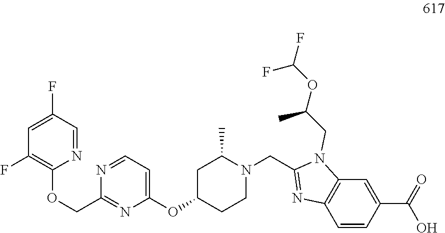 [SMILES: C[C@H](Cn1c(CN2CC[C@H](Oc3ccnc(COc4ncc(F)cc4F)n3)C[C@@H]2C)nc2ccc(C(=O)O)cc21)OC(F)F]

Example 617 was synthesized by following the synthetic route A-D for Example 554 using 591d. ¹H NMR (400 MHz, DMSO-d6) δ 12.79 (brs, 1H), 8.45 (d, 1H), 8.23 (s, 1H) 8.04 (ddd, 1H), 7.96 (d, 1H), 7.80 (dd, 1H), 7.64 (d, 1H), 6.74 (d, 1H), 6.24-6.65 (m, 1H), 5.50-5.60 (m, 2H), 4.70-4.84 (m, 2H), 4.57-4.68 (m, 1H), 4.40-4.55 (m, 2H), 2.62 (brd, 1H), 1.91-2.06 (m, 1H), 1.84 (brd, 1H), 1.72-1.80 (m, 1H), 1.39 (d, 4H), 1.15-1.32 (m, 5H); m/z (ESI, +ve ion)=619.3. [M+H]⁺.

### Example 618. 1-((R)-2-cyclopropoxypropyl)-2-(((2S,4S)-4-((2-((2,4-difluorophenoxy)methyl)pyrimidin-4-yl)oxy)-2-methylpiperidin-1-yl)methyl)-1H-benzo[d]imidazole-6-carboxylic acid (618)

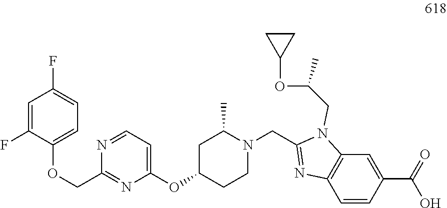 [SMILES: C[C@H](Cn1c(CN2CC[C@H](Oc3ccnc(COc4ccc(F)cc4F)n3)C[C@@H]2C)nc2ccc(C(=O)O)cc21)OC1CC1]

Example 618 was synthesized using same synthetic procedure as Example 612 from opposite enantiomer of 612a. ¹H NMR (400 MHz, CDCl₃) δ=8.43 (d, 1H), 8.20 (s, 1H), 8.04 (d, 1H), 7.79 (d, 1H), 7.01-6.85 (m, 2H), 6.77-6.70 (m, 1H), 6.57 (d, 1H), 5.23 (s, 2H), 5.11-5.00 (m, 1H), 4.70-4.55 (m, 2H), 4.19-4.11 (m, 1H), 4.05-3.95 (m, 1H), 3.48-3.41 (m, 1H), 3.13-3.06 (m, 1H), 2.75 (brdd, 1H), 2.47-2.37 (m, 1H), 2.25-2.16 (m, 1H), 2.14-2.05 (m, 2H), 1.99-1.90 (m, 2H), 1.38 (d, 3H), 1.29 (brd, 3H), 0.43-0.32 (m, 2H), 0.18-0.07 (m, 1H), −0.26-−0.35 (m, 1H); m/z (ESI, +ve ion)=608.2 [M+H]⁺.

### Example 619. 2-(((2S,4S)-4-((2-(((3,5-Difluoropyridin-2-yl)oxy)methyl)pyridin-4-yl)oxy)-2-methylpiperidin-1-yl)methyl)-4-fluoro-1-(((S)-oxetan-2-yl)methyl)-1H-benzo[d]imidazole-6-carboxylic acid (619)

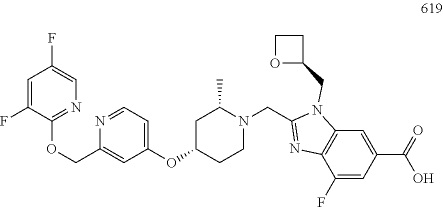 [SMILES: C[C@H]1C[C@@H](Oc2ccnc(COc3ncc(F)cc3F)c2)CCN1Cc1nc2c(F)cc(C(=O)O)cc2n1C[C@@H]1CCO1]

Example 619 was synthesized by following the synthetic route A-D for Example 606. ¹H NMR (400 MHz, DMSO-d6) δ ppm 13.08 (brd, 1H), 8.33 (d, 1H), 8.15 (s, 1H), 7.92-8.09 (m, 2H), 7.51 (d, 1H), 6.90-7.02 (m, 2H), 5.40 (s, 2H), 5.11-5.19 (m, 1H), 4.70-4.87 (m, 2H), 4.42-4.57 (m, 3H), 4.24 (dt, 1H), 3.61 (brd, 1H), 2.57-2.80 (m, 3H), 2.28-2.39 (m, 2H), 2.01-2.10 (m, 1H), 1.91-2.01 (m, 1H), 1.29-1.42 (m, 1H), 1.19-1.27 (m, 1H), 1.15 (brd, 3H); m/z (ESI, +ve ion)=598.3 [M+H]⁺.

### Example 620. 1-((R)-2-(difluoromethoxy)propyl)-2-(((2S,4S)-4-((2-(((3,5-difluoropyridin-2-yl)oxy)methyl)pyrimidin-4-yl)oxy)-2-methylpiperidin-1-yl)methyl)-4-fluoro-1H-benzo[d]imidazole-6-carboxylic acid (620)

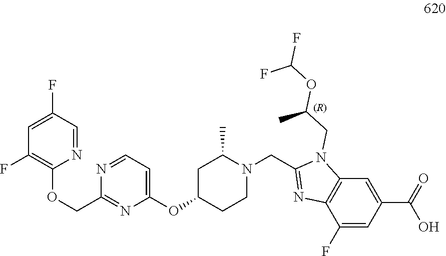

Example 620 was synthesized using similar procedure as Example 592. ¹H NMR (400 MHz, DMSO-d6) δ 8.44 (d, 1H), 8.11 (d, 1H), 8.07-7.99 (m, 1H), 7.95 (d, 1H), 7.50 (dd, 1H), 6.73 (d, 1H), 6.67-6.21 (m, 1H), 5.63-5.46 (m, 2H), 4.86-4.59 (m, 3H), 4.55-4.37 (m, 2H), 3.37 (brd, 1H), 2.61 (brd, 1H), 2.31-2.22 (m, 1H), 2.08-1.96 (m, 1H), 1.90-1.72 (m, 2H), 1.39 (brd, 4H), 1.31-1.13 (m, 4H); m/z (ESI, +ve ion)=637.3 [M+H]⁺.

### Example 621. 2-(((2S,4S)-4-((2-((2,4-Difluorophenoxy)methyl)pyrimidin-4-yl)oxy)-2-methylpiperidin-1-yl)methyl)-4-fluoro-1-(((S)-tetrahydrofuran-2-yl)methyl)-1H-benzo[d]imidazole-6-carboxylic acid (621)

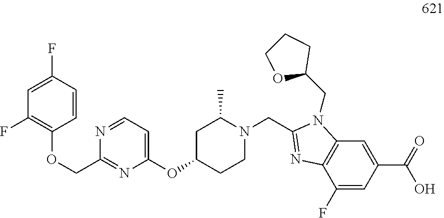 [SMILES: C[C@H]1C[C@@H](Oc2ccnc(COc3ccc(F)cc3F)n2)CCN1Cc1nc2c(F)cc(C(=O)O)cc2n1C[C@@H]1CCCO1]

Example 621 was prepared in a manner similar to Example 589. ¹H NMR (400 MHz, CD₃OD) δ ppm 8.43 (d, 1H), 8.14 (d, 1H), 7.59-7.67 (m, 1H), 6.98-7.11 (m, 2H), 6.78-6.87 (m, 1H), 6.72 (d, 1H), 5.25 (s, 2H), 5.03-5.13 (m, 1H), 4.81 (d, 1H), 4.45-4.60 (m, 2H), 4.27-4.36 (m, 1H), 3.84-3.92 (m, 1H), 3.69-3.81 (m, 2H), 2.86-2.97 (m, 1H), 2.59-2.73 (m, 1H), 2.33-2.49 (m, 1H), 2.12-2.20 (m, 1H), 2.04-2.12 (m, 1H), 1.95-2.02 (m, 1H), 1.85-1.94 (m, 2H), 1.63-1.73 (m, 2H), 1.48-1.59 (m, 1H), 1.25 (d, 3H); m/z (ESI, +ve ion)=612.2. [M+H]⁺.

### Example 622. 2-(((2S,4S)-4-((2-(((3,5-Difluoropyridin-2-yl)oxy)methyl)pyrimidin-4-yl)oxy)-2-methylpiperidin-1-yl)methyl)-4-fluoro-1-(((S)-tetrahydrofuran-2-yl)methyl)-1H-benzo[d]imidazole-6-carboxylic acid (622)

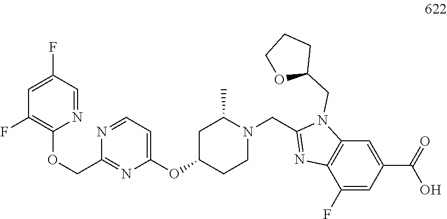 [SMILES: C[C@H]1C[C@@H](Oc2ccnc(COc3ncc(F)cc3F)n2)CCN1Cc1nc2c(F)cc(C(=O)O)cc2n1C[C@@H]1CCCO1]

Example 622 prepared in a route similar to Example 606. ¹H NMR (400 MHz, CDCl₃) δ ppm 8.41 (d, 1H), 8.03 (s, 1H), 7.66-7.78 (m, 2H), 7.28-7.35 (m, 1H), 6.54 (d, 1H), 5.58 (s, 2H), 4.90-5.01 (m, 1H), 4.83 (brd, 1H), 4.49 (brd, 1H), 4.39 (brdd, 1H), 4.28 (brdd, 1H), 3.81-3.91 (m, 1H), 3.68-3.79 (m, 2H), 2.82 (brd, 2H), 2.51 (brs, 2H), 2.31 (brt, 1H), 2.04-2.13 (m, 1H), 1.96-2.04 (m, 1H), 1.85-1.92 (m, 2H), 1.60 (brdd, 2H), 1.42-1.54 (m, 1H), 1.23 (brd, 3H); m/z (ESI, +ve ion)=613.2. [M+H]⁺.

### Example 623. 3-(2-(((2S,4S)-4-((2-((2,4-difluorophenoxy)methyl)pyrimidin-4-yl)oxy)-2-methylpiperidin-1-yl)methyl)-1-(((S)-oxetan-2-yl)methyl)-1H-benzo[d]imidazol-6-yl) propanoic acid (623)

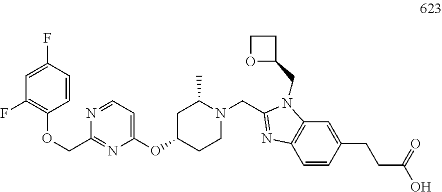 [SMILES: C[C@H]1C[C@@H](Oc2ccnc(COc3ccc(F)cc3F)n2)CCN1Cc1nc2ccc(CCC(=O)O)cc2n1C[C@@H]1CCO1]

### Step A: (2-(((2S,4S)-4-((2-((2,4-difluorophenoxy)methyl)pyrimidin-4-yl)oxy)-2-methylpiperidin-1-yl)methyl)-1-(((S)-oxetan-2-yl)methyl)-1H-benzo[d]imidazol-6-yl)methanol (Compound 639)

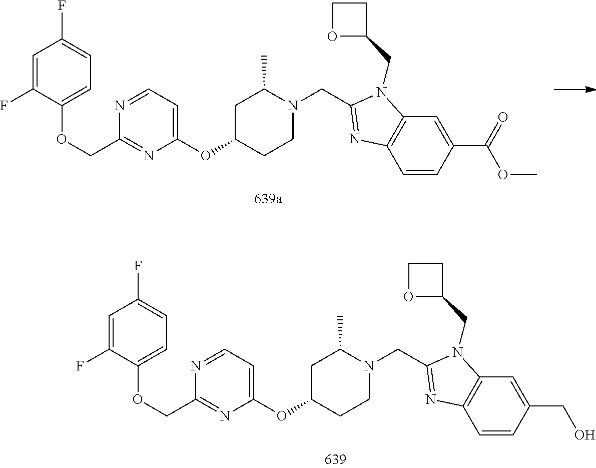 [SMILES: COC(=O)c1ccc2nc(CN3CC[C@H](Oc4ccnc(COc5ccc(F)cc5F)n4)C[C@@H]3C)n(C[C@@H]3CCO3)c2c1 ; C[C@H]1C[C@@H](Oc2ccnc(COc3ccc(F)cc3F)n2)CCN1Cc1nc2ccc(CO)cc2n1C[C@@H]1CCO1]

Example 639a (0.2 g, 0.337 mmol) (prepared using a similar procedure as 599f) was dissolved in toluene (3.4 mL) and was cooled to −78° C. DIBAl-H (1.0M in toluene, 0.674 mmol) was added dropwise and the resulting mixture stirred at −78° C. After 1H, DIBAl-H (1.0M in toluene, 0.169 mmol) was added dropwise and the mixture continued to stir at −78° C. Upon completion of the reaction, the mixture was allowed to warm to 0° C. The mixture was quenched with saturated Rochelle's salt, extracted with EtOAc (5 mL×3), dried with Na₂SO₄, filtered, and concentrated. The crude residue was purified via flash chromatography (0-5% MeOH in DCM) to afford Compound 639 (155 mg, 81% yield). m/z (ESI, +ve ion)=566.4 [M+H]⁺.

### Step B: 2-(((2S,4S)-4-((2-((2,4-difluorophenoxy)methyl)pyrimidin-4-yl)oxy)-2-methylpiperidin-1-yl)methyl)-1-(((S)-oxetan-2-yl)methyl)-1H-benzo[d]imidazole-6-carbaldehyde (623b)

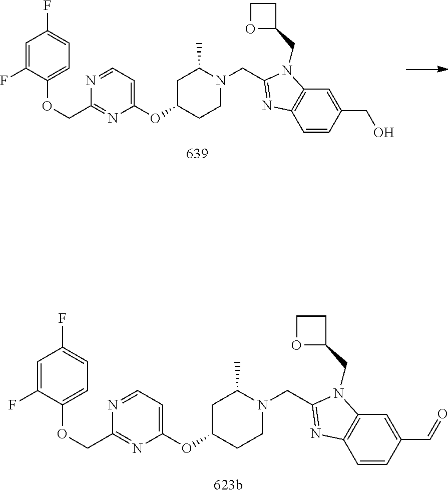 [SMILES: C[C@H]1C[C@@H](Oc2ccnc(COc3ccc(F)cc3F)n2)CCN1Cc1nc2ccc(CO)cc2n1C[C@@H]1CCO1 ; C[C@H]1C[C@@H](Oc2ccnc(COc3ccc(F)cc3F)n2)CCN1Cc1nc2ccc(C=O)cc2n1C[C@@H]1CCO1]

To a stirring solution of Compound 639 (42.5 mg, 0.0751 mmol) in MeCN:CHCl₃=1:1 (0.75 mL) was added MnO₂ (32.7 mg, 0.376 mmol). Upon completion of the reaction, the mixture was filtered, and the solid residue was washed with DCM (2 mL×3). The filtrate was concentrated to afford compound 623b (42.3 mg, 100% yield) to be used without further purification. (ESI, +ve ion)=564.4 [M+H]⁺.

### Step C: ethyl (E)-3-(2-(((2S,4S)-4-((2-((2,4-difluorophenoxy)methyl)pyrimidin-4-yl)oxy)-2-methylpiperidin-1-yl)methyl)-1-(((S)-oxetan-2-yl)methyl)-1H-benzo[d]imidazol-6-yl) acrylate (623c)

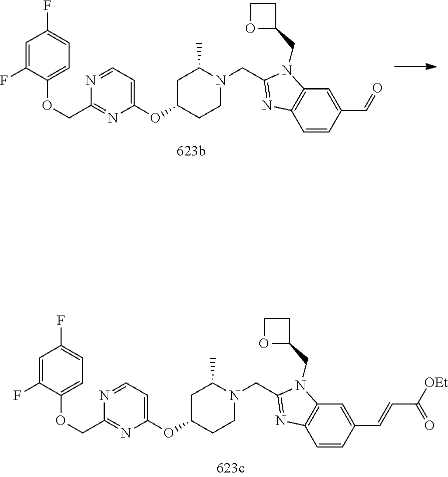 [SMILES: C[C@H]1C[C@@H](Oc2ccnc(COc3ccc(F)cc3F)n2)CCN1Cc1nc2ccc(C=O)cc2n1C[C@@H]1CCO1 ; CCOC(=O)/C=C/c1ccc2nc(CN3CC[C@H](Oc4ccnc(COc5ccc(F)cc5F)n4)C[C@@H]3C)n(C[C@@H]3CCO3)c2c1]

Compound 623b (42.3 mg, 0.0751 mmol) was dissolved in DCM (0.37 mL) and ethyl (triphenylphosphoranylidene)acetate (39.2 mg, 0.113 mmol) was then added. The resulting mixture was allowed to stir at ambient temperature. Upon completion of the reaction, the mixture was concentrated and purified via flash chromatography (0-100% EtOAc in hexanes) to afford compound 623c (13.7 mg, 29% yield). (ESI, +ve ion)=634.3 [M+H]⁺.

### Step D: 3-(2-(((2S,4S)-4-((2-((2,4-difluorophenoxy)methyl)pyrimidin-4-yl)oxy)-2-methylpiperidin-1-yl)methyl)-1-(((S)-oxetan-2-yl)methyl)-1H-benzo[d]imidazol-6-yl) propanoic acid (623)

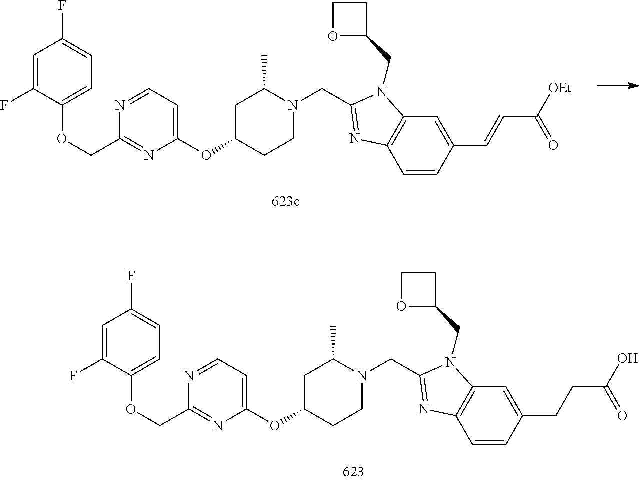 [SMILES: CCOC(=O)/C=C/c1ccc2nc(CN3CC[C@H](Oc4ccnc(COc5ccc(F)cc5F)n4)C[C@@H]3C)n(C[C@@H]3CCO3)c2c1 ; C[C@H]1C[C@@H](Oc2ccnc(COc3ccc(F)cc3F)n2)CCN1Cc1nc2ccc(CCC(=O)O)cc2n1C[C@@H]1CCO1]

623c (13.7 mg, 0.0022 mmol) was dissolved in anhydrous MeOH (1 mL) and the atmosphere was purged. 10% Pd/C (0.23 mg, 0.0002 mmol) was added, and the mixture was stirred under a H₂ atmosphere. Upon completion of the reaction, the mixture was filtered, and the solid residue was washed with MeOH (2 mL×3). The filtrate was concentrated to afford the reduced product (8.8 mg, 64% yield) to be used without further purification. (ESI, +ve ion)=636.4 [M+H]⁺. The reduced product (8.8 mg, 0.0014 mmol) was dissolved in THF:MeOH:H₂O=4:1:1 (0.6 mL) and lithium hydroxide monohydrate (2.9 mg, 0.0692 mmol) was added. Upon completion of the reaction, the solution was adjusted to pH=7 via the addition of acetic acid. Volatiles were evaporated and the resulting aqueous solution was extracted with EtOAc (5 mL×3). The organic layer was dried with Na₂SO₄, filtered, and concentrated. The crude residue was purified via flash chromatography (0-10% MeOH in DCM) to afford compound 523 (3.6 mg, 43% yield). ¹H NMR (400 MHz, CDCl₃) δ: 8.41 (d, 1H), 7.65 (d, 1H), 7.28 (s, 1H), 7.12 (d, 1H), 6.85-6.97 (m, 2H), 6.70-6.77 (m, 1H), 6.56 (d, 1H), 5.15-5.26 (m, 3H), 4.99-5.09 (m, 1H), 4.88 (dd, 1H), 4.50-4.61 (m, 2H), 4.37 (dd, 1H), 4.25 (dt, 1H), 3.52 (d, 1H), 3.11 (brt, 2H), 2.69-2.80 (m, 6H), 2.61-2.68 (m, 1H), 2.34-2.49 (m, 2H), 2.22 (brt, 1H), 2.03 (brd, 1H), 1.90 (brd, 1H), 1.40-1.59 (m, 2H), 1.22 (d, 3H); m/z (ESI, +ve ion)=608.4 [M+H]⁺.

### Example 624. 2-(((2S,4S)-4-((2-(((3-Chloro-5-fluoropyridin-2-yl)amino)methyl)pyrimidin-4-yl)oxy)-2-methylpiperidin-1-yl)methyl)-1-(((S)-oxetan-2-yl)methyl)-1H-benzo[d]imidazole-6-carboxylic acid (624)

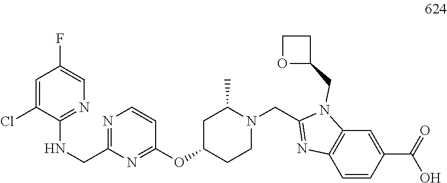 [SMILES: C[C@H]1C[C@@H](Oc2ccnc(CNc3ncc(F)cc3Cl)n2)CCN1Cc1nc2ccc(C(=O)O)cc2n1C[C@@H]1CCO1]

### Step A: tert-Butyl(2S,4S)-4-((2-formylpyrimidin-4-yl)oxy)-2-methylpiperidine-1-carboxylate (624a)

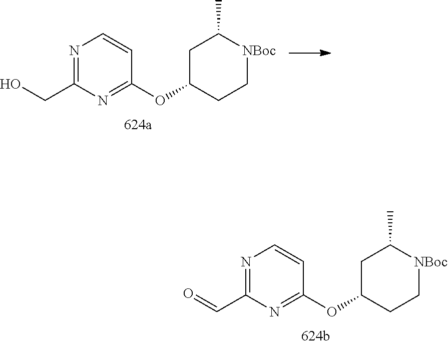 [SMILES: C[C@H]1C[C@@H](Oc2ccnc(CO)n2)CCN1C(=O)OC(C)(C)C ; C[C@H]1C[C@@H](Oc2ccnc(C=O)n2)CCN1C(=O)OC(C)(C)C]

To a solution of 624a (300 mg, 927.68 umol) in DCM (2 ml) was added TEMPO (14.59 mg, 92.77 umol) and iodobenzene diacetate (328.68 mg, 1.02 mmol). The mixture was stirred at 25° C. for 16 h. After completion, the residue was poured into Na₂SO₃ (10 ml) and water (20 ml). The aqueous phase was extracted with DCM (20 ml*3). The combined organic layer was dried over Na₂SO₄, concentrated and purified by prep-TLC (PE/EA=1:1) to afford the desired product 624b (226 mg, 75.81% yield) as a colorless oil. m/z (ESI, +ve ion)=340.3 [M+H]⁺.

### Step B: tert-Butyl(2S,4S)-4-((2-(((3-chloro-5-fluoropyridin-2-yl)amino)methyl)pyrimidin-4-yl)oxy)-2-methylpiperidine-1-carboxylate (624c)

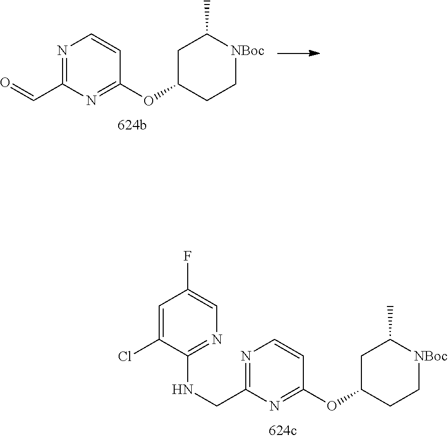 [SMILES: C[C@H]1C[C@@H](Oc2ccnc(C=O)n2)CCN1C(=O)OC(C)(C)C ; C[C@H]1C[C@@H](Oc2ccnc(CNc3ncc(F)cc3Cl)n2)CCN1C(=O)OC(C)(C)C]

A mixture of 624b (20 mg, 62.23 umol), 3-chloro-5-fluoro-pyridin-2-amine (18.24 mg, 124.47 umol) in ACN (2 ml) was added triethylsilane (53.55 mg, 460.53 umol, 73.56 uL) and TFA (53.93 mg, 472.97 umol, 35.02 ul) degassed and purged with N₂ for 3 times, and then the mixture was stirred at 70° C. for 4 h under N₂ atmosphere. After completion, the mixture was adjusted to pH=7 with saturated sodium bicarbonate solution water solution, extracted with ethyl acetate (20 ml*3). The combined organic layer was dried over Na₂SO₄, concentrated and purified by prep-TLC (PE/EA=3:1) to afford the desired product 624c (8 mg, 28.44% yield) as a white oil. m/z (ESI, +ve ion)=452.1 [M+H]⁺.

### Step C: Methyl 2-(((2S,4S)-4-((2-(((3-chloro-5-fluoropyridin-2-yl)amino)methyl)pyrimidin-4-yl)oxy)-2-methylpiperidin-1-yl)methyl)-1-(((S)-oxetan-2-yl)methyl)-1H-benzo[d]imidazole-6-carboxylate (624d)

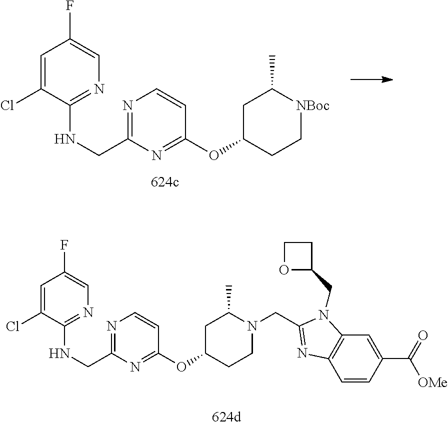 [SMILES: C[C@H]1C[C@@H](Oc2ccnc(CNc3ncc(F)cc3Cl)n2)CCN1C(=O)OC(C)(C)C ; COC(=O)c1ccc2nc(CN3CC[C@H](Oc4ccnc(CNc5ncc(F)cc5Cl)n4)C[C@@H]3C)n(C[C@@H]3CCO3)c2c1]

To a solution of 624c (60 mg, 132.77 umol) in TFA (0.2 ml) and DCM (0.6 ml). The mixture was stirred at 25° C. for 1H. After completion, the mixture was adjusted to pH=7 with saturated sodium bicarbonate water solution, extracted with DCM (20 ml*3). The combined organic layer was dried over Na₂SO₄ and concentrated to afford the crude product (46 mg, 98.48% yield) as a yellow oil, which was used in the next step reaction without further purification. m/z (ESI, +ve ion)=352.2 [M+H]⁺.

A mixture of amine (170 mg, 483.22 umol), methyl 2-(chloromethyl)-3-[[(2S)-oxetan-2-yl]methyl]benzimidazole-5-carboxylate (185.15 mg, 628.19 umol), DIPEA (312.26 mg, 2.42 mmol, 420.84 ul) in DMF (2 ml) was degassed and purged with N₂ for 3 times, and then the mixture was stirred at 85° C. for 16 h under N₂ atmosphere. After completion, the reaction mixture was filtrated and concentrated in vacuo. The resulting residue was purified by column chromatography (SiO₂, Petroleum ether/Ethyl acetate=50/1 to 0/1) to afford the desired product 624d (170 mg, 57.67% yield) as a yellow oil. m/z (ESI, +ve ion)=610.3 [M+H]⁺.

### Step D: 2-(((2S,4S)-4-((2-(((3-Chloro-5-fluoropyridin-2-yl)amino)methyl)pyrimidin-4-yl)oxy)-2-methylpiperidin-1-yl)methyl)-1-(((S)-oxetan-2-yl)methyl)-1H-benzo[d]imidazole-6-carboxylic acid

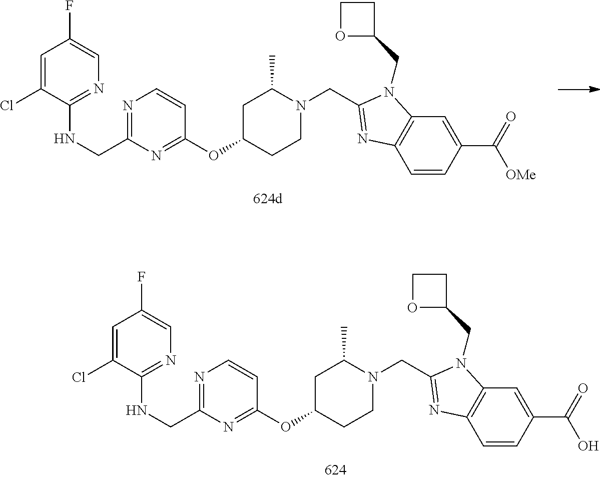 [SMILES: COC(=O)c1ccc2nc(CN3CC[C@H](Oc4ccnc(CNc5ncc(F)cc5Cl)n4)C[C@@H]3C)n(C[C@@H]3CCO3)c2c1 ; C[C@H]1C[C@@H](Oc2ccnc(CNc3ncc(F)cc3Cl)n2)CCN1Cc1nc2ccc(C(=O)O)cc2n1C[C@@H]1CCO1]

A mixture of 624d (170 mg, 278.65 umol), LiOH·H₂O (58.47 mg, 1.39 mmol) in THF (1 ml), MeOH (1 ml) and H₂O (1 ml) was degassed and purged with N₂ for 3 times, and then the mixture was stirred at 0-20° C. for 16 h under N₂ atmosphere. After completion, the reaction mixture was cooled to 0° C., and pH was then adjusted to 5 by the addition of 1N HCl carefully. Aqueous layer was extracted with EtOAc (10 mL×3). The combined organic layer was dried over Na₂SO₄, filtered, concentrated and purified by prep-HPLC (column: Waters Xbridge Prep OBD C18 150*40 mm*10 um; mobile phase: [water (NH₄HCO₃)-ACN]; B %: 10%-45%, 8 min) to afford the desired product 624 (65.5 mg, 39.44% yield). ¹H NMR (400 MHz, CDCl₃) δ ppm 8.42 (d, 1H), 8.24 (s, 1H), 8.08 (d, 1H), 7.95 (d, 1H), 7.85 (d, 1H), 7.37 (dd, 1H), 6.55 (d, 1H), 6.17 (brt, 1H), 5.31-5.24 (m, 1H), 5.18-5.10 (m, 1H), 5.03-4.96 (m, 1H), 4.75 (brd, 2H), 4.69-4.53 (m, 3H), 4.34-4.27 (m, 1H), 3.69 (brd, 1H), 2.87 (brd, 1H), 2.72 (brdd, 2H), 2.49-2.30 (m, 3H), 2.15 (brd, 1H), 2.05-1.98 (m, 1H), 1.70-1.50 (m, 2H), 1.27 (brd, 3H); m/z (ESI, +ve ion)=569.2 [M+H]⁺.

### Example 625. 2-(((2S,4S)-4-((2-(((5-Cyano-3-fluoropyridin-2-yl)oxy)methyl)pyridin-4-yl)oxy)-2-methylpiperidin-1-yl)methyl)-4-fluoro-1-(((S)-oxetan-2-yl)methyl)-1H-benzo[d]imidazole-6-carboxylic acid (625)

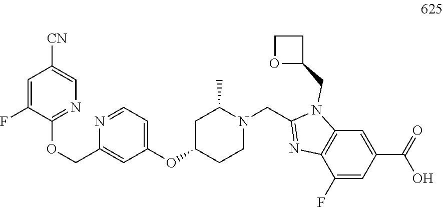 [SMILES: C[C@H]1C[C@@H](Oc2ccnc(COc3ncc(C#N)cc3F)c2)CCN1Cc1nc2c(F)cc(C(=O)O)cc2n1C[C@@H]1CCO1]

Example 625 was prepared in a manner similar to Example 606. ¹H NMR (400 MHz, CD₃OD) δ=8.35 (d, 1H), 8.32 (d, 1H), 8.15 (s, 1H), 7.97 (dd, 1H), 7.63 (d, 1H), 7.09 (d, 1H), 6.96 (dd, 1H), 5.54 (s, 2H), 5.33-5.24 (m, 1H), 4.97 (brd, 1H), 4.73 (brd, 1H), 4.64-4.56 (m, 4H), 4.32 (td, 1H), 3.65 (brd, 1H), 2.89-2.81 (m, 1H), 2.80-2.70 (m, 1H), 2.68-2.58 (m, 1H), 2.51-2.34 (m, 2H), 2.13 (brd, 1H), 2.09-2.01 (m, 1H), 1.69-1.50 (m, 2H), 1.25 (d, 3H); m/z (ESI, +ve ion)=605.2 [M+H]⁺.

### Example 626. 2-(((2S,4S)-4-((2-(((5-cyano-3-fluoropyridin-2-yl)oxy)methyl)pyrimidin-4-yl)oxy)-2-methylpiperidin-1-yl)methyl)-1-((R)-2-(difluoromethoxy)propyl)-1H-benzo[d]imidazole-6-carboxylic acid

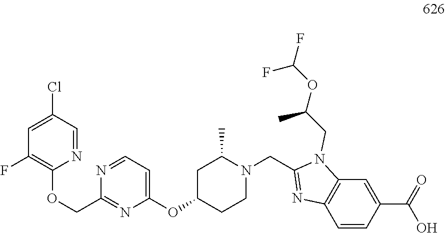 [SMILES: C[C@H](Cn1c(CN2CC[C@H](Oc3ccnc(COc4ncc(Cl)cc4F)n3)C[C@@H]2C)nc2ccc(C(=O)O)cc21)OC(F)F]

Example 626 was prepared in a manner similar to Example 606. ¹H NMR (400 MHz, CD₃OD) δ=8.38 (d, 1H), 8.26 (s, 2H), 8.05 (dd, 1H), 7.96 (d, 1H), 7.66 (d, 1H), 6.68 (d, 1H), 6.36-5.93 (m, 1H), 5.74-5.59 (m, 2H), 4.45-4.37 (m, 1H), 3.38 (d, 1H), 2.72-2.61 (m, 1H), 2.31 (brdd, 1H), 2.11-1.95 (m, 2H), 1.80 (brd, 1H), 1.56-1.37 (m, 5H), 1.31 (d, 3H); m/z (ESI, +ve ion)=626.2.

### Example 627. 2-(((2S,4S)-4-((2-(((3-Cyano-5-fluoropyridin-2-yl)oxy)methyl)pyrimidin-4-yl)oxy)-2-methylpiperidin-1-yl)methyl)-1-((R)-2-(difluoromethoxy)propyl)-1H-benzo[d]imidazole-6-carboxylic acid (627)

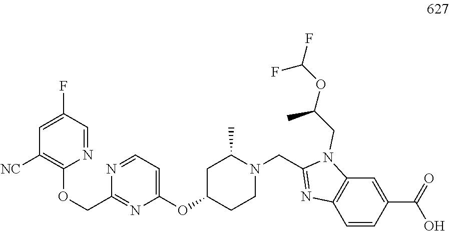 [SMILES: C[C@H](Cn1c(CN2CC[C@H](Oc3ccnc(COc4ncc(F)cc4C#N)n3)C[C@@H]2C)nc2ccc(C(=O)O)cc21)OC(F)F]

Example 627 was prepared in a manner similar to Example 606. ¹H NMR (400 MHz, DMSO-d6) δ=8.54 (dd, 1H), 8.48-8.38 (m, 2H), 8.21 (s, 1H), 7.80 (d, 1H), 7.63 (d, 1H), 6.74 (d, 1H), 6.44 (t, 1H), 5.71-5.51 (m, 2H), 4.87-4.67 (m, 2H), 4.66-4.56 (m, 1H), 4.55-4.36 (m, 2H), 2.72-2.57 (m, 1H), 2.35-2.19 (m, 1H), 2.04-1.95 (m, 1H), 1.87-1.63 (m, 2H), 1.43-1.33 (m, 4H), 1.28-1.21 (m, 2H), 1.18 (d, 3H); m/z (ESI, +ve ion)=626.1 [M+H]⁺.

### Example 628. (S)-2-((4-((2-(((3,5-difluoropyridin-2-yl)oxy)methyl)pyrimidin-4-yl)oxy)piperidin-1-yl)methyl)-4-fluoro-1-(oxetan-2-ylmethyl)-1H-benzo[d]imidazole-6-carboxylic acid (628)

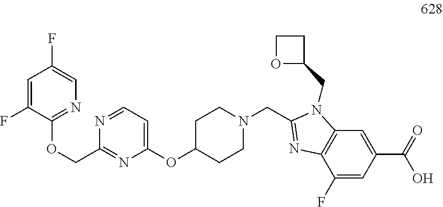 [SMILES: O=C(O)c1cc(F)c2nc(CN3CCC(Oc4ccnc(COc5ncc(F)cc5F)n4)CC3)n(C[C@@H]3CCO3)c2c1]

Example 628 was prepared in a manner similar to Example 606. ¹H NMR (400 MHz, CDCl₃) δ: 8.30-8.39 (m, 1H), 7.83-7.96 (m, 1H), 7.65-7.71 (m, 1H), 7.57-7.65 (m, 1H), 6.46-6.54 (m, 1H), 5.43-5.56 (m, 2H), 5.08-5.18 (m, 1H), 4.89-4.98 (m, 1H), 4.52-4.71 (m, 3H), 4.27-4.38 (m, 1H), 3.84-4.02 (m, 2H), 2.59-2.80 (m, 3H), 2.26-2.44 (m, 4H), 2.00-2.13 (m, 3H), 1.92-1.96 (m, 1H), 1.74-1.89 (m, 4H), 1.65-1.70 (m, 1H), 1.62-1.64 (m, 1H), 1.17 (s, 2H); m/z (ESI, +ve ion)=585.3 [M+H]⁺.

### Example 629. 2-(((2S,4S)-4-((2-(((3-cyano-5-fluoropyridin-2-yl)oxy)methyl)pyrimidin-4-yl)oxy)-2-methylpiperidin-1-yl)methyl)-4-fluoro-1-(((S)-oxetan-2-yl)methyl)-1H-benzo[d]imidazole-6-carboxylic acid (629)

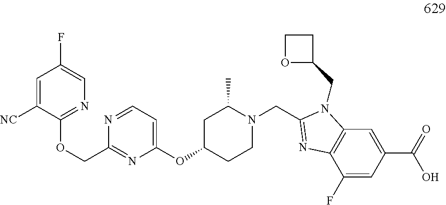 [SMILES: C[C@H]1C[C@@H](Oc2ccnc(COc3ncc(F)cc3C#N)n2)CCN1Cc1nc2c(F)cc(C(=O)O)cc2n1C[C@@H]1CCO1]

Example 629 was prepared in a manner similar to Example 606. ¹H NMR (400 MHz, CDCl₃) δ: 8.31 (d, 1H), 8.06 (d, 1H), 7.97 (s, 1H), 7.63-7.69 (m, 2H), 6.46 (d, 1H), 5.48-5.57 (m, 2H), 5.14-5.21 (m, 1H), 4.82-4.93 (m, 2H), 4.44-4.58 (m, 3H), 4.22 (dt, 1H), 3.62 (d, 1H), 2.59-2.75 (m, 2H), 2.26-2.48 (m, 3H), 2.14-2.25 (m, 2H), 2.10 (brs, 1H), 1.94 (brd, 2H), 1.80 (brd, 2H), 1.34-1.54 (m, 3H), 1.04-1.26 (m, 4H); m/z (ESI, +ve ion)=606.6 [M+H]⁺.

### Example 630. 2-(((2S,4S)-4-((2-(((3-Cyano-5-fluoropyridin-2-yl)oxy)methyl)pyrimidin-4-yl)oxy)-2-methylpiperidin-1-yl)methyl)-1-(((S)-oxetan-2-yl)methyl)-1H-benzo[d]imidazole-6-carboxylic acid (630)

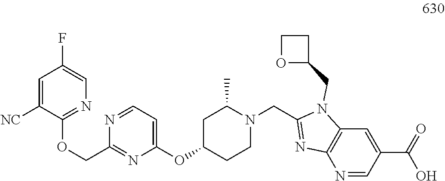 [SMILES: C[C@H]1C[C@@H](Oc2ccnc(COc3ncc(F)cc3C#N)n2)CCN1Cc1nc2ncc(C(=O)O)cc2n1C[C@@H]1CCO1]

Example 630 was prepared in a manner similar to Example 606. ¹H NMR (400 MHz, CDCl₃) δ: 8.28 (d, 1H), 8.14 (s, 1H), 8.05 (d, 1H), 7.97 (dd, 1H), 7.73 (d, 1H), 7.61 (dd, 1H), 6.45 (d, 1H), 5.46-5.55 (m, 2H), 5.14-5.22 (m, 1H), 4.81-4.91 (m, 2H), 4.42-4.56 (m, 3H), 4.20 (dt, 1H), 3.54 (d, 1H), 2.58-2.73 (m, 2H), 2.25-2.44 (m, 2H), 2.06-2.22 (m, 2H), 1.84-2.00 (m, 2H), 1.70-1.83 (m, 3H), 1.34-1.52 (m, 4H), 1.07-1.22 (m, 4H); m/z (ESI, +ve ion)=588.3 [M+H]⁺.

### Example 631. 2-(((2S,4S)-4-((2-(((5-cyano-3-fluoropyridin-2-yl)oxy)methyl)pyrimidin-4-yl)oxy)-2-methylpiperidin-1-yl)methyl)-1-(((S)-tetrahydrofuran-2-yl)methyl)-1H-benzo[d]imidazole-6-carboxylic acid (631)

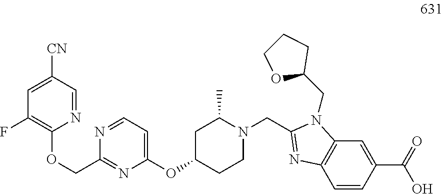 [SMILES: C[C@H]1C[C@@H](Oc2ccnc(COc3ncc(C#N)cc3F)n2)CCN1Cc1nc2ccc(C(=O)O)cc2n1C[C@@H]1CCCO1]

Example 631 was prepared in a manner similar to Example 606. ¹H NMR (400 MHz, CDCl₃) δ: 8.29 (d, 1H), 8.14 (s, 1H), 8.10 (d, 1H), 7.95 (dd, 1H), 7.71 (d, 1H), 7.52 (dd, 1H), 7.18 (s, 6H), 6.46 (d, 1H), 5.50-5.60 (m, 2H), 4.68-4.89 (m, 2H), 4.41 (d, 1H), 4.15-4.36 (m, 2H), 3.74-3.81 (m, 1H), 3.64-3.70 (m, 1H), 3.57 (d, 1H), 2.66-2.79 (m, 1H), 2.29-2.44 (m, 1H), 2.16 (brt, 1H), 1.87-2.01 (m, 3H), 1.70-1.85 (m, 4H), 1.36-1.62 (m, 6H), 1.04-1.29 (m, 5H); m/z (ESI, +ve ion)=602.4 [M+H]⁺.

### Example 632. 2-(((2S,4S)-4-((2-(((3,5-Difluoropyridin-2-yl)oxy)methyl)pyrimidin-4-yl)oxy)-2-methylpiperidin-1-yl)methyl)-1-(((S)-tetrahydrofuran-2-yl)methyl)-1H-benzo[d]imidazole-6-carboxylic acid (632)

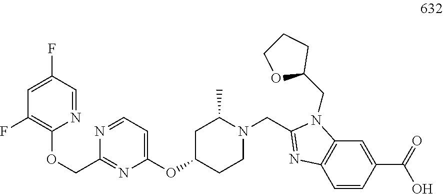 [SMILES: C[C@H]1C[C@@H](Oc2ccnc(COc3ncc(F)cc3F)n2)CCN1Cc1nc2ccc(C(=O)O)cc2n1C[C@@H]1CCCO1]

Example 632 was prepared in a manner similar to Example 606. ¹H NMR (400 MHz, CDCl₃) δ: 8.30 (d, 1H), 8.15 (s, 1H), 7.95 (d, 1H), 7.70 (d, 1H), 7.66 (d, 1H), 6.44 (d, 1H), 5.48 (s, 2H), 4.81-4.90 (m, 1H), 4.74 (brdd, 1H), 4.18-4.43 (m, 3H), 3.78 (q, 1H), 3.63-3.70 (m, 1H), 3.56 (d, 1H), 2.61-2.79 (m, 1H), 2.26-2.45 (m, 1H), 2.07-2.23 (m, 2H), 1.74-2.02 (m, 6H), 1.72-1.73 (m, 1H), 1.33-1.58 (m, 4H), 1.14 (d, 4H); m/z (ESI, +ve ion)=595.3 [M+H]⁺.

### Example 633. 2-(((2S,4S)-4-((2-((2,4-Difluorophenoxy)methyl)pyrimidin-4-yl)oxy)-2-methylpiperidin-1-yl)methyl)-1-(((S)-oxetan-2-yl)methyl)-1H-benzo[d]imidazole (633)

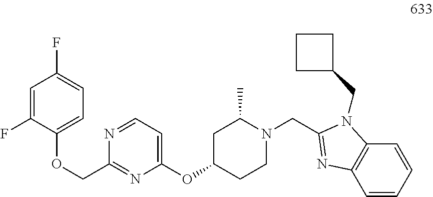

Example 633 was prepared in a manner similar to Example 599. ¹H NMR (400 MHz, CDCl₃) δ: 8.32 (d, 1H), 7.64-7.71 (m, 1H), 7.30-7.36 (m, 1H), 7.15-7.21 (m, 2H), 6.75-6.90 (m, 2H), 6.61-6.68 (m, 1H), 6.47 (d, 1H), 5.11-5.16 (m, 2H), 4.91-4.99 (m, 1H), 4.87 (dd, 1H), 4.46-4.55 (m, 2H), 4.32 (dd, 1H), 4.17 (dt, 1H), 3.42 (d, 1H), 2.71 (dt, 1H), 2.52-2.64 (m, 1H), 2.28-2.40 (m, 2H), 2.06-2.17 (m, 1H), 1.90-1.99 (m, 2H), 1.82 (brd, 1H), 1.31-1.52 (m, 2H), 1.14 (d, 3H). m/z (ESI, +ve ion)=536.0 [M+H]⁺.

### Example 634. (S)-2-((4-((2-(((5-Cyano-3-fluoropyridin-2-yl)oxy)methyl)pyrimidin-4-yl)oxy)piperidin-1-yl)methyl)-1-(oxetan-2-ylmethyl)-1H-benzo[d]imidazole-6-carboxylic acid (634)

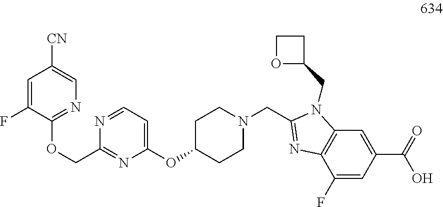

Example 634 was prepared in a manner similar to Example 606. ¹H NMR (400 MHz, CDCl₃) δ: 8.39 (d, 1H), 8.18 (d, 1H), 7.94 (brs, 1H), 7.58-7.68 (m, 2H), 7.49-7.57 (m, 1H), 6.58 (d, 1H), 5.63 (s, 2H), 5.15 (brs, 1H), 4.98 (brs, 1H), 4.55-4.68 (m, 2H), 4.34 (brs, 1H), 3.95 (brs, 2H), 2.68-2.79 (m, 2H), 2.39 (brs, 3H), 2.27 (brs, 3H), 2.05 (brs, 1H), 1.85-1.92 (m, 2H), 1.67-1.72 (m, 1H), 1.37-1.37 (m, 1H), 1.35-1.52 (m, 1H), 1.26 (s, 2H), 0.80-0.94 (m, 1H), 0.04-0.13 (m, 1H). m/z (ESI, +ve ion)=592.0 [M+H]⁺.

### Example 635. 2-(((2S,4S)-4-((2-(((5-Cyano-3-fluoropyridin-2-yl)oxy)methyl)pyrimidin-4-yl)oxy)-2-methylpiperidin-1-yl)methyl)-1-(2-(2,2,2-trifluoroethoxy)ethyl)-1H-benzo[d]imidazole-6-carboxylic acid (635)

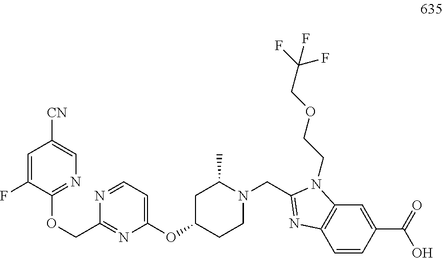 [SMILES: C[C@H]1C[C@@H](Oc2ccnc(COc3ncc(C#N)cc3F)n2)CCN1Cc1nc2ccc(C(=O)O)cc2n1CCOCC(F)(F)F]

Example 635 was prepared in a manner similar to Example 606. ¹H NMR (400 MHz, CDCl₃) δ: 8.29 (d, 1H), 8.09 (d, 2H), 7.97 (brd, 1H), 7.70 (brd, 1H), 7.53 (dd, 1H), 6.46 (d, 1H), 5.55 (s, 2H), 4.78-4.93 (m, 2H), 4.33-4.49 (m, 2H), 3.80-4.01 (m, 2H), 3.53-3.68 (m, 2H), 3.42-3.52 (m, 1H), 2.65 (brd, 1H), 2.32 (brs, 1H), 2.03-2.16 (m, 1H), 1.87-2.00 (m, 1H), 1.78 (brd, 1H), 1.33-1.49 (m, 2H), 1.17 (brd, 3H). m/z (ESI, +ve ion)=644.4 [M+H]⁺.

### Example 636. 2-(((2S,4S)-4-((2-(((5-cyano-3-fluoropyridin-2-yl)oxy)methyl)pyrimidin-4-yl)oxy)-2-methylpiperidin-1-yl)methyl)-4-fluoro-1-(2-(2,2,2-trifluoroethoxy)ethyl)-1H-benzo[d]imidazole-6-carboxylic acid (636)

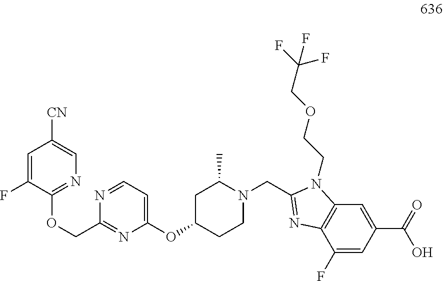 [SMILES: C[C@H]1C[C@@H](Oc2ccnc(COc3ncc(C#N)cc3F)n2)CCN1Cc1nc2c(F)cc(C(=O)O)cc2n1CCOCC(F)(F)F]

Example 636 was prepared in a manner similar to Example 606. ¹H NMR (400 MHz, CDCl₃) δ: 8.29 (d, 1H), 8.05-8.11 (m, 1H), 7.86 (brs, 1H), 7.52-7.65 (m, 2H), 6.46 (d, 1H), 5.55 (s, 2H), 4.73-4.92 (m, 2H), 4.39 (brd, 2H), 3.79-3.98 (m, 2H), 3.65 (brdd, 2H), 3.49 (brd, 1H), 2.63 (brd, 1H), 2.33 (brs, 1H), 2.11 (brt, 1H), 1.88-2.01 (m, 1H), 1.70-1.85 (m, 1H), 1.31-1.47 (m, 2H), 1.16 (brd, 3H). m/z (ESI, +ve ion)=662.3 [M+H]⁺.

### Example 637. 2-(2-(((2S,4S)-4-((2-((2,4-difluorophenoxy)methyl)pyrimidin-4-yl)oxy)-2-methylpiperidin-1-yl)methyl)-1-(((S)-oxetan-2-yl)methyl)-1H-benzo[d]imidazol-6-yl) acetic acid (637)

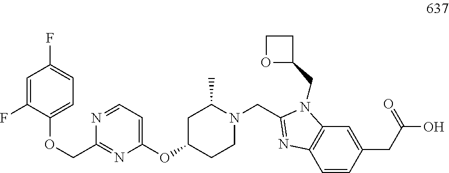 [SMILES: C[C@H]1C[C@@H](Oc2ccnc(COc3ccc(F)cc3F)n2)CCN1Cc1nc2ccc(CC(=O)O)cc2n1C[C@@H]1CCO1]

Example 637 was prepared in a manner similar to Example 599. ¹H NMR (400 MHz, CDCl₃) δ: 8.33 (d, 1H), 7.58-7.66 (m, 1H), 7.23-7.32 (m, 1H), 7.19 (s, 1H), 7.08-7.13 (m, 1H), 6.76-6.88 (m, 2H), 6.58-6.69 (m, 1H), 6.47 (d, 1H), 5.14 (s, 2H), 5.03-5.09 (m, 1H), 4.88-5.02 (m, 1H), 4.73-4.86 (m, 1H), 4.38-4.50 (m, 2H), 4.28 (brdd, 1H), 4.08-4.21 (m, 1H), 3.67 (s, 2H), 3.47 (brd, 1H), 2.68 (brd, 1H), 2.46-2.60 (m, 1H), 2.23-2.40 (m, 2H), 2.14 (brt, 1H), 1.88-2.02 (m, 1H), 1.80 (brd, 1H), 1.32-1.51 (m, 2H), 1.20-1.28 (m, 1H), 1.18 (brs, 1H), 1.12 (brd, 3H); m/z (ESI, +ve ion)=594.3 [M+H]⁺.

### Example 638. 2-(((2S,4S)-4-((2-((2,4-Difluorophenoxy)methyl)pyrimidin-4-yl)oxy)-2-methylpiperidin-1-yl)methyl)-1-(((S)-oxetan-2-yl)methyl)-N-(pyridin-3-yl)-1H-benzo[d]imidazole-6-carboxamide (638)

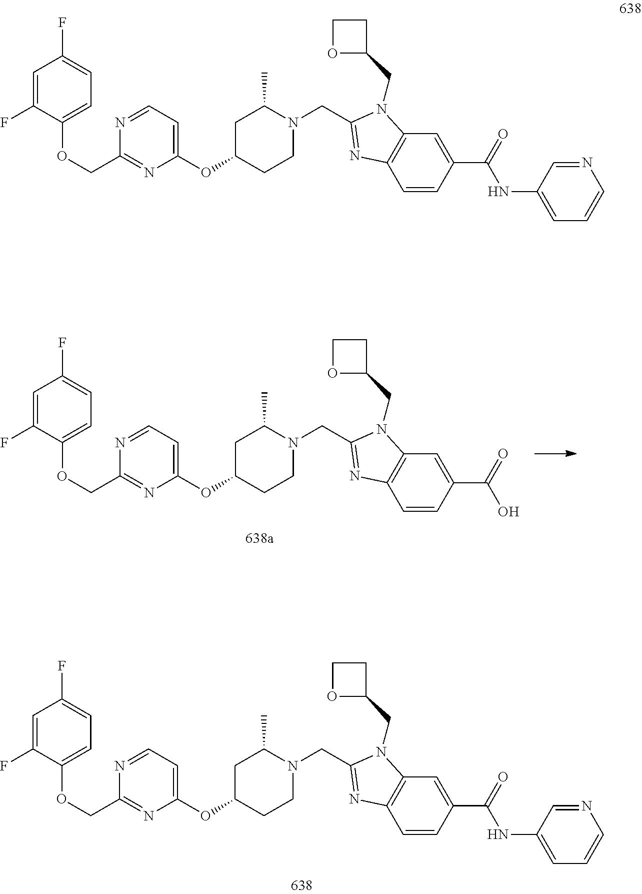 [SMILES: C[C@H]1C[C@@H](Oc2ccnc(COc3ccc(F)cc3F)n2)CCN1Cc1nc2ccc(C(=O)Nc3cccnc3)cc2n1C[C@@H]1CCO1 ; C[C@H]1C[C@@H](Oc2ccnc(COc3ccc(F)cc3F)n2)CCN1Cc1nc2ccc(C(=O)O)cc2n1C[C@@H]1CCO1 ; C[C@H]1C[C@@H](Oc2ccnc(COc3ccc(F)cc3F)n2)CCN1Cc1nc2ccc(C(=O)Nc3cccnc3)cc2n1C[C@@H]1CCO1]

DIPEA (0.018 mL, 0.104 mmol) was added to a stirring solution of Compound 638a (prepared as in Example 599) (20 mg, 0.035 mmol) in DMF (0.5 mL). After 5 minutes, HATU (16 mg, 0.069 mmol) was added, followed by the addition of pyridin-3-amine (3.6 mg, 0.038 mmol) in DMF (0.5 mL). The solution stirred o/n at rt. Pyridin-3-amine (7 mg, 0.076 mmol) was then added and the mixture stirred for another 4 h. The reaction was quenched with water, extracted with EtOAc (3×5 mL), dried with Na₂SO₄, filtered, and concentrated. Flash chromatographic purification (0-10% MeOH in DCM) afforded the desired product (7.9 mg, 35% yield). ¹H NMR (400 MHz, CDCl₃) δ: 8.74 (d, 1H), 8.36-8.43 (m, 3H), 8.33 (brd, 1H), 8.13 (s, 1H), 7.77-7.82 (m, 1H), 7.74 (d, 1H), 7.29-7.38 (m, 1H), 6.84-7.01 (m, 2H), 6.69-6.77 (m, 1H), 6.56 (d, 1H), 5.22 (s, 2H), 5.00-5.09 (m, 1H), 4.94 (dd, 1H), 4.51-4.64 (m, 3H), 4.30 (dt, 1H), 3.59 (d, 1H), 2.64-2.83 (m, 2H), 2.38-2.50 (m, 2H), 2.20-2.33 (m, 1H), 2.04 (brd, 1H), 1.92 (brd, 1H), 1.58 (qd, 1H), 1.41-1.49 (m, 2H), 1.21 (d, 3H). m/z (ESI, +ve ion)=656.4 [M+H]⁺.

### Example 640 and 641. 2-(((1r,4S)-4-((2-((4-cyano-2-fluorophenoxy)methyl)pyrimidin-4-yl)oxy)cyclohexyl)methyl)-1-(((S)-oxetan-2-yl)methyl)-1H-benzo[d]imidazole-6-carboxylic acid (640) and 2-(((1s,4R)-4-((2-((4-cyano-2-fluorophenoxy)methyl)pyrimidin-4-yl)oxy)cyclohexyl)methyl)-1-(((S)-oxetan-2-yl)methyl)-1H-benzo[d]imidazole-6-carboxylic acid (641)

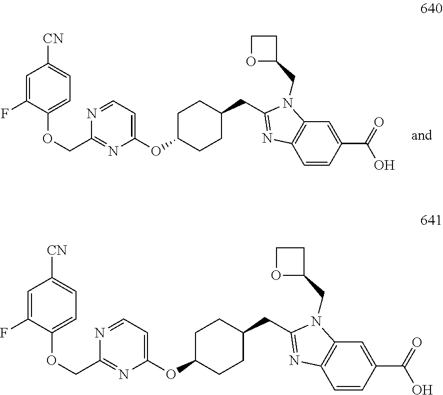 [SMILES: N#Cc1ccc(OCc2nccc(O[C@H]3CC[C@H](Cc4nc5ccc(C(=O)O)cc5n4C[C@@H]4CCO4)CC3)n2)c(F)c1 ; N#Cc1ccc(OCc2nccc(O[C@H]3CC[C@@H](Cc4nc5ccc(C(=O)O)cc5n4C[C@@H]4CCO4)CC3)n2)c(F)c1]

### Step A: 4-((4-Chloropyrimidin-2-yl) methoxy)-3-fluorobenzonitrile (640b)

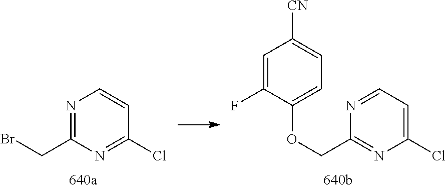 [SMILES: Clc1ccnc(CBr)n1 ; N#Cc1ccc(OCc2nccc(Cl)n2)c(F)c1]

A mixture of 640a (1.0 g, 4.8 mmol, 1.0 eq), 3-fluoro-4-hydroxybenzonitrile (0.66 g, 4.8 mmol, 1.0 eq), K₂CO₃ (1.99 g, 14.4 mmol, 3.0 eq) in DMF (10 mL) was kept stirring at 20° C. for 2H. The mixture was diluted with EA (20 mL), washed with H₂O (10 mL*2). The organic layer was dried over Na₂SO₄, filtrated and concentrated in vacuo. The residue was purified by SGC (UV254, EA/PE=1:10) to afford 640b (1.1 g, 78% yield); m/z (ESI, +ve ion)=264.0 [M+H]⁺.

### Step B: Methyl 2-(4-((2-((4-cyano-2-fluorophenoxy)methyl)pyrimidin-4-yl)oxy)cyclohexyl)acetate (640c)

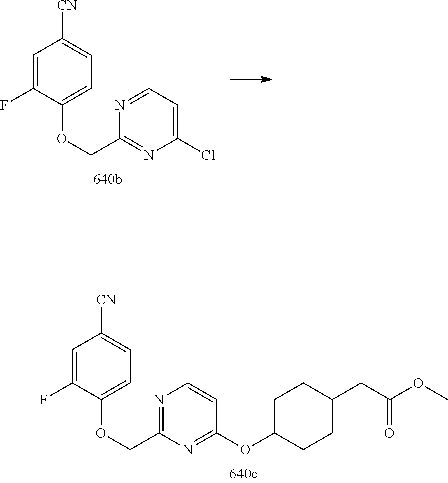 [SMILES: N#Cc1ccc(OCc2nccc(Cl)n2)c(F)c1 ; COC(=O)CC1CCC(Oc2ccnc(COc3ccc(C#N)cc3F)n2)CC1]

A mixture of 640b (270 mg, 1.024 mmol, 1.0 eq), methyl 2-(4-hydroxycyclohexyl)acetate (264 mg, 1.54 mmol, 1.1 eq) Pd₂(dba)₃ (94 mg, 0.10 mmol, 0.1 eq), BINAP (127 mg, 0.20 mmol, 0.2 eq), K₃PO₄ (652 mg, 3.07 mmol, 3.0 eq) in Tol (6 mL) was kept stirring at 100° C. for 18 h under N₂. The mixture was filtrated and concentrated in vacuo. The residue was purified by SGC (UV254, EA/PE=1:1) to afford 640c (364 mg, 66%) as a yellow gum; m/z (ESI, +ve ion)=400.1 [M+H]⁺.

### Step C: Methyl(S)-4-(2-(4-((2-((4-cyano-2-fluorophenoxy)methyl)pyrimidin-4-yl)oxy)cyclohexyl) acetamido)-3-((oxetan-2-ylmethyl)amino)benzoate (640d)

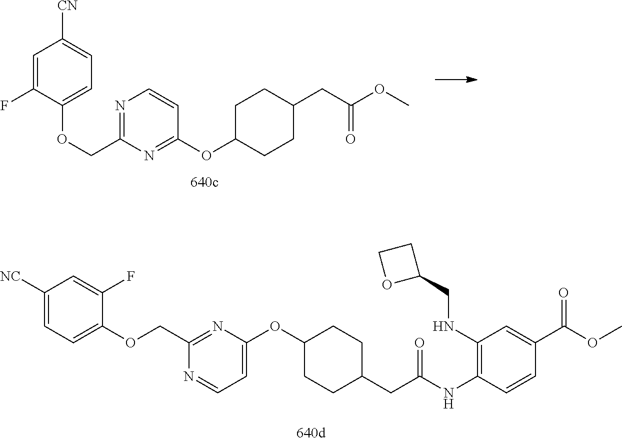 [SMILES: COC(=O)CC1CCC(Oc2ccnc(COc3ccc(C#N)cc3F)n2)CC1 ; COC(=O)c1ccc(NC(=O)CC2CCC(Oc3ccnc(COc4ccc(C#N)cc4F)n3)CC2)c(NC[C@@H]2CCO2)c1]

A mixture of 640c (280 mg, 0.701 mmol, 1.0 eq), LiOH (168 mg, 7.01 mmol, 10.0 eq) in a mix solution of THF/H₂O (2.5 mL/2.5 mL) was kept stirring at 30° C. for 18 h. The mixture was adjusted pH=6 with 6N HCl, extracted with EA (15 mL*2). The organic layer was dried over Na₂SO₄, filtrated and concentrated in vacuo to afford acid (202 mg, 60% yield). m/z (ESI, +ve ion)=386.1 [M+H]⁺.

A mixture of acid (202 mg, 0.847 mg, 1.0 eq), methyl 4-amino-3-{[(2S)-oxetan-2-ylmethyl]amino}benzoate (124 mg, 0.524 mmol, 1.0 eq), DIEA (203 mg, 1.572 mmol, 3.0 eq) in DCM (10 mL) was added T₃P (500 mg, 0.786 mmol, 1.5 eq, 50% in EA) at 0° C. The mixture was kept stirring at 20° C. for 1.5h. The mixture was concentrated and purified by Pre-TLC (UV254, EA/PE=2:1) to afford 640d (162 mg, 46% yield) as a yellow oil; m/z (ESI, +ve ion)=604.2.

### Step D: Methyl(S)-2-((4-((2-((4-cyano-2-fluorophenoxy)methyl)pyrimidin-4-yl)oxy)cyclohexyl)methyl)-1-(oxetan-2-ylmethyl)-1H-benzo[d]imidazole-6-carboxylate (640e)

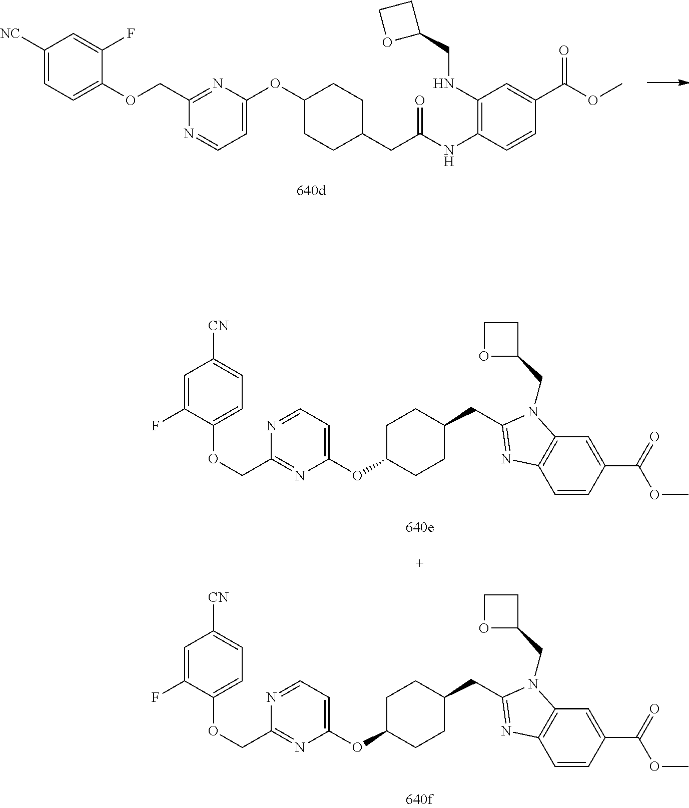 [SMILES: COC(=O)c1ccc(NC(=O)CC2CCC(Oc3ccnc(COc4ccc(C#N)cc4F)n3)CC2)c(NC[C@@H]2CCO2)c1 ; COC(=O)c1ccc2nc(C[C@H]3CC[C@H](Oc4ccnc(COc5ccc(C#N)cc5F)n4)CC3)n(C[C@@H]3CCO3)c2c1 ; COC(=O)c1ccc2nc(C[C@H]3CC[C@@H](Oc4ccnc(COc5ccc(C#N)cc5F)n4)CC3)n(C[C@@H]3CCO3)c2c1]

A mixture of 640d (162 mg, 0.268 mmol, 1.0 eq) in HOAc (3 mL) was kept stirring at 100° C. for 0.5h. The mixture was concentrated. The residue was purified by Pre-TLC (UV254, EA/PE=2:1) to afford less polar 640e (51 mg) and more polar 640f (28 mg). m/z (ESI, +ve ion)=585.9. Both diasteromers 640e and 640f were separated by SFC chromatography.

### Step E: 1-{[(2S)-Oxetan-2-yl]methyl}-2-{[(1r,4r)-4-({2-[(4-cyano-2-fluorophenoxy)methyl]pyrimidin-4-yl}oxy)cyclohexyl]methyl}-1H-1,3-benzodiazole-6-carboxylic acid (640)

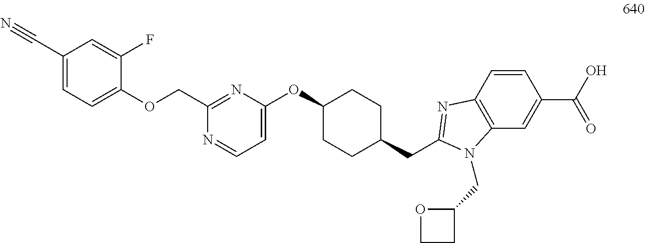 [SMILES: N#Cc1ccc(OCc2nccc(O[C@H]3CC[C@@H](Cc4nc5ccc(C(=O)O)cc5n4C[C@@H]4CCO4)CC3)n2)c(F)c1]

A mixture of 640e (58 mg, 0.099 mmol, 1.0 eq) in THF/H₂O (1:1) (3.5 mL) was added LiOH (43 mg, 1.485 mmol, 15.0 eq). The mixture was kept stirring at 30° C. for 32H. The mixture was adjusted pH=6 with 1N HCl. The mixture was extracted with EA (15 mL*2). The organic layer was concentrated and purified by reverse phase (UV214, CH₃CN/H₂O (0.5% NH₄OH)=29%) to afford 640 (19 mg, 31.92%). ¹H NMR (400 MHz, CD₃OD) δ ppm 8.42 (d, 1H), 8.17 (s, 1H), 7.96 (d, 1H), 7.61 (t, 2H), 7.50 (d, 1H), 7.26 (t, 1H), 6.71 (d, 1H), 5.42 (s, 2H), 5.24 (d, 1H), 4.93 (s, 1H), 4.71-4.61 (m, 2H), 4.53 (d, 1H), 4.49-4.42 (m, 1H), 2.98 (d, 2H), 2.81 (s, 1H), 2.55 (d, 1H), 2.06 (s, 1H), 1.99 (d, 2H), 1.86 (d, 2H), 1.44 (t, 2H), 1.23-1.10 (m, 2H); m/z (ESI, +ve ion)=571.9 [M+H]⁺.

### Step F: 1-{[(2S)-Oxetan-2-yl]methyl}-2-{[(1s,4s)-4-({2-[(4-cyano-2-fluorophenoxy)methyl]pyrimidin-4-yl}oxy)cyclohexyl]methyl}-1H-1,3-benzodiazole-6-carboxylic acid (641)

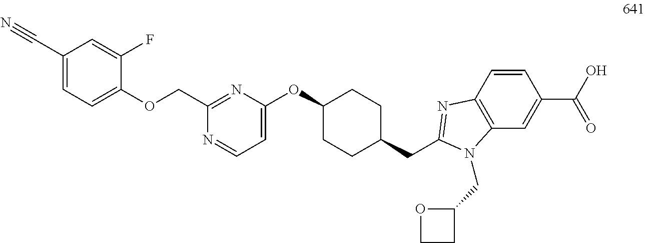 [SMILES: N#Cc1ccc(OCc2nccc(O[C@H]3CC[C@@H](Cc4nc5ccc(C(=O)O)cc5n4C[C@@H]4CCO4)CC3)n2)c(F)c1]

A mixture of 640f (28 mg, 0.0478 mmol, 1.0 eq) in a mix solution of THF/H₂O (1:1) (3.5 mL) was added LiOH (17 mg, 0.717 mmol, 15.0 eq). The mixture was kept stirring at 30° C. for 32 h. The mixture was adjusted pH=6 with 1N HCl. The mixture was extracted with EA (15 mL*2). The organic layer was concentrated and purified by reverse phase (UV214, CH₃CN/H₂O (0.5% NH₄OH)=27%) to afford 641 (6.2 mg, 21.55% yield). H NMR (400 MHz, CD₃OD) δ ppm 8.45 (d, 1H), 8.20 (s, 1H), 8.00-7.92 (m, 1H), 7.65-7.55 (m, 2H), 7.48 (d, 1H), 7.24 (t, 1H), 6.80 (d, 1H), 5.39 (s, 2H), 5.30-5.16 (m, 2H), 4.71-4.58 (m, 2H), 4.52 (dd, 1H), 4.42 (dt, 1H), 3.00 (dd, 2H), 2.86-2.74 (m, 1H), 2.58-2.45 (m, 1H), 2.18 (s, 1H), 1.96 (d, 2H), 1.59 (dt, 6H); m/z (ESI, +ve ion)=571.9 [M+H]⁺.

### Biological Example 1

### GLP-1R CAMP Assay

CAMP accumulation was measured in Chinese hamster ovary (CHO) cells (10,000 cells/well) stably overexpressing the human GLP-1 receptor using the HitHunter CAMP Assay for Small Molecules Kit (Eurofins). Briefly, cells were grown in Ham's F12 Nutrient Mix with 10% FBS, and lifted with Cell Dissociation Buffer, Enzyme free, PBS based (ThermoFisher). Cells were pelleted, and resuspended in Hank's buffered saline solution with 10 mM HEPES, and 625 μM 3-isobutyl-1-methylxanthine. The anti-cAMP antibody reagent was then added to cells at a 1:2 ratio and 5 μL of mixture was seeded in 384-well small volume white assay plates, at 10,000 cells/well. Cells were then treated with 50 nL compound using an ECHO 550 acoustic dispenser (Labcyte) in a 20-point dose response format in triplicate for 30 min. Cells were then lysed, and detection reagents added according to the manufacturer's protocol. After overnight incubation, luminescence was measured using a Perkin Elmer Envision plate reader. Dose response curves were analyzed using GraphPad Prism 9.0.

Results are reported in the Table below. In the Table, EC₅₀ values are designated as follows:
A: 0 nM<EC₅₀<0.1 nM;
B: 0.1 nM≤EC₅₀<1.0 nM;
C: 1.0 nM≤EC₅₀<10.0 nM;
D: 10.0 nM≤EC₅₀<100 nM;
E: 100 nM≤EC₅₀<500 nM; and
F: ≥500 nM

Molecular weights are calculated by standard techniques, and mass spectrometry results are reported according to the Examples above

TABLE 3

| Example | | | [M + H]⁺ |
|---|---|---|---|
| Compound | EC₅₀ | MW Calc. | Found |
| 552 | B | 590.588 | 591.3 |
| 553 | B | 590.588 | 591.3 |
| 554 | A | 597.04 | 597.2 |
| 555 | A | 613.06 | 613.2 |
| 556 | B | 587.612 | 588.2 |
| 557 | C | 621.07 | 621.2 |
| 558 | B | 600.651 | 601.3 |
| 559 | B | 594.62 | 595.2 |
| 560 | B | 601.639 | 602.2 |
| 561 | B | 586.624 | 587.2 |
| 562 | B | 579.605 | 580.3 |
| 563 | B | 656.71 | 657.2 |
| 564 | B | 682.74 | 683.4 |
| 565 | C | 579.605 | 580.3 |
| 566 | B | 580.593 | 581.3 |
| 567 | B | 569.622 | 570.2 |
| 568 | B | 593.632 | 594.3 |
| 569 | F | 611.647 | 612.3 |
| 570 | B | 605.643 | 606.3 |
| 571 | D | 583.62 | 584.3 |
| 572 | B | 597.595 | 598.1 |
| 573 | A | 604.615 | 605.2 |
| 574 | C | 587.612 | 588.2 |
| 575 | B | 593.632 | 594.2 |
| 576 | A | 597.595 | 598.3 |
| 577 | B | 609.631 | 610.3 |
| 578 | B | 600.651 | 601.2 |
| 579 | B | 600.651 | 601.3 |
| 580 | C | 573.585 | 574.2 |
| 581 | A | 597.595 | 598.2 |
| 582 | B | 597.595 | 598.3 |
| 583 | B | 629.613 | 630.3 |
| 584 | B | 593.632 | 594.2 |
| 585 | B | 593.632 | 594.1 |
| 586 | C | 600.651 | 601.1 |
| 587 | A | 600.651 | 601.1 |
| 588 | A | 635.592 | 636.5 |
| 589 | B | 593.631 | 594.2 |
| 590 | B | 577.632 | 578.2 |
| 591 | B | 617.601 | 618.2 |
| 592 | A | 617.601 | 618.2 |
| 594 | C | 649.619 | 650.2 |
| 595 | B | 653.032 | 654.3 |
| 596 | A | 642.612 | 643.3 |
| 597 | A | 615.035 | 615.3 |
| 598 | B | 649.619 | 650.2 |
| 599 | B | 640.596 | 641.2 |
| 600 | B | 607.659 | 608.3 |
| 601 | A | 642.612 | 643.3 |
| 602 | B | 635.041 | 635.2 |
| 603 | A | 597.044 | 597.2 |
| 604 | B | 587.612 | 588.3 |
| 605 | A | 601.008 | 601.1 |
| 606 | A | 598.583 | 599.2 |
| 607 | A | 605.603 | 606.2 |
| 608 | B | 611.071 | 611.2 |
| 609 | D | 611.071 | 611.2 |
| 610 | B | 627.664 | 628.2 |
| 611 | B | 627.664 | 628.2 |
| 612 | C | 607.659 | 608.2 |
| 613 | B | 600.020 | 600.0 |
| 614 | B | 578.621 | 579.2 |
| 615 | B | 596.056 | 596.2 |
| 616 | B | 596.056 | 596.2 |
| 617 | A | 618.590 | 619.3 |
| 618 | B | 607.659 | 608.2 |
| 619 | C | 597.595 | 598.3 |
| 620 | B | 636.580 | 637.3 |
| 621 | A | 611.622 | 612.2 |
| 622 | B | 612.610 | 613.2 |
| 623 | C | 607.659 | 608.4 |
| 624 | C | 596.060 | 596.2 |
| 625 | C | 604.615 | 605.2 |
| 626 | B | 625.609 | 626.2 |
| 627 | B | 625.609 | 626.1 |
| 628 | B | 584.556 | 585.3 |
| 629 | A | 605.603 | 606.3 |
| 630 | B | 587.612 | 588.3 |
| 631 | B | 601.639 | 602.4 |
| 632 | B | 594.620 | 595.4 |
| 633 | D | 535.596 | 536.0 |
| 634 | B | 591.576 | 592.0 |
| 635 | B | 643.600 | 644.4 |
| 636 | B | 661.590 | 662.3 |
| 637 | C | 593.632 | 594.3 |
| 638 | D | 655.707 | 656.4 |
| 639 | D | 565.622 | 566.4 |
| 640 | A | 571.609 | 571.9 |
| 641 | E | 571.609 | 571.9 |

In addition to potency, the compounds disclosed and claimed herein may have other beneficial and advantageous physiological properties that may enhance their utility as human or animal therapeutics. Those beneficial and advantageous properties may be determined using a variety of tests and assays known and available to the skilled person. For example, such tests and assays include but are not limited to the CAMP assay in serum; plasma protein binding-multiple species assays, ADME and Tox assays, including metabolic stability assays (LM stability assay for multiple species, Hepatocyte suspension stability assay for multiple species; Solubility assays-multiple pH or simulated fluid; Permeability assays-MDCK/Caco-2/Pampa; hERG Inhibition Assay-binding/patch-clamp; CYP Inhibition assays-Multiple isoforms; CYP induction assay; CYP TDI Assay; Liver transporter (substrate/inhibitor)-multiple species; Rat PK (IV/PO, cassette or discrete); Mouse PK (IV/PO, cassette or discrete) Dog PK (IV/PO, cassette or discrete); Cyno PK (IV/PO, cassette or discrete); huMouse Efficacy assays, Cyno Efficacy assays, Relay hepatocyte assays, Hepatocyte uptake assays (Drug Metab Dispos. 2007 June; 35 (6): 859-65); 2D Co-culture Hepatocyte assay-multiple species (24th North American ISSX Meeting: Broadening Our Horizons Sep. 13-17, 2021).

A number of vendors offer these assays as kits or services, including BioDuro-Sudia (9601 Jeronimo Road, Irvine, CA 92618), WuXi AppTec (288 Fute Zhong Road, Waigaoqiao FreeTrade Zone, Shanghai 200131, China), Eurofins Discovery X (42501 Albrae Street, Fremont, CA 94538, USA), Pharmaron (404 Wyman Street, Suite 405, Waltham, MA 02451), and Meadowhawk Biolabs (33 Locke Dr, Marlborough, MA, 01752).

### EQUIVALENTS

The disclosure set forth above may encompass multiple distinct embodiments with independent utility. Although each of these embodiments has been disclosed, the specific embodiments thereof as disclosed and illustrated herein are not to be considered in a limiting sense, because numerous variations are possible. The subject matter of the embodiments includes all novel and nonobvious combinations and subcombinations of the various elements, features, functions, and/or properties disclosed herein. The following claims particularly point out certain combinations and subcombinations regarded as novel and nonobvious. Alternative embodiments as in other combinations and subcombinations of features, functions, elements, and/or properties may be claimed in this application, in applications claiming priority from this application, or in related applications. Such claims, whether directed to a different embodiment or to the same embodiment, and whether broader, narrower, equal, or different in scope in comparison to the original claims, also are regarded as included within the subject matter of this disclosure.

One or more features from any embodiments described herein or in the FIGURES may be combined with one or more features of any other embodiments described herein or in the FIGURES without departing from the scope of this disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

1. A compound according to Formula (LVII), (LVIII), (LIX), (LX), (LXI), or (LXII), or Compounds 552, 553, 558, 561, 562, 572, 573, 576, 578, 581, 640, and 641; or a pharmaceutically acceptable salt, or stereoisomer thereof: 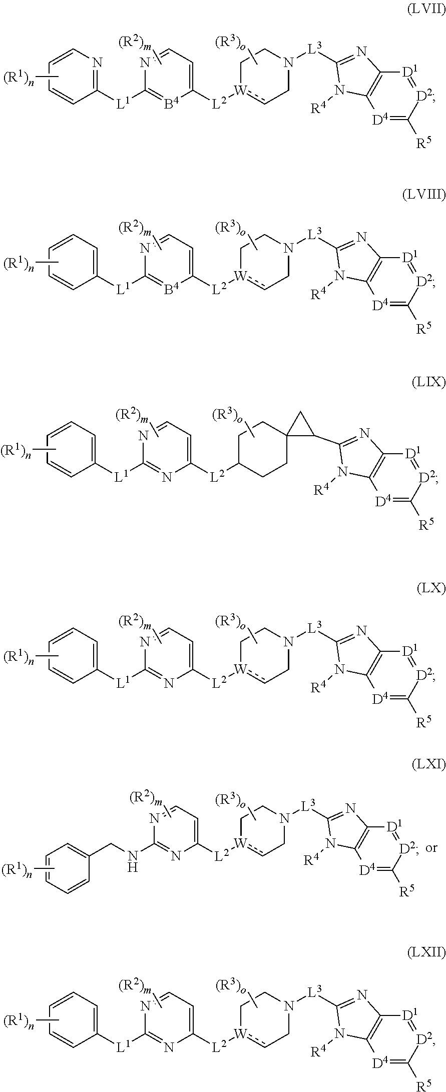
wherein:
when the compound is according to Formula (LVII), B⁴ is —CH—, —C(R²)—, or —N—;
D¹ is —CH— or —N—;
D² is —CH—, —N—, —C(CH₃)—;
D⁴ is —CH— or —N—;
W is N, CR¹⁴, or C; when W is CR¹⁴, the adjacent dashed line indicates a single bond; when W is C, the adjacent dashed line indicates a double bond or L² is —C(H)═;
L¹ is selected from the group consisting of —O—, —CH₂—, —NH—, —N(Me)-, —N(Et)-, —OCH₂—, —OC(H,Me)-, —CH₂O—, —NHCH₂—, —CH₂NH—, —N(Me)CH₂—, and —SO₂NH—;
L² is selected from the group consisting of a bond, —C(O)—, —CH₂—, —C(H)═, SO₂—, —O—, —NH—, —N(Me)-, and 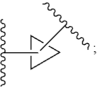
L³ is —CH₂—, —C(O)—, or —CH(CH₃)—;
each R¹ is independently selected from the group consisting of alkyl, substituted alkyl, halo, cyano, azido, alkoxy, haloalkoxy, and R¹¹R¹²NCO—;
each R² is independently selected from the group consisting of alkyl, substituted alkyl, halo, cyano, azido, alkoxy, haloalkoxy, and R¹¹R¹²NCO—;
each R³ is independently selected from the group consisting of alkyl, substituted alkyl, halo, cyano, azido, alkoxy, haloalkoxy, and R¹¹R¹²NCO—;
R⁴ is selected from the group consisting of alkyl, substituted alkyl, unsubstituted arylalkyl, substituted arylalkyl, heteroalkyl, substituted heteroalkyl, unsubstituted heteroarylalkyl, substituted heteroarylalkyl, unsubstituted heterocycloalkyl, and substituted heterocycloalkyl;
R⁵ is selected from the group consisting of —H, —CH₂OH, —COOH, —CH₂COOH, —(CH₂)₂COOH, —COCF₃, —C(OH)CF₃, —CONHCN, —CONHOH, CONHOMe, —CONHSO₂N(Me)₂, —PO₃H₂, —PO(Me)(OH), —SO₃H, —SO₂NH₂, —SO₂NHMe, —B(OH)₂, 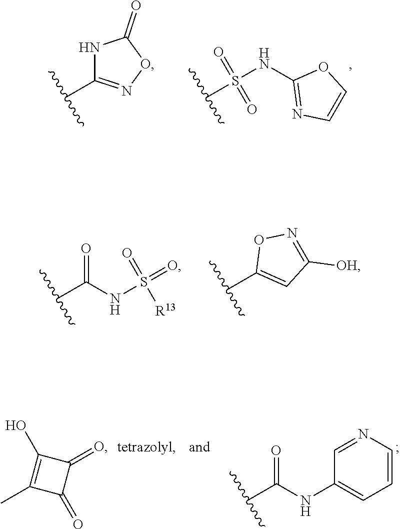
each R¹¹ is hydrogen, or alkyl;
each R¹² is hydrogen, or alkyl;
R¹³ is hydrogen, alkyl, or -cyclopropyl;
R¹⁴ is hydrogen or alkyl;
n is an integer from 0 to 4;
m is an integer from 0 to 3; and
o is an integer from 0 to 4;
wherein when the compound is according to Formula (LVIII), R³ is independently selected from the group consisting of —CH₂CH₃, —CH₂F, —CH₂CN, and —CF₃; and all the other variables are as in Formula (LVII); wherein when B⁴ is —C— and R³ is —CH₂F, then the compound is not 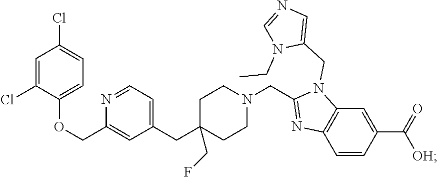 [SMILES: CCn1cncc1Cn1c(CN2CCC(CF)(Cc3ccnc(COc4ccc(Cl)cc4Cl)c3)CC2)nc2ccc(C(=O)O)cc21]
wherein when B⁴ is —N— and R³ is —CH₂F, then the compound is not 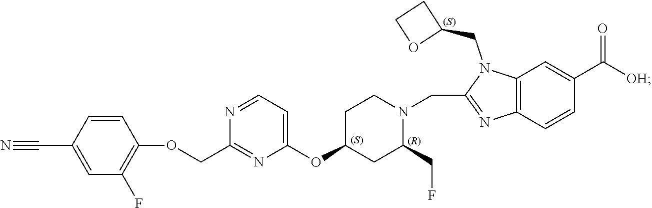
wherein when the compound is according to Formula (LIX), the variables are as in Formula (LVII);
wherein when the compound is according to Formula (LX), R⁴ is selected from the group consisting of: (1,1-dioxidothietan-2-yl)methyl, ((R)-1,1-dioxidothietan-2-yl)methyl, ((S)-1,1-dioxidothietan-2-yl)methyl, 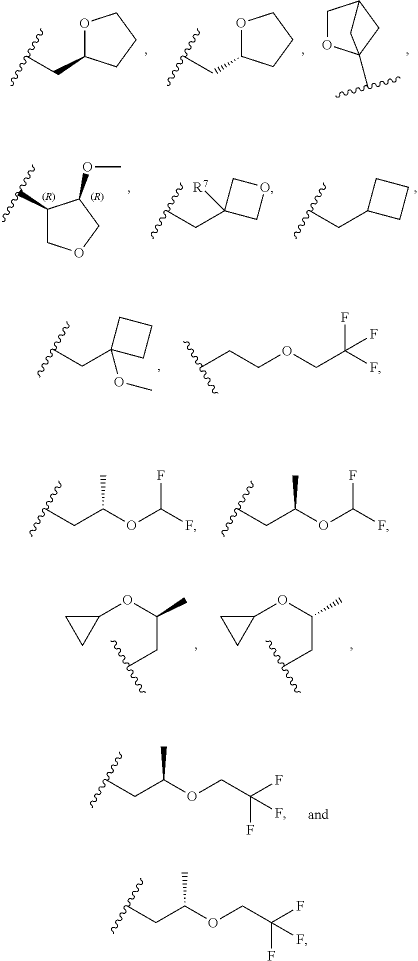
wherein R⁷ is —CH₂F or —CHF₂; and all the other variables are as in Formula (LVII);
wherein when the compound is according to Formula (LXI), the other variables are as in Formula (LVII);
wherein when the compound is according to Formula (LXII),
either (a), (b), (c), (d), or (e)
(a) wherein when D¹ is —N— and D² and D⁴ are —CH—, then the compound is not

| Com- | |
|---|---|
| pound No. | Structure |
| 486 | 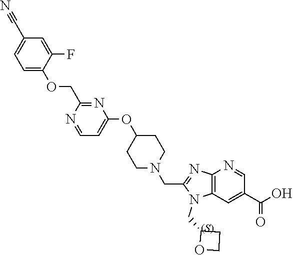 |
| 512 | 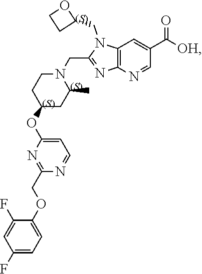 |
| | and |
| 513 | 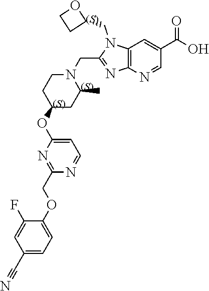 |

and all the other variables are as in Formula (LVII);
or
(b) wherein D¹ and D⁴ are —CH— and D² is —N— and all the other variables are as in Formula (LVII);
or
(c) wherein D¹ is —CH—, D⁴ is —CH— or —N—, and D² is —C(CH₃); and all the other variables are as in Formula (LVII);
or
(d) wherein D¹, D², and D⁴ are —CH— or —N—, wherein zero or one of D¹, D², and D⁴ is —N— and R⁵ is selected from group consisting of: H, CH₂OH, —CH₂COOH, —(CH₂)₂COOH, 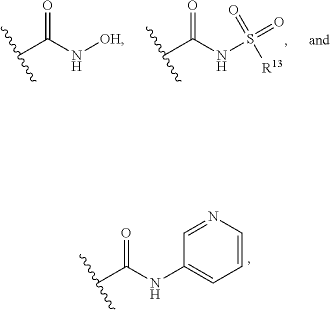
and R¹³ is —CH₃ or -cyclopropyl; and all the other variables are as in Formula (LVII);
or
(e) D¹, D², and D⁴ are —CH— or —N—, wherein zero or one of D¹, D², and D⁴ is —N— and L³ is —CH(CH₃)—; and all the other variables are as in Formula (LVII);
or

| Compound No. | Structure |
|---|---|
| 552 | 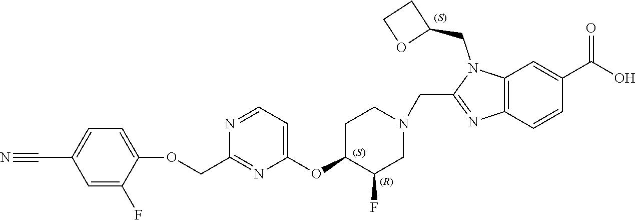 |
| 553 | 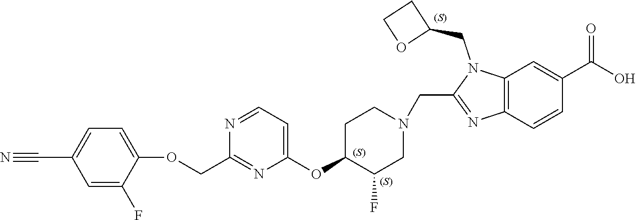 |
| 558 | 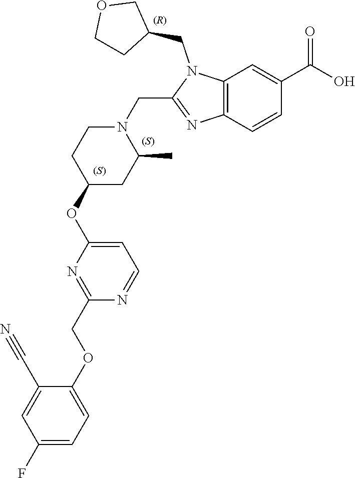 |
| 561 | 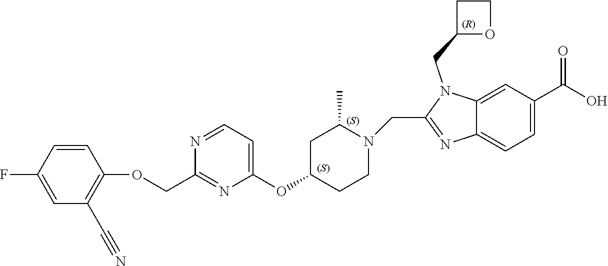 |
| 562 | 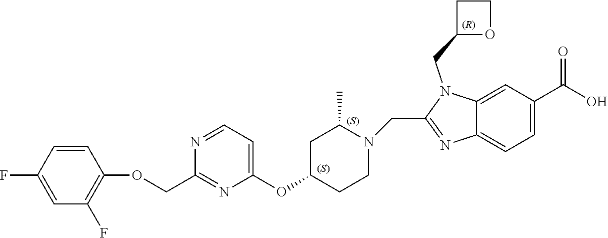 |
| 572 | 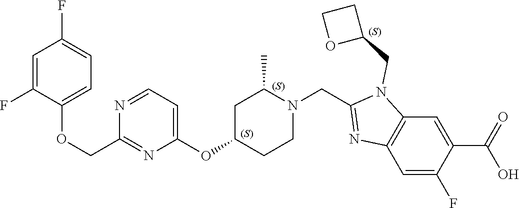 |
| 573 | 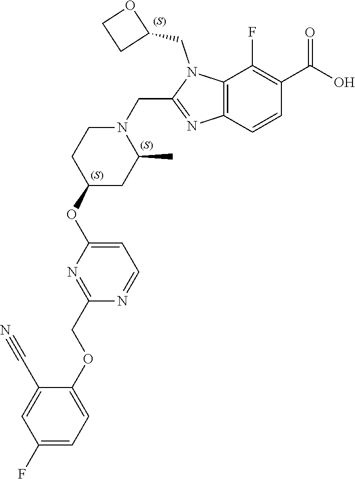 |
| 576 | 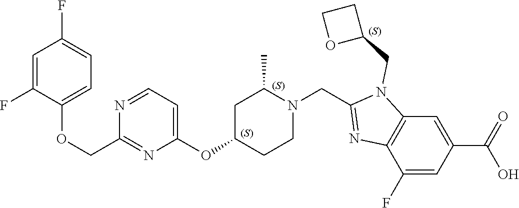 |
| 578 | 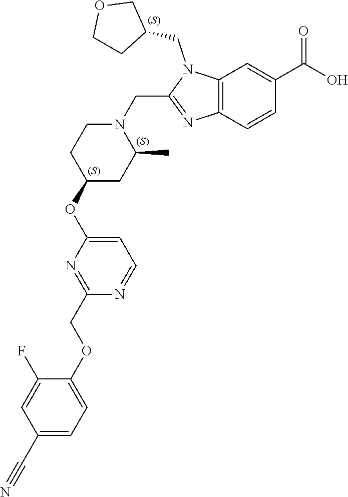 |
| 581 | 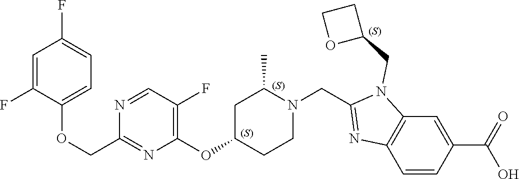 |
| 640 | 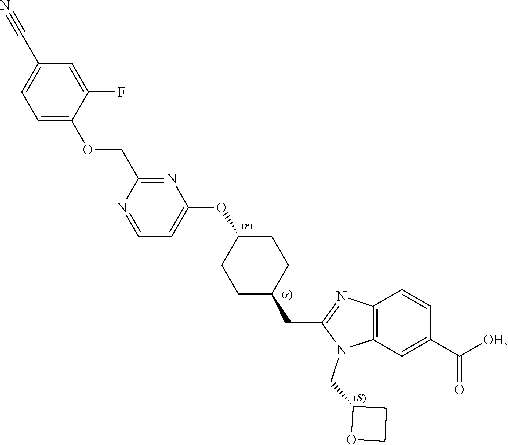 |
| | and |
| 641 | 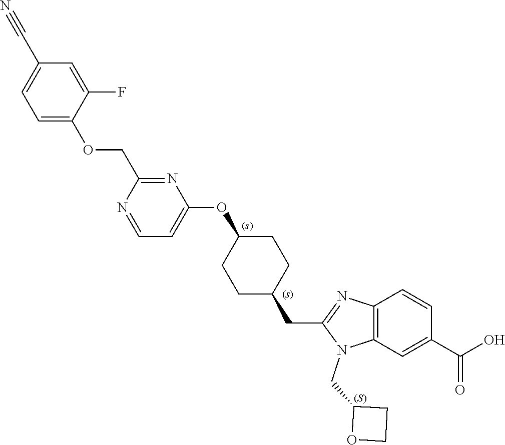 |

wherein when the compound is according to Formula (LVII), (LVIII), (LIX), (LX), or (LXII), L¹ is selected from the group consisting of —NH—, —N(Me)-, —N(Et)-, —OCH₂—, —OC(H,Me)-, —CH₂O—, —OCH₂(cyclopropyl)-, —O—CH₂—CH₂—, —CH₂—O—CH₂—, —CH₂—O—C((CH₃)₂), —NHCH₂—, —CH₂NH—, —N(Me)CH₂—, and —SO₂NH—.

2. A compound according to Formula LXIII or a pharmaceutically acceptable salt, or stereoisomer thereof: 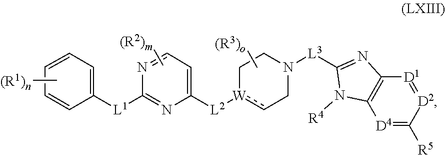
wherein:
D¹ is —CH— or —N—;
D² is —CH—, —N—, —C(CH₃)—;
D⁴ is —CH— or —N—;
W is N, CR¹⁴, or C; when W is CR¹⁴, the adjacent dashed line indicates a single bond; when W is C, the adjacent dashed line indicates a double bond or L² is —C(H)═;
L¹ is selected from the group consisting of —O—, —CH₂—, —NH—, —N(Me)-, —N(Et)-, —OCH₂—, —OC(H,Me)-, —CH₂O—, —NHCH₂—, —CH₂NH—, —N(Me)CH₂—, and —SO₂NH—;
L² is selected from the group consisting of a bond, —C(O)—, —CH₂—, —C(H)═, SO₂—, —O—, —NH—, —N(Me)-, and 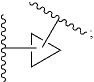
L³ is —CH₂—, —C(O)—, or —CH(CH₃)—;
each R¹ is independently selected from the group consisting of alkyl, substituted alkyl, halo, cyano, azido, alkoxy, haloalkoxy, and R¹¹R¹²NCO—;
each R² is independently selected from the group consisting of alkyl, substituted alkyl, halo, cyano, azido, alkoxy, haloalkoxy, and R¹¹R¹²NCO—;
each R³ is independently selected from the group consisting of alkyl, substituted alkyl, halo, cyano, azido, alkoxy, haloalkoxy, and R¹¹R¹²NCO—;
R⁴ is selected from the group consisting of: (1,1-dioxidothietan-2-yl)methyl, ((R)-1,1-dioxidothietan-2-yl)methyl, ((S)-1,1-dioxidothietan-2-yl)methyl, 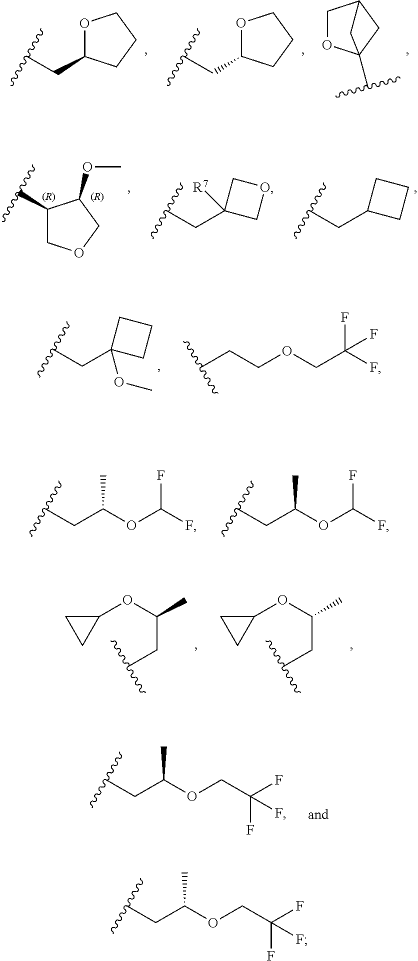
R⁵ is selected from the group consisting of —H, —CH₂OH, —COOH, —CH₂COOH, —(CH₂)₂COOH, —COCF₃, —C(OH)CF₃, —CONHCN, —CONHOH, CONHOMe, —CONHSO₂N(Me)₂, —PO₃H₂, —PO(Me)(OH), —SO₃H, —SO₂NH₂, —SO₂NHMe, —B(OH)₂, 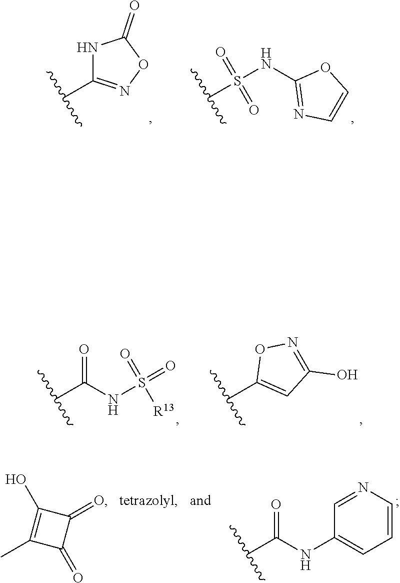
R⁷ is —CH₂F or —CHF₂;
each R¹¹ is hydrogen, or alkyl;
each R¹² is hydrogen, or alkyl;
R¹³ is hydrogen, alkyl, or -cyclopropyl;
R¹⁴ is hydrogen or alkyl;
n is an integer from 0 to 4;
m is an integer from 0 to 3; and
o is an integer from 0 to 4.

3. A compound according to Formula LXIV or a pharmaceutically acceptable salt, or stereoisomer thereof: 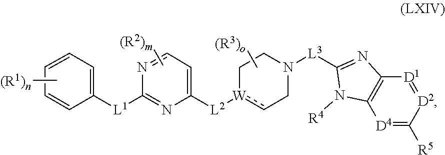
wherein:
D¹ is —CH— or —N—;
D² is —CH—, —N—, —C(CH₃)—;
D⁴ is —CH— or —N—;
W is N, CR¹⁴, or C; when W is CR¹⁴, the adjacent dashed line indicates a single bond; when W is C, the adjacent dashed line indicates a double bond or L² is —C(H)═;
L¹ is selected from the group consisting of —O—, —CH₂—, —NH—, —N(Me)-, —N(Et)-, —OCH₂—, —OC(H,Me)-, —CH₂O—, —NHCH₂—, —CH₂NH—, —N(Me)CH₂—, and —SO₂NH—;
L² is selected from the group consisting of a bond, —C(O)—, —CH₂—, —C(H)═, SO₂—, —O—, —NH—, —N(Me)-, and 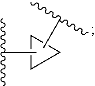
L³ is —CH₂—, —C(O)—, or —CH(CH₃)—;
each R¹ is independently selected from the group consisting of alkyl, substituted alkyl, halo, cyano, azido, alkoxy, haloalkoxy, and R¹¹R¹²NCO—;
each R² is independently selected from the group consisting of alkyl, substituted alkyl, halo, cyano, azido, alkoxy, haloalkoxy, and R¹¹R¹²NCO—;
each R³ is independently selected from the group consisting of alkyl, substituted alkyl, halo, cyano, azido, alkoxy, haloalkoxy, and R¹¹R¹²NCO—;
R⁴ is alkyl, substituted alkyl, unsubstituted arylalkylene, substituted arylalkylene, heteroalkyl, substituted heteroalkyl, unsubstituted heteroarylalkylene, substituted heteroarylalkylene, unsubstituted heterocycloalkyl, or substituted heterocycloalkyl;
R⁵ is selected from the group consisting of H, CH₂OH, —CH₂COOH, —(CH₂)₂COOH, 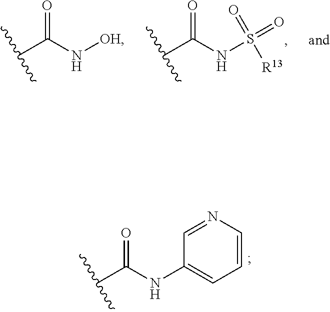
R⁷ is —CH₂F or —CHF₂;
each R¹¹ is hydrogen, or alkyl;
each R¹² is hydrogen, or alkyl;
R¹³ is hydrogen, alkyl, or -cyclopropyl;
R¹⁴ is hydrogen or alkyl;
n is an integer from 0 to 4;
m is an integer from 0 to 3; and
o is an integer from 0 to 4.

4. The compound any one of the previous claims, wherein each R² and R³ is H, or F.

5. The compound of any one of the previous claims, wherein m is 0, and o is 0.

6. The compound of any one of the previous claims, wherein W is N, or CH.

7. The compound of any one of the previous claims, wherein W is C.

8. The compound of any one of the previous claims, wherein W is CH.

9. The compound of any one of the previous claims, wherein W is N.

10. The compound of any one of the previous claims, wherein D⁴ is —CH—.

11. The compound of any one of the previous claims, wherein R⁴ is selected from the group consisting of azetidine-3-yl-methyl, (3R)-azetidine-3-yl-methyl, (3S)-azetidine-3-yl-methyl, (S)-buta-4-yl-1,3-diol, butan-1-yl-one, 1-(cyanomethyl)-cycloprop-1-yl-methyl, 3-(difluoromethyl)oxetan-3-yl-methyl, (1,1-dioxidothietan-2-yl)methyl, ((R)-1,1-dioxidothietan-2-yl)methyl, ((S)-1,1-dioxidothietan-2-yl)methyl, (1-ethyl-1H-imidazol-5-yl)-methyl, 1-(fluoromethyl)-cycloprop-1-yl-methyl, 3-fluorooxetan-3-yl-methyl, 1-hydroxyl-cycloprop-1-yl-methyl, isoxazol-5-yl-methyl, (3R,4R)-4-methoxytetrahydrofuran-3-yl, (1-methyl-1H-imidazol-5-yl)-methyl, 1-methoxy-cycloprop-1yl-methyl, methoxyethan-2-yl, 1-(2-methoxyethyl)-cycloprop-1-yl-methyl, {[1-(1,2-oxazole-3-carbonyl)azetidin-2-yl]methyl}, {[(2R)-1-(1,2-oxazole-3-carbonyl)azetidin-2-yl]methyl}, {[(2S)-1-(1,2-oxazole-3-carbonyl)azetidin-2-yl]methyl}, oxetan-2-yl-methyl, (2S)-oxetan-2-yl-methyl, oxelan-3-yl-methyl, (3R)-oxelan-3-yl-methyl, 1,3-oxazol-2-yl-methyl, (S)-1,3-oxazol-2-yl-methyl, (R)-1,3-oxazol-2-yl-methyl, 2-oxabicyclo[2.1.1]hexa-1-yl-methyl, 1-cyclobutylmethyl, (R)-2-cyclopropoxypropyl, (S)-2-cyclopropoxypropyl, (2R)-2-(difluoromethoxy)propyl, (2S)-2-(difluoromethoxy)propyl, (2S)-oxolan-2-yl-methyl, (1-methoxycyclobutyl)methyl, (2R)-2-(2,2,2-trifluoroethoxy)propyl, (2S)-2-(2,2,2-trifluoroethoxy)propyl, and 2-(2,2,2-trifluoroethoxy)ethyl.

12. The compound of any one of the previous claims, wherein R⁴ is selected from the group consisting of (1-ethyl-1H-imidazol-5-yl)-methyl, oxetan-2-yl-methyl, (2S)-oxetan-2-yl-methyl, oxelan-3-yl-methyl, (3R)-oxelan-3-yl-methyl, and 1,3-oxazol-2-yl-methyl.

13. The compound of any one of the previous claims, wherein R⁴ is selected from the group consisting of(S)-buta-4-yl-1,3-diol, 3-(difluoromethyl)oxetan-3-yl-methyl, (1,1-dioxidothietan-2-yl)methyl, ((R)-1,1-dioxidothietan-2-yl)methyl, ((S)-1,1-dioxidothietan-2-yl)methyl, (1-ethyl-1H-imidazol-5-yl)-methyl, 3-fluorooxetan-3-yl-methyl, (3R,4R)-4-methoxytetrahydrofuran-3-yl, oxetan-2-yl-methyl, (2S)-oxetan-2-yl-methyl, 1,3-oxazol-2-yl-methyl, (S)-1,3-oxazol-2-yl-methyl, (R)-1,3-oxazol-2-yl-methyl, 2-oxabicyclo[2.1.1]hexa-1-yl-methyl, 1-cyclobutylmethyl, (R)-2-cyclopropoxypropyl, (S)-2-cyclopropoxypropyl, (2R)-2-(difluoromethoxy)propyl, (2S)-2-(difluoromethoxy)propyl, (2S)-oxolan-2-yl-methyl, (1-methoxycyclobutyl)methyl, (2R)-2-(2,2,2-trifluoroethoxy)propyl, (2S)-2-(2,2,2-trifluoroethoxy)propyl, and 2-(2,2,2-trifluoroethoxy)ethyl.

14. The compound of any one of the previous claims, wherein R⁴ is (S)-buta-4-yl-1,3-diol.

15. The compound of any one of the previous claims, wherein R⁴ is 3-(difluoromethyl)oxetan-3-yl-methyl.

16. The compound of any one of the previous claims, wherein R⁴ is (1-ethyl-1H-imidazol-5-yl)-methyl.

17. The compound of any one of the previous claims, wherein R⁴ is 3-fluorooxetan-3-yl-methyl.

18. The compound of any one of the previous claims, wherein R⁴ is (3R,4R)-4-methoxytetrahydrofuran-3-yl.

19. The compound of any one of the previous claims, wherein R⁴ is oxetan-2-yl-methyl.

20. The compound of any one of the previous claims, wherein R⁴ is (2S)-oxetan-2-yl-methyl.

21. The compound of any one of the previous claims, wherein R⁴ is 1,3-oxazol-2-yl-methyl.

22. The compound of any one of the previous claims, wherein R⁴ is (S)-1,3-oxazol-2-yl-methyl.

23. The compound of any one of the previous claims, wherein R⁴ is (R)-1,3-oxazol-2-yl-methyl.

24. The compound of any one of the previous claims, wherein R⁴ is 2-oxabicyclo[2.1.1]hexa-1-yl-methyl.

25. The compound of any one of the previous claims, wherein R⁴ is ((R)-1,1-dioxidothietan-2-yl)methyl.

26. The compound of any one of the previous claims, wherein R⁴ is ((S)-1,1-dioxidothietan-2-yl)methyl.

27. The compound of any one of the previous claims, wherein R⁴ is 1-cyclobutylmethyl.

28. The compound of any one of the previous claims, wherein R⁴ is (R)-2-cyclopropoxypropyl.

29. The compound of any one of the previous claims, wherein R⁴ is (S)-2-cyclopropoxypropyl.

30. The compound of any one of the previous claims, wherein R⁴ is (2R)-2-(difluoromethoxy)propyl.

31. The compound of any one of the previous claims, wherein R⁴ is (2S)-2-(difluoromethoxy)propyl.

32. The compound of any one of the previous claims, wherein R⁴ is (2S)-oxolan-2-yl-methyl.

33. The compound of any one of the previous claims, wherein R⁴ is (1-methoxycyclobutyl)methyl.

34. The compound of any one of the previous claims, wherein R⁴ is (2R)-2-(2,2,2-trifluoroethoxy)propyl.

35. The compound of any one of the previous claims, wherein R⁴ is (2S)-2-(2,2,2-trifluoroethoxy)propyl.

36. The compound of any one of the previous claims, wherein R⁴ is 2-(2,2,2-trifluoroethoxy)ethyl.

37. The compound of any one of the previous claims, wherein R⁵ is —COOH.

38. The compound of any one of the previous claims, wherein R⁵ is

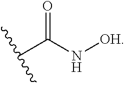 [SMILES: *C(=O)NO]

39. The compound of any one of the previous claims, wherein R⁵ is 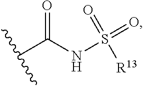
wherein R¹³ is —CH₃ or -cyclopropyl.

40. The compound of any one of the previous claims, wherein R⁵ is —CH₂COOH.

41. The compound of any one of the previous claims, wherein R⁵ is —(CH₂)₂COOH.

42. The compound of any one of the previous claims, wherein R⁵ is —H.

43. The compound of any one of the previous claims, wherein R⁵ is —CH₂OH.

44. The compound of any one of the previous claims, wherein R⁵ is

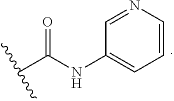 [SMILES: *C(=O)Nc1cccnc1]

45. The compound of any one of the previous claims 1-44, wherein L² is —O—.

46. The compound of any one of the previous claims 1-45, wherein L³ is —CH₂—.

47. The compound of any one of the previous claims 1-45, wherein L³ is —CH(CH₃)—.

48. The compound of any one of claims 1-47, wherein L¹ is —CH₂O—; and L² is —O—.

49. The compound of any one of claims 1-47, wherein L¹ is —OCH₂—; and L² is —O—.

50. The compound of any one of claims 1-47, wherein L¹ is —CH₂O—; and L³ is CH₂—.

51. The compound of any one of claims 1-47, wherein L¹ is —CH₂O—; and L³ is —CH(CH₃).

52. The compound of any one of claims 1-51, wherein L¹ is —O—.

53. The compound of any one of claims 1-50, 51, 53, and 54, wherein L¹ is —CH₂O—.

54. The compound of any one of claims 1-50 and 52, wherein L¹ is —OCH₂—.

55. A compound selected from any of the compounds in Table 1 and Table 2, or a pharmaceutically acceptable salt or solvate thereof:

56. A pharmaceutical composition comprising the compound of any of the previous claims, or a pharmaceutically acceptable salt thereof, and one, or more pharmaceutically acceptable carriers, excipients, or diluents.

57. A method of treating a metabolic disease or disorder comprising the step of administering to a patient in need thereof a therapeutically effective amount of a compound of any one of claims 1-55, or a pharmaceutically acceptable salt thereof, or a therapeutically effective amount of the pharmaceutical composition of claim 56.

58. The compound of any one of claims 1-55, or the pharmaceutical composition of claim 56 for use in therapy.

59. The compound of any of one claims 1-55, or the pharmaceutical composition of claim 56 for use in the treatment of a metabolic disease, or disorder.

60. Use of the compound of any one of claims 1-56, or the pharmaceutical composition of claim 56 in the manufacture of a medicament for the treatment of a metabolic disease, or disorder.
